(19) United States
(12) Patent Application Publication
(10) Pub. No.: US 2026/0125645 A1
(43) Pub. Date: May 7, 2026

(54) METHODS AND PHARMACOLOGICAL AGENTS FOR INCREASING EXPANSION, ENGRAFTMENT OR IMMUNE OUTPUT OF HUMAN HEMATOPOIETIC STEM CELLS IN TRANSPLANTATION AND DISEASE SETTINGS

(71) Applicant: VALORISATION HSJ, LIMITED PARTNERSHIP, Québec (CA)

(72) Inventors: Élie HADDAD, Hampstead (CA); Panojot BIFSHA, Clarens (CH)

(21) Appl. No.: 19/117,519

(22) PCT Filed: Oct. 3, 2023
(86) PCT No.: PCT/CA2023/051309

§ 371 Date: Apr. 1, 2025

Related U.S. Application Data
(60) Provisional application No. 63/412,944, filed on Oct. 4, 2022.

(51) Int. Cl. C12N 5/0789; A61K 31/197; A61K 31/403; A61K 31/454; A61K 31/4545; A61P 37/04; C12N 5/00

(52) U.S. Cl. CPC C12N 5/0647; A61K 31/197; A61K 31/403; A61K 31/454; A61K 31/4545; A61P 37/04; C12N 5/0018; C12N 2501/125; C12N 2501/145; C12N 2501/2306; C12N 2501/26; C12N 2501/395; C12N 2501/825; C12N 2510/00

(57) ABSTRACT

The present application relates to methods for expanding hematopoietic stem cells (HSC) and/or hematopoietic progenitor cells (HPC) ex vivo and/or in vivo. The methods comprise culturing the HSCs and/or HPCs in the presence of a selective 5-Hydroxytryptamine Receptor 1F (HTR1F) agonist and/or of a Thyroid Hormone Receptor Beta (THRB) agonist, and/or in an air-liquid interface (ALI) culture system. The expanded HSCs and/or HPCs, which maintain engraftment and immune reconstitution properties, may be administered to subjects suffering from various diseases including cancer, immunodeficiencies, myelodysplastic syndrome (MDS) or anemia. The present application also relates to the use of HTR1F and/or THRB agonists for stimulating HSC and/or HPC expansion, and for increasing the number of immune cells or for reconstituting the immune system, in vivo.

### CROSS REFERENCE TO RELATED APPLICATIONS

The present application claims the benefit of U.S. provisional patent application No. 63/412,944 filed on Oct. 4, 2022, which is incorporated herein by reference in its entirety.

### TECHNICAL FIELD

The present invention generally relates to the field of hematology, and more particularly to stem cell transplantation and treatment of blood or immunological disorders.

### BACKGROUND ART

Hematopoietic stem cells (HSCs) promote the lifelong production of all mature blood cell lineages through their unique capabilities of durable self-renewal and multilineage differentiation.

HSCs are present in donor-derived bone marrow (BM), cord blood (CB), and mobilized peripheral blood stem-cell products for allogeneic stem-cell transplantation (SCT) in patients with hematological malignancies and monogenic diseases. Patient-derived peripheral blood stem-cell products are extensively used for autologous SCT, which supports hematopoietic rescue after high-dose chemotherapy for various types of hematological malignancies, solid tumors, and autoimmune diseases.

However, in many diseases affecting blood cell lineages, the number of HSCs that can be obtained from a patient is very limited in number. Attrition of autologous HSCs during processing for gene therapy is also a problem. In addition, it has not been possible to maintain or expand human HSCs ex vivo. Under in vitro cell culture conditions, the number of HCS typically decline because HSCs either die or terminally differentiate, losing their stem cell properties. In many situations, the clinical outcome would be much better if one could expand or amplify the number, in the graft, of self-renewing and multipotent HSC capable of reconstituting immune system and maintaining hematopoiesis long-term.¹

Problems associated with current ex vivo stem cell expansion methods include loss of short-term repopulating activity of cultured cells and expansion of aberrant long-term repopulating cells with poor contribution to peripheral immune reconstitution.

Studies have shown that aryl hydrocarbon receptor antagonist², such as StemRegenin1 (SR1), notch ligand agonists³, and UM171/729⁴ can all promote HSC expansion and increase repopulating capacities, particularly from cord blood. Attempting to replicate state-of-the-art methodologies that use either SR1 or UM171/729, for the expansion of primitive HSC, led to the observation that these protocols commonly failed to maintain short-term repopulating activity in humanized mice experiments⁵. Furthermore, even though these culture methods reported a significant increase of long-term repopulating activity, they lacked robustness and were rarely outperforming the non-cultured HSC as to their ability to populate the bone-marrow and peripheral blood of immunodeficient mouse hosts.

In clinical transplantation, the presence of sufficient short-term repopulating activity (stemming from progenitors and primitive HSC) is of paramount importance for the survival of the patient.

Indeed, this short-term repopulating activity is responsible for the rapid increase of polymorphonuclear and platelets, which is crucial after transplantation. Also, the continuous lifetime production of blood cells is required and can only be maintained by primitive HSC⁶.

There is thus a need in the fields of HSCT and gene therapy for the development of strategies that would increase the numbers of engraftable primitive HSC without skewing the engraftment characteristics of the cells of origin⁷⁻¹⁰ to enhance regeneration of the blood system, notably in the treatment of hematological malignancies, solid tumors and autoimmune diseases.

The present description refers to a number of documents, the content of which is herein incorporated by reference in their entirety.

### SUMMARY

The present disclosure provides the following items 1 to 42:
1. A method for expanding hematopoietic stem cells (HSCs) and/or hematopoietic progenitor cells (HPCs) ex vivo comprising culturing said hematopoietic stem cells and/or hematopoietic progenitor cells in the presence of a selective 5-Hydroxytryptamine Receptor 1F (HTR_{1F}) agonist and/or of a Thyroid Hormone Receptor Beta (THRB) agonist under conditions suitable for HSC and/or HPC expansion.
2. The method of item 1, wherein the HSCs and/or HPCs are obtained from donor bone marrow, umbilical donor cord blood, or donor peripheral blood.
3. The method of item 1 or 2, wherein the HSCs and/or HPCs are from a subject suffering from a disease or condition.
4. The method of item 3, wherein the disease is cancer, an immunodeficiency, myelodysplastic syndrome (MDS) or anemia.
5. The method of item 4, wherein the cancer is a hematological cancer.
6. The method of any one of items 1 to 5, further comprising genetically modifying the HSCs and/or HPCs to generate genetically-modified HSCs and/or HPCs.
7. The method of any one of items 1 to 6, wherein said conditions suitable for HSC and/or HPC expansion comprises culturing the HSCs and/or HPCs at the air-liquid interface (ALI).
8. The method of any one of items 1 to 7, wherein said conditions suitable for HSC and/or HPC expansion comprises culturing the HSCs and/or HPCs in the presence of cytokines and/or growth factors.
9. The method of item 8, wherein the cytokines and/or growth factors comprises SCF, TPO, FLT3L and/or IL-6.
10. The method of any one of items 1 to 9, which expands the hematopoietic stem cells and/or hematopoietic progenitor cells by at least 10-fold.
11. The method of any one of items 1 to 10, wherein the selective HTR_{1F} agonist is N-[(3R)-3-(Dimethylamino)-2,3,4,9-tetrahydro-1H-carbazol-6-yl]-4-fluorobenzamide hydrochloride (LY 344864), 4-fluoro-N-(3-(1-methyl-4-piperidinyl)-1H-indol-5-yl)benzamide (LY 334370), 2,4,6-Trifluoro-N-[6-[(1-methyl-4-piperidinyl)carbonyl]-2-pyridinyl]benzamide (LY 573144, Lasmiditan), or 5-N-butyryloxy-N,N-dimethyltryptamine.
12. The method of any one of items 1 to 11, wherein the THRB agonist is triiodothyronine (T3).
13. A method for expanding hematopoietic stem cells (HSCs) and/or hematopoietic progenitor cells (HPCs) ex vivo comprising culturing said HSCs and/or HPCs in an air-liquid interface (ALI) culture system under conditions suitable for HSC and/or HPC expansion.
14. The method of item 13, wherein the HSCs and/or HPCs are obtained from donor bone marrow, umbilical donor cord blood, or donor peripheral blood.
15. The method of item 13 or 14, wherein the HSCs and/or HPCs are from a subject suffering from a disease or condition.
16. The method of item 15, wherein the disease is cancer, an immunodeficiency, myelodysplastic syndrome (MDS) or anemia.
17. The method of item 16, wherein the cancer is a hematological cancer.
18. The method of any one of items 13 to 17, further comprising genetically modifying the HSCs and/or HPCs to generate genetically-modified HSCs and/or HPCs.
19. The method of any one of items 13 to 18, wherein said conditions suitable for HSC and/or HPC expansion comprises culturing the HSCs and/or HPCs in the presence of cytokines and/or growth factors.
20. The method of item 19, wherein the cytokines and/or growth factors comprises SCF, TPO, FLT3L and/or IL-6.
21. A method for expanding hematopoietic stem cells (HSCs) and/or hematopoietic progenitor cells (HPCs) in a subject in need thereof comprising administering to the subject an effective amount of a selective 5-Hydroxytryptamine Receptor 1F (HTR_{1F}) agonist and/or of a Thyroid Hormone Receptor Beta (THRB) agonist.
22. The method of item 21, wherein the selective HTR_{1F} agonist is N-[(3R)-3-(Dimethylamino)-2,3,4,9-tetrahydro-1H-carbazol-6-yl]-4-fluorobenzamide hydrochloride (LY 344864), 4-fluoro-N-(3-(1-methyl-4-piperidinyl)-1H-indol-5-yl)benzamide (LY 334370), 2,4,6-Trifluoro-N-[6-[(1-methyl-4-piperidinyl)carbonyl]-2-pyridinyl]benzamide (LY 573144, Lasmiditan), or 5-N-butyryloxy-N,N-dimethyltryptamine.
23. The method of item 21 or 22, wherein the THRB agonist is triiodothyronine (T3).
24. The method of any one of items 21 to 23, wherein the subject is a stem-cell transplant recipient.
25. The method of any one of items 21 to 24, wherein the subject suffers from cancer, an immunodeficiency, myelodysplastic syndrome (MDS) or anemia.
26. The method of item 25, wherein the cancer is a hematological cancer.
27. The method of item 24, wherein the stem-cell transplant comprises hematopoietic stem cells (HSCs) and/or hematopoietic progenitor cells (HPCs) obtained by the method of any one of items 1 to 20.
28. Use of a selective 5-Hydroxytryptamine Receptor 1F (HTR_{1F}) agonist and/or of a Thyroid Hormone Receptor Beta (THRB) agonist for expanding hematopoietic stem cells (HSCs) and/or hematopoietic progenitor cells (HPCs) in a subject.
29. Use of a selective 5-Hydroxytryptamine Receptor 1F (HTR_{1F}) agonist and/or of a Thyroid Hormone Receptor Beta (THRB) agonist for the manufacture of a medicament for expanding hematopoietic stem cells (HSCs) and/or hematopoietic progenitor cells (HPCs) in a subject.
30. The use of item 28 or 29, wherein the selective HTR_{1F} agonist is N-[(3R)-3-(Dimethylamino)-2,3,4,9-tetrahydro-1H-carbazol-6-yl]-4-fluorobenzamide hydrochloride (LY 344864), 4-fluoro-N-(3-(1-methyl-4-piperidinyl)-1H-indol-5-yl)benzamide (LY 334370), 2,4,6-Trifluoro-N-[6-[(1-methyl-4-piperidinyl)carbonyl]-2-pyridinyl]benzamide (LY 573144, Lasmiditan), or 5-N-butyryloxy-N,N-dimethyltryptamine.
31. The use of any one of items 28 to 30, wherein the THRB agonist is triiodothyronine (T3).
32. The use of any one of items 28 to 31, wherein the subject is a stem-cell transplant recipient.
33. The method of any one of items 28 to 32, wherein the subject suffers from cancer, an immunodeficiency, myelodysplastic syndrome (MDS) or anemia.
34. The use of item 33, wherein the cancer is a hematological cancer.
35. The use of item 32, wherein the stem-cell transplant comprises HSCs and/or HPCs obtained by the method of any one of items 1 to 20.
36. An agent for use in expanding hematopoietic stem cells (HSCs) and/or hematopoietic progenitor cells (HPCs) in a subject, where the agent is a selective 5-Hydroxytryptamine Receptor 1F (HTR_{1F}) agonist and/or a Thyroid Hormone Receptor Beta (THRB) agonist.
37. The agent for use of item 36, wherein the selective HTR_{1F} agonist is N-[(3R)-3-(Dimethylamino)-2,3,4,9-tetrahydro-1H-carbazol-6-yl]-4-fluorobenzamide hydrochloride (LY 344864), 4-fluoro-N-(3-(1-methyl-4-piperidinyl)-1H-indol-5-yl)benzamide (LY 334370), 2,4,6-Trifluoro-N-[6-[(1-methyl-4-piperidinyl)carbonyl]-2-pyridinyl]benzamide (LY 573144, Lasmiditan), or 5-N-butyryloxy-N,N-dimethyltryptamine.
38. The agent for use of item 36 or 37, wherein the THRB agonist is triiodothyronine (T3).
39. The agent for use of any one of items 36 to 38, wherein the subject is a stem-cell transplant recipient.
40. The agent for use of any one of items 36 to 39, wherein the subject suffers from cancer, an immunodeficiency, myelodysplastic syndrome (MDS) or anemia.
41. The agent for use of item 40, wherein the cancer is a hematological cancer.
42. The agent for use of item 39, wherein the stem-cell transplant comprises HSCs and/or HPCs obtained by the method of any one of items 1 to 20.
43. A method for increasing the number of immune cells or for reconstituting the immune system in a subject in need thereof comprising administering to the subject an effective amount of a selective 5-Hydroxytryptamine Receptor 1F (HTR_{1F}) agonist and/or of a Thyroid Hormone Receptor Beta (THRB) agonist.
44. The method of item 43, wherein the selective HTR_{1F} agonist is N-[(3R)-3-(Dimethylamino)-2,3,4,9-tetrahydro-1H-carbazol-6-yl]-4-fluorobenzamide hydrochloride (LY 344864), 4-fluoro-N-(3-(1-methyl-4-piperidinyl)-1H-indol-5-yl)benzamide (LY 334370), 2,4,6-Trifluoro-N-[6-[(1-methyl-4-piperidinyl)carbonyl]-2-pyridinyl]benzamide (LY 573144, Lasmiditan), or 5-N-butyryloxy-N,N-dimethyltryptamine.
45. The method of item 43 or 44, wherein the THRB agonist is triiodothyronine (T3).
46. A method for increasing the number of immune cells or for reconstituting the immune system in a subject in need thereof comprising administering to the subject an effective amount of HSCs and/or HPCs obtained by the method of any one of items 1 to 20.
47. The method of any one of items 43 to 46, wherein the subject is immunosuppressed or immunodepleted.
48. Use of a selective 5-Hydroxytryptamine Receptor 1F (HTR_{1F}) agonist and/or of a Thyroid Hormone Receptor Beta (THRB) agonist for increasing the number of immune cells or for reconstituting the immune system in a subject.
49. Use of a selective 5-Hydroxytryptamine Receptor 1F (HTR_{1F}) agonist and/or of a Thyroid Hormone Receptor Beta (THRB) agonist for the manufacture of a medicament for increasing the number of immune cells or for reconstituting the immune system in a subject.
50. The use of item 48 or 49, wherein the selective HTR_{1F} agonist is N-[(3R)-3-(Dimethylamino)-2,3,4,9-tetrahydro-1H-carbazol-6-yl]-4-fluorobenzamide hydrochloride (LY 344864), 4-fluoro-N-(3-(1-methyl-4-piperidinyl)-1H-indol-5-yl)benzamide (LY 334370), 2,4,6-Trifluoro-N-[6-[(1-methyl-4-piperidinyl)carbonyl]-2-pyridinyl]benzamide (LY 573144, Lasmiditan), or 5-N-butyryloxy-N,N-dimethyltryptamine.
51. The use of any one of items 48 to 50, wherein the THRB agonist is triiodothyronine (T3).
52. Use of HSCs and/or HPCs obtained by the method of any one of items 1 to 20 for increasing the number of immune cells or for reconstituting the immune system in a subject.
53. Use of HSCs and/or HPCs obtained by the method of any one of items 1 to 20 for the manufacture of a medicament for increasing the number of immune cells or for reconstituting the immune system in a subject.
54. The use of any one of items 48 to 53, wherein the subject is immunosuppressed or immunodepleted.
55. An agent for use in increasing the number of immune cells or reconstituting the immune system in a subject, wherein the agent is a selective 5-Hydroxytryptamine Receptor 1F (HTR_{1F}) agonist and/or of a Thyroid Hormone Receptor Beta (THRB) agonist.
56. The agent for use of item 55, wherein the selective HTR_{1F} agonist is N-[(3R)-3-(Dimethylamino)-2,3,4,9-tetrahydro-1H-carbazol-6-yl]-4-fluorobenzamide hydrochloride (LY 344864), 4-fluoro-N-(3-(1-methyl-4-piperidinyl)-1H-indol-5-yl)benzamide (LY 334370), 2,4,6-Trifluoro-N-[6-[(1-methyl-4-piperidinyl)carbonyl]-2-pyridinyl]benzamide (LY 573144, Lasmiditan), or 5-N-butyryloxy-N,N-dimethyltryptamine.
57. The agent for use of item 55 or 56, wherein the THRB agonist is triiodothyronine (T3).
58. An agent for use in increasing the number of immune cells or for reconstituting the immune system in a subject, wherein the agent is HSCs and/or HPCs obtained by the method of any one of items 1 to 20.
59. The agent for use of any one of items 55 to 58, wherein the subject is immunosuppressed or immunodepleted.
60. A method for identifying an agent useful for expanding hematopoietic stem cells (HSCs) and/or hematopoietic progenitor cells (HPCs) ex vivo or in vivo, and/or for increasing the number of immune cells or for reconstituting the immune system in a subject, the method comprising determining whether the agent is a selective 5-Hydroxytryptamine Receptor 1F (HTR_{1F}) agonist and/or of a Thyroid Hormone Receptor Beta (THRB) agonist.
61. The method of item 60, comprising contacting a cell expressing HTR_{1F} and/or THRB with the agent, and measuring a signal or marker associated with HTR_{1F} and/or THRB activation.
62. A hematopoietic stem cell (HSC) and/or hematopoietic progenitor cell (HPC) culture medium comprising a selective 5-Hydroxytryptamine Receptor 1F (HTR_{1F}) agonist and/or of a Thyroid Hormone Receptor Beta (THRB) agonist.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 depicts the adapted air-liquid-interface (ALI) culture system to expand HSCs ex vivo. Between 5,000 and 50,000 CD34⁺ were plated on a 3.14 cm² polycarbonate cell culture insert (25 mm polycarbonate culture insert with 0.4 um porosity, Nunc #137060/Fisher #140640), and this insert was placed in a 10 cm wide petri dish containing 9.5 mL of expending media. Medium volume was adjusted at day 3 to ensure that cells are covered and changed at day 5. At day 10, cells were harvested and analyzed by flow cytometry before being injected in mice when indicated.

FIG. 2 shows the number of CD34⁺ harvested after 10 days of expansion when cultured in different culture systems. Cord-blood CD34⁺ cells were cultured under manual fed-batch (FB) or modified air-liquid interface (ALI) conditions for 10 days. Cells were cultured in cytokine-containing (SCF, TPO, FLT-3L) serum-free media (StemSpan™ SFEM II) where UM171 (40 nM) was added. CD34⁺ cells total number was assessed by flow cytometry using Courtright beads (BD).

FIG. 3 shows the differentiation capacity of cultured CD34⁺ with the different culture systems. Cord-blood CD34⁺ cells (20,000 cells) were cultured under manual fed-batch (FB) or modified air-liquid interface (ALI) conditions for 10 days. Cells were cultured in cytokine-containing (SCF, TPO, FLT-3L) serum-free media (StemSpan™ SFEM II). Cells were harvested after 10 days and 100 CD34⁺ cells were re-plated in 35 mm plate in MethoCult™ media. Total CFUs, granulocyte/macrophage (CFU-G, CFU-M and CFU-GM) and erythroid (CFU-E)/multi-lineage (CFU-GEMM) progenitors were enumerated after 16 days of culture. Uncultured cells were isolated from the same cord blood but were plated without being cultured.

FIG. 4 shows the results of human immune engraftment levels according to ex vivo culture methods used prior to HSC injection in mice. The progeny of 7,500 cord-blood CD34⁺ cells, isolated from a single donor, was cultured under manual fed-batch (FB) or modified air-liquid interface (ALI) conditions for 10 days and injected in irradiated adult NSG mice. Cells were cultured in cytokine-containing (SCF, TPO, FLT-3L) serum-free media (StemSpan™ SFEM, LDL) and UM171 (40 nM). An equal number (7,500 CD34⁺) of uncultured CD34⁺ cells from the same source were injected to determine baseline engraftment. Human immune reconstitution, expressed by the percentage of human CD45 (hCD45) over total CD45, was monitored in peripheral blood after 5,10,15 and 20 weeks post-transplantation by flow cytometry. Mean percentage of human CD45 cell engraftment +/−standard deviation (SD) is shown for 4 mice with FB-cultured cells, 5 mice with ALI-cultured cells and 2 mice with uncultured CB-CD34⁺ cells (as control) is shown.

FIG. 5 shows the effect of CD34⁺ purification after HSC ex vivo culture using ALI method on human immune engraftment levels when only. The progeny of 7,500 cord-blood CD34⁺ cells, isolated from a single donor, was cultured under modified air-liquid interface (ALI) conditions for 10 days. Cells were cultured in cytokine-containing (SCF, TPO, FLT-3L) serum-free media (StemSpan™ SFEM, LDL) and UM729 (500 nM). In one instance, the progeny was injected in irradiated adult NSG mice without purification of CD34⁺ cells, while in another instance the progeny underwent a magnetic column purification of CD34⁺ cells. (2 mice per condition). Human immune reconstitution, expressed by the percentage of human CD45 (hCD45) over total CD45, was monitored in peripheral blood after 7, 14 and 25 weeks post-transplantation by flow cytometry. Mean percentage of human CD45 cell engraftment+/−standard deviation (SD) are shown.

FIG. 6 shows that ex vivo cultured CD34⁺ cells using the ALI method maintains long-term reconstitution capacity of HSCs. The progeny of 7,500 cord-blood CD34⁺ cells, isolated from a single donor, was cultured under modified air-liquid interface (ALI) conditions for 10 days. Cells were cultured in cytokine-containing (SCF, TPO, FLT-3L) serum-free media (StemSpan™ SFEM, LDL) and UM729 (500 nM). The mouse was sacrificed at week 31 and bone marrow was harvested. Cells from the bone marrow were then injected in 2 irradiated NSG mice (approximately 3.6 million hCD45+ per mice, of which 3.8% were CD34⁺CD45RA⁻ and 1.8% CD34⁺CD45RA⁻). Human immune chimerism, expressed by the percentage of human CD45 (hCD45) over total CD45, was monitored in peripheral blood after 5, 7, 10, 21, and 26 weeks post-transplantation by flow cytometry. Mean percentage of human CD45 cell engraftment+/−standard deviation (SD) are shown.

FIG. 7 shows the effect of the addition of aryl hydrocarbon receptor (AhR) receptor antagonist in ALI culture system on human immune engraftment levels. The progeny of 7,500 cord-blood CD34+ cells was cultured under modified air-liquid interface (ALI) conditions for 10 days and injected in irradiated adult NSG mice. Cells were cultured in cytokine-containing (SCF, TPO, FLT-3L) serum-free media (StemSpan™ SFEM, LDL) containing UM729 (40 nM) with or without the addition of aryl hydrocarbon receptor (AhR) receptor antagonist CH-223191 (1 μM). Human immune reconstitution, expressed by the percentage of human CD45 (hCD45) over total CD45, was monitored in peripheral blood after 7, 14 and 25 weeks post-transplantation by flow cytometry. Mean percentage of human CD45 cell engraftment for 2 mice per condition is shown.

FIG. 8 shows the results of human immune engraftment levels of cultured human CD34⁺ isolated from cord blood units and cultured at different levels of oxygen. The progeny of 4,000 cord-blood CD34⁺ cells was cultured under manual fed-batch (FB) or modified air-liquid interface (ALI) conditions for 10 days and injected in irradiated (2 Gy) adult NSG mice (2 mice per condition). Cells were cultured in cytokine-containing (SCF, TPO, FLT-3L) serum-free media (StemSpan™ SFEM, LDL) supplemented with UM729 (40 nM) in an incubator with controlled oxygen levels (ambient 20% O₂ vs 5% O₂). Human immune reconstitution, expressed by the percentage of human CD45 (hCD45) over total CD45, was monitored in peripheral blood after 5, 7 and 10 weeks post-transplantation by flow cytometry. Relative human immune reconstitution (expressed as a fold of % hCD45⁺ lymphocytes observed in uncultured HSC conditions) are shown in the graph.

FIG. 9 shows the results of the use of a THRB agonist triiodothyronine (T3) in ex vivo culture on HSCs human immune engraftment. The progeny of 5,000 cord-blood CD34⁺ cells was cultured under manual fed-batch (FB) conditions for 10 days and injected in irradiated adult NSG mice (1 mouse per condition). Cells were cultured in cytokine-containing (SCF, TPO, FLT-3L) serum-free media (StemSpan™ SFEM, LDL) where UM171 (40 nM) or THRB-specific agonist triiodothyronine (T3-45 ng/mL) or AhR receptor antagonist CH-223191 (1 M) or UM171/T3 were added. Human immune reconstitution (% hCD45+ lymphocytes) was monitored in peripheral blood for several weeks post-transplantation.

FIG. 10A shows the results of the use of a HTR_{1F} agonist, LY344864, in ex vivo culture on HSCs human immune engraftment. The progeny of 7,500 cord-blood CD34⁺ cells was cultured under manual fed-batch (FB) or modified air-liquid interface (ALI) conditions for 10 days and injected in irradiated adult NSG mice. Cells were cultured in cytokine-containing (SCF, TPO, FLT-3L) serum-free media (StemSpan™ SFEM II) where UM171 (40 nM) or HTR1F-specific agonist LY344864 (at 500 nM final concentration) was added. An equal number of uncultured CD34⁺ cells from the same source were injected to determine baseline engraftment. Human immune reconstitution was monitored in peripheral blood after 7, 14 and 15 weeks post-transplantation as described above. Mean percentage of human CD45 cell engraftment for 2 mice per condition is shown.

FIG. 10B shows the results of the use of another HTR_{1F} agonist, LY573144 (Lasmiditan), in ex vivo culture on HSCs expansion and human immune engraftment. The progeny of 7,500 cord-blood CD34⁺ cells was cultured under manual fed-batch (FB) conditions for 6 days and injected in irradiated adult NSG mice. Cells were cultured in cytokine-containing (SCF, TPO, FLT-3L) serum-free media (StemSpan SFEM II) where UM729 (500 nM) or a HTR1F-specific agonist LY573144 (250 nM final concentration) was added. The progeny of an equal number of CD34⁺ cells cultured in the same conditions but without UM729 or LY573144 from the same source was used to determine baseline expansion (threshold indicated by the dotted line) and engraftment. Expansion fold, compared to untreated condition, of the CD34⁺ population after 6 days of ex vivo culture is shown in the left panel. Human immune reconstitution was monitored in peripheral blood (middle panel) and bone marrow (right panel) at week 25 post-transplantation as described above. Mean percentage of human CD45 cell engraftment for 2 mice per condition is shown.

FIG. 11 shows the results of the injections of the HTR_{1F} agonist LY573144 (Lasmiditan) on HSCs human immune engraftment in NSG mice. 38,000 human CB CD34+ were injected intravenously into irradiated NSG mice (n=8, 16 hrs after irradiation). Starting from 6 hours post-transplantation, half of the mice (n=4) received, by intraperitoneal injection, 0.3 mg of Lasmiditan diluted in 0.02% DMSO in PBS (drug), for a total of 4 doses in regular intervals (6 h, 24 h, 48 h and 72 h) after human HSC injection. The second half (control) received I.P. injection of vehicle (0.02% DMSO in PBS) (n=4; vehicle). Human chimerism was assessed on peripheral blood until 19 weeks post-transplantation as described above, after which time mice were sacrificed and spleen and bone marrow were analyzed. SEM are depicted.

FIG. 12 shows the relative expression of HTR_{1F} receptor in different subsets of hematopoietic cells. Publicly-available data on gene expression obtained by Affymetrix™ Human Genome U133 Plus 2.0 Array were interrogated for the level of expression of HTR1F in hematopoietic and immune cells (https://www.ncbi.nlm.nih.gov/geo/, Barrett T, et al. NCBI GEO: archive for functional genomics data sets—update. Nucleic Acids Res. 2013 January;41(Database issue):D991-5). Populations were define as followed: HSC, Hematopoietic stem cells from bone marrow (Lin-CD34⁺CD38⁻CD90⁺/CD34⁺CD33⁻CD38-Rho^{lo}c-kit⁺); early HPC, Hematopoietic progenitor cells from bone marrow (CD34⁺CD33⁻CD38-Rho^{hi}); CMP, Common myeloid progenitor cell (CD34⁺ CD123^{low} CD45RA⁻ CD110⁻); GMP, Granulocyte monocyte progenitors (CD34⁺ CD123^{low} CD45RA⁺ CD110⁻); MEP, Megakaryocyte-erythroid progenitor cell (CD34⁺ CD123^{low} CD45RA⁻ CD110⁺); PM, Promyelocyte from bone marrow (2-layer density gradient centrifugation, depletion of non-granulocytic cells by immunomagnetic sorting); MY, Myelocyte from bone marrow (2-layer density gradient centrifugation, depletion of non-granulocytic cells by immunomagnetic sorting); PMN, Polymorphonuclear cells from bone marrow (2-layer density gradient centrifugation, depletion of non-granulocytic cells by immunomagnetic sorting); PMN, Polymorphonuclear cells from peripheral blood (2-layer density gradient centrifugation, depletion of nongranulocytic cells by immunomagnetic sorting); CD14⁺ monocytes (Ficoll™ density gradient, CD14 pos. Selection/Human CD14 positive Selection Kit, Stem Cell Technologies); B cells, CD19⁺ B cells (EasySep® Human CD19⁺ B cells Enrichment Kit, Stem Cell Technologies); CD4⁺, CD4⁺ T cell (Monocytes-depleted fraction, EasySep® Human CD4 Positive Selection Kit, Stem Cell Technologies); CD8⁺, CD8⁺ T cells (Human CD8⁺ T cells Enrichment Kit, Stem Cell Technologies); NK cells, CD56⁺ Natural Killer cells (EasySep® Human CD56⁺ NK cells Enrichment Kit, Stem Cell Technologies); mDC, CD11c⁺ myeloid dendritic cells (EasySep® Human mDCs Enrichment Kit, Stem Cell Technologies); pDC, CD123⁺ plasmacytoid dendritic cells (EasySep® Human pDCs Enrichment Kit, Stem Cell Technologies). Human HSC cells are from GSE17054; Human GMP, MEP cells are from GSE19599; Human Monocytes cells are from GSE11864; Human Monocytes cells are from E-MEXP-1242. Data has been batch corrected.

FIG. 13 shows the relative expression of different 5-HT receptors in human hematopoietic stem cells. Gene expression data for Human HSC cells are from GSE17054 (Andersson A, et al. Gene expression signatures in childhood acute leukemias are largely unique and distinct from those of normal tissues and other malignancies. BMC Med Genomics 2010 Mar. 8; 3:6) and are defined as: Lin⁻CD34⁺CD38⁻CD90⁺/CD34⁺CD33⁻CD38⁻Rho^{lo}c-kit⁺.

FIGS. 14A-14E depict the structures of THRB agonists disclosed in WO 2022/057845.

FIGS. 15A-15B depict the structures of THRB agonists disclosed in WO 2021/018226.

FIGS. 16A-16B depict the structures of THRB agonists disclosed in WO 2021/244582.

FIGS. 17A-17C depict the structures of THRB agonists disclosed in WO 2021/057791.

FIGS. 18A-18C depict the structures of THRB agonists disclosed in WO 2021/043185.

FIGS. 19A-19B depict the structures of THRB agonists disclosed in WO 2020/228577.

FIGS. 20A-20J depict the structures of THRB agonists disclosed in WO 2022/127699.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies described herein, the present inventors have shown that ex vivo culturing human HSCs in an air-liquid-interface (ALI) system improves the engraftment properties of the cultured HSCs. The present inventors have shown that ex vivo culturing human HSCs in the presence of agonists of THRB and HTR_{1F} can elicit a significant increase of HSC engraftment, and that intra-peritoneal administration of HTR_{1F} agonists significantly increases engraftment and immune reconstitution in mice after a single injection of human HSCs that have not been previously cultured or amplified ex vivo.

Ex Vivo Expansion of Hematopoietic Stem Cells and/or Hematopoietic Progenitor Cells

The present disclosure provides a method for expanding hematopoietic stem cells (HSCs) and/or hematopoietic progenitor cells (HPCs) ex vivo comprising culturing said HSCs and/or HPCs in the presence of a selective 5-Hydroxytryptamine Receptor 1F (HTR_{1F}) agonist and/or of a Thyroid Hormone Receptor Beta (THRB) agonist under conditions suitable for HSC and/or HPC expansion. The present disclosure also provides the use of a selective HTR_{1F} agonist and/or of a THRB agonist for expanding HSC and/or HPC ex vivo.

The present disclosure also provides a method for expanding HSCs and/or HPCs ex vivo comprising culturing said HSCs and/or HPCs in an air-liquid interface (ALI) culture system under conditions suitable for HSC and/or HPC expansion. The present disclosure also provides the use of an air-liquid interface ALI culture system for expanding HSCs and/or HPCs ex vivo.

The present disclosure also provides a method for stimulating HSC engraftment and/or immune reconstitution in a subject undergoing HSC transplantation comprising culturing said HSC in an air-liquid interface (ALI) culture system prior to transplantation.

The present disclosure also provides a method for stimulating HSC engraftment and/or immune reconstitution in a subject undergoing HSC transplantation comprising culturing said HSCs in the presence of a selective 5-Hydroxytryptamine Receptor 1F (HTR_{1F}) agonist and/or of a Thyroid Hormone Receptor Beta (THRB) agonist prior to transplantation.

In an embodiment, the HSCs and/or HPCs are cultured in an ALI culture system in the presence of a selective HTR_{1F} agonist and/or of a THRB agonist. In another embodiment, the HSCs and/or HPCs are cultured in an ALI culture system in the presence of one or more other agents that stimulate HSC and/or HPC expansion ex vivo such as UM171, UM729, aryl hydrocarbon receptor antagonists (e.g., StemRegenin1, CH223191), Garcinol, Prostaglandin E₂ (PGE₂), histone deacetylase inhibitors (e.g., valproic acid, trichostatin A), DNA Methyltransferase inhibitors (e.g., UNC0638), and NOTCH signaling activators such as Delta-like ligands (e.g., DXI).

ALI culture system refers to a cell culture system in which only part of the cell is in contact with a suitable liquid culture medium (but not completely submerged in the medium) and the remaining part of the cell is in contact with air. This may be achieved, for example, by culturing the cells on a porous solid support (membrane, insert) placed on top of the culture medium. In another embodiment, the HSCs are cultured under conditions mimicking ALI culture conditions, e.g., higher effective oxygen concentration (used up by the cells), dilution of cell metabolic waste and nutrient renewal.

The term “HTR_{1F} agonist” as used herein refers to an agent that has the ability to bind to and activate HTR_{1F}, e.g., that has the ability to activate or increase signaling that occurs downstream of HTR_{1F}. The HTR_{1F} agonist may be a small molecule, a peptide, a polypeptide or an antibody or antigen-binding fragment thereof. Examples of HTR_{1F} agonists include Almotriptan Malate, Eletriptan Hydrobromide, Lasmiditan, CHEMBL3617549 (4-[3-(4-Fluorobenzamidyl)benzoyl]-1-methylpiperidine), CHEMBL3617550 (4-fluoro-N-[2-fluoro-3-(1-methylpiperidine-4-carbonyl)phenyl]benzamide), CHEMBL3617557 (4-fluoro-N-[6-(1-methylpiperidine-4-carbonyl)pyridin-2-yl]benzamide), CHEMBL177238 (2-chloro-4-fluoro-N-[3-(1-methylpiperidin-4-yl)furo[3,2-b]pyridin-5-yl]benzamide), CHEMBL177258 (4-fluoro-N-[3-(1-methylpiperidin-4-yl)furo[3,2-b]pyridin-5-yl]benzamide), CHEMBL369705 (2-chloro-6-fluoro-N-[3-(1-methylpiperidin-4-yl)furo[3,2-b]pyridin-5-yl]benzamide), CHEMBL173949 (2,4,6-trifluoro-N-[3-(1-methylpiperidin-4-yl)furo[3,2-b]pyridin-5-yl]benzamide), CHEMBL431041 (N-[3-(1-methylpiperidin-4-yl)-1H-pyrrolo[3,2-b]pyridin-5-yl]acetamide), CHEMBL104720 (N-[3-(1-methylpiperidin-4-yl)-1H-pyrrolo[3,2-b]pyridin-5-yl]cyclopropanecarboxamide), CHEMBL321080 (N-[3-(1-methylpiperidin-4-yl)-1H-pyrrolo[3,2-b]pyridin-5-yl]thiophene-2-carboxamide), CHEMBL105261 (N-[3-(1-methylpiperidin-4-yl)-1H-pyrrolo[3,2-b]pyridin-5-yl]propenamide), CHEMBL105955 (N-[3-(1-methylpiperidin-4-yl)-1H-pyrrolo[3,2-b]pyridin-5-yl]butanamide), CHEMBL102250 (N-[3-(1-methylpiperidin-4-yl)-1H-pyrrolo[3,2-b]pyridin-5-yl]furan-3-carboxamide), CHEMBL104753 (N-[3-(1-methylpiperidin-4-yl)-1H-pyrrolo[3,2-b]pyridin-5-yl]thiophene-3-carboxamide), CHEMBL105958 (N-[3-(1-methylpiperidin-4-yl)-1H-pyrrolo[3,2-b]pyridin-5-yl]pyridine-4-carboxamide, CHEMBL105722 (N-[3-(1-methylpiperidin-4-yl)-1H-pyrrolo[3,2-b]pyridin-5-yl]pyridine-2-carboxamide), CHEMBL105091 (N-[3-(1-methylpiperidin-4-yl)-1H-pyrrolo[3,2-b]pyridin-5-yl]furan-2-carboxamide), CHEMBL420475 (N-[3-(1-methylpiperidin-4-yl)-1H-pyrrolo[3,2-b]pyridin-5-yl]cyclobutanecarboxamide), CHEMBL186951 (2-chloro-4-fluoro-N-[3-(1-methylpiperidin-4-yl)-1,2-dihydrobenzimidazol-5-yl]benzamide, CHEMBL186662 (2,4-difluoro-N-[3-(1-methylpiperidin-4-yl)-1,2-benzoxazol-5-yl]benzamide), CHEMBL187581 (2,4,6-trifluoro-N-[3-(1-methylpiperidin-4-yl)-1,2-dihydrobenzimidazol-5-yl]benzamide), CHEMBL187308 (2-chloro-4-fluoro-N-[3-(1-methylpiperidin-4-yl)-1,2-benzoxazol-5-yl]benzamide), CHEMBL339980 (N-[3-[2-(dimethylamino)ethyl]-2-methyl-1H-indol-4-yl]-4-fluorobenzamide), Sumatriptan (1-[3-[2-(dimethylamino)ethyl]-1H-indol-5-yl]-N-methylmethanesulfonamide), 4-fluoro-N-(3-(1-methyl-4-piperidinyl)-1H-indol-5-yl)benzamide (LY-334,370), 5-N-butyryloxy-N,N-dimethyltryptamine (5-N-butyryloxy-DMT), BRL-54443 (3-(1-methylpiperidin-4-yl)-1H-indol-5-ol), N-[(3R)-3-(Dimethylamino)-2,3,4,9-tetrahydro-1H-carbazol-6-yl]-4-fluorobenzamide hydrochloride (LY-344,864), and Naratriptan.

The term “selective HTR_{1F} agonist” as used herein refers to an agent that has a higher affinity or activity on HTR1F relative to other receptors, and more particular other serotonin receptors. In other words, a “selective HTR_{1F} agonist” is an agent that may be used at a dose where it only binds to and activate HTR_{1F}, but not other receptors, and more particular other serotonin receptors. In an embodiment, the selective HTR_{1F} agonist has at least a 2-fold selectivity for HTR_{1F} relative to other receptors. In further embodiments, the selective HTR_{1F} agonist has at least a 5-fold, 10-fold, 20-fold, 30-fold, 40-fold or at least 50-fold selectivity for HTR_{1F} relative to other receptors.

Examples of selective HTR_{1F} agonists include N-[(3R)-3-(Dimethylamino)-2,3,4,9-tetrahydro-1H-carbazol-6-yl]-4-fluorobenzamide hydrochloride (LY-344,864, naratriptan), 4-fluoro-N-(3-(1-methyl-4-piperidinyl)-1H-indol-5-yl)benzamide (LY-334,370), 2,4,6-Trifluoro-N-[6-[(1-methyl-4-piperidinyl)carbonyl]-2-pyridinyl]benzamide (LY-573,144, Lasmiditan), 5-N-butyryloxy-N,N-dimethyltryptamine, 5-(4-flurorobenzamido)-2-methyl-N,N-dimethyltryptamine (LY-349,950).

The following HTR_{1F} agonists are disclosed in U.S. Pat. Nos. 5,708,187 and 5,814,653: 6-propanoyl-3-(methyl)amino-1,2,3,4-tetrahydro-9H-carbazole hydrobromide; 6-(2-methylpropanoyl)-3-(butyl)amino-1,2,3,4-tetrahydro-9H-carbazole fumarate; 6-butanoyl-3-(isobutyl)amino-1,2,3,4-tetrahydro-9H-carbazole; N-methyl-N-propyl-6-(sec-butanoyl)-3-amino-1,2,3,4-tetrahydro-9H-carbazole maleate; 6-(2-methylbutanoyl)-3-(isopropyl)amino-1,2,3,4-tetrahydro-9H-carbazole; 6-(3,3-dimethylbutanoyl)-3-(tert-butyl)amino-1,2,3,4-tetrahydro-9H-carbazole oxalate; 6-(3-chlorobenzoyl)-3-(dipropyl)amino-1,2,3,4-tetrahydro-9H-carbazole methanesulfonate; (+)-6-(2-bromobenzoyl)-3-(diethyl)amino-1,2,3,4-tetrahydro-9H-carbazole p-toluenesulfonate; 6-(2-ethoxybenzoyl)-3-(isobutyl)amino-1,2,3,4-tetrahydro-9H-carbazole tartarate; 6-(3-propoxybenzoyl)-3-(2-(1-methyl-1H-pyrazol-4-yl)ethyl)amino-1,2,3,4-tetrahydro-9H-carbazole trifluoromethanesulfonate; N-(2-(1-isopropyl-1H-pyrazol-4-yl)ethyl)-N-ethyl-6-(3-butoxybenzoyl)-3-amino-1,2,3,4-tetrahydro-9H-carbazole; 6-propanoyl-3-(methyl)aminomethyl-1,2,3,4-tetrahydro-9H-carbazole hydrobromide; (−)-6-(2-methylpropanoyl)-3-(isobutyl)aminomethyl-1,2,3,4-tetrahydro-9H-carbazole fumarate; 6-butanoyl-3-(isobutyl)aminomethyl-1,2,3,4-tetrahydro-9H-carbazole; N-methyl-N-propyl-6-(sec-butanoyl)-3-aminomethyl-1,2,3,4-tetrahydro-9H-carbazole maleate; 6-(2-methylbutanoyl)-3-(isopropyl)aminomethyl-1,2,3,4-tetrahydro-9H-carbazole; 6-(3,3-dimethylbutanoyl)-3-(tert-butyl)amino-1,2,3,4-tetrahydro-9H-carbazole oxalate; 6-(3-chlorobenzoyl)-3-(dimethyl)aminomethyl-1,2,3,4-tetrahydro-9H-carbazole methanesulfonate; 6-(2-bromobenzoyl)-3-(diethyl)aminomethyl-1,2,3,4-tetrahydro-9H-carbazole p-toluenesulfonate; 6-(2-ethoxybenzoyl)-3-(isobutyl)aminomethyl-1,2,3,4-tetrahydro-9H-carbazole tartarate; 6-(3-propoxybenzoyl)-3-(2-(1-methyl-1H-pyrazol-4-yl)ethyl)aminomethyl-1,2,3,4-tetrahydro-9H-carbazole trifluoromethanesulfonate; (+)—N-(2-(1-isopropyl-1H-pyrazol-4-yl)ethyl)-N-ethyl-6-(3-butoxybenzoyl)-3-aminomethyl-1,2,3,4-tetrahydro-9H-carbazole brosylate; 7-propanoyl-4-(methyl)aminocyclohepta[7,6-b]indole hydrobromide; 7-(2-methylpropanoyl)-4-(isopropyl)aminocyclohepta[7,6-b]indole fumarate; 7-butanoyl-4-(isobutyl)aminocyclohepta[7,6-b]indole; N-methyl-N-propyl-7-(sec-butanoyl)-4-aminocyclohepta-[7,6-b]indole maleate; 7-(2-methylbutanoyl)-4-(isopropyl)aminocyclohepta[7,6-b]indole; 7-(3,3-dimethylbutanoyl)-4-(tert-butyl)aminocyclohepta[7,6-b]indole oxalate; 7-(3-chlorobenzoyl)-4-(ethyl)aminocyclohepta[7,6-b]indole methanesulfonate; 7-(2-bromobenzoyl)-4-(diethyl)aminocyclohepta[7,6-b]indole p-toluenesulfonate; 7-(2-ethoxybenzoyl)-4-(isobutyl)aminocyclohepta[7,6-b]indole tartarate; 7-(3-propoxybenzoyl)-4-(2-(1-methyl-1H-pyrazol-4-yl)ethyl)aminocyclohepta[7,6-b]indole; trifluoromethanesulfonate; (−)—N-(2-(1-isopropyl-1H-pyrazol-4-yl)ethyl)-N-ethyl-7-(3-butoxybenzoyl)-4-aminocyclohepta[7,6-b]indole; 7-propanoyl-4-(methyl)aminomethylcyclohepta[7,6-b]indole hydrobromide; 7-(2-methylpropanoyl)-4-(isopropyl)aminomethylcyclohepta[7,6-b]indole fumarate; 7-butanoyl-4-(isobutyl)aminomethylcyclohepta[7,6-b]indole; N-methyl-N-propyl-7-(sec-butanoyl)-4-aminocyclohepta[7,6-b]indole maleate; 7-(2-methylbutanoyl)-4-(isopropyl) aminomethylcyclohepta[7,6-b]indole; 7-(3,3-dimethylbutanoyl)-4-(tert-butyl)aminomethylcyclohepta [7,6-blindole oxalate; 7-(3-chlorobenzoyl)-4-(butyl)aminomethylcyclohepta[7,6-b]indole methanesulfonate; 7-(2-bromobenzoyl)-4-(diethyl)aminomethylcyclohepta[7,6-b]indole p-toluenesulfonate; 7-(2-ethoxybenzoyl)-4-(isobutyl)aminomethylcyclohepta[7,6-b]indole tartarate; 7-(3-propoxybenzoyl)-4-(2-(1-methyl-1H-pyrazol-4-yl)ethyl)aminomethylcyclohepta[7,6-b]indole trifluoromethanesulfonate; N-(2-(1-isopropyl-1H-pyrazol-4-yl)ethyl)-N-ethyl-7-(3-butoxybenzoyl)-4-aminomethylcyclohepta[7,6-b]indole; 6-isopropoxycarbonylamino-3-(ethyl)amino-1,2,3,6-tetrahydropyridin-4-yl)-2-ethyl-1H-indole phosphate; 6-(1-buten-4-yloxy)carbonylamino-3-(isopropyl)amino-1,2,3,4-tetrahydro-9H-carbazole maleate; 6-(1-penten-5-yloxy)carbonylamino-3-(isobutyl)amino-1,2,3,4-tetrahydro-9H-carbazole malonate; N-isopropyl-N-methyl-6-(1-buten-4-yloxy)carbonylamino-3-amino-1,2,3,4-tetrahydro-9H-carbazole benzoate; 6-(3-penten-5-yloxy)carbonylamino-3-(tert-butyl)amino-1,2,3,4-tetrahydro-9H-carbazole; 6-(2-hexen-6-yloxy)carbonylamino-3-(2-pentyl)amino-1,2,3,4-tetrahydro-9H-carbazole 4-chlorobenzoate; 6-(2-chlorophenoxy)carbonylamino-3-(2-(1-isopropyl-1H-pyrazol-4-yl)ethyl)amino-1,2,3,4-tetrahydro-9H-carbazole glycollate; (+)-6-(3-fluorophenoxy) carbonylamino-3-(2-(1-isopropyl-1H-pyrazol-4-yl)ethy I)amino-1,2,3,4-tetrahydro-9H-carbazole; 6-(4-bromophenoxy)carbonylamino-3-(methyl)amino-1,2,3,4-tetrahydro-9H-carbazole hippurate; 6-isopropoxycarbonylamino-3-(ethyl)aminomethyl-1,2,3,4-tetrahydro-9H-carbazole phosphate; 6-(1-buten-4-yloxy)carbonylamino-3-(isopropyl)aminomethyl-1,2,3,4-tetrahydro-9H-carbazole maleate; 6-(1-penten-5-yloxy)carbonylamino-3-(isobutyl)aminomethyl-1,2,3,4-tetrahydro-9H-carbazole malonate; N-isopropyl-N-methyl-6-(1-buten-4-yloxy)carbonylamino-3-aminomethyl-1,2,3,4-tetrahydro-9H-carbazole benzoate; 6-(3-penten-5-yloxy)carbonylamino-3-(tert-butyl)aminomethyl-1,2,3,4-tetrahydro-9H-carbazole; 6-(2-hexen-6-yloxy)carbonylamino-3-(isobutyl)aminomethyl-1,2,3,4-tetrahydro-9H-carbazole 4-chlorobenzoate; 6-(2-chlorophenoxy)carbonylamino-3-(2-(1-isopropyl-1H-pyrazol-4-yl)ethyl)aminomethyl-1,2,3,4-tetrahydro-9H-carbazole glycollate; 6-(3-fluorophenoxy)carbonylamino-3-(2-(1-isopropyl-1H-pyrazol-4-yl)ethyl)aminomethyl-1,2,3,4-tetrahydro-9H-carbazole; 6-(4-bromophenoxy) carbonylamino-3-(methyl)aminomethyl-1,2,3,4-tetrahydro-9H-carbazole hippurate; 7-isopropoxycarbonylamino-4-(ethyl)amino-cyclohepta[7,6-b]indole phosphate; (−)-7-(1-buten-4-yloxy)carbonylamino-4-(isopropyl)aminocyclohepta[7,6-b]indole maleate; 7-(1-penten-5-yloxy)carbonylamino-4-(isobutyl)aminocyclohepta[7,6-b]indole malonate; N-isopropyl-N-methyl-7-(1-buten-4-yloxy)carbonylamino-4-amino-cyclohepta[7,6-b]indole benzoate; 7-(3-penten-5-yloxy)carbonylamino-4-(tert-butyl)aminocyclohepta[7,6-b]indole; 7-(2-hexen-6-yloxy) carbonylamino-4-amino-cyclohepta[7,6-b]indole 4-chlorobenzoate; 7-(2-chlorophenoxy) carbonylamino-4-(2-(1-isopropyl-1H-pyrazol-4-yl)ethyl)amino-cyclohepta[7,6-b]indole glycollate; 7-(3-fluorophenoxy)carbonylamino-4-(2-(1-methyl-1H-pyrazol-4-yl)ethyl)amino-cyclohepta[7,6-b]indole; 7-(4-bromophenoxy)carbonylamino-4-(methyl)aminocyclohepta[7,6-b]indole hippurate; 7-isopropoxycarbonylamino-4-(ethyl)aminomethylcyclohepta[7,6-b]indole phosphate; 7-(1-buten-4-yloxy)carbonylamino-4-(isopropyl)aminomethylcyclohepta[7,6-b]indole maleate; 7-(1-penten-5-yloxy)carbonylamino-4-(isobutyl)aminomethylcyclohepta[7,6-b]indole malonate; N-isopropyl-N-methyl-7-(1-buten-4-yloxy)carbonylamino-4-aminomethylcyclohep ta[7,6-b]indole benzoate; 7-(3-penten-5-yloxy)carbonylamino-4-(tert-butyl)aminomethylcyclohepta[7,6-b]indole; (+)-7-(2-hexen-7-yloxy)carbonylamino-4-aminomethylcyclohepta[7,6-b]indole 4-chlorobenzoate; 7-(2-chlorophenoxy)carbonylamino-4-(2-(1-isopropyl-1H-pyrazol-4-yl)ethyl)aminomethylcyclohepta[7,6-b]indole glycollate; 7-(3-fluorophenoxy)carbonylamino-4-(2-(1-isopropyl-1H-pyrazol-4-yl)ethyl)aminomethylcyclohepta[7,6-b]indole; 7-(4-bromophenoxy) carbonylamino-4-(methyl)aminomethylcyclohepta[7,6-b]indole hippurate; N-ethyl-N′-(3-(methyl)amino-1,2,3,4-tetrahydro-9H-carbazol-6-yl)thiourea citrate; N-isopropyl-N′-(3-(sec-butyl)amino-1,2,3,4-tetrahydro-9H-carbazol-6-yl)thiourea acetate; N-propyl-N′-(3-(isobutyl)amino-1,2,3,4-tetrahydro-9H-carbazol-6-yl)thiourea decanoate; N-butyl-N′-(3-(isopropyl)amino-1,2,3,4-tetrahydro-9H-carbazol-6-yl)thiourea formate; N-pentyl-N′-(3-(2-(1-isopropyl-1H-pyrazol-4-yl)ethyl)amino-1,2,3,4-tetrahydro-9H-carbazol-6-yl)urea caproate; N-(3-chloro)phenyl-N′-(dipropyl)amino-1,2,3,4-tetrahydro-9H-carbazol-6-yl)urea; N-(4-bromo)phenyl-N′-(3-((sec-butyl)amino-1,2,3,4-tetrahydro-9H-carbazol-6-yl)urea; (−)—N-hexyl-N′-(3-amino-1,2,3,4-tetrahydro-9H-carbazol-6-yl)urea; N-ethyl-N′-(3-(methyl)aminomethyl-1,2,3,4-tetrahydro-9H-carbazol-6-yl)thiourea citrate; N-isopropyl-N′-(3-(sec-butyl)aminomethyl-1,2,3,4-tetrahydro-9H-carbazol-6-yl)thiourea acetate; N-propyl-N′-(3-(diethyl)aminomethyl-1,2,3,4-tetrahydro-9H-carbazol-6-yl)thiourea decanoate; N-butyl-N′-(3-(isopropyl)aminomethyl-1,2,3,4-tetrahydro-9H-carbazol-6-yl)thiourea formate; N-pentyl-N′-(3-(2-(1-isopropyl-1H-pyrazol-4-yl)ethyl)aminomethyl-1,2,3,4-tetrahydro-9H-carbazo-6-yl)urea caproate; N-(3-chloro)phenyl-N′-(dipropyl)aminomethyl-1,2,3,4-tetrahydro-9H-carbazol-6-yl)urea; N-(4-bromo)phenyl-N′-(3-((sec-butyl)aminomethyl-1,2,3,4-tetrahydro-9H-carbazol-6-yl)urea; N-hexyl-N′-(3-aminomethyl-1,2,3,4-tetrahydro-9H-carbazol-6-yl)urea; N-ethyl-N′-(4-(methyl)aminocyclohepta[7,6-b]indol-7-yl)thiourea citrate; N-isopropyl-N′-(4-(sec-butyl)aminocyclohepta[7,6-b]indol-7-yl)thiourea acetate; N-propyl-N′-(4-(isobutyl)aminocyclohepta[7,6-b]indol-7-yl)thiourea decanoate; N-butyl-N′-(4-(isopropyl)aminocyclohepta[7,6-b]indol-7-yl)thiourea formate; N-pentyl-N′-(4-(2-(1-isopropyl-1H-pyrazol-4-yl)ethyl)aminocyclohepta[7,6-b]indol-7-yl)urea caproate; N-(3-chloro)phenyl-N′-(4-(dipropyl)aminocyclohepta[7,6-b]indol-7-yl)urea; N-(4-bromo)phenyl-N′-(4-((sec-butyl)aminocyclohepta[7,6-b]indol-7-yl)urea; N-hexyl-N′-(4-aminocyclohepta[7,6-b]indol-7-yl)urea; N-ethyl-N′-(4-(methyl)aminomethylcyclohepta[7,6-b]indol-7-yl)thiourea citrate; (+)—N-isopropyl-N′-(4-(sec-butyl)aminomethylcyclohepta[7,6-b]indol-7-yl)thiourea acetate; N-propyl-N′-(4-(dibutyl)aminomethylcyclohepta[7,6-b]-indol-7-yl)thiourea decanoate; N-butyl-N′-(4-(isopropyl)aminomethylcyclohepta[7,6-b]indol-7-yl)thiourea formate; N-pentyl-N′-(4-(2-(1-isopropyl-1H-pyrazol-4-yl)ethyl)aminomethylcyclohepta[7,6-b]indol-7-yl)urea caproate; N-(3-chloro)phenyl-N′-(4-(dipropyl)aminomethylcyclohepta[7,6-b]indol-7-yl)urea; (−)—N-(4-bromo)phenyl-N′-(4-((sec-butyl)aminomethylcyclohepta[7,6-b]indol-7-yl)urea; N-hexyl-N′-(4-aminomethylcyclohepta[7,6-b]indol-7-yl)urea; 6-(acetyl)amino-3-amino-1,2,3,4-tetrahydro-9H-carbazole phenylacetate; 6-(butyroyl)amino-3-(diethyl)amino-1,2,3,4-tetrahydro-9H-carbazole bisulfate; 6-(pentanoyl)amino-3-(propyl)amino-1,2,3,4-tetrahydro-9H-carbazole hydrochloride; 6-(2-methylbutanoyl)amino-3-(1-phenyleth-2-yl)amino-1,2,3,4-tetrahydro-9H-carbazole hydrochloride; 6-(2,2-dimethylpropanoyl) amino-3-(isobutyl)amino-1,2,3,4-tetrahydro-9H-carbazole phthalate; 6-(heptanoyl)amino-3-(isopropyl)amino-1,2,3,4-tetrahydro-9H-carbazole hydrobromide; 6-(4-phenylbutanoyl)amino-3-(dipropyl)amino-1,2,3,4-tetrahydro-9H-carbazole xylenesulfonate; 6-(phenoxyacetyl)amino-3-(1-(2-chlorophenyl)eth-2-yl)amino-1,2,3,4-tetrahydro-9H-carbazole mandelate; 6-(phenoxybutanoyl)amino-3-(1-(3-iodophenyl)eth-2-yl)amino-1,2,3,4-tetrahydro-9H-carbazole; 6-(butoxyacetyl)amino-3-(1-(4-bromophenyl)eth-2-yl)amino-1,2,3,4-tetrahydro-9H-carbazole; 6-(butoxycarbonylacetyl)amino-3-(2-(1-isopropyl-1H-pyrazol-4-yl)ethyl)amino-1,2,3,4-tetrahydro-9H-carbazole; N-methyl-N-propyl-6-(4-fluorobenzoyl)amino-3-amino-1,2,3,4-tetrahydro-9H-carbazole; 6-(2-chlorobenzoyl)amino-3-(diethyl)amino-1,2,3,4-tetrahydro-9H-carbazole; 6-(3-ethylbenzoyl)amino-3-(propyl)amino-1,2,3,4-tetrahydro-9H-carbazole naphthalene-1-sulfonate; 6-(4-ethoxybenzoyl)amino-3-(sec-butyl)amino-1,2,3,4-tetrahydro-9H-carbazole; (+)—N-methyl-N-(2-(1-ethyl-1H-pyrazol-4-yl)ethyl)-6-(2-butoxybenzoyl)amino-3-amino-1,2,3,4-tetrahydro-9H-carbazole; 6-(3-thiomethylbenzoyl)amino-3-(isopropyl)amino-1,2,3,4-tetrahydro-9H-carbazole; 6-(4-thiopropylbenzoyl)amino-3-amino-1,2,3,4-tetrahydro-9H-carbazole; 6-(2-nitrobenzoyl)amino-3-(isopropyl)amino-1,2,3,4-tetrahydro-9H-carbazole; 6-(2-cyanobenzoyl)amino-3-(dimethyl)amino-1,2,3,4-tetrahydro-9H-carbazole; 6-(2-(dimethylamino)benzoyl)amino-3-amino-1,2,3,4-tetrahydro-9H-carbazole; 6-(2-trifluoromethoxybenzoyl)amino-3-(diethyl)amino-1,2,3,4-tetrahydro-9H-carbazole; 6-(3-trifluoromethoxybenzoyl)amino-3-amino-1,2,3,4-tetrahydro-9H-carbazole hydroiodide; 6-(2-thienoyl)amino-3-(phenethyl)amino-1,2,3,4-tetrahydro-9H-carbazole 6-(3-thienoyl)amino-3-(dimethyl)amino-1,2,3,4-tetrahydro-9H-carbazole; 6-(2-furoyl)amino-3-(propyl)amino-1,2,3,4-tetrahydro-9H-carbazole; 6-(3-furoyl)amino-3-(diethyl)amino-1,2,3,4-tetrahydro-9H-carbazole; 6-(2-chloro-4-fluoro)benzoyl-3-(methyl)amino-1,2,3,4-tetrahydro-9H-carbazole; 6-(3-pyridinoyl)amino-3-(diethyl)amino-1,2,3,4-tetrahydro-9H-carbazole; 6-(3-chloro-2-pyridinoyl)amino-3-(diethyl)amino-1,2,3,4-tetrahydro-9H-carbazole; 6-(acetyl)amino-3-(methyl)aminomethyl-1,2,3,4-tetrahydro-9H-carbazole phenylacetate; 6-(butyroyl)amino-3-(butyl)aminomethyl-1,2,3,4-tetrahydro-9H-carbazole bisulfate; 6-(pentanoyl)amino-3-(phenethyl)aminomethyl-1,2,3,4-tetrahydro-9H-carbazole hydrochloride; 6-(2-methylbutanoyl) amino-3-(1-phenyleth-2-yl)aminomethyl-1,2,3,4-tetrahydro-9H-carbazole hydrochloride; 6-(2,2-dimethylpropanoyl)amino-3-(isobutyl)aminomethyl-1,2,3,4-tetrahydro-9H-carbazole phthalate; (−)-6-(heptanoyl)amino-3-(isopropyl)aminomethyl-1,2,3,4-tetrahydro-9H-carbazole hydrobromide; 6-(4-phenylbutanoyl)amino-3-(dipropyl)aminomethyl-1,2,3,4-tetrahydro-9H-carbazole xylenesulfonate; 6-(phenoxyacetyl)amino-3-(phenethyl)aminomethyl-1,2,3,4-tetrahydro-9H-carbazole mandelate; 6-(phenoxybutanoyl)amino-3-(isopropyl)aminomethyl-1,2,3,4-tetrahydro-9H-carbazole; 6-(butoxyacetyl)amino-3-(1-(4-bromophenyl)eth-2-yl)aminomethyl-1,2,3,4-tetrahydro-9H-carbazole; 6-(butoxycarbonylacetyl)amino-3-(2-(1-isopropyl-1H-pyrazol-4-yl)ethyl)aminomethyl-1,2,3,4-tetrahydro-9H-carbazole; N-methyl-N-propyl-6-(4-fluorobenzoyl)amino-3-aminomethyl-1,2,3,4-tetrahydro-9H-carbazole; 6-(2-chlorobenzoyl)amino-3-(diethyl)aminomethyl-1,2,3,4-tetrahydro-9H-carbazole; 6-(3-ethylbenzoyl)amino-3-(propyl)aminomethyl-1,2,3,4-tetrahydro-9H-carbazole naphthalene-1-sulfonate; 6-(4-ethoxybenzoyl)amino-3-(sec-butyl)aminomethyl-1,2,3,4-tetrahydro-9H-carbazole; N-methyl-N-(2-(1-ethyl-1H-pyrazol-4-yl)ethyl)-6-(2-butoxybenzoyl)amino-3-aminomethyl-1,2,3,4-tetrahydro-9H-carbazole; 6-(3-thiomethylbenzoyl)amino-3-(isopropyl)aminomethyl-1,2,3,4-tetrahydro-9H-carbazole; 6-(4-thiopropylbenzoyl)amino-3-aminomethyl-1,2,3,4-tetrahydro-9H-carbazole; 6-(2-nitrobenzoyl)amino-3-(isopropyl)aminomethyl-1,2,3,4-tetrahydro-9H-carbazole; 6-(2-cyanobenzoyl)amino-3-(dimethyl)aminomethyl-1,2,3,4-tetrahydro-9H-carbazole; 6-(2-(dimethylamino)benzoyl)amino-3-(dipropyl)aminomethyl-1,2,3,4-tetrahydro-9H-carbazole; 6-(2-trifluoromethoxybenzoyl)amino-3-(diethyl)aminomethyl-1,2,3,4-tetrahydro-9H-carbazole; 6-(3-trifluoromethoxybenzoyl)amino-3-aminomethyl-1,2,3,4-tetrahydro-9H-carbazole hydroiodide; 6-(2-thienoyl)amino-3-(propyl)aminomethyl-1,2,3,4-tetrahydro-9H-carbazole; (+)-6-(3-thienoyl)amino-3-(dimethyl)aminomethyl-1,2,3,4-tetrahydro-9H-carbazole; 6-(2-furoyl)amino-3-(propyl)aminomethyl-1,2,3,4-tetrahydro-9H-carbazole; 6-(3-furoyl)amino-3-(diethyl)aminomethyl-1,2,3,4-tetrahydro-9H-carbazole; 6-(2-chloro-4-fluoro)benzoyl-3-(methyl)aminomethyl-1,2,3,4-tetrahydro-9H-carbazole; 6-(3-pyridinoyl)amino-3-(diethyl)aminomethyl-1,2,3,4-tetrahydro-9H-carbazole; 6-(3-chloro-2-pyridinoyl)amino-3-(diethyl)aminomethyl-1,2,3,4-tetrahydro-9H-carbazole; 7-(acetyl)amino-4-(phenethyl)aminocyclohepta[7,6-b]indole phenylacetate; 7-(butyroyl)amino-4-aminocyclohepta[7,6-b]indole bisulfate; 7-(pentanoyl)amino-4-aminocyclohepta[7,6-b]indole hydrochloride; 7-(2-methylbutanoyl)amino-4-(1-phenyleth-2-yl)aminocyclohepta[7,6-b]indole hydrochloride; 7-(2,2-dimethylpropanoyl)amino-4-(isobutyl)aminocyclohepta[7,6-b]indole phthalate; 7-(heptanoyl)amino-4-(isopropyl)aminocyclohepta[7,6-b]indole hydrobromide; -(4-phenylbutanoyl)amino-4-(dipropyl)aminocyclohepta[7,6-b]indole xylenesulfonate; 7-(phenoxyacetyl)amino-4-aminocyclohepta[7,6-b]indole mandelate; 7-(phenoxybutanoyl)amino-4-(methyl)aminocyclohepta [7,6-b]indole; 7-(butoxyacetyl)amino-4-(1-(4-bromophenyl)eth-2-yl)aminocyclohepta[7,6-b]indole; 7-(butoxycarbonylacetyl)amino-4-(2-(1-isopropyl-1H-pyrazol-4-yl)ethyl)amino cyclohepta[7,6-b]indole; N-methyl-N-propyl-7-(4-fluorobenzoyl)amino-4-aminocyclohepta[7,6-b]indole; (+)-7-(2-chlorobenzoyl)amino-4-(diethyl)aminocyclohepta[7,6-b]indole; 7-(3-ethylbenzoyl)amino-4-(propyl)aminocyclohepta[7,6-b]indole naphthalene-1-sulfonate; 7-(4-ethoxybenzoyl)amino-4-(sec-butyl)aminocyclohepta[7,6-b]indole; N-methyl-N-(2-(1-ethyl-1H-pyrazol-4-yl)ethyl)-7-(2-butoxybenzoyl)amino-4-am inocyclohepta[7,6-b]indole; 7-(3-thiomethylbenzoyl)amino-4-(isopropyl)aminocyclohepta[7,6-b]indole; 7-(4-thiopropylbenzoyl)amino-4-aminocyclohepta[7,6-b]indole; 7-(2-nitrobenzoyl)amino-4-(isopropyl)aminocyclohepta[7,6-b]indole; 7-(2-cyanobenzoyl)amino-4-(dimethyl)aminocyclohepta[7,6-b]indole; 7-(2-(dimethylamino)benzoyl) amino-4-(isobutyl)aminocyclohepta[7,6-b]indole; 7-(2-trifluoromethoxybenzoyl)amino-4-(diethyl)aminocyclohepta[7,6-b]indole; 7-(3-trifluoromethoxybenzoyl)amino-4-aminocyclohepta[7,6-b]indole hydroiodide; (−)-7-(2-thienoyl)amino-4-(methyl)aminocyclohepta[7,6-b]indole; 7-(3-thienoyl)amino-4-(dimethyl)aminocyclohepta[7,6-b]indole; 7-(2-furoyl)amino-4-(propyl)aminocyclohepta[7,6-b]indole; 7-(3-furoyl)amino-4-(diethyl)aminocyclohepta[7,6-b]indole; 7-(2-chloro-4-fluoro)benzoyl-4-(methyl)aminocyclohepta[7,6-b]indole; 7-(3-pyridinoyl)amino-4-(diethyl)aminocyclohepta[7,6-b]indole; 7-(3-chloro-2-pyridinoyl)amino-4-(diethyl)aminocyclohepta [7,6-b]indole; 7-(acetyl)amino-4-(phenethyl)aminomethylcyclohepta[7,6-b]indole phenylacetate; 7-(butyroyl)amino-4-(sec-butyl)aminomethylcyclohepta[7,6-b]indole bisulfate; 7-(pentanoyl)amino-4-aminomethylcyclohepta[7,6-b]indole hydrochloride; 7-(2-methylbutanoyl)amino-4-(1-phenyleth-2-yl)aminomethylcyclohepta[7,6-b]indole hydrochloride; 7-(2,2-dimethylpropanoyl)amino-4-(isobutyl)aminomethylcyclohepta[7,6-b]indole phthalate; 7-(heptanoyl)amino-4-(isopropyl)aminomethylcyclohepta[7,6-b]indole hydrobromide; 7-(4-phenylbutanoyl)amino-4-(dipropyl)aminocyclohepta[7,6-b]indole xylenesulfonate; N-methyl-N-propyl-7-(phenoxyacetyl)amino-4-aminomethylcyclohepta-[7,6-b]indole mandelate; 7-(butanoyl)amino-4-aminomethylcyclohepta-[7,6-b]indole; 7-(acetyl)amino-4-(1-(4-bromophenyl)eth-2-yl)aminomethylcyclohepta[7,6-b]indole; 7-(acetyl)amino-4-(2-(1-isopropyl-1H-pyrazol-4-yl)ethyl)aminomethylcyclohep ta[7,6-b]indole; N-methyl-N-propyl-7-(4-fluorobenzoyl)amino-4-aminomethylcyclohepta[7,6-b]indole; 7-(2-chlorobenzoyl)amino-4-(diethyl)aminomethylcyclohepta [7,6-b]indole; 7-(3-ethylbenzoyl)amino-4-(propyl)aminomethylcyclohepta[7,6-b]indole naphthalene-1-sulfonate; (+)-7-(4-ethoxybenzoyl)amino-4-(sec-butyl)aminocyclohepta[7,6-b]indole; N-methyl-N-(2-(1-ethyl-1H-pyrazol-4-yl)ethyl)-7-(2-butoxybenzoyl)amino-4-aminomethylcyclohepta[7,6-b]indole; 7-(3-thiomethylbenzoyl)amino-4-(isopropyl)aminomethylcyclohepta[7,6-b]indole; 7-(4-thiopropylbenzoyl)amino-4-aminomethylcyclohepta[7,6-b]indole; 7-(2-nitrobenzoyl)amino-4-(isopropyl)aminocyclohepta[7,6-b]indole; 7-(2-cyanobenzoyl)amino-4-(dimethyl) aminomethylcyclohepta[7,6-b]indole; 7-(2-(dimethylamino)benzoyl)amino-4-(t-butyl) aminomethylcyclohepta[7,6-b]indole; 7-(2-trifluoromethoxybenzoyl)amino-4-(diethyl) aminomethylcyclohepta[7,6-b]indole; 7-(3-trifluoromethoxybenzoyl)amino-4-aminocyclohepta[7,6-b]indole hydroiodide; 7-(2-thienoyl)amino-4-(butyl)aminomethylcyclohepta [7,6-b]indole; 7-(3-thienoyl)amino-4-(dimethyl)aminomethylcyclohepta[7,6-b]indole; (+)-7-(2-furoyl)amino-4-(propyl)aminomethylcyclohepta[7,6-b]indole; 7-(3-furoyl)amino-4-(diethyl)aminomethylcyclohepta [7,6-b]indole; 7-(2-chloro-4-fluoro)benzoyl-4-(methyl)aminocyclohepta[7,6-b]indole; 7-(3-pyridinoyl)amino-4-(diethyl)aminomethylcyclohepta[7,6-b]indole; and 7-(3-chloro-2-pyridinoyl)amino-4-(diethyl)aminocyclohepta[7,6-b]indole The following HTR_{1F} agonists are disclosed in U.S. Pat. Nos. 5,521,196 and 5,721,252: 5-benzyloxy-3-[1-[2-[1-benzyl-1H-pyrazol-4-yl]ethyl]-4-piperidinyl]-1H-indole; 5-methoxy-3-[1-[2-[1-hexyl-1H-pyrazol-4-yl]ethyl]-4-piperidinyl]-1H-indole; 5-sec-butoxy-3-[1-[3-[1-cyclopentyl-1H-pyrazol-4-yl]propyl]-4-piperidinyl]-1H-indole; 5-propoxy-3-[1-[2-[1-ethyl-1H-pyrazol-4-yl]ethyl]-4-piperidinyl]-1H-indole; 5-propyl-3-[1-[2-[1-cyclohexylmethyl-1H-pyrazol-4-yl]ethyl]-4-piperidinyl]-1H-indole; 5-isopropoxy-3-[1-[2-[1H-pyrazol-4-yl]ethyl]-4-piperidinyl]-1H-indole; 5-iodo-3-[1-[4-[1-cyclopentyl-1H-pyrazol-4-yl]butyl]-4-piperidinyl]-1H-indole; 5-carboxamido-3-[1-[2-[1-cyclobutylmethyl-1H-pyrazol-4-yl]ethyl]-4-piperidinyl]-1H-indole; 5-ethylthio-3-[1-[2-[1-cycloheptylmethyl-1H-pyrazol-4-yl]ethyl]-4-piperidinyl]-1H-indole; 5-butoxy-3-[1-[2-[1-(4-isobutylphenyl)-1H-pyrazol-4-yl]ethyl]-4-piperidinyl]-1H-indole; 5-bromo-3-[1-[3-[1-(4-ethoxyphenyl)-1H-pyrazol-4-yl]propyl]-4-piperidinyl]-1H-indole; 5-chloro-3-[1-[2-[1-(3-propylphenyl)-1H-pyrazol-4-yl]ethyl]-4-piperidinyl]-1H-indole phthalate; 5-carboxamido-3-[1-[3-[1-(3-ethylphenyl)-1H-pyrazol-4-yl]propyl]-4-piperidinyl]-1H-indole succinate; 5-methoxy-3-[1-[2-[1-(3-isobutoxyphenyl)-1H-pyrazol-4-yl]ethyl]-4-piperidinyl]-1H-indole maleate; 5-propoxy-3-[1-[4-[1-(2-methoxyphenyl)-1H-pyrazol-4-yl]butyl]-4-piperidinyl]-1H-indole; 5-isopropoxy-3-[1-[2-[1-neopentyl-1H-pyrazol-4-yl]ethyl]-4-piperidinyl]-1H-indole hydrochloride; 5-benzyloxy-3-[1-[1,2,3,6-tetrahydro-[2-[1-benzyl-1H-pyrazol-4-yl]ethyl]-4-pyridinyl]-1H-indole; 5-methyl-3-[1-[1,2,3,6-tetrahydro-[2-[1-hexyl-1H-pyrazol-4-yl]ethyl]-4-pyridinyl]-1H-indole; 5-sec-butoxy-3-[1,2,3,6-tetrahydro-1-[3-[1-cyclopentyl-1H-pyrazol-4-yl]propyl]-4-pyridinyl]-1H-indole p-toluenesulfonate; 5-propoxy-3-[1-1,2,3,6-tetrahydro-1-[2-[1-ethyl-1H-pyrazol-4-yl]ethyl]-4-pyridinyl]-1H-indole; 5-hydroxy-3-[1,2,3,6-tetrahydro-1-[2-[1-cyclohexylmethyl-1H-pyrazol-4-yl]ethyl]-4-pyridinyl]-1H-indole; 5-isopropoxy-3-[1,2,3,6-tetrahydro-1-[2-[1H-pyrazol-4-yl]ethyl]-4-pyridinyl]-1H-indole hydrochloride; 5-iodo-3-[1,2,3,6-tetrahydro-1-[4-[1-cyclopentyl-1H-pyrazol-4-yl]butyl]-4-pyridinyl]-1H-indole; 5-carboxamido-3-[1,2,3,6-tetrahydro-1-[2-[1-cyclobutylmethyl-1H-pyrazol-4-yl]ethyl]-4-pyridinyl]-1H-indole; 5-methylthio-3-[1,2,3,6-tetrahydro-1-[2-[1-cycloheptylmethyl-1H-pyrazol-4-yl]ethyl]-4-pyridinyl]-1H-indole; 5-butoxy-3-[1,2,3,6-tetrahydro-1-[2-[1-(4-isobutylphenyl)-1H-pyrazol-4-yl]ethyl]-4-pyridinyl]-1H-indole; 5-bromo-3-[1,2,3,6-tetrahydro-1-[3-[1-(4-ethoxyphenyl)-1H-pyrazol-4-yl]propyl]-4-pyridinyl]-1H-indole hydrobromide; 5-chloro-3-[1,2,3,6-tetrahydro-1-[2-[1-(3-propylphenyl)-1H-pyrazol-4-yl]ethyl]-4-pyridinyl]-1H-indole phthalate; 5-carboxamido-3-[1,2,3,6-tetrahydro-1-[3-[1-(3-ethylphenyl)-1H-pyrazol-4-yl]propyl]-4-pyridinyl]-1H-indole succinate; 5-methoxy-3-[1,2,3,6-tetrahydro-1-[2-[1-(isobutoxyphenyl)-1H-pyrazol-4-yl]ethyl]-4-pyridinyl]-1H-indole maleate; 5-propoxy-3-[1,2,3,6-tetrahydro-1-[4-[1-(2-methoxyphenyl)-1H-pyrazol-4-yl]butyl]-4-pyridinyl]-1H-indole sulfate; and 5-isopropoxy-3-[1,2,3,6-tetrahydro-1-[2-[1-neopentyl-1H-pyrazol-4-yl]ethyl]-4-pyridinyl]-1H-indole hydrochloride.

The following HTR_{1F} agonists are disclosed in U.S. Pat. No. 5,521,197: 3-[1-[2-phenylethyl]-4-piperidinyl]-1H-indole hydrochloride; 5-methoxy-3-[1-[3-phenyl]propyl]-4-piperidinyl]-1H-indole hydrochloride; 5-chloro-3-[1-[2-[1-naphthyl]-ethyl]-4-piperidinyl]-1-H-indole oxalate; 5-bromo-3-[1-[3-[4-trifluoromethylphenyl]propyl]-4-piperidinyl]-1H-indole mandelate; 5-carboxamido-3-[1-[2-phenyl]ethyl]-4-piperidinyl]-1H-indole hydrobromide; 5-chloro-3-[1-[2-[2-naphthyl]ethyl]-4-piperidinyl]-1H-indole p-toluenesulfonate; 5-hydroxy-3-[1-[2-[3-thiomethylphenyl]ethyl]-4-piperidinyl]-1H-indole; 5-propyl-3-[1-[3-[4-methoxyphenyl]ethyl]-4-piperidinyl]-1H-indole; 5-iodo-3-[1-[4-[2-chlorophenyl]butyl]-4-piperidinyl]-1H-indole benzoate; 5-butoxy-3-[1-[2-[4-ethoxyphenyl]ethyl]-4-piperidinyl]-1H-indole methanesulfonate; 5-ethyl-3-[1-[4-thioethylphenylmethyl]-4-piperidinyl]-1H-indole; 5-isobutyl-3-[1-[3-[2-trifluoromethylphenyl]propyl]-4-piperidinyl]-1H-indole; 5-butyl-3-[1-[2-[3-fluorophenyl]ethyl]-4-piperidinyl]-1H-indole hydrochloride; 5-ethyl-3-[1-[2-[4-isopropylphenyl]-ethyl]-4-[1,2,3,6-tetrahydropyridinyl]]-1H-indole; 5-methyl-3-[1-[2-naphthylmethyl]-4-[1,2,3,6-tetrahydropyridinyl]]-1H-indole; 5-isopropoxy-3-[1-[2-[2-methylphenyl]ethyl]-4-[1,2,3,6-tetrahydropyridinyl]]-1H-indole hydrobromide; 5-isopropyl-3-[1-[2-[4-benzyloxyphenyl]ethyl]-4-[1,2,3,6-tetrahydropyridiny I]]-1H-indole maleate; 5-ethoxy-3-[1-[2-[4-isopropylphenyl]ethyl]-4-[1,2,3,6-tetrahydropyridinyl]]-1H-indole; 5-(sec-butoxy)-3-[1-[2-[3-bromophenyl]ethyl]-4-[1,2,3,6-tetrahydropyridinyl]]-1H-indole; 5-iodo-3-[1-[4-[2-chlorophenyl]butyl]-4-[1,2,3,6-tetrahydropyridinyl]]-1H-indole benzoate; 5-fluoro-3-[1-[2-phenyl]ethyl]-4-piperidinyl]-1H-indole hydrochloride; 5-chloro-3-[1-[3-phenyl]propyl]-4-piperidinyl]-1H-indole hydrochloride; 3-[1-[3-[4-trifluoromethylphenyl]propyl]-4-[1,2,3,6-tetrahydropyridinyl]]-1H-indole mandelate; 5-fluoro-3-[1-[2-phenyl]ethyl]-4-[1,2,3,6-tetrahydropyridinyl]]-1H-indole hydrobromide; 5-chloro-3-[1-[2-[3-thiomethylphenyl]ethyl]-4-[1,2,3,6-tetrahydropyridinyl]]-1H-indole p-toluenesulfonate; and 5-methoxy-3-[1-[4-[2-chlorophenyl]butyl]-4-[1,2,3,6-tetrahydropyridinyl]]-1H-indole benzoate The following HTR_{1F} agonists are disclosed in PCT publication No. WO 96/29075: 5-(4-fluorophenyl)thio-3-(1,2,3,6-tetrahydropyridin-4-yl)-1H-indole; 5-(3-chlorophenyl)thio-3-(1-(pent-2-yl)-1,2,3,6-tetrahydropyridin-4-yl)-2-methyl-1H-indole; 5-(2-bromophenyl)thio-3-(1-pentyl-1,2,3,6-tetrahydropyridin-4-yl)-1H-indole; 5-(2-iodophenyl)thio-3-(1-isobutyl-1,2,3,6-tetrahydropyridin-4-yl)-1H-indole; 5-(3-methoxyphenyl)thio-3-(1-(sec-butyl)-1,2,3,6-tetrahydropyridin-4-yl)-1H-indole; 5-(4-iodophenyl)thio-3-(1-(pent-2-yl)-1,2,3,6-tetrahydropyridin-4-yl)-1H-indole; 5-(2-ethoxyphenyl)thio-3-(1-(tert-butyl)-1,2,3,6-tetrahydropyridin-4-yl)-1H-indole; 5-(4-ethoxyphenyl)thio-3-(1-isopropyl-1,2,3,6-tetrahydropyridin-4-yl)-1H-indole; 5-(3-propoxyphenyl)thio-3-(1-butyl-1,2,3,6-tetrahydropyridin-4-yl)-1H-indole hydrochloride; 5-(2-isopropoxyphenyl)thio-3-(1-ethyl-1,2,3,6-tetrahydropyridin-4-yl)-1H-indole; 5-(4-isopropoxyphenyl)thio-3-(1-(isobutyl)-1,2,3,6-tetrahydropyridin-4-yl)-1H-indole hydrobromide; 5-(3-butoxyphenyl)thio-3-(1-butyl-1,2,3,6-tetrahydropyridin-4-yl)-1H-indole hydroiodide; 5-(2-isobutoxyphenyl)thio-3-(1-isobutyl-1,2,3,6-tetrahydropyridin-4-yl)-1H-indole; 5-(4-isobutoxyphenyl)thio-3-(1-(tert-butyl)-1,2,3,6-tetrahydropyridin-4-yl)-1H-indole acetate; 5-(3-sec-butoxyphenyl)thio-3-(1-butyl-1,2,3,6-tetrahydropyridin-4-yl)-1H-indole; 5-(2-tert-butoxyphenyl)thio-3-(1-ethyl-1,2,3,6-tetrahydropyridin-4-yl)-1H-indole propionate; 5-(4-tert-butoxyphenyl)thio-3-(1-isobutyl-1,2,3,6-tetrahydropyridin-4-yl)-1H-indole; 5-(2-pyridinyl)thio-3-(1-(tert-butyl)-1,2,3,6-tetrahydropyridin-4-yl)-1H-indole decanoate; 5-(4-pyridinyl)thio-3-(1-isobutyl-1,2,3,6-tetrahydropyridin-4-yl)-1H-indole; 5-(2-phenethyl)thio-3-(1-isopropyl-1,2,3,6-tetrahydropyridin-4-yl)-1H-indole caprylate; 5-(4-phenbutyl)thio-3-(1-ethyl-1,2,3,6-tetrahydropyridin-4-yl)-1H-indole; 5-(2-chlorophenyl)thio-3-(1-(neopentyl)piperidin-4-yl)-1H-indole acrylate; 5-(4-bromophenyl)thio-3-(1-(pent-3-yl)piperidin-4-yl)-1H-indole; 5-(2-bromophenyl)thio-3-(1-(sec-butyl)piperidin-4-yl)-1H-indole formate; 5-(3-iodophenyl)thio-3-(1-(tert-butyl)piperidin-4-yl)-1H-indole; 5-(2-methoxyphenyl)thio-3-(1-butylpiperidin-4-yl)-1H-indole isobutyrate; 5-(4-methoxyphenyl)thio-3-(1-pentylpiperidin-4-yl)-1H-indole; 5-(3-ethoxyphenyl)thio-3-(1-isobutylpiperidin-4-yl)-2-methyl-1H-indole caproate; 5-(2-propoxyphenyl)thio-3-(1-propylpiperidin-4-yl)-1H-indole; 5-(4-propoxyphenyl)thio-3-(1-(sec-butyl)piperidin-4-yl)-1H-indole heptanoate; 5-(3-isopropoxyphenyl)thio-3-(1-isopropylpiperidin-4-yl)-1H-indole; 5-(2-butoxyphenyl)thio-3-(1-propylpiperidin-4-yl)-1H-indole propiolate; 5-(4-butoxyphenyl)thio-3-(piperidin-4-yl)-1H-indole; 5-(2-sec-butoxyphenyl)thio-3-(1-propylpiperidin-4-yl)-1H-indole oxalate; 5-(4-sec-butoxyphenyl)thio-3-(1-(sec-butyl)piperidin-4-yl)-1H-indole; 5-(2-tert-butoxyphenyl)thio-3-(1-isopropylpiperidin-4-yl)-1H-indole malonate; 5-(3-pyridinyl)thio-3-(1-(sec-butyl)piperidin-4-yl)-1H-indole; 5-benzylthio-3-(1-butylpiperidin-4-yl)-1H-indole succinate; 5-(3-phenpropyl)thio-3-(1-propylpiperidin-4-yl)-1H-indole 5-propanoyl-3-(1-hexyl-1,2,3,6-tetrahydropyridin-4-yl)-1H-indole suberate; 5-(2-methylpropanoyl)-3-(1-pentyl-1,2,3,6-tetrahydropyridin-4-yl)-1H-indole; 5-butanoyl-3-(1-isobutyl-1,2,3,6-tetrahydropyridin-4-yl)-1H-indole sebacate; 5-(sec-butanoyl)-3-(1-propyl-1,2,3,6-tetrahydropyridin-4-yl)-1H-indole; 5-(2-methylbutanoyl)-3-(1-isopropyl-1,2,3,6-tetrahydropyridin-4-yl)-2-methyl-1H-indole fumarate; 5-(3,3-dimethylbutanoyl)-3-(1-(tert-butyl)-1,2,3,6-tetrahydropyridin-4-yl)-1H-indole; 5-heptanoyl-3-(1-(2-pentyl)-1,2,3,6-tetrahydropyridin-4-yl)-1H-indole butyne-1,4-dioate; 5-(3-chlorobenzoyl)-3-(1-hexyl-1,2,3,6-tetrahydropyridin-4-yl)-1H-indole; 5-(3-fluorobenzoyl)-3-(1-pentyl-1,2,3,6-tetrahydropyridin-4-yl)-1H-indole; 5-(3-bromobenzoyl)-3-(1-butyl-1,2,3,6-tetrahydropyridin-4-yl)-1H-indole hydrobromide; 5-(2-bromobenzoyl)-3-(1-ethyl-1,2,3,6-tetrahydropyridin-4-yl)-1H-indole; 5-(3-iodobenzoyl)-3-(1-isopropyl-1,2,3,6-tetrahydropyridin-4-yl)-1H-indole; 5-(3-methoxybenzoyl)-3-(1-(tert-butyl)-1,2,3,6-tetrahydropyridin-4-yl)-1H-indole; 5-(2-ethoxybenzoyl)-3-(1-isobutyl-1,2,3,6-tetrahydropyridin-4-yl)-1H-indole; 5-(4-ethoxybenzoyl)-3-(1-neopentyl-1,2,3,6-tetrahydropyridin-4-yl)-1H-indole; 5-(2-propoxybenzoyl)-3-(1-hexyl-1,2,3,6-tetrahydropyridin-4-yl)-1H-indole hydrochloride; 5-(3-propoxybenzoyl)-3-(1,2,3,6-tetrahydropyridin-4-yl)-1H-indole; 5-(3-butoxybenzoyl)-3-(1-pentyl-1,2,3,6-tetrahydropyridin-4-yl)-1H-indole; 5-(2-methylbenzoyl)-3-(1-propyl-1,2,3,6-tetrahydropyridin-4-yl)-1H-indole; 5-(3-ethylbenzoyl)-3-(1-isopropyl-1,2,3,6-tetrahydropyridin-4-yl)-1H-indole; 5-(4-propylbenzoyl)-3-(1-(tert-butyl)-1,2,3,6-tetrahydropyridin-4-yl)-1H-indole; 5-(2-propylbenzoyl)-3-(1-neopentyl-1,2,3,6-tetrahydropyridin-4-yl)-1H-indole; 5-(3-butylbenzoyl)-3-(1-(3-pentyl)-1,2,3,6-tetrahydropyridin-4-yl)-1H-indole; 5-(3-trifluoromethylbenzoyl)-3-(1-isobutyl-1,2,3,6-tetrahydropyridin-4-yl)-1H-indole; 5-(3-trifluoromethoxybenzoyl)-3-(1-ethyl-1,2,3,6-tetrahydropyridin-4-yl)-1H-indole; 5-(3-dimethylaminobenzoyl)-3-(1-isopropyl-1,2,3,6-tetrahydropyridin-4-yl)-1H-indole; 5-(3-phenylpropanoyl)-3-(1-isobutyl-1,2,3,6-tetrahydropyridin-4-yl)-1H-indole; 5-(5-phenylpentanoyl)-3-(1-(3-pentyl)-1,2,3,6-tetrahydropyridin-4-yl)-1H-indole; 5-(3-pyridinecarbonyl)-3-(1-propyl-1,2,3,6-tetrahydropyridin-4-yl)-1H-indole; 5-(3-phenylpropanoyl)-3-(1-isobutyl-1,2,3,6-tetrahydropyridin-4-yl)-1H-indole; 5-butanoyl-3-(1-(3-pentyl)piperidin-4-yl)-1H-indole; 5-(3-methyl)butanoyl-3-(1-butylpiperidin-4-yl)-2-methyl-1H-indole; 5-(2,2-dimethyl)propanoyl-3-(1-(2-pentyl)piperidin-4-yl)-1H-indole fumarate; 5-hexanoyl-3-(1-ethylpiperidin-4-yl)-1H-indole; 5-(2-ethyl)butanoyl-3-(1-(sec-butyl)piperidin-4-yl)-1H-indole; 5-(2-chlorobenzoyl)-3-(piperidin-4-yl)-1H-indole; 5-(2-fluorobenzoyl)-3-(1-isobutylpiperidin-4-yl)-1H-indole; 5-(2-bromobenzoyl)-3-(1-propylpiperidin-4-yl)-1H-indole 5-(2-iodobenzoyl)-3-(1-ethylpiperidin-4-yl)-1H-indole; 5-(4-iodobenzoyl)-3-(1-(sec-butyl)piperidin-4-yl)-1H-indole; 5-(2-methoxybenzoyl)-3-(1-(2-pentyl)piperidin-4-yl)-1H-indole; 5-(3-ethoxybenzoyl)-3-(1-pentylpiperidin-4-yl)-1H-indole; 5-(4-propoxybenzoyl)-3-(1-butylpiperidin-4-yl)-1H-indole; 5-(2-butoxybenzoyl)-3-(1-isobutylpiperidin-4-yl)-1H-indole hexyne-1,6-dioate; 5-(4-butoxybenzoyl)-3-(1-(3-pentyl)piperidin-4-yl)-1H-indole; 5-(3-methylbenzoyl)-3-(1-butylpiperidin-4-yl)-1H-indole; 5-(4-ethylbenzoyl)-3-(1-ethylpiperidin-4-yl)-1H-indole; 5-(2-ethylbenzoyl)-3-(1-(sec-butyl)piperidin-4-yl)-1H-indole benzoate; 5-(3-propylbenzoyl)-3-(1-(2-pentyl)piperidin-4-yl)-1H-indole; 5-(2-butylbenzoyl)-3-(1-pentylpiperidin-4-yl)-1H-indole; 5-(4-butylbenzoyl)-3-(1-hexylpiperidin-4-yl)-1H-indole 5-(2-trifluoromethylbenzoyl)-3-(1-butylpiperidin-4-yl)-1H-indole chlorobenzoate; 5-(3-trifluoromethoxybenzoyl)-3-(1-propylpiperidin-4-yl)-1H-indole; 5-(2-dimethylaminobenzoyl)-3-(1-(sec-butyl)pioeridin-4-yl)-1H-indole; 5-(4-phenylbutanoyl)-3-(1-pentylpiperidin-4-yl)-1H-indole 4-methylbenzoate; 5-(1-naphthoyl)-3-(1-hexylpiperidin-4-yl)-1H-indole; 5-(4-pyridinecarbonyl)-3-(1-ethylpiperidin-4-yl)-1H-indole; 5-(N-phenyl)carboxamido-3-(1,2,3,6-tetrahydropyridin-4-yl)-2-methyl-1H-indole 2,4-dinitrobenzoate; 5-(N-benzyl)carboxamido-3-(1-isobutyl-1,2,3,6-tetrahydropyridin-4-yl)-1H-indole; 5-(N-(2-(4-chlorophenyl)ethyl))carboxamido-3-(1-ethyl-1,2,3,6-tetrahydropyridin-4-yl)-1H-indole 4-hydroxybenzoate; 5-(N-(2-(3-methylphenyl)ethyl))carboxamido-3-(1-neopentyl-1,2,3,6-tetrahydropyridin-4-yl)-1H-indole; 5-(N-(3-(2-methoxyphenyl)propyl))carboxamido-3-(1-(tert-butyl)-1,2,3,6-tetrahydropyridin-4-yl)-1H-indole; 5-(N-(4-(4-trifluoromethylphenyl)butyl))carboxamido-3-(1-isopropyl-1,2,3,6-tetrahydropyridin-4-yl)-1H-indole 3-methoxybenzoate; 5-(N-(4-chlorophenyl))carboxamido-3-(1-(2-pentyl)piperidin-4-yl)-1H-indole; 5-(N-benzyl)carboxamido-3-(1-(3-pentyl)piperidin-4-yl)-1H-indole; 5-(N-(2-phenethyl))carboxamido-3-(1-(sec-butyl)piperidin-4-yl)-2-methyl-1H-indole; 5-(N-(3-phenpropyl))carboxamido-3-(piperidin-4-yl)-1H-indole; 5-(N-(3-phenpropyl))carboxamido-3-(1-hexylpiperidin-4-yl)-1H-indole phthalate; 5-(N-(4-phenbutyl))carboxamido-3-(1-pentylpiperidin-4-yl)-1H-indole; 5-(N-methyl-N-ethanesulfonyl)amino-3-(1-hexyl-1,2,3,6-tetrahydropyridin-4-yl)-1H-indole; 5-(N-ethyl-N-propanesulfonyl)amino-3-(1-(2-pentyl)-1,2,3,6-tetrahydropyridin-4-yl)-1H-indole; 5-(N-isopropanesulfonyl)amino-3-(1-(3-pentyl)-1,2,3,6-tetrahydropyridin-4-yl)-1H-indole; 5-(N-propyl-N-butanesulfonyl)amino-3-(1-(tert-butyl)-1,2,3,6-tetrahydropyridin-4-yl)-1H-indole; 5-(N-isobutanesulfonyl)amino-3-(1,2,3,6-tetrahydropyridin-4-yl)-1H-indole methanesulfonate; 5-(N-sec-butanesulfonyl)amino-3-(1-isobutyl-1,2,3,6-tetrahydropyridin-4-yl)-1H-indole; 5-(N-tert-butanesulfonyl)amino-3-(1-(sec-butyl)-1,2,3,6-tetrahydropyridin-4-yl)-1H-indole; 5-(N-butyl-N-benzenesulfonyl)amino-3-(1-isopropyl-1,2,3,6-tetrahydropyridin-4-yl)-1H-indole; 5-(N,N-diethylaminosulfonyl)amino-3-(1-propyl-1,2,3,6-tetrahydropyridin-4-yl)-2-methyl-1H-indole, 5-(N,N-dipropylaminosulfonyl)amino-3-(1-butyl-1,2,3,6-tetrahydropyridin-4-yl)-1H-indole; 5-(N,N-diisopropylaminosulfonyl)amino-3-(1-neopentyl-1,2,3,6-tetrahydropyridin-4-yl)-1H-indole; 5-(N,N-dibutylaminosulfonyl)amino-3-(1-(3-pentyl)-1,2,3,6-tetrahydropyridin-4-yl)-1H-indole; 5-(N-ethanesulfonyl)amino-3-(1-(3-pentyl)piperidin-4-yl)-1H-indole phenylsulfonate; 5-(N-propanesulfonyl)amino-3-(1-neopentylpiperidin-4-yl)-1H-indole; 5-(N-isopropanesulfonyl)amino-3-(1-pentylpiperidin-4-yl)-1H-indole; 5-(N-butanesulfonyl)amino-3-(piperidin-4-yl)-1H-indole; 5-(N-isobutanesulfonyl)amino-3-(1-(sec-butyl)piperidin-4-yl)-1H-indole; 5-(N-isopropyl-N-sec-butanesulfonyl)amino-3-(1-(tert-butyl)piperidin-4-yl)-1H-indole; 5-(N-(tert-butyl)sulfonyl)amino-3-(1-butylpiperidin-4-yl)-1H-indole; 5-(N,N-diethylaminosulfonyl)amino-3-(1-isobutyl-piperidin-4-yl)-1H-indole; 5-(N,N-dipropylaminosulfonyl)amino-3-(1-ethyl-piperidin-4-yl)-2-methyl-1H-indole; 5-(N,N-diisopropylaminosulfonyl)amino-3-(1-(2-pentyl)-piperidin-4-yl)-1H-indole; 5-(N,N-dibutylaminosulfonyl)amino-3-(1-hexylpiperidin-4-yl)-1H-indole; N-ethyl-N′-(3-(1-hexyl-1,2,3,6-tetrahydropyridin-4-yl)-1H-indol-5-yl)thiourea; N-isopropyl-N′-(3-(1-(sec-butyl)-1,2,3,6-tetrahydropyridin-4-yl)-1H-indol-5-yl)thiourea; N-(3-methoxy)phenyl-N′-(3-(1-isopropyl-1,2,3,6-tetrahydropyridin-4-yl)-1H-indol-5-yl)thiourea; N-(2-ethoxy)phenyl-N′-(2-methyl-3-(1-methyl-1,2,3,6-tetrahydropyridin-4-yl)-1H-indol-5-yl)thiourea; N-(2-ethoxy)phenyl-N′-(3-(1-(2-pentyl)-1,2,3,6-tetrahydropyridin-4-yl)-1H-indol-5-yl)thiourea; N-(3-ethoxy)phenyl-N′-(3-(1-(2-hexyl)-1,2,3,6-tetrahydropyridin-4-yl)-1H-indol-5-yl)thiourea; N-(3-propoxy)phenyl-N′-(3-(1,2,3,6-tetrahydropyridin-4-yl)-1H-indol-5-yl)thiourea; N-{3-isopropoxy)phenyl-N′-(3-(1-propyl-1,2,3,6-tetrahydropyridin-4-yl)-1H-indol-5-yl)thiourea; N-(4-isopropoxy)phenyl-N′-(3-(1-(3-pentyl)-1,2,3,6-tetrahydropyridin-4-yl)-1H-indol-5-yl)thiourea phenylacetate; N-(3-butoxy)phenyl-N′-(3-(1-(2-pentyl)-1,2,3,6-tetrahydropyridin-4-yl)-1H-indol-5-yl)thiourea hydrochloride; N-(2,3-dibromo)phenyl-N′-(3-(1-isopropyl-1,2,3,6-tetrahydropyridin-4-yl)-1H-indol-5-yl)thiourea; N-(2-bromo-3-iodo)phenyl-N′-(3-(1-(2-pentyl)-1,2,3,6-tetrahydropyridin-4-yl)-1H-indol-5-yl)thiourea; N-(3,4-difluoro)phenyl-N′-(3-(1-butyl-1,2,3,6-tetrahydropyridin-4-yl)-1H-indol-5-yl)thiourea; N-(3-chloro-4-bromo)phenyl-N′-(3-(1-(3-pentyl)-1,2,3,6-tetrahydropyridin-4-yl)-1H-indol-5-yl)thiourea; N-(2-bromo-4-fluoro)phenyl-N′-(3-(1-methyl-1,2,3,6-tetrahydropyridin-4-yl)-1H-indol-5-yl)thiourea; N-(2,4-diiodo)phenyl-N′-(3-(1-(sec-butyl)-1,2,3,6-tetrahydropyridin-4-yl)-1H-indol-5-yl)thiourea; N-(2-chloro-5-iodo)phenyl-N′-(3-(1-hexyl-1,2,3,6-tetrahydropyridin-4-yl)-1H-indol-5-yl)thiourea; N-(2-fluoro-6-iodo)phenyl-N′-(3-(1-(tert-butyl)-1,2,3,6-tetrahydropyridin-4-yl)-1H-indol-5-yl)thiourea; N-(3-fluoro-5-chloro)phenyl-N′-(3-(1-(2(pentyl)-1,2,3,6-tetrahydropyridin-4-yl)-1H-indol-5-yl)thiourea; N-phenethyl-N′-(3-(1-isopropyl-1,2,3,6-tetrahydropyridin-4-yl)-1H-indol-5-yl)thiourea N-(4-phenbutyl)-N′-(3-(1-(3-pentyl)-1,2,3,6-tetrahydropyridin-4-yl)-1H-indol-5-yl)thiourea; N-(2-trifluoromethyl)phenyl-N′-(3-(1-(2-pentyl)-1,2,3,6-tetrahydropyridin-4-yl)-1H-indol-5-yl)thiourea; N-(3-phenyl)phenyl-N′-(3-(1-(2-pentyl)-1,2,3,6-tetrahydropyridin-4-yl)-1H-indol-5-yl)thiourea; N-propyl-N′-(3-(1-(2-pentyl)piperidin-4-yl)-1H-indol-5-yl)thiourea; N-butyl-N′-(3-(1-isopropylpiperidin-4-yl)-1H-indol-5-yl)thiourea; N-(2-methoxy)phenyl-N′-(3-(1-(sec-butyl)pjperidin-4-yl)-1H-indol-5-yl)thiourea; N—(4-ethoxy)phenyl-N′-(3-(1-(2-pentyl)piperidin-4-yl)-1H-indol-5-yl)thiourea; N-(4-propoxy)phenyl-N′-(2-methyl-3-(1-ethylpiperidin-4-yl)-1H-indol-5-yl)thiourea; N-(2-propoxy)phenyl-N′-(3-(1-(tert-butyl)pjperidin-4-yl)-1H-indol-5-yl)thiourea; N-(2-isopropoxy)phenyl-N′-(3-(1-isobutylpiperidin-4-yl)-1H-indol-5-yl)thiourea; N-(4-butoxy)phenyl-N′-(3-piperidin-4-yl)-1H-indol-5-yl)thiourea; N-(2-butoxy)phenyl-N′-(3-(1-hexylpiperidin-4-yl)-1H-indol-5-yl)thiourea fumarate; N-(2-methoxy)phenyl-N′-(3-(1-(sec-butyl)piperidin-4-yl)-1H-indol-5-yl)thiourea; N-(2,3-dibromo)phenyl-N′-(3-(1-isopropylpiperidin-4-yl)-1H-indol-5-yl)thiourea; N-(2-bromo-3-iodo)phenyl-N″-(3-(1-(2-pentyl)piperidin-4-yl)-1H-indol-5-yl)thiourea; N-(3,4-difluoro)phenyl-N′-(3-(1-butylpiperidin-4-yl)-1H-indol-5-yl)thiourea; N-(3-chloro-4-bromo)phenyl-N′-(3-(1-(3-pentyl)piperidin-4-yl)-1H-indol-5-yl)thiourea; N-(2-bromo-4-fluoro)phenyl-N′-(3-(1-methylpiperidin-4-yl)-1H-indol-5-yl)thiourea; N-(2,4-diiodo)phenyl-N′-(3-(1-(sec-butyl)piperidin-4-yl)-1H-indol-5-yl)thiourea; N-(2-chloro-5-iodo)phenyl-N′-(3-(1-hexylpiperidin-4-yl)-1H-indol-5-yl)thiourea; N-(2-fluoro-6-iodo)phenyl-N′-(3-(1-(tert-butyl)piperidin-4-yl)-1H-indol-5-yl)thiourea; N-(3-fluoro-5-chloro)phenyl-N′-(3-(1-(2-pentyl)piperidin-4-yl)-1H-indol-5-yl)thiourea; N-(3-phenpropyl)-N′-(3-(1-(sec-butyl)piperidin-4-yl)-1H-indol-5-yl)thiourea; N-(4-trifluoromethyl)phenyl-N′-(3-(1-neopentylpiperidin-4-yl)-1H-indol-5-yl)thiourea; N-(4-phenyl)phenyl-N′-(3-(1-pentylpiperidin-4-yl)-1H-indol-5-yl)thiourea; N-pentyl-N′-(3-(1-ethyl-1,2,3,4-tetrahydropyridin-4-yl)-1H-indol-5-yl)urea; N-(1-buten-4-yl)-N′-(3-(1-isopropyl-1,2,3,4-tetrahydropyridin-4-yl)-1H-indol-5-yl)urea; N-(1-penten-5-yl)-N′-(3-(1-isobutyl-1,2,3,4-tetrahydropyridin-4-yl)-1H-indol-5-yl)urea hydrochloride; N-(3-penten-5-yl)-N′-(3-(1-(tert-butyl)-1,2,3,4-tetrahydropyridin-4-yl)-1H-indol-5-yl)urea; N-(2-hexen-6-yl)-N′-(3-(1-(2-pentyl)-1,2,3,4-tetrahydropyridin-4-yl)-1H-indol-5-yl)urea; N-(4-hexen-6-yl)-N′-(3-(1-neopentyl-1,2,3,4-tetrahydropyridin-4-yl)-1H-indol-5-yl)urea; N-cyclobutyl-N′-(3-(1,2,3,4-tetrahydropyridin-4-yl)-1H-indol-5-yl)urea; N-cycloheptyl-N′-{3-(1-pentyl-1,2,3,4-tetrahydropyridin-4-yl)-1H-indol-5-yl)urea; N-(3-chloro)phenyl-N′-(2-methyl-3-(1-propyl-1,2,3,4-tetrahydropyridin-4-yl)-1H-indol-5-yl)urea; N-(4-bromo)phenyl-N′—’3-(1-(sec-butyl)-1,2,3,4-tetrahydropyridin-4-yl)-1H-indol-5-yl)urea; N-(2-bromo)phenyl-N′-(3-(1-(3-pentyl)-1,2,3,4-tetrahydropyridin-4-yl)-1H-indol-5-yl)urea; N-(2-fluoro)phenyl-N′-(3-(1-(2-pentyl)-1,2,3,4-tetrahydropyridin-4-yl)-1H-indol-5-yl)urea; N-(2-iodo)phenyl-N′-(3-(1-ethyl-1,2,3,4-tetrahydropyridin-4-yl)-1H-indol-5-yl)urea; N-(4-iodo)phenyl-N′-(3-(1-(tert-butyl)-1,2,3,4-tetrahydropyridin-4-yl)-1H-indol-5-yl)urea; N-(3-methoxy)phenyl-N′-(3-(1-butyl-1,2,3,4-tetrahydropyridin-4-yl)-1H-indol-5-yl)urea; N-(3-ethoxy)phenyl-N′-(3-(1-pentyl-1,2,3,4-tetrahydropyridin-4-yl)-1H-indol-5-yl)urea; N-(2-propoxy)phenyl-N′-(3-(1-(tert-butyl)-1,2,3,4-tetrahydropyridin-4-yl)-1H-indol-5-yl)urea; N-(4-propoxy)phenyl-N′-(3-(1-hexyl-1,2,3,4-tetrahydropyridin-4-yl)-1H-indol-5-yl)urea; N-(3-isopropoxy)phenyl-N′-(3-(1-isopropyl-1,2,3,4-tetrahydropyridin-4-yl)-1H-indol-5-yl)urea; N-(4-butoxy)phenyl-N′-(3-(1-methyl-1,2,3,4-tetrahydropyridin-4-yl)-1H-indol-5-yl)urea; N-(2-butoxy)phenyl-N′-(3-(1-butyl-1,2,3,4-tetrahydropyridin-4-yl)-1H-indol-5-yl)urea; N-(3-formyl)phenyl-N′-(3-(1-pentyl-1,2,3,4-tetrahydropyridin-4-yl)-1H-indol-5-yl)urea; N-(4-acetyl)phenyl-N′-(3-(1-hexyl-1,2,3,4-tetrahydropyridin-4-yl)-1H-indol-5-yl)urea; N-(2-propanoyl)phenyl-N′-(3-(1-isobutyl-1,2,3,4-tetrahydropyridin-4-yl)-1H-indol-5-yl)urea; N-(4-propanoyl)phenyl-N′-(3-(1-neopentyl-1,2,3,4-tetrahydropyridin-4-yl)-1H-indol-5-yl)urea; N-(2-methylthio)phenyl-N′-(3-(1-butyl-1,2,3,4-tetrahydropyridin-4-yl)-1H-indol-5-yl)urea; N-(3-ethylthio)phenyl-N′-(3-(1-pentyl-1,2,3,4-tetrahydropyridin-4-yl)-1H-indol-5-yl)urea; N-(2-propylthio)phenyl-N′-(3-(1-(tert-butyl-1,2,3,4-tetrahydropyridin-4-yl)-1H-indol-5-yl)urea fumarate; N-(4-propylthio)phenyl-N′-(3-(1-hexyl-1,2,3,4-tetrahydropyridin-4-yl)-1H-indol-5-yl)urea; N-(2-butylthio)phenyl-N′-(3-(1-ethyl-1,2,3,4-tetrahydropyridin-4-yl)-1H-indol-5-yl)urea; N-(4-butylthio)phenyl-N′-(3-(1-isobutyl-1,2,3,4-tetrahydropyridin-4-yl)-1H-indol-5-yl)urea; N-(3-methyl)phenyl-N′-(3-(1-propyl-1.2,3,4-tetrahydropyridin-4-yl)-1H-indol-5-yl)urea; N-(4-ethyl)phenyl-N′-(3-(1-(sec-butyl)-1,2,3,4-tetrahydropyridin-4-yl)-1H-indol-5-yl)urea; N-(2-ethyl)phenyl-N′-(3-(1-(3-pentyl)-1,2,3,4-tetrahydropyridin-4-yl)-1H-indol-5-yl)urea; N-(3-propyl)phenyl-N′-(3-(1-(2-pentyl)-1,2,3,4-tetrahydropyridin-4-yl)-1H-indol-5-yl)urea; N-(2-isopropyl)phenyl-N′-(3-(1-ethyl-1,2,3,4-tetrahydropyridin-4-yl)-1H-indol-5-yl)urea; N-(3-butyl)phenyl-N′-(3-(1-propyl-1,2,3,4-tetrahydropyridin-4-yl)-1H-indol-5-yl)urea; N-(4-methoxycarbonyl)phenyl-N′-(3-(1-(sec-butyl)-1,2,3,4-tetrahydropyridin-4-yl)-1H-indol-5-yl)urea; N-(3-ethoxycarbonyl)phenyl-N′-(3-(1-(2-pentyl)-1,2,3,4-tetrahydropyridin-4-yl)-1H-indol-5-yl)urea; N-(2-propoxycarbonyl)phenyl-N′-(3-(1-ethyl-1,2,3,4-tetrahydropyridin-4-yl)-1H-indol-5-yl)urea; N-(4-propoxycarbonyl)phenyl-N′-(3-(1-isobutyl-1,2,3,4-tetrahydropyridin-4-yl)-1H-indol-5-yl)urea; N-(3-butoxycarbonyl)phenyl-N′-(3-(1-neopentyl-1,2,3,4-tetrahydropyridin-4-yl)-1H-indol-5-yl)urea; N-(2,3-dibromo)phenyl-N′-(3-(1-isopropyl-1,2,3,6-tetrahydropyridin-4-yl)-1H-indol-5-yl)urea; N-(2-bromo-3-iodo)phenyl-N′-(3-(1-(2-pentyl)-1,2,3,6-tetrahydropyridin-4-yl)-1H-indol-5-yl)urea; N-(3,4-difluoro)phenyl-N′-(3-(1-butyl-1,2,3,6-tetrahydropyridin-4-yl)-1H-indol-5-yl)urea; N-(3-chloro-4-bromo)phenyl-N′-(3-(1-(3-pentyl)-1,2,3,6-tetrahydropyridin-4-yl)-1H-indol-5-yl)urea; N-(2-bromo-4-fluoro)phenyl-N′-(3-(1-methyl-1,2,3,6-tetrahydropyridin-4-yl)-1H-indol-5-yl)urea; N-(2,4-diiodo)phenyl-N′-(3-(1-(sec-butyl)-1,2,3,6-tetrahydropyridin-4-yl)-1H-indol-5-yl)urea; N-(2-chloro-5-iodo)phenyl-N′-(3-(1-hexyl-1,2,3,6-tetrahydropyridin-4-yl)-1H-indol-5-yl)urea; N-(2-fluoro-6-iodo)phenyl-N′-(3-(1-(tert.-butyl)-1,2,3,6-tetrahydropyridin-4-yl)-1H-indol-5-yl)urea; N-(3-fluoro-5-chloro)phenyl-N′-(3-(1-(2-pentyl)-1,2,3,6-tetrahydropyridin-4-yl)-1H-indol-5-yl)urea; N-(4-phenbutyl)-N′-(3-(1-isopropyl-1,2,3,6-tetrahydropyridin-4-yl)-1H-indol-5-yl)urea; N-phenyl-N-propyl-N′-(3-(1-isobutyl-1,2,3,6-tetrahydropyridin-4-yl)-1H-indol-5-yl)urea; N-phenyl-N-butyl-N′-(3-(1-(tert-butyl)-1,2,3,6-tetrahydropyridin-4-yl)-1H-indol-5-yl)urea; N-methyl-N-propyl-N′-(3-(1-(3-pentyl)-1,2,3,6-tetrahydropyridin-4-yl)-1H-indol-5-yl)urea; N-ethyl-N-isopropyl-N′-(3-(1-(3-pentyl)-1,2,3,6-tetrahydropyridin-4-yl)-1H-indol-5-yl)urea; N,N-dipropyl-N′-(3-(1-isobutyl-1,2,3,6-tetrahydropyridin-4-yl)-1H-indol-5-yl)urea; N-butyl-N-propyl-N′-(3-(1-isobutyl-1,2,3,6-tetrahydropyridin-4-yl)-1H-indol-5-yl)urea; N-butyl-N-isopropyl-N′-(3-(1-isopropyl-1,2,3,6-tetrahydropyridin-4-yl)-1H-indol-5-yl)urea; N-hexyl-N′-(3-(1-propylpiperidin-4-yl)-1H-indol-5-yllurea; N-(2-buten-4-yl)-N′-(3-(1-butylpiperidin-4-yl)-1H-indol-5-yl)urea; N-(2-penten-5-yl)-N′-(3-(1-(sec-butyl)piperidin-4-yl)-1H-indol-5-yl)urea; N-(1-hexen-6-yl)-N′-(3-(1-pentylpiperidin-4-yl)-1H-indol-5-yl)urea; N-(3-hexen-6-yl)-N′-(3-(1-(3-pentyl)piperidin-4-yl)-1H-indol-5-yl)urea; N-cyclopropyl-N′-(3-(1-hexylpiperidin-4-yl)-1H-indol-5-yl)urea; N-cyclopentyl-N′-(3-(1-(3-pentyl)piperidin-4-yl)-1H-indol-5-yl)urea; N-cyclooctyl-N′-(3-(1-(tert-butyl)piperidin-4-yl)-1H-indol-5-yl)urea; N-(2-chloro)phenyl-N′-(3-(1-butylpiperidin-4-yl)-1H-indol-5-yl)urea; N-(3-bromo)phenyl-N′-(3-(1-pentylpiperidin-4-yl)-1H-indol-5-yl)urea; N-(3-fluoro)phenyl-N′-(3-(1-hexylpiperidin-4-yl)-1H-indol-5-yl)urea; N-(3-iodo)phenyl-N′-(2-methyl-3-(1-butylpiperidin-4-yl)-1H-indol-5-yl)urea; N-(3-phenyl)phenyl-N′-(3-(1-propylpiperidin-4-yl)-1H-indol-5-yl)urea; N-(4-ethoxy)phenyl-N′-(3-(1-(sec-butyl)pjperidin-4-yl)-1H-indol-5-yl)urea; N-(2-ethoxy)phenyl-N′-(3-(1-neopentylpiperidin-4-yl)-1H-indol-5-yl)urea; N-(3-propoxy)phenyl-N′-(3-(1-(2-pentyl)piperidin-4-yl)-1H-indol-5-yl)urea; N-(2-isopropoxy)phenyl-N′-(3-(1-ethylpiperidin-4-yl)-1H-indol-5-yl)urea; N-(4-isopropoxy)phenyl-N′-(3-(1-isobutylpiperidin-4-yl)-1H-indol-5-yl)urea; N-(3-butoxy)phenyl-N′-(3-(1-propylpiperidin-4-yl)-1H-indol-5-yl)urea; N-(4-formyl)phenyl-N′-(3-(1-(sec-butyl)piperidin-4-yl)-1H-indol-5-yl)urea; N-(2-formyl)phenyl-N′-(3-(1-(3-pentyl)piperidin-4-yl)-1H-indol-5-yl)urea; N-(3-acetyl)phenyl-N′-(3-(1-(2-pentyl)piperidin-4-yl)-1H-indol-5-yl)urea phenylpropionate; N-(3-propanoyl)phenyl-N′-(3-(1-pentylpiperidin-4-yl)-1H-indol-5-yl)urea; N-(3-ethylthio)phenyl-N′-(3-(1-propylpiperidin-4-yl)-1H-indol-5-yl)urea; N-(2-ethylthio)phenyl-N′-(3-(1-(3-pentyl)piperidin-4-yl)-1H-indol-5-yl)urea; N-(3-propylthio)phenyl-N′-(3-piperidin-4-yl)-1H-indol-5-yl)urea; N-(3-isopropylthio)phenyl-N′-(3-(1-isopropylpiperidin-4-yl)-1H-indol-5-yl)urea; N-(2-methyl)phenyl-N′-(3-(1-propylpiperidin-4-yl)-1H-indol-5-yl)urea; N-(3-ethyl)phenyl-N′-(3-(1-pentylpiperidin-4-yl)-1H-indol-5-yl)urea; N-(2-propyl)phenyl-N′-(3-(1-(tert-butyl)piperidin-4-yl)-1H-indol-5-yl)urea; N-(3-isopropyl)phenyl-N′-(3-(1-isopropylpiperidin-4-yl)-1H-indol-5-yl)urea; N-(4-butyl)phenyl-N′-(3-(1-methylpiperidin-4-yl)-1H-indol-5-yl)urea; N-(2-butyl)phenyl-N′-(3-(1-butylpiperidin-4-yl)-1H-indol-5-yl)urea; N-(3-methoxycarbonyl)phenyl-N′-(3-(1-pentylpiperidin-4-yl)-1H-indol-5-yl)urea; N-(2-ethoxycarbonyl)phenyl-N′-(3-(1-(tert-butyl)piperidin-4-yl)-1H-indol-5-yl)urea; N-(4-ethoxycarbonyl)phenyl-N′-(3-(1-hexylpiperidin-4-yl)-1H-indol-5-yl)urea; N-(3-propoxycarbonyl)phenyl-N′-(3-(1-isobutylpiperidin-4-yl)-1H-indol-5-yl)urea; N-(2-butoxycarbonyl)phenyl-N′-(3-(1-propylpiperidin-4-yl)-1H-indol-5-yl)urea; N-(2,3-dibromo)phenyl-N′-(3-(1-isopropylpiperidin-4-yl)-1H-indol-5-yl)urea; N-(2-bromo-3-iodo)phenyl-N′-(3-(1-(2-pentyl)piperidin-4-yl)-1H-indol-5-yl)urea; N-(3,4-difluoro)phenyl-N′-(3-(1-butylpiperidin-4-yl)-1H-indol-5-yl)urea; N-(3-chloro-4-bromo)phenyl-N′-(3-(1-(3-pentyl)piperidin-4-yl)-1H-indol-5-yl)urea; N-(2-bromo-4-fluoro)phenyl-N′-(3-(1-methylpiperidin-4-yl)-1H-indol-5-yl)urea; N-(2,4-diiodo)phenyl-N′-(3-(1-(sec-butyl)piperidin-4-yl)-1H-indol-5-yl)urea; N-(2-chloro-5-iodo)phenyl-N′-(3-(1-hexylpiperidin-4-yl)-1H-indol-5-yl)urea; N-(2-fluoro-6-iodo)phenyl-N′-(3-(1-(tert-butyl)piperidin-4-yl)-1H-indol-5-yl)urea; N-(3-fluoro-5-chloro)phenyl-N′-(3-(1-(2-pentyl)piperidin-4-yl)-1H-indol-5-yl)urea; N-(3-phenpropyl)-N′-(3-(1-propylpiperidin-4-yl)-1H-indol-5-yl)urea; N-ethyl-N-phenyl-N′-(3-(1-butylpiperidin-4-yl)-1H-indol-5-yl)urea; N-isopropyl-N-phenyl-N′-(3-(1-(sec-butyl)piperidin-4-yl)-1H-indol-5-yl)urea; N-ethyl-N-methyl-N′-(3-(1-(2-pentyl)piperidin-4-yl)-1H-indol-5-yl)urea; N-methyl-N-isopropyl-N′-(3-(1-neopentylpiperidin-4-yl)-1H-indol-5-yl)urea; N-ethyl-N-propyl-N′-(3-(1-neopentylpiperidin-4-yl)-1H-indol-5-yl)urea; N-ethyl-N-butyl-N′-(3-(1-(2-pentylpiperidin-4-yl)-1H-indol-5-yl)urea; N-propyl-N-isopropyl-N′-(3-(1-isobutylpiperidin-4-yl)-1H-indol-5-yl)urea; N,N-diisopropyl-N′-(3-(1-butylpiperidin-4-yl)-1H-indol-5-yl)urea; N,N-dibutyl-N′-(3-(1-butylpiperidin-4-yl)-1H-indol-5-yl)urea; 5-isopropoxycarbonylamino-3-(1-ethyl-1,2,3,6-tetrahydropyridin-4-yl)-2-ethyl-1H-indole; 5-(1-buten-4-yloxy)carbonylamino-3-(1-isopropyl-1,2,3,6-tetrahydropyridin-4-yl)-1H-indole hydrochloride; 5-(1-penten-5-yloxy)carbonylamino-3-(1-isobutyl-1,2,3,6-tetrahydropyridin-4-yl)-1H-indole; 5-(1-buten-4-yloxy)carbonylamino-3-(1-isopropyl-1,2,3,6-tetrahydropyridin-4-yl)-1H-indole; 5-(3-penten-5-yloxy)carbonylamino-3-(1-1,2,3,6-tetrahydropyridin-4-yl)-1H-indole; 5-(2-hexen-6-yloxy)carbonylamino-3-(1-(2-pentyl)-1,2,3,6-tetrahydropyridin-4-yl)-1H-indole; 5-(4-hexen-6-yloxy)carbonylamino-3-(1-(neopentyl)-1,2,3,6-tetrahydropyridin-4-yl)-1H-indole; 5-(2-chlorophenoxy)carbonylamino-3-(1-(3-pentyl)-1,2,3,6-tetrahydropyridin-4-yl)-1H-indole; 5-(3-fluorophenoxy)carbonylamino-3-(1-(sec-butyl)-1,2,3,6-tetrahydropyridin-4-yl)-1H-indole; 5-(3-bromophenoxy)carbonylamino-3-(1-methyl-1,2,3,6-tetrahydropyridin-4-yl)-1H-indole; 5-(2-bromophenoxy)carbonylamino-3-(1-neopentyl-1,2,3,6-tetrahydropyridin-4-yl)-1H-indole; 5-(3-iodophenoxy)carbonylamino-3-(1-(tert-butyl)-1,2,3,6-tetrahydropyridin-4-yl)-1H-indole; 5-(3-methoxyphenoxy)carbonylamino-3-(1-isopropyl-1,2,3,6-tetrahydropyridin-4-yl)-1H-indole; 5-(4-ethoxyphenoxy)carbonylamino-3-(1-methyl-1,2,3,6-tetrahydropyridin-4-yl)-1H-indole; 5-(2-ethoxy)phenoxycarbonylamino-3-(1-butyl-1,2,3,6-tetrahydropyridin-4-yl)-1H-indole; 5-(3-propoxyphenoxy)carbonylamino-3-(1-pentyl-1,2,3,6-tetrahydropyridin-4-yl)-1H-indole; 5-(2-butoxyphenoxy)carbonylamino-3-(1-(2-pentyl)-1,2,3,6-tetrahydropyridin-4-yl)-1H-indole; 5-(4-butoxyphenoxy)carbonylamino-3-(1-hexyl-1,2,3,6-tetrahydropyridin-4-yl)-1H-indole; 5-cyclobutoxycarbonylamino-3-(1-(sec-butyl)-1,2,3,6-tetrahydropyridin-4-yl)-1H-indole; 5-cyclooctyloxycarbonylamino-3-(1-butyl-1,2,3,6-tetrahydropyridin-4-yl)-1H-indole; 5-(butoxymethoxy)carbonylamino-3-(1-propyl-1,2,3,6-tetrahydropyridin-4-yl)-1H-indole; 5-(ethoxypropoxy)carbonylamino-3-(1-methyl-1,2,3,6-tetrahydropyridin-4-yl)-1H-indole; 5-butoxycarbonylamino-3-(1-propylpiperidin-4-yl)-2-propyl-1H-indole; 5-(2-buten-4-yloxy)carbonylamino-3-(piperidin-4-yl)-1H-indole; 5-(2-penten-5-yloxy)carbonylamino-3-(1-(sec-butyl)piperidin-4-yl)-1H-indole; 5-(1-hexen-6-yloxy)carbonylamino-3-(1-pentylpiperidin-4-yl)-1H-indole; 5-(3-hexen-6-yloxy)carbonylamino-3-(1-(3-pentyl)piperidin-4-yl)-1H-indole; 5-(3-chlorophenoxy)carbonylamino-3-(1-hexylpiperidin-4-yl)-1H-indole; 5-(2-fluorophenoxy) carbonylamino-3-(1-butylpiperidin-4-yl)-1H-indole; 5-(4-fluorophenoxy)carbonylamino-3-(1-pentylpiperidin-4-yl)-1H-indole; 5-(4-bromophenoxy)carbonylamino-3-(1-propylpiperidin-4-yl)-1H-indole; 5-(2-iodophenoxy)carbonylamino-3-(1-isobutylpiperidin-4-yl)-1H-indole; 5-(4-iodophenoxy)carbonylamino-3-(1-(2-pentyl)piperidin-4-yl)-1H-indole; 5-(3-chlorophenoxy) carbonylamino-3-(1-hexylpiperidin-4-yl)-1H-indole; 5-(2-methoxyphenoxy)carbonylamino-3-(1-hexylpiperidin-4-yl)-2-ethyl-1H-indole citrate; 5-(3-ethoxyphenoxy)carbonylamino-3-(1-propylpiperidin-4-yl)-1H-indole; 5-(4-propoxyphenoxy)carbonylamino-3-(1-(sec-butyl)piperidin-4-yl)-1H-indole; 5-(2-propoxyphenoxy)carbonylamino-3-(1-(2-pentyl)piperidin-4-yl)-1H-indole; 5-(3-butoxyphenoxy)carbonylamino-3-(1-neopentylpiperidin-4-yl)-1H-indole; 5-cyclopropoxy carbonylamino-3-(1-(tert-butyl)piperidin-4-yl)-1H-indole; 5-cyclohexyloxycarbonylamino-3-(1-isobutylpiperidin-4-yl)-1H-indole; 5-cyclooctyloxycarbonylamino-3-(1-isopropylpiperidin-4-yl)-1H-indole; 5-(propoxyethoxy)carbonylamino-3-(1-ethylpiperidin-4-yl)-1H-indole; 5-(4-methoxybutoxy)carbonylamino(1-ethylpiperidin-4-yl)-1H-indole; 5-(acetyl)amino-3-(1-hexyl-1,2,3,6-tetrahydropyridin-4-yl)-1H-indole; 5-(butyroyl)amino-3-(1-(2-pentyl)-1,2,3,6-tetrahydropyridin-4-yl)-1H-indole; 5-(pentanoyl)amino-3-(1-pentyl-1,2,3,6-tetrahydropyridin-4-yl)-1H-indole glycollate; 5-(2-methylbutanoyl)amino-3-(1-(sec-butyl)-1,2,3,6-tetrahydropyridin-4-yl)-1H-indole; 5-(2,2-dimethylpropanoyl)amino-3-(1-isobutyl-1,2,3,6-tetrahydropyridin-4-yl)-1H-indole; 5-(heptanoyl)amino-3-(1-isopropyl-1,2,3,6-tetrahydropyridin-4-yl)-1H-indole; 5-(cyclooctylcarbonyl) amino-3-(1-isobutyl-1,2,3,6-tetrahydropyridin-4-yl)-1H-indole; 5-(4-phenylbutanoyl)amino-3-(1-pentyl-1,2,3,6-tetrahydropyridin-4-yl)-1H-indole; 5-(phenoxyacetyl)amino-3-(1-(3-pentyl)-1,2,3,6-tetrahydropyridin-4-yl)-1H-indole; 5-(4-phenoxybutanoyl)amino-3-(1-hexyl-1,2,3,6-tetrahydropyridin-4-yl)-1H-indole; 5-(butoxyacetyl)amino-3-(1-(3-pentyl)-1,2,3,6-tetrahydropyridin-4-yl)-1H-indole; 5-(4-ethoxybutanoyl)amino-3-(1,2,3,6-tetrahydropyridin-4-yl)-1H-indole tartrate; 5-(butoxycarbonylacetyl)amino-3-(1-(sec-butyl)-1,2,3,6-tetrahydropyridin-4-yl)-1H-indole; 5-(4-butoxycarbonylbutanoyl)amino-3-(1-hexyl-1,2,3,6-tetrahydropyridin-4-yl)-1H-indole; 5-benzoylamino-3-(1-ethyl-1,2,3,6-tetrahydropyridin-4-yl)-1H-indole hydrochloride; 5-benzoylamino-3-(1-propyl-1,2,3,6-tetrahydropyridin-4-yl)-1H-indole; 5-benzoylamino-3-(1-isopropyl-1,2,3,6-tetrahydropyridin-4-yl)-1H-indole; 5-benzoylamino-3-(1-butyl-1,2,3,6-tetrahydropyridin-4-yl)-1H-indole; 5-benzoylamino-3-(1-isobutyl-1,2,3,6-tetrahydropyridin-4-yl)-1H-indole; 5-benzoylamino-3-(1-(sec-butyl)-1,2,3,6-tetrahydropyridin-4-yl)-1H-indole; 5-benzoylamino-3-(1-(tert-butyl)-1,2,3,6-tetrahydropyridin-4-yl)-1H-indole; 5-benzoylamino-3-(1-pentyl-1,2,3,6-tetrahydropyridin-4-yl)-1H-indole; 5-benzoylamino-3-(1-(2-pentyl)-1,2,3,6-tetrahydropyridin-4-yl)-1H-indole; 5-benzoylamino-3-(1-(3-pentyl)-1,2,3,6-tetrahydropyridin-4-yl)-1H-indole; 5-benzoylamino-3-(1-neopentyl-1,2,3,6-tetrahydropyridin-4-yl)-1H-indole propanesulfonate; 5-benzoylamino-3-(1-hexyl-1,2,3,6-tetrahydropyridin-4-yl)-1H-indole; 5-(4-fluorobenzoyl)amino-3-(1-ethyl-1,2,3,6-tetrahydropyridin-4-yl)-1H-indole fumarate; 5-(4-fluorobenzoyl)amino-3-(1-propyl-1,2,3,6-tetrahydropyridin-4-yl)-1H-indole; 5-(4-fluorobenzoyl)amino-3-(1-isopropyl-1,2,3,6-tetrahydropyridin-4-yl)-1H-indole; 5-(4-fluorobenzoyl)amino-3-(1-butyl-1,2,3,6-tetrahydropyridin-4-yl)-1H-indole; 5-(4-fluorobenzoyl)amino-3-(1-isobutyl-1,2,3,6-tetrahydropyridin-4-yl)-1H-indole; 5-(4-fluorobenzoyl)amino-3-(1-(sec-butyl)-1,2,3,6-tetrahydropyridin-4-yl)-1H-indole; 5-(4-fluorobenzoyl)amino-3-(1-(tert-butyl)-1,2,3,6-tetrahydropyridin-4-yl)-1H-indole; 5-(4-fluorobenzoyl)-N-methylamino-3-(1-pentyl-1,2,3,6-tetrahydropyridin-4-yl)-1H-indole naphthalene-1-sulfonate; 5-(4-fluorobenzoyl)amino-3-(1-(2-pentyl)-1,2,3,6-tetrahydropyridin-4-yl)-1H-indole; 5-(4-fluorobenzoyl)amino-3-(1-(3-pentyl)-1,2,3,6-tetrahydropyridin-4-yl)-1H-indole 5-(4-fluorobenzoyl)amino-3-(1-neopentyl-1,2,3,6-tetrahydropyridin-4-yl)-1H-indole; 5-(4-fluorobenzoyl)amino-3-(1-hexyl-1,2,3,6-tetrahydropyridin-4-yl)-1H-indole; 5-(2-chlorobenzoyl)amino-3-(1-ethyl-1,2,3,6-tetrahydropyridin-4-yl)-1H-indole; 5-(2-chlorobenzoyl)amino-3-(1-propyl-1,2,3,6-tetrahydropyridin-4-yl)-1H-indole; 5-(2-chlorobenzoyl)amino-3-(1-isopropyl-1,2,3,6-tetrahydropyridin-4-yl)-1H-indole; 5-(2-chlorobenzoyl)amino-3-(1-butyl-1,2,3,6-tetrahydropyridin-4-yl)-1H-indole; 5-(2-chlorobenzoyl)amino-3-(1-isobutyl-1,2,3,6-tetrahydropyridin-4-yl)-1H-indole; 5-(2-chlorobenzoyl)amino-3-(1-(sec-butyl)-1,2,3,6-tetrahydropyridin-4-yl)-1H-indole; 5-(2-chlorobenzoyl)amino-3-(1-(tert-butyl)-1,2,3,6-tetrahydropyridin-4-yl)-1H-indole; 5-(2-chlorobenzoyl)-N-ethylamino-3-(1-pentyl-1,2,3,6-tetrahydropyridin-4-yl)-1H-indole; 5-(2-chlorobenzoyl)amino-3-(1-(2-pentyl)-1,2,3,6-tetrahydropyridin-4-yl)-1H-indole; 5-(2-chlorobenzoyl)amino-3-(1-(3-pentyl)-1,2,3,6-tetrahydropyridin-4-yl)-1H-indole; 5-(2-chlorobenzoyl)amino-3-(1-neopentyl-1.2,3,6-tetrahydropyridin-4-yl)-1H-indole; 5-(2-chlorobenzoyl)amino-3-(1-hexyl-1,2,3,6-tetrahydropyridin-4-yl)-1H-indole; 5-(3-bromobenzoyl)amino-3-(1-propyl-1,2,3,6-tetrahydropyridin-4-yl)-1H-indole; 5-(2-iodobenzoyl)-N-propylamino-3-(1-(sec-butyl)-1,2,3,6-tetrahydropyridin-4-yl)-1H-indole; 5-(3-ethylbenzoyl)amino-3-(1-propyl-1,2,3,6-tetrahydropyridin-4-yl)-1H-indole; 5-(4-propylbenzoyl)amino-3-(1-(tert-butyl)-1,2,3,6-tetrahydropyridin-4-yl)-1H-indole; 5-(2-propylbenzoyl)amino-3-(1-neopentyl-1,2,3,6-tetrahydropyridin-4-yl)-1H-indole; 5-(3-butylbenzoyl)amino-3-(1-propyl-1,2,3,6-tetrahydropyridin-4-yl)-1H-indole; 5-(4-ethoxybenzoyl)amino-3-(1-(sec-butyl)-1,2,3,6-tetrahydropyridin-4-yl)-1H-indole; 5-(2-ethoxybenzoyl)-N-isopropylamino-3-(1-(3-pentyl) 1,2,3,6-tetrahydropyridin-4-yl)-1H-indole; 5-(3-propoxybenzoyl)amino-3-(1-(2-pentyl)-1,2,3,6-tetrahydropyridin-4-yl)-1H-indole; 5-(2-butoxybenzoyl)amino-3-(1-ethyl-1,2,3,6-tetrahydropyridin-4-yl)-1H-indole; 5-(4-butoxybenzoyl)amino-3-(1-isobutyl-1,2,3,6-tetrahydropyridin-4-yl)-1H-indole; 5-(3-pentoxybenzoyl)amino-3-(1-propyl-1,2,3,6-tetrahydropyridin-4-yl)-1H-indole; 5-(3-hexyloxybenzoyl)amino-3-(1-(sec-butyl)-1,2,3,6-tetrahydropyridin-4-yl)-1H-indole; 5-(3-heptyloxybenzoyl)amino-3-(1-hexyl-1,2,3,6-tetrahydropyridin-4-yl)-1H-indole; 5-(2-octyloxybenzoyl)amino-3-(1-neopentyl-1,2,3,6-tetrahydropyridin-4-yl)-1H-indole; 5-(4-octyloxybenzoyl)amino-3-(1-(2-pentyl)-1,2,3,6-tetrahydropyridin-4-yl)-1H-indole; 5-(3-octyloxybenzoyl)amino-3-(1-(tert-butyl)-1,2,3,6-tetrahydropyridin-4-yl)-1H-indole; 5-(3-methylthiobenzoyl)amino-3-(1-isopropyl-1,2,3,6-tetrahydropyridin-4-yl)-1H-indole; 5-(4-propylthiobenzoyl)-N-butylamino-3-(1-methyl-1,2,3,6-tetrahydropyridin-4-yl)-1H-indole; 5-(2-propylthiobenzoyl)amino-3-(1-butyl-1,2,3,6-tetrahydropyridin-4-yl)-1H-indole; 5-(3-butylthiobenzoyl)amino-3-(1-hexyl-1,2,3,6-tetrahydropyridin-4-yl)-1H-indole; 5-(2-nitrobenzoyl)amino-3-(1-isopropyl-1,2,3,6-tetrahydropyridin-4-yl)-1H-indole; 5-(2-cyanobenzoyl)amino-3-(1-isopropyl-1,2,3,6-tetrahydropyridin-4-yl)-1H-indole; 5-(2-(dimethylamino)benzoyl)amino-3-(1-neopentyl-1,2,3,6-tetrahydropyridin-4-yl)-1H-indole; 5-(3-(diethylamino)benzoyl)amino-3-(1-(sec-butyl)-1,2,3,6-tetrahydropyridin-4-yl)-1H-indole; 5-(4-(dipropylamino)benzoyl)amino-3-(1-propyl-1,2,3,6-tetrahydropyridin-4-yl)-1H-indole; 5-(2-trifluoromethoxybenzoyl)amino-3-(1-ethyl-1,2,3,6-tetrahydropyridin-4-yl)-1H-indole; 5-(3-trifluoromethoxybenzoyl)amino-3-(1-isobutyl-1,2,3,6-tetrahydropyridin-4-yl)-1H-indole; 5-(3-formylbenzoyl)amino-3-(1-(2-pentyl)-1,2,3,6-tetrahydropyridin-4-yl)-1H-indole; 5-(3-acetylbenzoyl)amino-3-(1-hexyl-1,2,3,6-tetrahydropyridin-4-yl)-1H-indole; 5-(2-(propanoyl)benzoyl)amino-3-(1-butyl-1,2,3,6-tetrahydropyridin-4-yl)-1H-indole; 5-(4-(propanoyl)benzoyl)amino-3-(1-pentyl-1,2,3,6-tetrahydropyridin-4-yl)-1H-indole; 5-(3-(butanoyl)benzoyl)amino-3-(1-methyl-1,2,3,6-tetrahydropyridin-4-yl)-1H-indole; 5-(3-(benzoyl)benzoyl)amino-3-(1-(3-pentyl)-1,2,3,6-tetrahydropyridin-4-yl)-1H-indole; 5-(3-methanesulfonylbenzoyl)amino-3-(1-(sec-butyl)-1,2,3,6-tetrahydropyridin-4-yl)-1H-indole; 5-(3-ethanesulfonylbenzoyl)amino-3-(1-propyl-1,2,3,6-tetrahydropyridin-4-yl)-1H-indole; 5-(2-butanesulfonylbenzoyl)amino-3-(1-ethyl-1,2,3,6-tetrahydropyridin-4-yl)-1H-indole; 5-(3-butanesulfonylbenzoyl)amino-3-(1-isobutyl-1,2,3,6-tetrahydropyridin-4-yl)-1H-indole; 5-(2-phenylbenzoyl)amino-3-(1-(2-pentyl)-1,2,3,6-tetrahydropyridin-4-yl)-1H-indole; 5-(2,3-dibromo)benzoylamino-3-(1-isopropyl-1,2,3,6-tetrahydropyridin-4-yl)-1H-indole; 5-(2-bromo-3-iodo)benzoylamino-3-(1-(2-pentyl)-1,2,3,6-tetrahydropyridin-4-yl)-1H-indole; 5-(3,4-difluoro)benzoylamino-3-(1-butyl-1,2,3,6-tetrahydropyridin-4-yl)-1H-indole; 5-(3-chloro-4-bromo)benzoylamino-3-(1-(3-pentyl)-1,2,3,6-tetrahydropyridin-4-yl)-1H-indole; 5-(2-bromo-4-fluoro)benzoylamino-3-(1-methyl-1,2,3,6-tetrahydropyridin-4-yl)-1H-indole; 5-(2,4-diiodo)benzoylamino-3-(1-(sec-butyl)-1,2,3,6-tetrahydropyridin-4-yl)-1H-indole; 5-(2-chloro-5-iodo)benzoylamino-3-(1-hexyl-1,2,3,6-tetrahydropyridin-4-yl)-1H-indole; 5-(2-fluoro-6-iodo)benzoylamino-3-(1-(tert-butyl)-1,2,3,6-tetrahydropyridin-4-yl)-1H-indole; 5-(3-fluoro-5-chloro)benzoylamino-3-(1-(2-pentyl)-1,2,3,6-tetrahydropyridin-4-yl)-1H-indole; 5-(2-thienoyl)amino-3-(1-ethyl-1,2,3,6-tetrahydropyridin-4-yl)-1H-indole; 5-(2-thienoyl)amino-3-(1-propyl-1,2,3,6-tetrahydropyridin-4-yl)-1H-indole; 5-(2-thienoyl)amino-3-(1-isopropyl-1,2,3,6-tetrahydropyridin-4-yl)-1H-indole; 5-(2-thienoyl)amino-3-(1-butyl-1,2,3,6-tetrahydropyridin-4-yl)-1H-indole; 5-(2-thienoyl)amino-3-(1-isobutyl-1,2,3,6-tetrahydropyridin-4-yl)-1H-indole; 5-(2-thienoyl)amino-3-(1-(sec-butyl)-1,2,3,6-tetrahydropyridin-4-yl)-1H-indole; 5-(2-thienoyl)amino-3-(1-(tert-butyl)-1,2,3,6-tetrahydropyridin-4-yl)-1H-indole; 5-(2-thienoyl)amino-3-(1-pentyl-1,2,3,6-tetrahydropyridin-4-yl)-1H-indole; 5-(2-thienoyl)amino-3-(1-(2-pentyl)-1,2,3,6-tetrahydropyridin-4-yl)-1H-indole; 5-(2-thienoyl)amino-3-(1-(3-pentyl)-1,2,3,6-tetrahydropyridin-4-yl)-1H-indole; 5-(2-thienoyl)amino-3-(1-neopentyl-1,2,3,6-tetrahydropyridin-4-yl)-1H-indole; 5-(2-thienoyl)amino-3-(1-hexyl-1,2,3,6-tetrahydropyridin-4-yl)-1H-indole; 5-(3-thienoyl)amino-3-(1-ethyl-1,2,3,6-tetrahydropyridin-4-yl)-1H-indole; 5-(3-thienoyl)amino-3-(1-propyl-1,2,3,6-tetrahydropyridin-4-yl)-1H-indole; 5-(3-thienoyl)amino-3-(1-isopropyl-1,2,3,6-tetrahydropyridin-4-yl)-1H-indole; 5-(3-thienoyl)amino-3-(1-butyl-1,2,3,6-tetrahydropyridin-4-yl)-1H-indole; 5-(3-thienoyl)amino-3-(1-isobutyl-1,2,3,6-tetrahydropyridin-4-yl)-1H-indole; 5-(3-thienoyl)amino-3-(1-(sec-butyl)-1,2,3,6-tetrahydropyridin-4-yl)-1H-indole; 5-(3-thienoyl)amino-3-(1-(tert-butyl)-1,2,3,6-tetrahydropyridin-4-yl)-1H-indole; 5-(3-thienoyl)amino-3-(1-pentyl-1,2,3,6-tetrahydropyridin-4-yl)-1H-indole; 5-(3-thienoyl)amino-3-(1-(2-pentyl)-1,2,3,6-tetrahydropyridin-4-yl)-1H-indole; 5-(3-thienoyl)amino-3-(1-(3-pentyl)-1,2,3,6-tetrahydropyridin-4-yl)-1H-indole; 5-(3-thienoyl)amino-3-(1-neopentyl-1,2,3,6-tetrahydropyridin-4-yl)-1H-indole; 5-(3-thienoyl)amino-3-(1-hexyl-1,2,3,6-tetrahydropyridin-4-yl)-1H-indole; 5-(2-furoyl)amino-3-(1-ethyl-1,2,3,6-tetrahydropyridin-4-yl)-1H-indole; 5-(2-furoyl)amino-3-(1-propyl-1,2,3,6-tetrahydropyridin-4-yl)-1H-indole; 5-(2-furoyl)amino-3-(1-isopropyl-1,2,3,6-tetrahydropyridin-4-yl)-1H-indole; 5-(2-furoyl)amino-3-(1-butyl-1,2,3,6-tetrahydropyridin-4-yl)-1H-indole; 5-(2-furoyl)amino-3-(1-isobutyl-1,2,3,6-tetrahydropyridin-4-yl)-1H-indole; 5-(2-furoyl)amino-3-(1-(sec-butyl)-1,2,3,6-tetrahydropyridin-4-yl)-1H-indole; 5-(2-furoyl)amino-3-(1-(tert-butyl)piperidin-4-yl)-1H-indole; 5-(2-furoyl)amino-3-(1-pentyl-1,2,3,6-tetrahydropyridin-4-yl)-1H-indole; 5-(2-furoyl)amino-3-(1-(2-pentyl)-1,2,3,6-tetrahydropyridin-4-yl)-1H-indole; 5-(2-furoyl)amino-3-(1-(3-pentyl)-1,2,3,6-tetrahydropyridin-4-yl)-1H-indole; 5-(2-furoyl)amino-3-(1-neopentyl-1,2,3,6-tetrahydropyridin-4-yl)-1H-indole; 5-(2-furoyl)amino-3-(1-hexyl-1,2,3,6-tetrahydropyridin-4-yl)-1H-indole; 5-(3-furoyl)amino-3-(1-ethyl-1,2,3,6-tetrahydropyridin-4-yl)-1H-indole; 5-(3-furoyl)amino-3-(1-propyl-1,2,3,6-tetrahydropyridin-4-yl)-1H-indole; 5-(3-furoyl)amino-3-(1-isopropyl-1,2,3,6-tetrahydropyridin-4-yl)-1H-indole; 5-(3-furoyl)amino-3-(1-butyl-1,2,3,6-tetrahydropyridin-4-yl)-1H-indole; 5-(3-furoyl)amino-3-(1-isobutyl-1,2,3,6-tetrahydropyridin-4-yl)-1H-indole; 5-(3-furoyl)amino-3-(1-(sec-butyl)-1,2,3,6-tetrahydropyridin-4-yl)-1H-indole; 5-(3-furoyl)amino-3-(1-(tert-butyl)-1,2,3,6-tetrahydropyridin-4-yl)-1H-indole; 5-(3-furoyl)amino-3-(1-pentyl-1,2,3,6-tetrahydropyridin-4-yl)-1H-indole; 5-(3-furoyl)amino-3-(1-(2-pentyl)-1,2,3,6-tetrahydropyridin-4-yl)-1H-indole naphthalene-2-sulfonate; 5-(3-furoyl)amino-3-(1-(3-pentyl)-1,2,3,6-tetrahydropyridin-4-yl)-1H-indole; 5-(3-furoyl)amino-3-(1-neopentyl-1,2,3,6-tetrahydropyridin-4-yl)-1H-indole; 5-(3-furoyl)amino-3-(1-hexyl-1,2,3,6-tetrahydropyridin-4-yl)-1H-indole; 5-(propanoyl)amino-3-(1-neopentylpiperidin-4-yl)-1H-indole mandalate; 5-(2-methylpropanoyl)amino-3-(1-(3-pentyl)piperidin-4-yl)-1H-indole; 5-(2-methyl-4-butyn-1-oyl)amino-3-(1-(tert-butyl)piperidin-4-yl)-1H-indole; 5-(2-methylbutanoyl)-N-methylamino-3-(1-ethylpiperidin-4-yl)-1H-indole; 5-(hex-3-enoyl)amino-3-(1-propylpiperidin-4-yl)-1H-indole; 5-(cyclohexaneacetyl)amino-3-(1-isopropylpiperidin-4-yl)-1H-indole; 5-(cycloheptylcarbonyl)amino-3-(1-butylpiperidin-4-yl)-1H-indole; 5-(4-phenylbutanoyl)amino-3-(1-isopropylpiperidin-4-yl)-1H-indole; 5-(5-phenylpentanoyl)amino-3-(1-(2-pentyl)piperidin-4-yl)-1H-indole; 5-(3-phenoxypropanoyl)amino-3-(1-neopentylpiperidin-4-yl)-1H-indole; 5-(5-phenoxypentanoyl)amino-3-(1-neopentylpiperidin-4-yl)-1H-indole; 5-(3-propoxypropanoyl)amino-3-(1-isopropylpiperidin-4-yl)-1H-indole; 5-(5-methoxypentanoyl)amino-3-(1-isopropylpiperidin-4-yl)-1H-indole; 5-((3-propoxycarbonyl)propanoyl)amino-3-(1-isobutylpiperidin-4-yl)-1H-indole; 5-((5-methoxycarbonyl)pentanoyl)amino-3-(1-isopropylpiperidin-4-yl)-1H-indole; 5-(benzoyl-N-ethyl)amino-3-(1-ethylpiperidin-4-yl)-1H-indole; 5-benzoylamino-3-(1-propylpiperidin-4-yl)-1H-indole; 5-benzoylamino-3-(1-isopropylpiperidin-4-yl)-1H-indole; 5-benzoylamino-3-(1-butylpiperidin-4-yl)-1H-indole; 5-benzoylamino-3-(piperidin-4-yl)-1H-indole; 5-benzoylamino-3-(1-isobutylpiperidin-4-yl)-1H-indole; 5-benzoylamino-3-(1-(sec-butyl)piperidin-4-yl)-1H-indole; 5-benzoylamino-3-(1-(tert-butyl)piperidin-4-yl)-1H-indole hydrochloride; 5-benzoyl-N-propylamino-3-(1-pentylpiperidin-4-yl)-1H-indole; 5-benzoylamino-3-(1-(2-pentyl)piperidin-4-yl)-1H-indole; 5-benzoylamino-3-(1-(3-pentyl)piperidin-4-yl)-1H-indole; 5-benzoylamino-3-(1-neopentylpiperidin-4-yl)-1H-indole; 5-benzoylamino-3-(1-hexylpiperidin-4-yl)-1H-indole; 5-(4-fluorobenzoyl)amino-3-(1-ethylpiperidin-4-yl)-1H-indole; 5-(4-fluorobenzoyl)amino-3-(1-propylpiperidin-4-yl)-1H-indole 5-(4-fluorobenzoyl)amino-3-(1-isopropylpiperidin-4-yl)-1H-indole; 5-(4-fluorobenzoyl)amino-3-(1-butylpiperidin-4-yl)-1H-indole; 5-(4-fluorobenzoyl)amino-3-(1-isobutylpiperidin-4-yl)-1H-indole; 5-(4-fluorobenzoyl)amino-3-(1-(sec-butyl)piperidin-4-yl)-1H-indole; 5-(4-fluorobenzoyl)amino-3-(1-(tert-butyl)piperidin-4-yl)-1H-indole; 5-(4-fluorobenzoyl)amino-3-(1-pentylpiperidin-4-yl)-1H-indole; 5-(4-fluorobenzoyl)amino-3-(1-(2-pentyl)piperidin-4-yl)-1H-indole; 5-(4-fluorobenzoyl)amino-3-(1-(3-pentyl)piperidin-4-yl)-1H-indole; 5-(4-fluorobenzoyl)amino-3-(1-neopentylpiperidin-4-yl)-1H-indole fumarate; 5-(4-fluorobenzoyl)amino-3-(1-hexylpiperidin-4-yl)-1H-indole; 5-(2-chlorobenzoyl)amino-3-(1-ethylpiperidin-4-yl)-1H-indole; 5-(2-chlorobenzoyl)amino-3-(1-propylpiperidin-4-yl)-1H-indole; 5-(2-chlorobenzoyl)amino-3-(1-isopropylpiperidin-4-yl)-1H-indole; 5-(2-chlorobenzoyl)amino-3-(1-butylpiperidin-4-yl)-1H-indole; 5-(2-chlorobenzoyl)amino-3-(1-isobutylpiperidin-4-yl)-1H-indole; 5-(2-chlorobenzoyl)amino-3-(1-(sec-butyl)piperidin-4-yl)-1H-indole; 5-(2-chlorobenzoyl)amino-3-(1-(tert-butyl)piperidin-4-yl)-1H-indole; 5-(2-chlorobenzoyl)amino-3-(1-pentylpiperidin-4-yl)-1H-indole; 5-(2-chlorobenzoyl)amino-3-(1-(2-pentyl)piperidin-4-yl)-1H-indole; 5-(2-chlorobenzoyl)amino-3-(1-(3-pentyl)piperidin-4-yl)-1H-indole; 5-(2-chlorobenzoyl)amino-3-(1-neopentylpiperidin-4-yl)-1H-indole; 5-(2-chlorobenzoyl)amino-3-(1-hexylpiperidin-4-yl)-1H-indole; 5-(2-bromobenzoyl)amino-3-(1-butylpiperidin-4-yl)-1H-indole; 5-(4-ethylbenzoyl)amino-3-(1-ethylpiperidin-4-yl)-1H-indole; 5-(2-ethylbenzoyl)amino-3-(1-isobutylpiperidin-4-yl)-1H-indole; 5-(3-propylbenzoyl)amino-3-(1-(2-pentyl)piperidin-4-yl)-1H-indole; 5-(4-butylbenzoyl)amino-3-(1-methylpiperidin-4-yl)-1H-indole 5-(2-butylbenzoyl)amino-3-(1-butylpiperidin-4-yl)-1H-indole; 5-(3-ethoxybenzoyl)amino-3-(1-pentylpiperidin-4-yl)-1H-indole; 5-(2-propoxybenzoyl)amino-3-(1-(tert-butyl)pjperidin-4-yl)-1H-indole; 5-(3-butoxybenzoyl)amino-3-(1-isopropylpiperidin-4-yl)-1H-indole; 5-(4-pentyloxybenzoyl)amino-3-(1-methylpiperidin-4-yl)-1H-indole; 5-(2-pentyloxybenzoyl)amino-3-(1-butylpiperidin-4-yl)-1H-indole; 5-(2-hexyloxybenzoyl)amino-3-(1-pentylpiperidin-4-yl)-1H-indole; 5-(4-hexyloxybenzoyl)amino-3-(1-(3-pentyl)piperidin-4-yl)-1H-indole; 5-(2-methylthiobenzoyl)amino-3-(1-isobutylpiperidin-4-yl)-1H-indole; 5-(2-ethylthiobenzoyl)amino-3-(1-ethylpiperidin-4-yl)-1H-indole; 5-(3-propylthiobenzoyl)amino-3-(1-propylpiperidin-4-yl)-1H-indole; 5-(3-nitrobenzoyl)amino-3-(1-isobutylpiperidin-4-yl)-1H-indole; 5-(3-cyanobenzoyl)amino-3-(1-(2-pentyl)piperidin-4-yl)-1H-indole; 5-(4-(dimethylamino)benzoyl)amino-3-(1-isobutylpiperidin-4-yl)-1H-indole; 5-(2-(diethylamino)benzoyl)-N-propylamino-3-(1-butylpiperidin-4-yl)-1H-indole; 5-(4-(diethylamino)benzoyl)amino-3-(1-pentylpiperidin-4-yl)-1H-indole; 5-(3-(dibutylamino)benzoyl)amino-3-(1-methylpiperidin-4-yl)-1H-indole; 5-(4-trifluoromethoxybenzoyl)amino-3-(1-isopropylpiperidin-4-yl)-1H-indole; 5-(4-(formyl)benzoyl)amino-3-(1-(sec-butyl)piperidin-4-yl)-1H-indole; 5-(2-(formyl)benzoyl)amino-3-(1-neopentylpiperidin-4-yl)-1H-indole; 5-(2-(acetyl)benzoyl)amino-3-(1-(3-pentyl)piperidin-4-yl)-1H-indole; 5-(3-(propanoyl)benzoyl)amino-3-(1-pentylpiperidin-4-yl)-1H-indole; 5-(3-(butanoyl)benzoyl)amino-3-(1-methylpiperidin-4-yl)-1H-indole; 5-(2-(butanoyl)benzoyl)amino-3-(1-neopentylpiperidin-4-yl)-1H-indole; 5-(2-(benzoyl)benzoyl)amino-3-(1-pentylpiperidin-4-yl)-1H-indole; 5-(2-(methanesulfonyl)benzoyl)amino-3-(1-butylpiperidin-4-yl)-1H-indole; 5-(3-(propanesulfonyl)benzoyl)amino-3-(1-methylpiperidin-4-yl)-1H-indole; 5-(2-butanesulfonylbenzoyl)amino-3-(1-isopropylpiperidin-4-yl)-1H-indole; 5-(3-phenylbenzoyl)amino-3-(1-(tert-butyl)piperidin-4-yl)-1H-indole; 5-(2,3-dibromo)benzoyl-N-isopropylamino-3-(1-isopropylpiperidin-4-yl)-1H-indole 5-(2-bromo-3-iodo)benzoylamino-3-(1-(2-pentyl)piperidin-4-yl)-1H-indole; 5-(3,4-difluoro)benzoylamino-3-(1-butylpiperidin-4-yl)-1H-indole; 5-(3-chloro-4-bromo)benzoylamino-3-(1-(3-pentyl)piperidin-4-yl)-1H-indole; 5-(2-bromo-4-fluoro)benzoylamino-(3-(1-methylpiperidin-4-yl)-1H-indole; 5-(2,4-diiodo)benzoylamino-(3-(1-(sec-butyl)piperidin-4-yl)-1H-indole; 5-(2-chloro-5-iodo)benzoylamino-(3-(1-hexylpiperidin-4-yl)-1H-indole; 5-(2-fluoro-6-iodo)benzoylamino-(3-(1-(tert-butyl)piperidin-4-yl)-1H-indole; 5-(3-fluoro-5-chloro)benzoylamino-3-(1-(2-pentyl)piperidin-4-yl)-1H-indole; 5-(2-thienoyl)-N-butylamino-3-(1-ethylpiperidin-4-yl)-1H-indole; 5-(2-thienoyl)amino-3-(1-propylpiperidin-4-yl)-1H-indole; 5-(2-thienoyl)amino-3-(1-isopropylpiperidin-4-yl)-1H-indole; 5-(2-thienoyl)amino-3-(1-butylpiperidin-4-yl)-1H-indole; 5-(2-thienoyl)amino-3-(1-isobutylpiperidin-4-yl)-1H-indole; 5-(2-thienoyl)amino-3-(1-(sec-butyl)pjperidin-4-yl)-1H-indole; 5-(2-thienoyl)amino-3-(1-(tert-butyl)pjperidin-4-yl)-1H-indole; 5-(2-thienoyl)amino-3-(1-pentylpiperidin-4-yl)-1H-indole; 5-(2-thienoyl)amino-3-(1-(2-pentyl)piperidin-4-yl)-1H-indole; 5-(2-thienoyl)amino-3-(1-(3-pentyl)piperidin-4-yl)-1H-indole; 5-(2-thienoyl)amino-3-(1-neopentylpiperidin-4-yl)-1H-indole; 5-(2-thienoyl)amino-3-(1-hexylpiperidin-4-yl)-1H-indole; 5-(3-thienoyl)amino-3-(1-ethylpiperidin-4-yl)-1H-indole; 5-(3-thienoyl)amino-3-(1-propylpiperidin-4-yl)-1H-indole; 5-(3-thienoyl)amino-3-(1-isopropylpiperidin-4-yl)-1H-indole; 5-(3-thienoyl)amino-3-(1-butylpiperidin-4-yl)-1H-indole; 5-(3-thienoyl)amino-3-(1-isobutylpiperidin-4-yl)-1H-indole; 5-(3-thienoyl)amino-3-(1-(sec-butyl)piperidin-4-yl)-1H-indole; 5-(3-thienoyl)amino-3-(1-(tert-butyl)piperidin-4-yl)-1H-indole; 5-(3-thienoyl)amino-3-(1-pentylpiperidin-4-yl)-1H-indole; 5-(3-thienoyl)amino-3-(1-(2-pentyl)piperidin-4-yl)-1H-indole; 5-(3-thienoyl)amino-3-(1-(3-pentyl)piperidin-4-yl)-1H-indole; 5-(3-thienoyl)amino-3-(1-neopentylpiperidin-4-yl)-1H-indole; 5-(3-thienoyl)amino-3-(1-hexylpiperidin-4-yl)-1H-indole; 5-(2-furoyl)amino-3-(1-ethylpiperidin-4-yl)-1H-indole; 5-(2-furoyl)amino-3-(1-propylpiperidin-4-yl)-1H-indole; 5-(2-furoyl)amino-3-(1-isopropylpiperidin-4-yl)-1H-indole; 5-(2-furoyl)amino-3-(1-butylpiperidin-4-yl)-1H-indole; 5-(2-furoyl)amino-3-(1-isobutylpiperidin-4-yl)-1H-indole; 5-(2-furoyl)amino-3-(1-(sec-butyl)pioeridin-4-yl)-1H-indole; 5-(2-furoyl)amino-3-(1-(tert-butyl)piperidin-4-yl)-1H-indole; 5-(2-furoyl)amino-3-(1-pentylpiperidin-4-yl)-1H-indole; 5-(2-furoyl)amino-3-(1-(2-pentyl)piperidin-4-yl)-1H-indole; 5-(2-furoyl)amino-3-(1-(3-pentyl)piperidin-4-yl)-1H-indole; 5-(2-furoyl)amino-3-(1-neopentylpiperidin-4-yl)-1H-indole; 5-(2-furoyl)amino-3-(1-hexylpiperidin-4-yl)-1H-indole; 5-(3-furoyl)amino-3-(1-ethylpiperidin-4-yl)-1H-indole; 5-(3-furoyl)amino-3-(1-propylpiperidin-4-yl)-1H-indole; 5-(3-furoyl)amino-3-(1-isopropylpiperidin-4-yl)-1H-indole; 5-(3-furoyl)amino-3-(1-butylpiperidin-4-yl)-1H-indole; 5-(3-furoyl)amino-3-(1-isobutylpiperidin-4-yl)-1H-indole; 5-(3-furoyl)amino-3-(1-(sec-butyl)piperidin-4-yl)-1H-indole; 5-(3-furoyl)amino-3-(1-(tert-butyl)piperidin-4-yl)-1H-indole; 5-(3-furoyl)amino-3-(1-pentylpiperidin-4-yl)-1H-indole; 5-(3-furoyl)amino-3-(1-(2-pentyl)piperidin-4-yl)-1H-indole; 5-(3-furoyl)amino-3-(1-(3-pentyl)piperidin-4-yl)-1H-indole; 5-(3-furoyl)amino-3-(1-neopentylpiperidin-4-yl)-1H-indole; 5-(3-furoyl)amino-3-(1-hexylpiperidin-4-yl)-1H-indole; N-[pyridin-2-yl]-5-carboxamido-3-(1-methyl-1,2,5,6-tetrahydropyridin-4-yl)-1H-indole; N-[fur-3-yl]-5-carboxamido-3-(1-methyl-1,2,5,6-tetrahydropyridin-4-yl)-1H-indole; N-[pyrazol-3-yl]-5-carboxamido-3-(1-methyl-1,2,5,6-tetrahydropyridin-4-yl)-1H-indole; N-[thiazol-2-yl]-5-carboxamido-3-(1-methyl-1,2,5,6-tetrahydropyridin-4-yl)-1H-indole; N-tquinolin-4-yl]-5-carboxamido-3-(1-methyl-1,2,5,6-tetrahydropyridin-4-yl)-1H-indole; N-[imidazol-4-yl]-5-carboxamido-3-(1-methylpiperidin-4-yl)-1H-indole; N-[fur-3-yl]-5-carboxamido-3-(1-methyl-1,2,5,6-tetrahydropyridin-4-yl)-1H-indole; N-[pyrimidin-5-yl]-5-carboxamido-3-(1-methylpiperidin-4-yl)-1H-indole; N-[indol-2-yl]-5-carboxamido-3-(1-methylpiperidin-4-yl)-1H-indole; and N-[isoxazol-5-yl]-5-carboxamido-3-(1-methylpiperidin-4-yl)-1H-indole.

The following HTR_{1F} agonists are disclosed in PCT publication No. WO 97/13512: N-[2-methyl-3-(2-[N′,N′-diethylamino]ethyl)-1H-indol-5-yl]-4-propanesulfonylbenzamide hydrochloride; N-[2-ethyl-3-(2-[N′-methyl-N′-isopropylamino]ethyl)-1H-indol-5-yl]-3-ethylthiobenzamidehydroiodide; N-[2-propyl-3-(2-[N′-ethyl-N′-cyclopentylpropylamino]-ethyl)-1H-indol-5-yl]-4-ethyl-2-propoxycarbonylbenzamide hydrobromide; N-[2-isopropyl-3-(2-[N′,N′-dibutylamino]ethyl)-1H-indol-5-yl]-4-(N″,N″-dipropylamino)benzamide oxalate; N-[2-n-butyl-3-(2-[N′-methyl-N′-benzylamino]ethyl)-1H-indol-5-yl]-4-isopropylbenzamide sulfate; N-[2-isobutyl-3-(2-[N′-methyl-N′-cyclopropylmethyl-amino]ethyl)-1H-indol-5-yl]-4-(N″-ethyl-N″-butanoyl)amino-benzamide acetate; N-[2-s-butyl-3-(2-[N′-methyl-N′-(2-[1-propylpyrazol-4-yl]ethyl)amino]ethyl)-1H-indol-5-yl]-2-nitrobenzamide phosphate; N-[2-t-butyl-3-(2-[N′-methyl-N′-(1-ethylpyrazol-4-ylmethyl)amino]ethyl)-1H-indol-5-yl]-4-isobutylsulfonyl-benzamidemalonate; N-[2-methyl-3-(2-[N′-methyl-N′-isobutylamino]ethyl)-1H-indol-5-yl]-3-ethylbenzamide tartrate; N-[2-methyl-3-(2-[N′-methyl-N′-(2-[pyridin-4-yl]-ethyl)amino]ethyl)-1H-indol-5-yl]-3-t-butoxybenzamide citrate; N-[2-methyl-3-(2-[N″-methyl-N′-s-butylamino]ethyl)-1H-indol-5-yl]-4-formylamino-2-propylbenzamide 4-toluenesulfonate; N-[2-methyl-3-(2-[N′-methyl-N′-(2-[4-bromopyridin-3-yl]-ethyl)amino]ethyl)-1H-indol-5-yl]-3-t-butoxybenzamide benzoate; N-[2-methyl-3-(2-[N′-methyl-N′-(2-[1-isopropylpyrazol-4-yl]ethyl)amino]ethyl)-1H-indol-5-yl]-4-isopropylthio-benzamide fumarate; N-[2-ethyl-3-(2-[N′,N′-diethylamino]ethyl)-1H-indol-5-yl]-4-fluorobenzamidenaphthalene-1-sulfonate; N-[2-ethyl-3-(2-[N′-methyl-N′-isopropylamino]ethyl)-1H-indol-5-yl]-4-fluorobenzamide; N-[2-propyl-3-(2-[N′-ethyl-N′-cyclopentylpropylamino]-ethyl)-1H-indol-5-yl]-4-bromobenzamidephthalate; N-[2-isopropyl-3-(2-[N′,N′-dibutylamino]ethyl)-1H-indol-5-yl]-4-fluorobenzamidemethanesulfonate; N-[2-n-butyl-3-(2-[N′-methyl-N′-benzylamino]ethyl)-1H-indol-5-yl]-4-fluorobenzamide; N-[2-isobutyl-3-(2-[N′-methyl-N′-cyclopropylmethyl-amino]ethyl)-1H-indol-5-yl]-4-iodobenzamide naphthalene-1-sulfonate; N-[2-s-butyl-3-(2-[N′-methyl-N′-(2-[1-propylpyrazol-4-yl]ethyl)amino]ethyl)-1H-indol-5-yl]-4-fluorobenzamide ditoluoyltartrate; N-[2-1-butyl-3-(2-[N′-methyl-N′-(1-ethylpyrazol-4-ylmethyl)amino]ethyl)-1H-indol-5-yl]-4-fluorobenzamide; N-[2-methyl-3-(2-[N′-methyl-N′-isobutylamino]ethyl)-1H-indol-5-yl]-2-bromo-4-fluorobenzamide; N-[2-methyl-3-(2-[N′-methyl-N′-(2-[pyridin-4-yl]-ethyl)amino]ethyl)-1H-indol-5-yl]-4-fluorobenzamide; N-[2-methyl-3-(2-[N′-methyl-N′-s-butylamino]ethyl)-1H-indol-5-yl]-isobutyramide; N-[2-methyl-3-(2-[N′-methyl-N′-(2-[pyridin-4-yl]-ethyl)amino]ethyl)-1H-indol-5-yl]-4-fluorobenzamidemalonate; N-[2-methyl-3-(2-[N′-methyl-N′-(2-[1-isopropylpyrazol-4-yl]ethyl)amino]ethyl)-1H-indol-5-yl]-butyramidemandelate; N-[3-(2-[N′-methyl-N′-([4-bromothien-2-yl]-methyl)amino]ethyl)-1H-indol-5-yl]-4-fluorobenzamide hydrochloride; N-[2-ethyl-3-(2-[N′-ethyl-N′-(2-[3-methylthiobenzofur-5-yl]ethyl)amino]ethyl)-1H-indol-5-yl]pyridine-2-carboxamide; N-[2-propyl-3-(2-[N′-isopropyl-N′-(3-[isobenzofur-2-yl]-propyl)amino]ethyl)-1H-indol-5-yl]-4-fluorobenzamide; N-[2-methyl-3-(2-[N′-butyl-N′-([pyrrol-3-yl]methyl)-amino]ethyl)-1H-indol-5-yl]-4-fluorobenzamidemaleate; N-[2-methyl-3-(2-[N′-methyl-N′-([5-cyanoimidazol-2-yl]-methyl)amino]ethyl)-1H-indol-5-yl]acetamide trifluoroacetate; N-[2-methyl-3-(2-[N′-methyl-N′-([6-carboxamidopyrazin-2-yl]methyl)amino]ethyl)-1H-indol-5-yl]propanamide; N-[2-methyl-3-(2-[N′-methyl-N′-([5-nitropyrimidin-2-yl]-methyl)amino]ethyl)-1H-indol-5-yl]-2-propanamide; N-[2-methyl-3-(2-[N′-methyl-N′-([5-dimethylaminopyridazin-3-yl]methyl)amino]ethyl)-1H-indol-5-yl]butyramide benzoate; N-[2-methyl-3-(2-[N′-methyl-N′-([indazol-5-yl]methyl)-amino]ethyl)-1H-indol-5-yl]pentanamide; N-[2-methyl-3-(2-[N′-methyl-N′-([quinolin-4-yl]methyl)-amino]ethyl)-1H-indol-5-yl]cyclopropanecarboxamide; N-[2-methyl-3-(2-[N′-methyl-N′-([isoquinolin-7-yl]-methyl)amino]ethyl)-1H-indol-5-yl]cyclobutanecarboxamide; N-[2-methyl-3-(2-[N′-methyl-N′-([quinoxalin-2-yl]-methyl)amino]ethyl)-1H-indol-5-yl]cyclopentanecarboxamide hexanoate; N-[2-methyl-3-(2-[N′-methyl-N′-([quinazolin-5-yl]-methyl)amino]ethyl)-1H-indol-5-yl]cyclohexanecarboxamide; N-[2-methyl-3-(2-[N′-methyl-N′-([thiazol-2-yl]methyl)-amino]ethyl)-1H-indol-5-yl]cycloheptanecarboxamide; N-[2-methyl-3-(2-[N′-methyl-N′-([2-aminobenzothiazol-5-yl]methyl)amino]ethyl)-1H-indol-5-yl]-4-fluorobenzamide trifluoromethanesulfonate; N-[2-methyl-3-(2-[N′-methyl-N′-([oxazol-5-yl]methyl)-amino]ethyl)-1H-indol-5-yl]-3-iodobenzamide; N-[2-methyl-3-(2-[N′-methyl-N′-([6-nitrobenzoxazol-2-yl]methyl)amino]ethyl)-1H-indol-5-yl]-2-chlorobenzamide hydrobromide; N-[2-methyl-3-(2-[N′-methyl-N′—([1,4-benzodioxan-6-yl]-methyl)amino]ethyl)-1H-indol-5-yl]-2-chloropyridine-3-carboxamide; N-[2-isopropyl-3-(2-[N′-methyl-N′-([isoxazol-4-yl]-methyl)amino]ethyl)-1H-indol-5-yl]benzamide; N-[2-methyl-3-(2-[N′-methyl-N′-([benzisoxazol-3-yl]-methyl)amino]ethyl)-1H-indol-5-yl]thiophene-2-carboxamide; N-[2-methyl-3-(2-[N′-methyl-N′—([1,3,4-oxadiazol-2-yl]-methyl)amino]ethyl)-1H-indol-5-yl]furan-3-carboxamide; N-[2-methyl-3-(2-[N′-methyl-N′—([1,2,3-triazol-4-yl]-methyl)amino]ethyl)-1H-indol-5-yl]-4-fluorobenzamide tosylate; N-[3-(2-[N′-methyl-N′-((4-bromothien-2-yl)meth-yl)amino]ethyl)-1H-indol-5-yl)]-4-fluorobenzamide hydrochloride; N-[2-ethyl-3-(2-[N′-ethyl-N′-((3-methylthiobenzofur-5-yl)ethyl)amino]ethyl)-1H-indol-5-yl]pyridine-2-carboxamide; N-[2-propyl-3-(2-[N′-isopropyl-N′-1-((isobenzofur-2-yl)prop-3-yl)amino]ethyl)-1H-indol-5-yl]-4-fluorobenzamide; N-[2-methyl-3-(2-[N′-butyl-N′-(pyrrol-3-yl)methyl)-amino]ethyl)-1H-indol-5-yl]-4-fluorobenzamidemaleate; N-[2-methyl-3-(2-[N′-methyl-N′-((5-cyanoimidazol-2-yl)methyl)amino]ethyl)-1H-indul-5-yl]-4-acetamide trifluoroacetate; N-[2-methyl-3-(2-[N′-methyl-N′-((6-carboxamidopyrazin-2-yl)methyl)amino]ethyl)-1H-indol-5-yl]propanamide; N-[2-methyl-3-(2-[N′-methyl-N′-((5-nitropyrimidin-2-yl)methyl)amino]ethyl)-1H-indol-5-yl]-2-propanamide; N-[2-methyl-3-(2-[N′-methyl-N′-((5-dimethylaminopyrida-zin-3-yl)methyl)amino]ethyl)-1H-indol-5-yl]butyramide benzoate; N-[2-methyl-3-(2-[N′-methyl-N′-((indazol-5-yl)meth-yl)amino]ethyl)-1H-indol-5-yl]pentanamide; N-[2-methyl-3-(2-[N′-methyl-N′-((quinolin-4-yl)methyl)-amino]ethyl)-1H-indol-5-yl]cyclopropanecarboxamide; N-[2-methyl-3-(2-[N′-methyl-N′-((isoquinolin-7-yl)meth-yl)amino]ethyl)-1H-indol-5-yl]cyclobutanecarboxamide; N-[2-methyl-3-(2-[N′-methyl-N′-((quinoxalin-2-yl)-methyl)amino]ethyl)-1H-indol-5-yl]cyclopentanecarboxamide acetate; N-[2-methyl-3-(2-[N′-methyl-N′-((quinazolin-5-yl)-methyl)amino]ethyl)-1H-indol-5-yl]cyclohexanecarboxamide; N-[2-methyl-3-(2-[N′-methyl-N′-((thiazol-2-yl)methyl)-amino]ethyl)-1H-indol-5-yl]cycloheptanecarboxamide; N-[2-methyl-3-(2-[N′-methyl-N′-((2-aminobenzothiazol-5-yl)methyl)amino]ethyl)-1H-indol-5-yl]-4-fluorobenzamide trifluoromethanesulfonate; N-[2-methyl-3-(2-[N′-methyl-N′-(2-[pyridin-4-yl]-ethyl)amino]ethyl)-1H-indol-5-yl]-N″-ethylurea; N-[2-methyl-3-(2-[N′-methyl-N′-s-butylamino]ethyl)-1H-indol-5-yl]-N″-isopropylurea; N-[2-methyl-3-(2-[N′-methyl-N′-(2-[pyridin-4-yl]-ethyl)amino]ethyl)-1H-indol-5-yl]-N″-[(3-methoxy)phenyl]urea malonate; N-[3-(2-[N′-(2-[1-isopropylpyrazol-4-yl]ethyl)amino]ethyl)-1H-indol-5-yl]-N″-[(2-ethoxy)phenyl]-ureamandelate; N-[3-(2-[N′-methyl-N′-([4-bromothien-2-yl]-methyl)amino]ethyl)-1H-indol-5-yl]-N″-[(4-isopropoxy)phenyl]-urea hydrochloride; N-[2-ethyl-3-(2-[N′-(2-[3-methylthiobenzofur-5-yl]ethyl)amino]ethyl)-1H-indol-5-yl]-N″-[2,3-dibromophenyl]-urea; N-[2-propyl-3-(2-[N′-isopropyl-N′-(3-[isobenzofur-2-yl]-propyl)amino]ethyl)-1H-indol-5-yl]-N″-[(2-bromo-3-iodo)phen-yl]urea; N-[2-methyl-3-(2-[N′-butyl-N′-([pyrrol-3-yl]methyl)-amino]ethyl)-1H-indol-5-yl]-N″-benzylureamaleate; N-[2-methyl-3-(2-[N′-methyl-N′-([5-cyanoimidazol-2-yl]-methyl)amino]ethyl)-1H-indol-5-yl]-N″-phenethylurea trifluoroacetate; N-[2-methyl-3-(2-[N′-methyl-N′-([6-carboxamidopyrazin-2-yl]methyl)amino]ethyl)-1H-indol-5-yl]-N″-[4-phenbutyl]urea; N-[2-methyl-3-(2-[N′-methyl-N′-([5-nitropyrimidin-2-yl]-methyl)amino]ethyl)-1H-indol-5-yl]-N″-[(2-trifluoromethyl)-phenyl]urea; N-[2-methyl-3-(2-[N′-methyl-N′-([5-dimethylaminopyrida-zin-3-yl]methyl)amino]ethyl)-1H-indol-5-yl]-N″-[(3-phenyl)-phenyl]urea benzoate; 1-{[2-methyl-3-(2-[N′-methyl-N′-([indazol-5-yl]methyl)-amino]ethyl)-1H-indol-5-yl]carbonyl}pyrrolidine; 1-{[2-methyl-3-(2-[N′-methyl-N′-([quinolin-4-yl]methyl)-amino]ethyl)-1H-indol-5-yl]carbonyl}piperidine; 1-{[2-methyl-3-(2-[N′-methyl-N′-([isoquinolin-7-yl]-methyl)amino]ethyl)-1H-indol-5-yl]carbonyl}piperazine; 1-{[2-methyl-3-(2-[N′-methyl-N′-([quinoxalin-2-yl]-methyl)amino]ethyl)-1H-indol-5-yl]carbonyl}-4-methylpipera-zine hexanoate; 1-{[2-isopropyl-3-(2-[N′-methyl-N′-([quinazolin-5-yl]-methyl)amino]ethyl)-1H-indol-5-yl]carbonyl}-4-phenylpiperazine; 1-{[3-(2-[N′-([thiazol-2-yl]methyl)amino]ethyl)-1H-indol-5-yl]carbonyl}-4-benzylpiperazine; 1-{[2-methyl-3-(2-[N′-([2-aminobenzothiazol-5-yl]-methyl)amino]ethyl)-1H-indol-5-yl]carbonyl}-4-(2,4-dichlorophenyl)piperazine trifluoromethanesulfonate; 1-{[3-(2-[N′-methyl-N′-([oxazol-5-yl]methyl)-amino]ethyl)-1H-indol-5-yl]carbonyl}morpholine; 1-{[2-methyl-3-(2-[N′-methyl-N′-([6-nitrobenzoxazol-2-yl]methyl)amino]ethyl)-1H-indol-5-yl]carbonyl}thiomorpholine hydrobromide; N-[2-methyl-3-(2-[N′-methyl-N′-(2-[pyridin-4-yl]-ethyl)amino]ethyl)-1H-indol-5-yl]-N″-ethylthiourea; N-[2-methyl-3-(2-[N′-methyl-N′-s_-butylamino]ethyl)-1H-indol-5-yl]-N″-isopropylthiourea; N-[2-methyl-3-(2-[N′-methyl-N′-(2-[pyridin-4-yl]-ethyl)amino]ethyl)-1H-indol-5-yl]-N″-[(3-methoxy)phenyl]thioureamalonate; N-[2-methyl-3-(2-[N′-methyl-N′-(2-[1-isopropylpyrazol-4-yl]ethyl)amino]ethyl)-1H-indol-5-yl]-N″-[(2-ethoxy)phenyl]-thioureamandelate; N-[2-phenyl-3-(2-[N′-methyl-N′-([4-bromothien-2-yl]-methyl)amino]ethyl)-1H-indol-5-yl]-N″-[(4-isopropoxy)phenyl]-thiourea hydrochloride; N-[2-ethyl-3-(2-[N′-ethyl-N′-(2-[3-methylthiobenzofur-5-yl]ethyl)amino]ethyl)-1H-indol-5-yl]-N″-[2,3-dibromophenyl]-thiourea; N-[2-propyl-3-(2-[N′-isopropyl-N′-(3-[isobenzofur-2-yl]•propyl)amino]ethyl)-1H-indol-5-yl]-N″-[(2-bromo-3-iodo)phen-yl]thiourea; N-[3-(2-[N′-([pyrrol-3-yl]methyl)amino]ethyl)-1H-indol-5-yl]-N″-benzylthioureamaleate; N-[3-(2-[N′-methyl-N′-([5-cyanoimidazol-2-yl]methyl)-amino]ethyl)-1H-indol-5-yl]-N″-phenethylthiourea trifluoroacetate; N-[2-methyl-3-(2-[N′-([6-carboxamidopyrazin-2-yl]-methyl)amino]ethyl)-1H-indol-5-yl]-N″-[4-phenbutyl]thiourea; N-[2-methyl-3-(2-[N′-methyl-N′-([5-nitropyrimidin-2-yl]-methyl)amino]ethyl)-1H-indol-5-yl]-N″-[(2-trifluoromethyl)-phenyl]thiourea; N-[2-methyl-3-(2-[N′-methyl-N′-([5-dimethylaminopyrida-zin-3-yl]methyl)amino]ethyl)-1H-indol-5-yl]-N″-[(3-phenyl)-phenyl]thioureabenzoate; 1-{N-[2-methyl-3-(2-[N′-methyl-N′-([indazol-5-yl]-methyl)amino]ethyl)-1H-indol-5-yl]aminothiocarbonyl}pyrrolidine; 1-{N-[2-methyl-3-(2-[N′-methyl-N′-([quinolin-4-yl]-methyl)amino]ethyl)-1H-indol-5-yl]aminothiocarbonyl}piperidine; 1-{N-[2-methyl-3-(2-[N′-methyl-N′-([isoquinolin-7-yl]-methyl)amino]ethyl)-1H-indol-5-yl]aminothiocarbonyl}piperazine; 1-{N-[2-methyl-3-(2-[N′-([quinoxalin-2-yl]methyl)-amino]ethyl)-1H-indol-5-yl]ammothiocarbonyl}-4-methylpipera-zinehexanoate; 1-{N-[2-methyl-3-(2-[N′-methyl-N′-([quinazolin-5-yl]-methyl)amino]ethyl)-1H-indol-5-yl]amm othiocarbonyl}-4-phenylpiperazine; 1-{N-[2-methyl-3-(2-[N′-methyl-N′-([thiazol-2-yl]-methyl)amino]ethyl)-1H-indol-5-yl]amm othiocarbonyl}-4-benzylpiperazine; 1-{N-[2-methyl-3-(2-[N′-methyl-N′-([2-aminobenzothiazol-5-yl]methyl)amino]ethyl)-1H-indol-5-yl]aminothiocarbonyl}-4-(2,4-dichlorophenyl)piperazine trifluoromethanesulfonate; 1-{N-[2-methyl-3-(2-[N′-methyl-N′-([oxazol-5-yl]methyl)-amino]ethyl)-1H-indol-5-yl]ammothiocarbonyl}morpholine; 1-{N-[2-methyl-3-(2-[N′-methyl-N′-([6-nitrobenzoxazol-2-yl]methyl)amino]ethyl)-1H-indol-5-yl]ammothiocarbonyl}thio-morpholine hydrobromide; N-[2-methyl-3-(2-[N′-([benzisoxazol-3-yl]methyl)-amino]ethyl)-1H-indol-5-yl]thiophene-2-carboxamide; N-[2-methyl-3-(2-[N′—([1,3,4-oxadiazol-2-yl]methyl)-amino]ethyl)-1H-indol-5-yl]furan-3-carboxamide; N-[2-methyl-3-(2-[N′—([1,2,3-triazol-4-yl]methyl)-amino]ethyl)-1H-indol-5-yl]-4-fluorobenzamide tosylate; N-[2-phenyl-3-(2-[N′-((4-bromothien-2-yl)meth-yl)amino]ethyl)-1H-indol-5-yl)]-4-fluorobenzamide hydrochloride; N-[2-ethyl-3-(2-[N′-((3-methylthiobenzofur-5-yl)ethyl)amino]ethyl)-1H-indol-5-yl]pyridine-2-carboxamide; N-[2-propyl-3-(2-[N′-1-((isobenzofur-2-yl)prop-3-yl)amino]ethyl)-1H-indol-5-yl]-4-fluorobenzamide; N-[2-methyl-3-(2-[N′-(pyrrol-3-yl)methyl)amino]ethyl)-1H-indol-5-yl]-4-fluorobenzamidemaleate; N-[2-methyl-3-(2-[N′-((5-cyanoimidazol-2-yl)methyl)amino]ethyl)-1H-indol-5-yl]-4-acetamide trifluoroacetate; N-[2-methyl-3-(2-[N′-((6-carboxamidopyrazin-2-yl)methyl)amino]ethyl)-1H-indol-5-yl]propanamide; 5-(N,N-dibutylaminosulfonyl)amino-2-methyl-3-(2-[N′-methyl-N′-((5-nitropyrimidin-2-yl)methyl)amino]ethyl)-1H-indole; 5-((N-isopropyl-N-butylamino)sulfonyl)amino-2-methyl-3-(2-[N′-methyl-N′-((5-dimethylaminopyridazin-3-yl)methyl)-amino]ethyl)-1H-indolebenzoate; 5-(dimethylaminosulfonyl)amino-2-methyl-3-(2-[N′-methyl-N′-((indazol-5-yl)methyl)amino]ethyl)-1H-indole; N-[2-methyl-3-(2-[N′-methyl-N′-((quinolin-4-yl)methyl)-amino]ethyl)-1H-indol-5-yl]-4-chlorophenylsulfonamide; N-[2-methyl-3-(2-[N′-methyl-N′-((isoquinolin-7-yl)meth-yl)amino]ethyl)-1H-indol-5-yl]phenylsulfonamide; N-[2-methyl-3-(2-[N′-methyl-N′-((quinoxalin-2-yl)-methyl) amino]ethyl)-1H-indol-5-yl]butanesulfonamide acetate; N-[2-methyl-3-(2-[N′-methyl-N′-((quinazolin-5-yl)-methyl)amino]ethyl)-1H-indol-5-yl]isopropanesulfonamide; N-[2-methyl-3-(2-[N′-methyl-N′-((thiazol-2-yl)methyl)-amino]ethyl)-1H-indol-5-yl]propanesulfonamide; N-[2-methyl-3-(2-[N′-((2-aminobenzothiazol-5-yl)methyl)amino]ethyl)-1H-indol-5-yl]ethanesulfonamide tri-fluoromethanesulfonate; 5-isopropoxycarbonylamino-2-methyl-3-(2-[N′-methyl-N′-(2-[1-isopropylpyrazol-4-yl]ethyl)amino]ethyl)-1H-indole mandelate; 5-methoxycarbonylamino-(2-[N′-methyl-N′-([4-bromothien-2-yl]methyl)amino]ethyl)-1H-indole hydrochloride; 5-(tert-butoxvcarbonyl)amino-2-ethyl-3-(2-[N′-ethyl-N′-(2-[3-methylthiobenzofur-5-yl]ethyl)amino]ethyl)-1H-indole; 5-(1-penten-5-yloxy)carbonylamino-2-propyl-3-(2-[N′-isopropyl-N′-(3-[isobenzofur-2-yl]propyl)amino]ethyl)-1H-indole; 5-(1-buten-4-yloxy)carbonylamino-2-methyl-3-(2-[N′-butyl-N′-([pyrrol-3-yl]methyl)amino]ethyl)-1H-indolemaleate; 5-(4-hexen-6-yloxy)carbonylamino-3-(2-[N′-methyl-N′-([5-cyanoimidazol-2-yl]methyl)amino]ethyl)-1H-indole trifluoroacetate; 5-(2-chlorophenoxy)carbonylamino-2-methyl-3-(2-[N′-([6-carboxamidopyrazin-2-yl]methyl)amino]ethyl)-1H-indole; 5-(3-bromophenoxy)carbonylamino-3-(2-[N′-([5-nitropyrimidin-2-yl]methyl)amino]ethyl)-1H-indole; 5-(3-methoxyphenoxy)carbonylamino-2-methyl-3-(2-[N′-methyl-N′-([5-dimethylaminopyridazin-3-yl]methyl)-amino]ethyl)-1H-indolebenzoate; 5-cyclopropoxycarbonyl amino-2-methyl-3-(2-[N′-methyl-N′-([indazol-5-yl]methyl)amino]ethyl)-1H-indole; 5-cyclohexyloxy carbonylamino-2-methyl-3-(2-[N′-methyl-N′-([quinolin-4-yl]methyl)amino]ethyl)-1H-indole; 5-cyclo octyloxycarbonylamino-2-methyl-3-(2-[N′-methyl-N′-([isoquinolin-7-yl]methyl)amino]ethyl)-1H-indole; 5-(butoxymethoxy)carbonylamino-2-methyl-3-(2-[N′-methyl-N′-([quinoxalin-2-yl]methyl)amino]ethyl)-1H-indolehexanoate; and 5-(ethoxypropoxy)carbonylamino-N-[2-methyl-3-(2-[N′-methyl-N′-([quinazolin-5-yl]methyl)amino]ethyl)-1H-indole.

The following HTR_{1F} agonists are disclosed in PCT publication No. WO 98/46570: 5-amino-3-(1,2,3,4,5,8-hexahydroindolizin-7-yl)-1H-indole 5-amino-3-(octahydroindolizin-7-yl)-1H-indole; 5-amino-3-(1,2,3,4,5,8-hexahydroindolizin-7-yl)-benzofuran; 5-amino-3-(octahydroindolizin-7-yl)-benzofuran; 5-amino-3-(1,2,3,4,5,8-hexahydroindolizin-7-yl)-benzothiophene; 5-amino-3-(octahydroindolizin-7-yl)-benzothiophene; 5-(4-fluorophenyl)thio-3-(1,2,3,4,5,8-hexahydroindolizin-7-yl)-1H-indole; 5-(3-chlorophenyl)thio-3-(1,4,5,6,7,8,9-heptahydroquinolizin-2-yl)-2-methyl-benzofuran; 5-(2-bromophenyl)thio-3-(1-azabicyclo[5.4.0]undec-3-en-4-yl)-benzothiophene; 5-(2-iodophenyl)thio-3-(1,2,3,4,5,8-hexahydroindolizin-7-yl)-1H-indole; 5-(3-methoxyphenyl)thio-3-(1,4,5,6,7,8,9-heptahydroquinolizin-2-yl)-benzofuran; 5-(4-iodophenyl)thio-3-(1-azabicyclo[5.4.0]undec-3-en-4-yl)-benzothiophene; 5-(2-ethoxyphenyl)thio-3-(1,2,3,4,5,8-hexahydroindolizin-7-yl)-1H-indole; 5-(4-ethoxyphenyl)thio-3-(1,4,5,6,7,8,9-heptahydroquinolizin-2-yl)-benzofuran; 5-(3-propoxyphenyl)thio-3-(1-azabicyclo[5.4.0]undec-3-en-4-yl)-benzothiophene hydrochloride; 5-(2-isopropoxyphenyl)thio-3-(1,2,3,4,5,8-hexahydroindolizin-7-yl)-1H-indole; 5-(4-isopropoxyphenyl) thio-3-(1,4,5,6,7,8,9-heptahydroquinolizin-2-yl)-benzofuran hydrobromide; 5-(3-butoxyphenyl)thio-3-(1-azabicyclo[5.4.0]undec-3-en-4-yl)-benzothiophene hydroiodide; 5-(2-isobutoxyphenyl)thio-3-(1,2,3,4,5,8-hexahydroindolizin-7-yl)-1H-indole; 5-(4-isobutoxyphenyl)thio-3-(1,4,5,6,7,8,9-heptahydroquinolizin-2-yl)-benzofuran acetate; 5-(3-sec-butoxyphenyl)thio-3-(1-azabicyclo[5.4.0]undec-3-en-4-yl)-benzothiophene; 5-(2-tert-butoxyphenyl)thio-3-(1,2,3,4,5,8-hexahydroindolizin-7-yl)-1H-indole propionate; 5-(4-tert-butoxyphenyl)thio-3-(1,4,5,6,7,8,9-heptahydroquinolizin-2-yl)-benzofuran; 5-(2-pyridinyl)thio-3-(1-azabicyclo[5.4.0]undec-3-en-4-yl)-benzothiophene decanoate; 5-(4-pyridinyl)thio-3-(1,2,3,4,5,8-hexahydroindolizin-7-yl)-1H-indole; 5-(2-phenethyl)thio-3-(1,4,5,6,7,8,9-heptahydroquinolizin-2-yl)-benzofuran caprylate; 5-(4-phenbutyl)thio-3-(1-azabicyclo[5.4.0]undec-3-en-4-yl)-benzothiophene; 5-(2-chlorophenyl)thio-3-(octahydroindolizin-7-yl)-1H-indole acrylate; 5-(4-bromophenyl)thio-3-(octahydro-2H-quinolizin-2-yl)-benzofuran; 5-(2-bromophenyl)thio-3-(1-azabicyclo[5.4.0]undecan-4-yl)-benzothiophene formate; 5-(3-iodophenyl)thio-3-(octahydroindolizin-7-yl)-1H-indole; 5-(2-methoxyphenyl)thio-3-(octahydro-2H-quinolizin-2-yl) benzofuran isobutyrate; 5-(4-methoxyphenyl)thio-3-(1-azabicyclo[5.4.0]undecan-4-yl)-benzothiophene; 5-(3-ethoxyphenyl)thio-3-(octahydroindolizin-7-yl)-2-methyl-1H-indole caproate; 5-(2-propoxyphenyl)thio-3-(octahydro-2H-quinolizin-2-yl)-benzofuran; 5-(4-propoxyphenyl)thio-3-(1-azabicyclo[5.4.0]undecan-4-yl)-benzothiophene heptanoate; 5-(3-isopropoxyphenyl)thio-3-(octahydroindolizin-7-yl)-1H-indole; 5-(2-butoxyphenyl)thio-3-(octahydro-2H-quinolizin-2-yl)-benzofuran propiolate; 5-(4-butoxyphenyl)thio-3-(1-azabicyclo[5.4.0]undecan-4-yl)-benzothiophene; 5-(2-sec-butoxyphenyl)thio-3-(octahydroindolizin-7-yl)-1H-indole oxalate; 5-(4-sec-butoxyphenyl)thio-3-(octahydro-2H-quinolizin-2-yl)-benzofuran 5-(2-tert-butoxyphenyl)thio-3-(1-azabicyclo[5.4.0]undecan-4-yl)-benzothiophene malonate; 5-(3-pyridinyl)thio-3-(octahydroindolizin-7-yl)-1H-indole; 5-benzylthio-3-(octahydro-2H-quinolizin-2-yl)-benzofuran succinate; 5-(3-phenpropyl)thio-3-(1-azabicyclo[5.4.0]undecan-4-yl)-benzothiophene; 5-propanoyl-3-(1,2,3,4,5,8-hexahydroindolizin-7-yl)-1H-indole suberate; 5-(2-methylpropanoyl)-3-(1,4,5,6,7,8,9-heptahydroquinolizin-2-yl)-benzofuran; 5-butanoyl-3-(1-azabicyclo[5.4.0]undec-3-en-4-yl)-benzothiophene sebacate; 5-(sec-butanoyl)-3-(1,2,3,4,5,8-hexahydroindolizin-7-yl)-1H-indole; 5-(2-methylbutanoyl)-3-(1,4,5,6,7,8,9-heptahydroquinolizin-2-yl)-2-methyl-benzofuran fumarate; 5-(3,3-dimethylbutanoyl)-3-(1-azabicyclo[5.4.0]undec-3-en-4-yl)-benzothiophene; 5-heptanoyl-3-(1,2,3,4,5,8-hexahydroindolizin-7-yl)-1H-indole butyne-1,4-dioate; 5-(3-chlorobenzoyl)-3-(1,4,5,6,7,8,9-heptahydroquinolizin-2-yl)-benzofuran; 5-(3-fluorobenzoyl)-3-(1-azabicyclo[5.4.0]undec-3-en-4-yl)-benzothiophene; 5-(3-bromobenzoyl)-3-(1,2,3,4,5,8-hexahydroindolizin-7-yl)-1H-indole hydrobromide; 5-(2-bromobenzoyl)-3-(1,4,5,6,7,8,9-heptahydroquinolizin-2-yl)-benzofuran; 5-(3-iodobenzoyl)-3-(1-azabicyclo[5.4.0]undec-3-en-4-yl)-benzothiophene; 5-(3-methoxybenzoyl)-3-(1,2,3,4,5,8-hexahydroindolizin-7-yl)-1H-indole; 5-(2-ethoxybenzoyl)-3-(1,4,5,6,7,8,9-heptahydroquinolizin-2-yl)-benzofuran; 5-(4-ethoxybenzoyl)-3-(1-azabicyclo[5.4.0]undec-3-en-4-yl)-benzothiophene; 5-(2-propoxybenzoyl)-3-(1,2,3,4,5,8-hexahydroindolizin-7-yl)-1H-indole hydrochloride; 5-(3-propoxybenzoyl)-3-(1,4,5,6,7,8,9-heptahydroquinolizin-2-yl)-benzofuran; 5-(3-butoxybenzoyl)-3-(1-azabicyclo[5.4.0]undec-3-en-4-yl)-benzothiophene; 5-(2-methylbenzoyl)-3-(1,2,3,4,5,8-hexahydroindolizin-7-yl)-1H-indole; 5-(3-ethylbenzoyl)-3-(1,4,5,6,7,8,9-heptahydroquinolizin-2-yl)-benzofuran; 5-(4-propylbenzoyl)-3-(1-azabicyclo[5.4.0]undec-3-en-4-yl)-benzothiophene; 5-(2-propylbenzoyl)-3-(1,2,3,4,5,8-hexahydroindolizin-7-yl)-1H-indole; 5-(3-butylbenzoyl)-3-(1,4,5,6,7,8,9-heptahydroquinolizin-2-yl)-benzofuran; 5-(3-trifluoromethylbenzoyl)-3-(1-azabicyclo[5.4.0]undec-3-en-4-yl)-benzothiophene; 5-(3-trifluoromethoxybenzoyl)-3-(1,2,3,4,5,8-hexahydroindolizin-7-yl)-1H-indole; 5-(3-dimethylaminobenzoyl)-3-(1,4,5,6,7,8,9-heptahydroquinolizin-2-yl)-benzofuran; 5-(3-phenylpropanoyl)-3-(1-azabicyclo[5.4.0]undec-3-en-4-yl)-benzothiophene; 5-(5-phenylpentanoyl)-3-(1,2,3,4,5,8-hexahydroindolizin-7-yl)-1H-indole; 5-(3-pyridinecarbonyl)-3-(1,4,5,6,7,8,9-heptahydroquinolizin-2-yl)-benzofuran; 5-(3-phenylpropanoyl)-3-(1-azabicyclo[5.4.0]undec-3-en-4-yl)-benzothiophene; 5-butanoyl-3-(octahydroindolizin-7-yl)-1H-indole; 5-(3-methyl)butanoyl-3-(octahydro-2H-quinolizin-2-yl)-2-methyl-benzofuran; 5-(2,2-dimethyl)propanoyl-3-(1-azabicyclo[5.4.0]undecan-4-yl)-benzothiophene fumarate; 5-hexanoyl-3-(octahydroindolizin-7-yl)-1H-indole; 5-(2-ethyl)butanoyl-3-(octahydro-2H-quinolizin-2-yl)-benzofuran; 5-(2-chlorobenzoyl)-3-(1-azabicyclo[5.4.0]undecan-4-yl)-benzothiophene; 5-(2-fluorobenzoyl)-3-(octahydroindolizin-7-yl)-1H-indole 5-(2-bromobenzoyl)-3-(octahydro-2H-quinolizin-2-yl)-benzofuran; 5-(2-iodobenzoyl)-3-(1-azabicyclo[5.4.0]undecan-4-yl)-benzothiophene; 5-(4-iodobenzoyl)-3-(octahydroindolizin-7-yl)-1H-indole; 5-(2-methoxybenzoyl)-3-(octahydro-2H-quinolizin-2-yl)-benzofuran; 5-(3-ethoxybenzoyl)-3-(1-azabicyclo[5.4.0]undecan-4-yl)-benzothiophene; 5-(4-propoxybenzoyl)-3-(octahydroindolizin-7-yl)-1H-indole; 5-(2-butoxybenzoyl)-3-(octahydro-2H-quinolizin-2-yl)-benzofuran hexyne-1,6-dioate; 5-(4-butoxybenzoyl)-3-(1-azabicyclo[5.4.0]undecan-4-yl)-benzothiophene; 5-(3-methylbenzoyl)-3-(octahydroindolizin-7-yl)-1H-indole; 5-(4-ethylbenzoyl)-3-(octahydro-2H-quinolizin-2-yl)-benzofuran; 5-(2-ethylbenzoyl)-3-(1-azabicyclo[5.4.0]undecan-4-yl)-benzothiophene benzoate; 5-(3-propylbenzoyl)-3-(octahydroindolizin-7-yl)-1H-indole; 5-(2-butylbenzoyl)-3-(octahydro-2H-quinolizin-2-yl)-benzofuran; 5-(4-butylbenzoyl)-3-(1-azabicyclo[5.4.0]undecan-4-yl)-benzothiophene; 5-(2-trifluoromethyl benzoyl)-3-(octahydroindolizin-7-yl)-1H-indole chlorobenzoate; 5-(3-trifluoromethoxybenzoyl)-3-(octahydro-2H-quinolizin-2-yl)-benzofuran; 5-(2-dimethylaminobenzoyl)-3-(1-azabicyclo[5.4.0]undecan-4-yl)-benzothiophene; 5-(4-phenylbutanoyl)-3-(octahydroindolizin-7-yl)-1H-indole 4-methylbenzoate; 5-(1-naphthoyl)-3-(octahydro-2H-quinolizin-2-yl)-benzofuran; 5-(4-pyridinecarbonyl)-3-(1-azabicyclo[5.4.0]undecan-4-yl)-benzothiophene; 5-(N-phenyl)carboxamido-3-(1,2,3,4,5,8-hexahydroindolizin-7-yl)-2-methyl-1H-indole 2,4-dinitrobenzoate; 5-(N-benzyl)carboxamido-3-(1,4,5,6,7,8,9-heptahydroquinolizin-2-yl)-benzofuran; 5-(N-(2-(4-chlorophenyl)ethyl))carboxamido-3-(1-azabicyclo[5.4.0]undec-3-en-4-yl)-benzothiophene 4-hydroxybenzoate; 5-(N-(2-(3-methylphenyl)ethyl))carboxamido-3-(1,2,3,4,5,8-hexahydroindolizin-7-yl)-1H-indole; 5-(N-(3-(2-methoxyphenyl)propyl))carboxamido-3-(1,4,5,6,7,8,9-heptahydroquinolizin-2-yl)-benzofuran; 5-(N-(4-(4-trifluoromethylphenyl)butyl))carboxamido-3-(1-azabicyclo[5.4.0]undec-3-en-4-yl)-benzothiophene 3-methoxybenzoate; 5-(N-(4-chlorophenyl))carboxamido-3-(octahydroindolizin-7-yl)-1H-indole; 5-(N-benzyl)carboxamido-3-(octahydro-2H-quinolizin-2-yl)-benzofuran; 5-(N-(2-phenethyl))carboxamido-3-(1-azabicyclo[5.4.0]undecan-4-yl)-2-methyl-benzothiophene; 5-(N-(3-phenpropyl))carboxamido-3-(octahydroindolizin-7-yl)-1H-indole; 5-(N-(3-phenpropyl))carboxamido-3-(octahydro-2H-quinolizin-2-yl)-benzofuran phthalate; 5-(N-(4-phenbutyl))carboxamido-3-(1-azabicyclo[5.4.0]undecan-4-yl)-benzothiophene; 5-(N-methyl-N-ethanesulfonyl)amino-3-(1,2,3,4,5,8-hexahydroindolizin-7-yl)-1H-indole; 5-(N-ethyl-N-propanesulfonyl)amino-3-(1,4,5,6,7,8,9-heptahydroquinolizin-2-yl)-benzofuran; 5-(N-isopropanesulfonyl)amino-3-(1-azabicyclo[5.4.0]undec-3-en-4-yl)-benzothiophene; 5-(N-propyl-N-butanesulfonyl)amino-3-(1,2,3,4,5,8-hexahydroindolizin-7-yl)-1H-indole; 5-(N-isobutanesulfonyl)amino-3-(1,4,5,6,7,8,9-heptahydroquinolizin-2-yl)-benzofuran methanesulfonate; 5-(N-sec-butanesulfonyl)amino-3-(1-azabicyclo[5.4.0]undec-3-en-4-yl)-benzothiophene; 5-(N-tert-butanesulfonyl)amino-3-(1,2,3,4,5,8-hexahydroindolizin-7-yl)-1H-indole; 5-(N-butyl-N-benzenesulfonyl)amino-3-(1,4,5,6,7,8,9-heptahydroquinolizin-2-yl)-benzofuran; 5-(N,N-diethylaminosulfonyl)amino-3-(1-azabicyclo[5.4.0]undec-3-en-4-yl)-2-methyl-benzothiophene; 5-(N,N-dipropylaminosulfonyl)amino-3-(1,2,3,4,5,8-hexahydroindolizin-7-yl)-1H-indole; 5-(N,N-diisopropylaminosulfonyl)amino-3-(1,4,5,6,7,8,9-heptahydroquinolizin-2-yl)-benzofuran; 5-(N,N-dibutylaminosulfonyl)amino-3-(1-azabicyclo[5.4.0]undec-3-en-4-yl)-benzothiophene; 5-(N-ethanesulfonyl)amino-3-(octahydroindolizin-7-yl)-1H-indole phenylsulfonate; 5-(N-propanesulfonyl)amino-3-(octahydro-2H-quinolizin-2-yl)-benzofuran; 5-(N-isopropanesulfonyl)amino-3-(1-azabicyclo[5.4.0]undecan-4-yl)-benzothiophene; 5-(N-butanesulfonyl)amino-3-(octahydroindolizin-7-yl)-1H-indole; 5-(N-isobutanesulfonyl)amino-3-(octahydro-2H-quinolizin-2-yl)-benzofuran; 5-(N-isopropyl-N-sec-butanesulfonyl)amino-3-(1-azabicyclo[5.4.0]undecan-4-yl)-benzothiophene; 5-(N-(tert-butyl)sulfonyl)amino-3-(octahydroindolizin-7-yl)-1H-indole; 5-(N,N-diethylaminosulfonyl)amino-3-(octahydro-2H-quinolizin-2-yl)-benzofuran; 5-(N,N-dipropylaminosulfonyl)amino-3-(1-azabicyclo[5.4.0]undecan-4-yl)-2-methyl-benzothiophene; 5-(N,N-diisopropylaminosulfonyl)amino-3-(octahydroindolizin-7-yl)-1H-indole; 5-(N,N-dibutylaminosulfonyl)amino-3-(octahydro-2H-quinolizin-2-yl)-benzofuran; N-ethyl-N′-(3-(1,2,3,4,5,8-hexahydroindolizin-7-yl)-1H-indol-5-yl)thiourea; N-isopropyl-N′-(3-(1,4,5,6,7,8,9-heptahydroquinolizin-2-yl)-1H-indol-5-yl)thiourea; N-(3-methoxy)phenyl-N′-(3-(1-azabicyclo[5.4.0]undec-3-en-4-yl)-1H-indol-5-yl)thiourea; N-(2-ethoxy)phenyl-N′-(2-methyl-3-(1,2,3,4,5,8-hexahydroindolizin-7-yl)-1H-indol-5-yl)thiourea; N-(2-ethoxy)phenyl-N′-(3-(1,4,5,6,7,8,9-heptahydroquinolizin-2-yl)-1H-indol-5-yl)thiourea; N-(3-ethoxy)phenyl-N′-(3-(1-azabicyclo[5.4.0]undec-3-en-4-yl)-1H-indol-5-yl)thiourea; N-(3-propoxy)phenyl-N′-(3-(1,2,3,4,5,8-hexahydroindolizin-7-yl)-1H-indol-5-yl)thiourea; N-(3-isopropoxy)phenyl-N′-(3-(1,4,5,6,7,8,9-heptahydroquinolizin-2-yl)-1H-indol-5-yl)thiourea; N-(4-isopropoxy)phenyl-N′-(3-(1-azabicyclo[5.4.0]undec-3-en-4-yl)-1H-indol-5-yl)thiourea phenylacetate; N-(3-butoxy)phenyl-N′-(3-(1,2,3,4,5,8-hexahydroindolizin-7-yl)-1H-indol-5-yl)thiourea hydrochloride; N-(2,3-dibromo)phenyl-N′-(3-(1,4,5,6,7,8,9-heptahydroquinolizin-2-yl)-1H-indol-5-yl)thiourea; N-(2-bromo-3-iodo)phenyl-N′-(3-(1-azabicyclo[5.4.0]undec-3-en-4-yl)-1H-indol-5-yl)thiourea; N-(3,4-difluoro)phenyl-N′-(3-(1,2,3,4,5,8-hexahydroindolizin-7-yl)-1H-indol-5-yl)thiourea; N-(3-chloro-4-bromo)phenyl-N′-(3-(1,4,5,6,7,8,9-heptahydroquinolizin-2-yl)-1H-indol-5-yl)thiourea; N-(2-bromo-4-fluoro)phenyl-N′-(3-(1-azabicyclo[5.4.0]undec-3-en-4-yl)-1H-indol-5-yl)thiourea; N-(2,4-diiodo)phenyl-N′-(3-(1,2,3,4,5,8-hexahydroindolizin-7-yl)-1H-indol-5-yl)thiourea; N-(2-chloro-5-iodo)phenyl-N′-(3-(1,2,3,4,5,8-hexahydroindolizin-7-yl)-1H-indol-5-yl)thiourea; N-(2-fluoro-6-iodo)phenyl-N′-(3-(1,4,5,6,7,8,9-heptahydroquinolizin-2-yl)-1H-indol-5-yl)thiourea; N-(3-fluoro-5-chloro)phenyl-N′-(3-(1-azabicyclo[5.4.0]undec-3-en-4-yl)-1H-indol-5-yl)thiourea; N-phenethyl-N′-(3-[1,2,3,4,5,8-hexahydroindolizin-7-yl]-1H-indol-5-yl)thiourea; N-(4-phenbutyl)-N′-(3-(1,4,5,6,7,8,9-heptahydroquinolizin-2-yl)-1H-indol-5-yl)thiourea; N-(2-trifluoromethyl)phenyl-N′-(3-(1-azabicyclo[5.4.0]undec-3-en-4-yl)-1H-indol-5-yl)thiourea; N-(3-phenyl)phenyl-N′-(3-(1,2,3,4,5,8-hexahydroindolizin-7-yl)-1H-indol-5-yl)thiourea; N-propyl-N′-(3-(1-azabicyclo[5.4.0]undecan-4-yl)-1H-indol-5-yl)thiourea; N-butyl-N′-(3-(octahydroindolizin-7-yl)-1H-indol-5-yl)thiourea; N-(2-methoxy)phenyl-N′-(3-(octahydro-2H-quinolizin-2-yl)-1H-indol-5-yl)thiourea; N-(4-ethoxy)phenyl-N′-(3-(1-azabicyclo[5.4.0]undecan-4-yl)-1H-indol-5-yl)thiourea; N-(4-propoxy)phenyl-N′-(2-methyl-3-(octahydroindolizin-7-yl)-1H-indol-5-yl)thiourea; N-(2-propoxy)phenyl-N′-(3-(octahydro-2H-quinolizin-2-yl)-1H-indol-5-yl)thiourea; N-(2-isopropoxy)phenyl-N′-(3-(1-azabicyclo[5.4.0]undecan-4-yl)-1H-indol-5-yl)thiourea; N-(4-butoxy)phenyl-N′-(3-(octahydroindolizin-7-yl)-1H-indol-5-yl)thiourea; N-(2-butoxy)phenyl-N′-(3-(octahydro-2H-quinolizin-2-yl)-1H-indol-5-yl)thiourea fumarate; N-(2-methoxy)phenyl-N′-(3-(1-azabicyclo[5.4.0]undecan-4-yl)-1H-indol-5-yl)thiourea; N-(2,3-dibromo)phenyl-N′-(3-(octahydroindolizin-7-yl)-1H-indol-5-yl)thiourea; N-(2-bromo-3-iodo)phenyl-N′-(3-(octahydro-2H-quinolizin-2-yl)-1H-indol-5-yl)thiourea; N-(3,4-difluoro)phenyl-N′-(3-(1-azabicyclo[5.4.0]undecan-4-yl)-1H-indol-5-yl)thiourea; N-(3-chloro-4-bromo)phenyl-N′-(3-(octahydroindolizin-7-yl)-1H-indol-5-yl)thiourea; N-(2-bromo-4-fluoro)phenyl-N′-(3-(octahydro-2H-quinolizin-2-yl)-1H-indol-5-yl)thiourea; N-(2,4-diiodo)phenyl-N′-(3-(1-azabicyclo[5.4.0]undecan-4-yl)-1H-indol-5-yl)thiourea; N-(2-chloro-5-iodo)phenyl-N′-(3-(octahydroindolizin-7-yl)-1H-indol-5-yl)thiourea; N-(2-fluoro-6-iodo)phenyl-N′-(3-(octahydro-2H-quinolizin-2-yl)-1H-indol-5-yl)thiourea; N-(3-fluoro-5-chloro)phenyl-N′-(3-(1-azabicyclo[5.4.0]undecan-4-yl)-1H-indol-5-yl)thiourea; N-(3-phenpropyl)-N′-(3-(octahydroindolizin-7-yl)-1H-indol-5-yl)thiourea; N-(4-trifluoromethyl)phenyl-N′-(3-(octahydro-2H-quinolizin-2-yl)-1H-indol-5-yl)thiourea; N-(4-phenyl)phenyl-N′-(3-(1-azabicyclo[5.4.0]undecan-4-yl)-1H-indol-5-yl)thiourea; N-(2-bromo-3-iodo)phenyl-N′-(3-(1-azabicyclo[5.4.0]undec-3-en-4-yl)-1H-indol-5-yl)urea; N-(3,4-difluoro)phenyl-N′-(3-(1,2,3,4,5,8-hexahydroindolizin-7-yl)-1H-indol-5-yl)urea; N-(3-chloro-4-bromo)phenyl-N′-(3-(1,4,5,6,7,8,9-heptahydroquinolizin-2-yl)-1H-indol-5-yl)urea; N-(2-bromo-4-fluoro)phenyl-N′-(3-(1-azabicyclo[5.4.0]undec-3-en-4-yl)-1H-indol-5-yl)urea; N-(2,4-diiodo)phenyl-N′-(3-(1,2,3,4,5,8-hexahydroindolizin-7-yl)-1H-indol-5-yl)urea; N-(2-chloro-5-iodo)phenyl-N′-(3-(1,4,5,6,7,8,9-heptahydroquinolizin-2-yl)-1H-indol-5-yl)urea; N-(2-fluoro-6-iodo)phenyl-N′-(3-(1-azabicyclo[5.4.0]undec-3-en-4-yl)-1H-indol-5-yl)urea; N-(3-fluoro-5-chloro)phenyl-N′-(3-(1,2,3,4,5,8-hexahydroindolizin-7-yl)-1H-indol-5-yl)urea; N-(4-phenbutyl)-N′-(3-(1,4,5,6,7,8,9-heptahydroquinolizin-2-yl)-1H-indol-5-yl)urea; N-phenyl-N-propyl-N′-(3-(1-azabicyclo[5.4.0]undec-3-en-4-yl)-1H-indol-5-yl)urea; N-phenyl-N-butyl-N′-(3-(1,2,3,4,5,8-hexahydroindolizin-7-yl)-1H-indol-5-yl)urea; N-methyl-N-propyl-N′-(3-(1,4,5,6,7,8,9-heptahydroquinolizin-2-yl)-1H-indol-5-yl)urea; N-ethyl-N-isopropyl-N′-(3-(1-azabicyclo[5.4.0]undec-3-en-4-yl)-1H-indol-5-yl)urea; N,N-dipropyl-N′-(3-(1,2,3,4,5,8-hexahydroindolizin-7-yl)-1H-indol-5-yl)urea; N-butyl-N-propyl-N′-(3-(1,4,5,6,7,8,9-heptahydroquinolizin-2-yl)-1H-indol-5-yl)urea; N-butyl-N-isopropyl-N′-(3-(1-azabicyclo[5.4.0]undec-3-en-4-yl)-1H-indol-5-yl)urea; N-hexyl-N′-(3-(octahydroindolizin-7-yl)-1H-indol-5-yl)urea; N-(2-buten-4-yl)-N′-(3-(octahydro-2H-quinolizin-2-yl)-1H-indol-5-yl)urea; N-(2-penten-5-yl)-N′-(3-(1-azabicyclo[5.4.0]undecan-4-yl)-1H-indol-5-yl)urea; N-(1-hexen-6-yl)-N′-(3-(octahydroindolizin-7-yl)-1H-indol-5-yl)urea; N-(3-hexen-6-yl)-N′-(3-(1-(octahydro-2H-quinolizin-2-yl)-1H-indol-5-yl)urea; N-cyclopropyl-N′-(3-(1-azabicyclo[5.4.0]undecan-4-yl)-1H-indol-5-yl)urea; N-cyclopentyl-N′-(3-(octahydroindolizin-7-yl)-1H-indol-5-yl)urea; N-cyclooctyl-N′-(3-(octahydro-2H-quinolizin-2-yl)-1H-indol-5-yl)urea; N-(2-chloro)phenyl-N′-(3-(1-azabicyclo[5.4.0]undecan-4-yl)-1H-indol-5-yl)urea; N-(3-bromo)phenyl-N′-(3-(octahydroindolizin-7-yl)-1H-indol-5-yl)urea; N-(3-fluoro)phenyl-N′-(3-(octahydro-2H-quinolizin-2-yl)-1H-indol-5-yl)urea; N-(3-iodo)phenyl-N′-(2-methyl-3-(1-azabicyclo[5.4.0]undecan-4-yl)-1H-indol-5-yl)urea; N-(3-phenyl)phenyl-N′-(3-(octahydroindolizin-7-yl)-1H-indol-5-yl)urea; N-(4-ethoxy)phenyl-N′-(3-(octahydro-2H-qu-nolizin-2-yl)-1H-indol-5-yl)urea; N-(2-ethoxy)phenyl-N′-(3-(1-azabicyclo[5.4.0]undecan-4-yl)-1H-indol-5-yl)urea; N-(3-propoxy)phenyl-N′-(3-(octahydroindolizin-7-yl)-1H-indol-5-yl)urea; N-(2-isopropoxy)phenyl-N′-(3-(octahydro-2H-quinolizin-2-yl)-1H-indol-5-yl)urea; N-(4-isopropoxy)phenyl-N′-(3-(1-azabicyclo[5.4.0]undecan-4-yl)-1H-indol-5-yl)urea; N-(3-butoxy)phenyl-N′-(3-(octahydroindolizin-7-yl)-1H-indol-5-yl)urea; N-(4-formyl)phenyl-N′-(3-(octahydro-2H-quinolizin-2-yl)-1H-indol-5-yl)urea; N-(2-formyl)phenyl-N′-(3-(1-azabicyclo[5.4.0]undecan-4-yl)-1H-indol-5-yl)urea; N-(3-acetyl)phenyl-N′-(3-(octahydroindolizin-7-yl)-1H-indol-5-yl)urea phenylpropionate; N-(3-propanoyl)phenyl-N′-(3-(octahydro-2H-quinolizin-2-yl)-1H-indol-5-yl)urea; N-(3-ethylthio)phenyl-N′-(3-(1-azabicyclo[5.4.0]undecan-4-yl)-1H-indol-5-yl)urea; N-(2-ethylthio)phenyl-N′-(3-(octahydroindolizin-7-yl)-1H-indol-5-yl)urea; N-(3-propylthio)phenyl-N′-(3-(octahydro-2H-quinolizin-2-yl)-1H-indol-5-yl)urea; N-(3-isopropylthio)phenyl-N′-(3-(1-azabicyclo[5.4.0]undecan-4-yl)-1H-indol-5-yl)urea; N-(2-methyl)phenyl-N′-(3-(octahydroindolizin-7-yl)-1H-indol-5-yl)urea; N-(3-ethyl)phenyl-N′-(3-(octahydro-2H-quinolizin-2-yl)-1H-indol-5-yl)urea; -(2-propyl)phenyl-N′-(3-(1-azabicyclo[5.4.0]undecan-4-yl)-1H-indol-5-yl)urea; N-(3-isopropyl)phenyl-N′-(3-(octahydroindolizin-7-yl)-1H-indol-5-yl)urea; N-(4-butyl)phenyl-N′-(3-(octahydro-2H-quinolizin-2-yl)-1H-indol-5-yl)urea; N-(2-butyl)phenyl-N′-(3-(1-azabicyclo[5.4.0]undecan-4-yl)-1H-indol-5-yl)urea; N-(3-methoxycarbonyl)phenyl-N′-(3-(octahydroindolizin-7-yl)-1H-indol-5-yl)urea; N-(2-ethoxycarbonyl)phenyl-N′-(3-(octahydro-2H-quinolizin-2-yl)-1H-indol-5-yl)urea; N-(4-ethoxycarbonyl)phenyl-N′-(3-(1-azabicyclo[5.4.0]undecan-4-yl)-1H-indol-5-yl)urea; N-(3-propoxycarbonyl)phenyl-N′-(3-(octahydroindolizin-7-yl)-1H-indol-5-yl)urea; N-(2-butoxycarbonyl)phenyl-N′-(3-(octahydro-2H-quinolizin-2-yl)-1H-indol-5-yl)urea; N-(2,3-dibromo)phenyl-N′-(3-(1-azabicyclo[5.4.0]undecan-4-yl)-1H-indol-5-yl)urea; N-(2-bromo-3-iodo)phenyl-N′-(3-(octahydroindolizin-7-yl)-1H-indol-5-yl)urea; N-(3,4-difluoro)phenyl-N′-(3-(octahydro-2H-quinolizin-2-yl)-1H-indol-5-yl)urea; N-(3-chloro-4-bromo)phenyl-N′-(3-(1-azabicyclo[5.4.0]undecan-4-yl)-1H-indol-5-yl)urea; N-(2-bromo-4-fluoro)phenyl-N′-(3-(octahydroindolizin-7-yl)-1H-indol-5-yl)urea; N-(2,4-diiodo)phenyl-N′-(3-(octahydro-2H-quinolizin-2-yl)-1H-indol-5-yl)urea; N-(2-chloro-5-iodo)phenyl-N′-(3-(1-azabicyclo[5.4.0]undecan-4-yl)-1H-indol-5-yl)urea; N-(2-fluoro-6-iodo)phenyl-N′-(3-(octahydroindolizin-7-yl)-1H-indol-5-yl)urea; N-(3-fluoro-5-chloro)phenyl-N′-(3-(octahydro-2H-quinolizin-2-yl)-1H-indol-5-yl)urea; N-(3-phenpropyl)-N′-(3-(1-azabicyclo[5.4.0]undecan-4-yl)-1H-indol-5-yl)urea; N-ethyl-N-phenyl-N′-(3-(octahydroindolizin-7-yl)-1H-indol-5-yl)urea; N-isopropyl-N-phenyl-N′-(3-(octahydro-2H-quinolizin-2-yl)-1H-indol-5-yl)urea; N-ethyl-N-methyl-N′-(3-(1-azabicyclo[5.4.0]undecan-4-yl)-1H-indol-5-yl)urea; N-methyl-N-isopropyl-N′-(3-(octahydroindolizin-7-yl)-1H-indol-5-yl)urea; N-ethyl-N-propyl-N′-(3-(octahydro-2H-quinolizin-2-yl)-1H-indol-5-yl)urea; N-ethyl-N-butyl-N′-(3-(1-azabicyclo[5.4.0]undecan-4-yl)-1H-indol-5-yl)urea; N-propyl-N-isopropyl-N′-(3-(octahydroindolizin-7-yl)-1H-indol-5-yl)urea; N,N-diisopropyl-N′-(3-(octahydro-2H-quinolizin-2-yl)-1H-indol-5-yl)urea; N,N-dibutyl-N′-(3-(1-azabicyclo[5.4.0]undecan-4-yl)-1H-indol-5-yl)urea; 5-isopropoxycarbonylamino-3-(1,2,3,4,5,8-hexahydroindolizin-7-yl)-2-ethyl-benzothiophene; 5-(1-buten-4-yloxy)carbonylamino-3-(1,4,5,6,7,8,9-heptahydroquinolizin-2-yl)-1H-indole hydrochloride; 5-(1-penten-5-yloxy)carbonylamino-3-(1-azabicyclo[5.4.0]undec-3-en-4-yl)-benzofuran; 5-(1-buten-4-yloxy)carbonylamino-3-(1,2,3,4,5,8-hexahydroindolizin-7-yl)-benzothiophene; 5-(3-penten-5-yloxy)carbonylamino-3-(1,4,5,6,7,8,9-heptahydroquinolizin-2-yl)-1H-indole; 5-(2-hexen-6-yloxy)carbonylamino-3-(1-azabicyclo[5.4.0]undec-3-en-4-yl)-benzofuran; 5-(4-hexen-6-yloxy)carbonylamino-3-(1,2,3,4,5,8-hexahydroindolizin-7-yl)-benzothiophene; 5-(2-chlorophenoxy)carbonylamino-3-(1,4,5,6,7,8,9-heptahydroquinolizin-2-yl)-1H-indole; 5-(3-fluorophenoxy)carbonylamino-3-(1-azabicyclo[5.4.0]undec-3-en-4-yl)-benzofuran; 5-(3-bromophenoxy)carbonylamino-3-(1,2,3,4,5,8-hexahydroindolizin-7-yl)-benzothiophene; 5-(2-bromophenoxy)carbonylamino-3-(1,4,5,6,7,8,9-heptahydroquinolizin-2-yl)-1H-indole; 5-(3-iodophenoxy)carbonylamino-3-(1-azabicyclo[5.4.0]undec-3-en-4-yl)-benzofuran; 5-(3-methoxyphenoxy)carbonylamino-3-(1,2,3,4,5,8-hexahydroindolizin-7-yl)-benzothiophene; 5-(4-ethoxyphenoxy)carbonylamino-3-(1,4,5,6,7,8,9-heptahydroquinolizin-2-yl)-1H-indole; 5-(2-ethoxy)phenoxycarbonylamino-3-(1-azabicyclo[5.4.0]undec-3-en-4-yl)-benzofuran; 5-(3-propoxyphenoxy)carbonylamino-3-(1,2,3,4,5,8-hexahydroindolizin-7-yl)-benzothiophene; 5-(2-butoxyphenoxy)carbonylamino-3-(1,4,5,6,7,8,9-heptahydroquinolizin-2-yl)-1H-indole; 5-(4-butoxyphenoxy)carbonylamino-3-(1-azabicyclo[5.4.0]undec-3-en-4-yl)-benzofuran; 5-cyclobutoxycarbonylamino-3-(1,2,3,4,5,8-hexahydroindolizin-7-yl)-benzothiophene; 5-cyclooctyloxycarbonylamino-3-(1,4,5,6,7,8,9-heptahydroquinolizin-2-yl)-1H-indole; 5-(butoxymethoxy)carbonylamino-3-(1-azabicyclo[5.4.0]undec-3-en-4-yl)-benzofuran; 5-(ethoxypropoxy)carbonylamino-3-(1,2,3,4,5,8-hexahydroindolizin-7-yl)-benzothiophene; 5-butoxycarbonylamino-3-(octahydroindolizin-7-yl)-2-propyl-1H-indole; 5-(2-buten-4-yloxy)carbonylamino-3-(octahydro-2H-quinolizin-2-yl)-benzofuran; 5-(2-penten-5-yloxy)carbonylamino-3-(1-azabicyclo[5.4.0]undecan-4-yl)-benzothiophene; 5-(1-hexen-6-yloxy)carbonylamino-3-(octahydroindolizin-7-yl)-1H-indole; 5-(3-hexen-6-yloxy)carbonylamino-3-(octahydro-2H-quinolizin-2-yl)-benzofuran; 5-(3-chlorophenoxy)carbonylamino-3-(1-azabicyclo[5.4.0]undecan-4-yl)-benzothiophene; 5-(2-fluorophenoxy)carbonylamino-3-(octahydroindolizin-7-yl)-1H-indole; 5-(4-fluorophenoxy)carbonylamino-3-(octahydro-2H-quinolizin-2-yl)-benzofuran; 5-(4-bromophenoxy)carbonylamino-3-(1-azabicyclo[5.4.0]undecan-4-yl)-benzothiophene; 5-(2-iodophenoxy)carbonylamino-3-(octahydroindolizin-7-yl)-1H-indole; 5-(4-iodophenoxy)carbonylamino-3-(octahydro-2H-quinolizin-2-yl)-benzofuran; 5-(3-chlorophenoxy)carbonylamino-3-(1-azabicyclo[5.4.0]undecan-4-yl)-benzothiophene; 5-(2-methoxyphenoxy)carbonylamino-3-(octahydroindolizin-7-yl)-2-ethyl-1H-indole citrate; 5-(3-ethoxyphenoxy)carbonylamino-3-(octahydro-2H-quinolizin-2-yl)-benzofuran; 5-(4-propoxyphenoxy)carbonylamino-3-(1-azabicyclo[5.4.0]undecan-4-yl)-benzothiophene; 5-(2-propoxyphenoxy)carbonylamino-3-(octahydroindolizin-7-yl)-1H-indole; 5-(3-butoxyphenoxy)carbonylamino-3-(octahydro-2H-quinolizin-2-yl)-benzofuran; 5-cyclopropoxycarbonylamino-3-(1-azabicyclo[5.4.0]undecan-4-yl)-benzothiophene; 5-cyclohexyloxycarbonylamino-3-(octahydroindolizin-7-yl)-1H-indole; 5-cyclooctyloxycarbonylamino-3-(octahydro-2H-quinolizin-2-yl)-benzofuran; 5-(propoxyethoxy)carbonylamino-3-(1-azabicyclo[5.4.0]undecan-4-yl)-benzothiophene; 5-(4-methoxybutoxy)carbonylamino (octahydroindolizin-7-yl)-1H-indole; 5-(acetyl)amino-3-(1,4,5,6,7,8,9-heptahydroquinolizin-2-yl)-benzofuran; 5-(butyroyl)amino-3-(1-azabicyclo[5.4.0]undec-3-en-4-yl)-benzothiophene; 5-(pentanoyl)amino-3-(1,2,3,4,5,8-hexahydroindolizin-7-yl)-1H-indole glycollate; 5-(2-methylbutanoyl)amino-3-(1,4,5,6,7,8,9-heptahydroquinolizin-2-yl)-benzofuran; 5-(2,2-dimethylpropanoyl)amino-3-(1-azabicyclo[5.4.0]undec-3-en-4-yl)-benzothiophene; 5-(heptanoyl)amino-3-(1,2,3,4,5,8-hexahydroindolizin-7-yl)-1H-indole; 5-(cyclooctylcarbonyl)amino-3-(1,4,5,6,7,8,9-heptahydroquinolizin-2-yl)-benzofuran; 5-(4-phenylbutanoyl)amino-3-(1-azabicyclo[5.4.0]undec-3-en-4-yl)-benzothiophene; 5-(phenoxyacetyl)amino-3-(1,2,3,4,5,8-hexahydroindolizin-7-yl)-1H-indole; 5-(4-phenoxybutanoyl)amino-3-(1,4,5,6,7,8,9-heptahydroquinolizin-2-yl)-benzofuran; 5-(butoxyacetyl)amino-3-(1-azabicyclo[5.4.0]undec-3-en-4-yl)-benzothiophene; 5-(4-ethoxybutanoyl)amino-3-(1,2,3,4,5,8-hexahydroindolizin-7-yl)-1H-indole tartrate; 5-(butoxycarbonylacetyl)amino-3-(1,4,5,6,7,8,9-heptahydroquinolizin-2-yl)-benzofuran; 5-(4-butoxycarbonylbutanoyl)amino-3-(1-azabicyclo[5.4.0]undec-3-en-4-yl)-benzothiophene; 5-benzoylamino-3-(1,2,3,4,5,8-hexahydroindolizin-7-yl)-1H-indole hydrochloride;5-benzoylamino-3-(1,4,5,6,7,8,9-heptahydroquinolizin-2-yl)-benzofuran; 5-benzoylamino-3-(1-azabicyclo[5.4.0]undec-3-en-4-yl)-benzothiophene; 5-benzoylamino-3-(1,2,3,4,5,8-hexahydroindolizin-7-yl)-1H-indole; 5-benzoylamino-3-(1,4,5,6,7,8,9-heptahydroquinolizin-2-yl)-benzofuran; 5-benzoylamino-3-(1-azabicyclo[5.4.0]undec-3-en-4-yl)-benzothiophene; 5-benzoylamino-3-(1,2,3,4,5,8-hexahydroindolizin-7-yl)-1H-indole; 5-benzoylamino-3-(1,4,5,6,7,8,9-heptahydroquinolizin-2-yl)-benzofuran; 5-benzoylamino-3-(1-azabicyclo[5.4.0]undec-3-en-4-yl)-benzothiophene; 5-benzoylamino-3-(1,2,3,4,5,8-hexahydroindolizin-7-yl)-1H-indole; 5-benzoylamino-3-(1,4,5,6,7,8,9-heptahydroquinolizin-2-yl)-benzofuran propanesulfonate; 5-benzoylamino-3-(1-azabicyclo[5.4.0]undec-3-en-4-yl)-benzothiophene; 5-(4-fluorobenzoyl)amino-3-(1,2,3,4,5,8-hexahydroindolizin-7-yl)-1H-indole fumarate; 5-(4-fluorobenzoyl)amino-3-(1,4,5,6,7,8,9-heptahydroquinolizin-2-yl)-benzofuran; 5-(4-fluorobenzoyl)amino-3-(1-azabicyclo[5.4.0]undec-3-en-4-yl)-benzothiophene; 5-(4-fluorobenzoyl)amino-3-(1,2,3,4,5,8-hexahydroindolizin-7-yl)-1H-indole; 5-(4-fluorobenzoyl)amino-3-(1,4,5,6,7,8,9-heptahydroquinolizin-2-yl)-benzofuran; 5-(4-fluorobenzoyl)amino-3-(1-azabicyclo[5.4.0]undec-3-en-4-yl)-benzothiophene; 5-(4-fluorobenzoyl)amino-3-(1,2,3,4,5,8-hexahydroindolizin-7-yl)-1H-indole; 5-(4-fluorobenzoyl)-N-methylamino-3-(1,4,5,6,7,8,9-heptahydroquinolizin-2-yl)-benzofuran naphthalene-1-sulfonate; 5-(4-fluorobenzoyl)amino-3-(1-azabicyclo[5.4.0]undec-3-en-4-yl)-benzothiophene; 5-(4-fluorobenzoyl)amino-3-(1,2,3,4,5,8-hexahydroindolizin-7-yl)-1H-indole; 5-(4-fluorobenzoyl)amino-3-(1,4,5,6,7,8,9-heptahydroquinolizin-2-yl)-benzofuran; 5-(4-fluorobenzoyl)amino-3-(1-azabicyclo[5.4.0]undec-3-en-4-yl)-benzothiophene; 5-(2-chlorobenzoyl)amino-3-(1,2,3,4,5,8-hexahydroindolizin-7-yl)-1H-indole; 5-(2-chlorobenzoyl)amino-3-(1,4,5,6,7,8,9-heptahydroquinolizin-2-yl)-benzofuran; 5-(2-chlorobenzoyl)amino-3-(1-azabicyclo[5.4.0]undec-3-en-4-yl)-benzothiophene; 5-(2-chlorobenzoyl)amino-3-(1,2,3,4,5,8-hexahydroindolizin-7-yl)-1H-indole; 5-(2-chlorobenzoyl)amino-3-(1,4,5,6,7,8,9-heptahydroquinolizin-2-yl)-benzofuran; 5-(2-chlorobenzoyl)amino-3-(1-azabicyclo[5.4.0]undec-3-en-4-yl)-benzothiophene; 5-(2-chlorobenzoyl)amino-3-(1,2,3,4,5,8-hexahydroindolizin-7-yl)-1H-indole; 5-(2-chlorobenzoyl)-N-ethylamino-3-(1,4,5,6,7,8,9-heptahydroquinolizin-2-yl)-benzofuran; 5-(2-chlorobenzoyl)amino-3-(1-azabicyclo[5.4.0]undec-3-en-4-yl)-benzothiophene; 5-(2-chlorobenzoyl)amino-3-(1,2,3,4,5,8-hexahydroindolizin-7-yl)-1H-indole; 5-(2-chlorobenzoyl)amino-3-(1,4,5,6,7,8,9-heptahydroquinolizin-2-yl)-benzofuran; 5-(2-chlorobenzoyl)amino-3-(1-azabicyclo[5.4.0]undec-3-en-4-yl)-benzothiophene; 5-(3-bromobenzoyl)amino-3-(1,2,3,4,5,8-hexahydroindolizin-7-yl)-1H-indole; 5-(2-iodobenzoyl)-N-propylamino-3-(1,4,5,6,7,8,9-heptahydroquinolizin-2-yl)-benzofuran; 5-(3-ethylbenzoyl)amino-3-(1-azabicyclo[5.4.0]undec-3-en-4-yl)-benzothiophene; 5-(4-propylbenzoyl)amino-3-(1,2,3,4,5,8-hexahydroindolizin-7-yl)-1H-indole; 5-(2-propylbenzoyl)amino-3-(1,4,5,6,7,8,9-heptahydroquinolizin-2-yl)-benzofuran; 5-(3-butylbenzoyl)amino-3-(1-azabicyclo[5.4.0]undec-3-en-4-yl)-benzothiophene; 5-(4-ethoxybenzoyl)amino-3-(1,2,3,4,5,8-hexahydroindolizin-7-yl)-1H-indole; 5-(2-ethoxybenzoyl)-N-isopropylamino-3-(1,4,5,6,7,8,9-heptahydroquinolizin-2-yl)-benzofuran; 5-(3-propoxybenzoyl)amino-3-(1-azabicyclo[5.4.0]undec-3-en-4-yl)-benzothiophene; 5-(2-butoxybenzoyl)amino-3-(1,2,3,4,5,8-hexahydroindolizin-7-yl)-benzofuran; 5-(4-butoxybenzoyl)amino-3-(1,4,5,6,7,8,9-heptahydroquinolizin-2-yl)-1H-indole; 5-(3-pentoxybenzoyl)amino-3-(1-azabicyclo[5.4.0]undec-3-en-4-yl)-benzothiophene; 5-(3-hexyloxybenzoyl)amino-3-(1,2,3,4,5,8-hexahydroindolizin-7-yl)-benzofuran; 5-(3-heptyloxybenzoyl)amino-3-(1,4,5,6,7,8,9-heptahydroquinolizin-2-yl)-1H-indole; 5-(2-octyloxybenzoyl)amino-3-(1-azabicyclo[5.4.0]undec-3-en-4-yl)-benzothiophene; 5-(4-octyloxybenzoyl)amino-3-(1,2,3,4,5,8-hexahydroindolizin-7-yl)-benzofuran; 5-(3-octyloxybenzoyl)amino-3-(1,4,5,6,7,8,9-heptahydroquinolizin-2-yl)-1H-indole; 5-(3-methylthiobenzoyl)amino-3-(1-azabicyclo[5.4.0]undec-3-en-4-yl)-benzothiophene; 5-(4-propylthiobenzoyl)-N-butylamino-3-(1,2,3,4,5,8-hexahydroindolizin-7-yl)-benzofuran; 5-(2-propylthiobenzoyl)amino-3-(1,4,5,6,7,8,9-heptahydroquinolizin-2-yl)-1H-indole; 5-(3-butylthiobenzoyl)amino-3-(1-azabicyclo[5.4.0]undec-3-en-4-yl)-benzothiophene; 5-(2-nitrobenzoyl)amino-3-(1,2,3,4,5,8-hexahydroindolizin-7-yl)-benzofuran; 5-(2-cyanobenzoyl)amino-3-(1,4,5,6,7,8,9-heptahydroquinolizin-2-yl)-1H-indole; 5-(2-(dimethylamino)benzoyl)amino-3-(1-azabicyclo[5.4.0]undec-3-en-4-yl)-benzothiophene; 5-(3-(diethylamino)benzoyl)amino-3-(1,2,3,4,5,8-hexahydroindolizin-7-yl)-benzofuran; 5-(4-(dipropylamino)benzoyl)amino-3-(1,4,5,6,7,8,9-heptahydroquinolizin-2-yl)-1H-indole; 5-(2-trifluoromethoxybenzoyl)amino-3-(1-azabicyclo[5.4.0]undec-3-en-4-yl)-benzothiophene; 5-(3-trifluoromethoxybenzoyl)amino-3-(1,2,3,4,5,8-hexahydroindolizin-7-yl)-benzofuran 5-(3-formylbenzoyl)amino-3-(1,4,5,6,7,8,9-heptahydroquinolizin-2-yl)-1H-indole; 5-(3-acetylbenzoyl)amino-3-(1-azabicyclo[5.4.0]undec-3-en-4-yl)-benzothiophene; 5-(2-(propanoyl)benzoyl)amino-3-(1,2,3,4,5,8-hexahydroindolizin-7-yl)-benzofuran; 5-(4-(propanoyl)benzoyl)amino-3-(1,4,5,6,7,8,9-heptahydroquinolizin-2-yl)-1H-indole; 5-(3-(butanoyl)benzoyl)amino-3-(1-azabicyclo[5.4.0]undec-3-en-4-yl)-benzothiophene; 5-(3-(benzoyl)benzoyl)amino-3-(1,2,3,4,5,8-hexahydroindolizin-7-yl)-benzofuran; 5-(3-methanesulfonylbenzoyl)amino-3-(1,4,5,6,7,8,9-heptahydroquinolizin-2-yl)-1H-indole; 5-(3-ethanesulfonylbenzoyl)amino-3-(1-azabicyclo[5.4.0]undec-3-en-4-yl)-benzothiophene; 5-(2-butanesulfonylbenzoyl)amino-3-(1,2,3,4,5,8-hexahydroindolizin-7-yl)-benzofuran; 5-(3-butanesulfonylbenzoyl)amino-3-(1,4,5,6,7,8,9-heptahydroquinolizin-2-yl)-1H-indole; 5-(2-phenylbenzoyl)amino-3-(1-azabicyclo[5.4.0]undec-3-en-4-yl)-benzothiophene; 5-(2,3-dibromo)benzoylamino-3-(1,2,3,4,5,8-hexahydroindolizin-7-yl)-benzofuran; 5-(2-bromo-3-iodo)benzoylamino-3-(1,4,5,6,7,8,9-heptahydroquinolizin-2-yl)-1H-indole; 5-(3,4-difluoro)benzoylamino-3-(1-azabicyclo[5.4.0]undec-3-en-4-yl)-benzothiophene; 5-(3-chloro-4-bromo)benzoylamino-3-(1,2,3,4,5,8-hexahydroindolizin-7-yl)-benzofuran; 5-(2-bromo-4-fluoro)benzoylamino-3-(1,4,5,6,7,8,9-heptahydroquinolizin-2-yl)-1H-indole; 5-(2,4-diiodo)benzoylamino-3-(1-azabicyclo[5.4.0]undec-3-en-4-yl)-benzothiophene; 5-(2-chloro-5-iodo)benzoylamino-3-(1,2,3,4,5,8-hexahydroindolizin-7-yl)-benzofuran; 5-(2-fluoro-6-iodo)benzoylamino-3-(1,4,5,6,7,8,9-heptahydroquinolizin-2-yl)-1H-indole; 5-(3-fluoro-5-chloro)benzoylamino-3-(1-azabicyclo[5.4.0]undec-3-en-4-yl)-benzothiophene; 5-(2-thienoyl)amino-3-(1,2,3,4,5,8-hexahydroindolizin-7-yl)-benzofuran; 5-(2-thienoyl)amino-3-(1,4,5,6,7,8,9-heptahydroquinolizin-2-yl)-1H-indole; 5-(2-thienoyl)amino-3-(1-azabicyclo[5.4.0]undec-3-en-4-yl)-benzothiophene; 5-(2-thienoyl)amino-3-(1,2,3,4,5,8-hexahydroindolizin-7-yl)-benzofuran; 5-(2-thienoyl)amino-3-(1,4,5,6,7,8,9-heptahydroquinolizin-2-yl)-1H-indole; 5-(2-thienoyl)amino-3-(1-azabicyclo[5.4.0]undec-3-en-4-yl)-benzothiophene; 5-(2-thienoyl)amino-3-(1,2,3,4,5,8-hexahydroindolizin-7-yl)-benzofuran; 5-(2-thienoyl)amino-3-(1,4,5,6,7,8,9-heptahydroquinolizin-2-yl)-1H-indole; 5-(2-thienoyl)amino-3-(1-azabicyclo[5.4.0]undec-3-en-4-yl)-benzothiophene; 5-(2-thienoyl)amino-3-(1,2,3,4,5,8-hexahydroindolizin-7-yl)-benzofuran; 5-(2-thienoyl)amino-3-(1,4,5,6,7,8,9-heptahydroquinolizin-2-yl)-1H-indole; 5-(2-thienoyl)amino-3-(1-azabicyclo[5.4.0]undec-3-en-4-yl)-benzothiophene; 5-(3-thienoyl)amino-3-(1,2,3,4,5,8-hexahydroindolizin-7-yl)-benzofuran; 5-(3-thienoyl)amino-3-(1,4,5,6,7,8,9-heptahydroquinolizin-2-yl)-1H-indole; 5-(3-thienoyl)amino-3-(1-azabicyclo[5.4.0]undec-3-en-4-yl)-benzothiophene; 5-(3-thienoyl)amino-3-(1,2,3,4,5,8-hexahydroindolizin-7-yl)-benzofuran; 5-(3-thienoyl)amino-3-(1,4,5,6,7,8,9-heptahydroquinolizin-2-yl)-1H-indole; 5-(3-thienoyl)amino-3-(1-azabicyclo[5.4.0]undec-3-en-4-yl)-benzothiophene; 5-(3-thienoyl)amino-3-(1,2,3,4,5,8-hexahydroindolizin-7-yl)-benzofuran; 5-(3-thienoyl)amino-3-(1,4,5,6,7,8,9-heptahydroquinolizin-2-yl)-1H-indole; 5-(3-thienoyl)amino-3-(1-azabicyclo[5.4.0]undec-3-en-4-yl)-benzothiophene; 5-(3-thienoyl)amino-3-(1,2,3,4,5,8-hexahydroindolizin-7-yl)-benzofuran; 5-(3-thienoyl)amino-3-(1,4,5,6,7,8,9-heptahydroquinolizin-2-yl)-1H-indole; 5-(3-thienoyl)amino-3-(1-azabicyclo[5.4.0]undec-3-en-4-yl)-benzothiophene; 5-(2-furoyl)amino-3-(1,2,3,4,5,8-hexahydroindolizin-7-yl)-benzofuran; 5-(2-furoyl)amino-3-(1,4,5,6,7,8,9-heptahydroquinolizin-2-yl)-1H-indole; 5-(2-furoyl)amino-3-(1-azabicyclo[5.4.0]undec-3-en-4-yl)-benzothiophene; 5-(2-furoyl)amino-3-(1,2,3,4,5,8-hexahydroindolizin-7-yl)-benzofuran; 5-(2-furoyl)amino-3-(1,4,5,6,7,8,9-heptahydroquinolizin-2-yl)-1H-indole; 5-(2-furoyl)amino-3-(1-azabicyclo[5.4.0]undec-3-en-4-yl)-benzothiophene; 5-(2-furoyl)amino-3-(octahydro-2H-quinolizin-2-yl)-benzofuran; 5-(2-furoyl)amino-3-(1,2,3,4,5,8-hexahydroindolizin-7-yl)-1H-indole; 5-(2-furoyl)amino-3-(1,4,5,6,7,8,9-heptahydroquinolizin-2-yl)-benzothiophene; 5-(2-furoyl)amino-3-(1-azabicyclo[5.4.0]undec-3-en-4-yl)-benzofuran; 5-(2-furoyl)amino-3-(1,2,3,4,5,8-hexahydroindolizin-7-yl)-1H-indole; 5-(2-furoyl)amino-3-(1,4,5,6,7,8,9-heptahydroquinolizin-2-yl)-benzothiophene; 5-(3-furoyl)amino-3-(1-azabicyclo[5.4.0]undec-3-en-4-yl)-benzofuran; 5-(3-furoyl)amino-3-(1,2,3,4,5,8-hexahydroindolizin-7-yl)-1H-indole; 5-(3-furoyl)amino-3-(1,4,5,6,7,8,9-heptahydroquinolizin-2-yl)-benzothiophene; 5-(3-furoyl)amino-3-(1-azabicyclo[5.4.0]undec-3-en-4-yl)-benzofuran; 5-(3-furoyl)amino-3-(1,2,3,4,5,8-hexahydroindolizin-7-yl)-1H-indole; 5-(3-furoyl)amino-3-(1,4,5,6,7,8,9-heptahydroquinolizin-2-yl)-benzothiophene; 5-(3-furoyl)amino-3-(1-azabicyclo[5.4.0]undec-3-en-4-yl)-benzofuran; 5-(3-furoyl)amino-3-(1,2,3,4,5,8-hexahydroindolizin-7-yl)-1H-indole; 5-(3-furoyl)amino-3-(1,4,5,6,7,8,9-heptahydroquinolizin-2-yl)-benzothiophene naphthalene-2-sulfonate; 5-(3-furoyl)amino-3-(1-azabicyclo[5.4.0]undec-3-en-4-yl)-benzofuran; 5-(3-furoyl)amino-3-(1,2,3,4,5,8-hexahydroindolizin-7-yl)-1H-indole; 5-(3-furoyl)amino-3-(1,4,5,6,7,8,9-heptahydroquinolizin-2-yl)-benzothiophene; 5-(propanoyl)amino-3-(octahydroindolizin-7-yl)-benzofuran mandalate; 5-(2-methylpropanoyl)amino-3-(octahydro-2H-quinolizin-2-yl)-1H-indole; 5-(2-methyl-4-butyn-1-oyl)amino-3-(1-azabicyclo[5.4.0]undecan-4-yl)-benzothiophene; 5-(2-methylbutanoyl)-N-methylamino-3-(octahydroindolizin-7-yl)-benzofuran; 5-(hex-3-enoyl)amino-3-(octahydro-2H-quinolizin-2-yl)-1H-indole; 5-(cyclohexaneacetyl)amino-3-(1-azabicyclo[5.4.0]undecan-4-yl)-benzothiophene; 5-(cycloheptylcarbonyl)amino-3-(octahydroindolizin-7-yl)-benzofuran; 5-(4-phenylbutanoyl)amino-3-(octahydro-2H-quinolizin-2-yl)-1H-indole; 5-(5-phenylpentanoyl)amino-3-(1-azabicyclo[5.4.0]undecan-4-yl)-benzothiophene; 5-(3-phenoxypropanoyl)amino-3-(octahydroindolizin-7-yl)-benzofuran; 5-(5-phenoxypentanoyl)amino-3-(octahydro-2H-quinolizin-2-yl)-1H-indole; 5-(3-propoxypropanoyl)amino-3-(1-azabicyclo[5.4.0]undecan-4-yl)-benzothiophene; 5-(5-methoxypentanoyl)amino-3-(octahydroindolizin-7-yl)-benzofuran; 5-((3-propoxycarbonyl)propanoyl)amino-3-(octahydro-2H-quinolizin-2-yl)-1H-indole; 5-((5-methoxycarbonyl)pentanoyl)amino-3-(1-azabicyclo[5.4.0]undecan-4-yl)-benzothiophene; 5-(benzoyl-N-ethyl)amino-3-(octahydroindolizin-7-yl)-benzofuran; 5-benzoylamino-3-(octahydro-2H-quinolizin-2-yl)-1H-indole 5-benzoylamino-3-(1-azabicyclo[5.4.0]undecan-4-yl)-benzothiophene; 5-benzoylamino-3-(octahydroindolizin-7-yl)-benzofuran; 5-benzoylamino-3-(octahydro-2H-quinolizin-2-yl)-1H-indole; 5-benzoylamino-3-(1-azabicyclo[5.4.0]undecan-4-yl)-benzothiophene; 5-benzoylamino-3-(octahydroindolizin-7-yl)-benzofuran; 5-benzoylamino-3-(octahydro-2H-quinolizin-2-yl)-1H-indole hydrochloride; 5-benzoyl-N-propylamino-3-(1-azabicyclo[5.4.0]undecan-4-yl)-benzothiophene; 5-benzoylamino-3-(octahydroindolizin-7-yl)-benzofuran; 5-benzoylamino-3-(octahydro-2H-quinolizin-2-yl)-1H-indole 5-benzoylamino-3-(1-azabicyclo[5.4.0]undecan-4-yl)-benzothiophene; 5-benzoylamino-3-(octahydroindolizin-7-yl)-benzofuran; 5-(4-fluorobenzoyl)amino-3-(octahydro-2H-quinolizin-2-yl)-1H-indole; 5-(4-fluorobenzoyl)amino-3-(1-azabicyclo[5.4.0]undecan-4-yl)-benzothiophene; 5-(4-fluorobenzoyl)amino-3-(octahydroindolizin-7-yl)-benzofuran; 5-(4-fluorobenzoyl)amino-3-octahydro-2H-quinolizin-2-yl)-1H-indole; 5-(4-fluorobenzoyl)amino-3-1-azabicyclo[5.4.0]undecan-4-yl)-benzothiophene; 5-(4-fluorobenzoyl)amino-3-octahydroindolizin-7-yl)-benzofuran; 5-(4-fluorobenzoyl)amino-3-octahydro-2H-quinolizin-2-yl)-1H-indole; 5-(4-fluorobenzoyl)amino-3-1-azabicyclo[5.4.0]undecan-4-yl)-benzothiophene; 5-(4-fluorobenzoyl)amino-3-octahydroindolizin-7-yl)-benzofuran; 5-(4-fluorobenzoyl)amino-3-octahydro-2H-quinolizin-2-yl)-1H-indole; 5-(4-fluorobenzoyl)amino-3-1-azabicyclo[5.4.0]undecan-4-yl)-benzothiophene fumarate; 5-(4-fluorobenzoyl)amino-3-octahydroindolizin-7-yl)-benzofuran; 5-(2-chlorobenzoyl)amino-3-octahydro-2H-quinolizin-2-yl)-1H-indole; 5-(2-chlorobenzoyl)amino-3-1-azabicyclo[5.4.0]undecan-4-yl)-benzothiophene; 5-(2-chlorobenzoyl)amino-3-octahydroindolizin-7-yl)-benzofuran; 5-(2-chlorobenzoyl)amino-3-octahydro-2H-quinolizin-2-yl)-1H-indole; 5-(2-chlorobenzoyl)amino-3-1-azabicyclo[5.4.0]undecan-4-yl)-benzothiophene; 5-(2-chlorobenzoyl)amino-3-octahydroindolizin-7-yl)-benzofuran; 5-(2-chlorobenzoyl)amino-3-octahydro-2H-quinolizin-2-yl)-1H-indole; 5-(2-chlorobenzoyl)amino-3-(1-azabicyclo[5.4.0]undecan-4-yl)-benzothiophene; 5-(2-chlorobenzoyl)amino-3-(octahydroindolizin-7-yl)-benzofuran; 5-(2-chlorobenzoyl)amino-3-(octahydro-2H-quinolizin-2-yl)-1H-indole; 5-(2-chlorobenzoyl)amino-3-(1-azabicyclo[5.4.0]undecan-4-yl)-benzothiophene; 5-(2-chlorobenzoyl)amino-3-(octahydroindolizin-7-yl)-benzofuran; 5-(2-bromobenzoyl)amino-3-(octahydro-2H-quinolizin-2-yl)-1H-indole; 5-(4-ethylbenzoyl)amino-3-(1-azabicyclo[5.4.0]undecan-4-yl)-benzothiophene; 5-(2-ethylbenzoyl)amino-3-(octahydroindolizin-7-yl)-benzofuran; 5-(3-propylbenzoyl)amino-3-(octahydro-2H-quinolizin-2-yl)-1H-indole; 5-(4-butylbenzoyl)amino-3-(1-azabicyclo[5.4.0]undecan-4-yl)-benzothiophene; 5-(2-butylbenzoyl)amino-3-(octahydroindolizin-7-yl)-benzofuran; 5-(3-ethoxybenzoyl)amino-3-(octahydro-2H-quinolizin-2-yl)-1H-indole; 5-(2-propoxybenzoyl)amino-3-(1-azabicyclo[5.4.0]undecan-4-yl)-benzothiophene; 5-(3-butoxybenzoyl)amino-3-(octahydroindolizin-7-yl)-benzofuran; 5-(4-pentyloxybenzoyl)amino-3-(octahydro-2H-quinolizin-2-yl)-1H-indole; 5-(2-pentyloxybenzoyl)amino-3-(1-azabicyclo [5.4.0]undecan-4-yl)-benzothiophene; 5-(2-hexyloxybenzoyl)amino-3-(octahydroindolizin-7-yl)-benzofuran; 5-(4-hexyloxybenzoyl)amino-3-(octahydro-2H-quinolizin-2-yl)-1H-indole; 5-(2-methylthiobenzoyl)amino-3-(1-azabicyclo[5.4.0]undecan-4-yl)-benzothiophene; 5-(2-ethylthiobenzoyl)amino-3-(octahydroindolizin-7-yl)-benzofuran; 5-(3-propylthiobenzoyl)amino-3-(octahydro-2H-quinolizin-2-yl)-1H-indole; 5-(3-nitrobenzoyl)amino-3-(1-azabicyclo[5.4.0]undecan-4-yl)-benzothiophene; 5-(3-cyanobenzoyl)amino-3-(octahydroindolizin-7-yl)-benzofuran; 5-(4-(dimethylamino)benzoyl)amino-3-(octahydro-2H-quinolizin-2-yl)-1H-indole; 5-(2-(diethylamino)benzoyl)-N-propylamino-3-(1-azabicyclo[5.4.0]undecan-4-yl)-benzothiophene; 5-(4-(diethylamino)benzoyl)amino-3-(octahydroindolizin-7-yl)-benzofuran; 5-(3-(dibutylamino)benzoyl)amino-3-(octahydro-2H-quinolizin-2-yl)-1H-indole; 5-(4-trifluoromethoxybenzoyl)amino-3-(1-azabicyclo[5.4.0]undecan-4-yl)-benzothiophene; 5-(4-(formyl)benzoyl)amino-3-(octahydroindolizin-7-yl)-benzofuran; 5-(2-(formyl)benzoyl)amino-3-(octahydro-2H-quinolizin-2-yl)-1H-indole; 5-(2-(acetyl)benzoyl)amino-3-(1-azabicyclo[5.4.0]undecan-4-yl)-benzothiophene; 5-(3-(propanoyl)benzoyl)amino-3-(octahydroindolizin-7-yl)-benzofuran; 5-(3-(butanoyl)benzoyl)amino-3-(octahydro-2H-quinolizin-2-yl)-1H-indole; 5-(2-(butanoyl)benzoyl)amino-3-(1-azabicyclo[5.4.0]undecan-4-yl)-benzothiophene; 5-(2-(benzoyl)benzoyl)amino-3-(octahydroindolizin-7-yl)-benzofuran; 5-(2-(methanesulfonyl)benzoyl)amino-3-(octahydro-2H-quinolizin-2-yl)-1H-indole; 5-(3-(propanesulfonyl)benzoyl)amino-3-(1-azabicyclo[5.4.0]undecan-4-yl)-benzothiophene; 5-(2-butanesulfonylbenzoyl)amino-3-(octahydroindolizin-7-yl)-benzofuran; 5-(3-phenylbenzoyl)amino-3-(octahydro-2H-quinolizin-2-yl)-1H-indole; 5-(2,3-dibromo)benzoyl-N-isopropylamino-3-(1-azabicyclo[5.4.0]undecan-4-yl)-benzothiophene; 5-(2-bromo-3-iodo)benzoylamino-3-(octahydroindolizin-7-yl)-benzofuran; 5-(3,4-difluoro)benzoylamino-3-(octahydro-2H-quinolizin-2-yl)-1H-indole; 5-(3-chloro-4-bromo)benzoylamino-3-(1-azabicyclo[5.4.0]undecan-4-yl)-benzothiophene; 5-(2-bromo-4-fluoro)benzoylamino-(3-(octahydroindolizin-7-yl)-benzofuran; 5-(2,4-diiodo)benzoylamino-(3-(octahydro-2H-quinolizin-2-yl)-1H-indole; 5-(2-chloro-5-iodo)benzoylamino-(3-(1-azabicyclo[5.4.0]undecan-4-yl)-benzothiophene; 5-(2-fluoro-6-iodo)benzoylamino-(3-(octahydroindolizin-7-yl)-benzofuran; 5-(3-fluoro-5-chloro)benzoylamino-3-(octahydro-2H-quinolizin-2-yl)-1H-indole 5-(2-thienoyl)-N-butylamino-3-(1-azabicyclo[5.4.0]undecan-4-yl)-benzothiophene; 5-(2-thienoyl)amino-3-(octahydroindolizin-7-yl)-benzofuran; 5-(2-thienoyl)amino-3-(octahydro-2H-quinolizin-2-yl)-1H-indole; 5-(2-thienoyl)amino-3-(1-azabicyclo[5.4.0]undecan-4-yl)-benzothiophene; 5-(2-thienoyl)amino-3-(octahydroindolizin-7-yl)-benzofuran; 5-(2-thienoyl)amino-3-(octahydro-2H-quinolizin-2-yl)-1H-indole; 5-(2-thienoyl)amino-3-(1-azabicyclo[5.4.0]undecan-4-yl)-benzothiophene; 5-(2-thienoyl)amino-3-(octahydroindolizin-7-yl)-benzofuran; 5-(2-thienoyl)amino-3-(octahydro-2H-quinolizin-2-yl)-1H-indole; 5-(2-thienoyl)amino-3-(1-azabicyclo[5.4.0]undecan-4-yl)-benzothiophene; 5-(2-thienoyl)amino-3-(octahydroindolizin-7-yl)-benzofuran; 5-(2-thienoyl)amino-3-(octahydro-2H-quinolizin-2-yl)-1H-indole; 5-(3-thienoyl)amino-3-(1-azabicyclo[5.4.0]undecan-4-yl)-benzothiophene; 5-(3-thienoyl)amino-3-(octahydroindolizin-7-yl)-benzofuran; 5-(3-thienoyl)amino-3-(octahydro-2H-quinolizin-2-yl)-1H-indole; 5-(3-thienoyl)amino-3-(1-azabicyclo[5.4.0]undecan-4-yl)-benzothiophene; 5-(3-thienoyl)amino-3-(octahydroindolizin-7-yl)-benzofuran; 5-(3-thienoyl)amino-3-(octahydro-2H-quinolizin-2-yl)-1H-indole; 5-(3-thienoyl)amino-3-(1-azabicyclo[5.4.0]undecan-4-yl)-benzothiophene; 5-(3-thienoyl)amino-3-(octahydroindolizin-7-yl)-benzofuran; 5-(3-thienoyl)amino-3-(octahydro-2H-quinolizin-2-yl)-1H-indole; 5-(3-thienoyl)amino-3-(1-azabicyclo[5.4.0]undecan-4-yl)-benzothiophene; 5-(3-thienoyl)amino-3-(octahydroindolizin-7-yl)-benzofuran; 5-(3-thienoyl)amino-3-(octahydro-2H-quinolizin-2-yl)-1H-indole; 5-(2-furoyl)amino-3-(1-azabicyclo[5.4.0]undecan-4-yl)-benzothiophene; 5-(2-furoyl)amino-3-(octahydroindolizin-7-yl)-benzofuran 5-(2-furoyl)amino-3-(octahydro-2H-quinolizin-2-yl)-1H-indole; 5-(2-furoyl)amino-3-(1-azabicyclo[5.4.0]undecan-4-yl)-benzothiophene; 5-(2-furoyl)amino-3-(octahydroindolizin-7-yl)-benzofuran 5-(2-furoyl)amino-3-(octahydro-2H-quinolizin-2-yl)-1H-indole; 5-(2-furoyl)amino-3-(1-azabicyclo[5.4.0]undecan-4-yl)-benzothiophene; 5-(2-furoyl)amino-3-(octahydroindolizin-7-yl)-benzofuran; 5-(2-furoyl)amino-3-(octahydro-2H-quinolizin-2-yl)-1H-indole; 5-(2-furoyl)amino-3-(1-azabicyclo[5.4.0]undecan-4-yl)-benzothiophene; 5-(2-furoyl)amino-3-(octahydroindolizin-7-yl)-benzofuran; 5-(2-furoyl)amino-3-(octahydro-2H-quinolizin-2-yl)-1H-indole; 5-(3-furoyl)amino-3-1-azabicyclo[5.4.0]undecan-4-yl)-benzothiophene; 5-(3-furoyl)amino-3-octahydroindolizin-7-yl)-benzofuran; 5-(3-furoyl)amino-3-octahydro-2H-quinolizin-2-yl)-1H-indole; 5-(3-furoyl)amino-3-1-azabicyclo[5.4.0]undecan-4-yl)-benzothiophene; 5-(3-furoyl)amino-3-octahydroindolizin-7-yl)-benzofuran; 5-(3-furoyl)amino-3-octahydro-2H-quinolizin-2-yl)-1H-indole; 5-(3-furoyl)amino-3-1-azabicyclo[5.4.0]undecan-4-yl)-benzothiophene; 5-(3-furoyl)amino-3-octahydroindolizin-7-yl)-benzofuran; 5-(3-furoyl)amino-3-octahydro-2H-quinolizin-2-yl)-1H-indole; 5-(3-furoyl)amino-3-1-azabicyclo[5.4.0]undecan-4-yl)-benzothiophene; 5-(3-furoyl)amino-3-octahydroindolizin-7-yl)-1H-indole; 5-(3-furoyl)amino-3-octahydro-2H-quinolizin-2-yl)-benzofuran; N-[pyridin-2-yl]-5-carboxamido-3-(1,2,5,6-tetrahydropyridin-4-yl)-1H-indole; N-[fur-3-yl]-5-carboxamido-3-(1,2,5,6-tetrahydropyridin-4-yl)-benzothiophene; N-[pyrazol-3-yl]-5-carboxamido-3-(1,2,5,6-tetrahydropyridin-4-yl)-benzofuran; N-[thiazol-2-yl]-5-carboxamido-3-(1,2,5,6-tetrahydropyridin-4-yl)-1H-indole; N-[quinolin-4-yl]-5-carboxamido-3-(1,2,5,6-tetrahydropyridin-4-yl)-benzothiophene; N-[imidazol-4-yl]-5-carboxamido-3-(1-azabicyclo[5.4.0]undecan-4-yl)-benzofuran; N-[fur-3-yl]-5-carboxamido-3-(1,2,5,6-tetrahydropyridin-4-yl)-1H-indole; N-[pyrimidin-5-yl]-5-carboxamido-3-(octahydroindolizin-7-yl)-benzothiophene; N-[indol-2-yl]-5-carboxamido-3-(octahydro-2H-quinolizin-2-yl)-benzofuran; N-[isoxazol-5-yl]-5-carboxamido-3-(1-azabicyclo[5.4.0]undecan-4-yl)-1H-indole; 5-amino-3-(1,2,3,4,5,8-hexahydroindolizin-7-yl)pyrrolo[3,2-b]pyridine; 5-(3-chlorophenyl)thio-3-(1,4,5,6,7,8,9-heptahydroquinolizin-2-yl)-2-methylpyrrolo[3,2-b]pyridine; 5-(2-pyridinyl)thio-3-(1-azabicyclo[5.4.0]undec-3-en-4-yl)pyrrolo[3,2-b]pyridine decanoate; 5-propanoyl-3-(1,2,3,4,5,8-hexahydroindolizin-7-yl)pyrrolo[3,2-b]pyridine suberate; 5-(3,3-dimethylbutanoyl)-3-(1-azabicyclo[5.4.0]undec-3-en-4-yl)pyrrolo[3,2-b]pyridine; 5-(3-fluorobenzoyl)-3-(1-azabicyclo[5.4.0]undec-3-en-4-yl)pyrrolo[3,2-b]pyridine; 5-(3-pyridinecarbonyl)-3-(1,4,5,6,7,8,9-heptahydroquinolizin-2-yl)pyrrolo[3,2-b]pyridine; 5-(N-phenyl)carboxamido-3-(1,2,3,4,5,8-hexahydroindolizin-7-yl)-2-methylpyrrolo[3,2-b]pyridine 2,4-dinitrobenzoate; 5-(N-benzyl)carboxamido-3-(1,4,5,6,7,8,9-heptahydroquinolizin-2-yl)pyrrolo[3,2-b]pyridine; 5-(N-(2-(4-chlorophenyl)ethyl))carboxamido-3-(1-azabicyclo[5.4.0]undec-3-en-4-yl)pyrrolo[3,2-b]pyridine 4-hydroxybenzoate; 5-(N-isopropyl-N-sec-butanesulfonyl)amino-3-(1-azabicyclo[5.4.0]undecan-4-yl)pyrrolo[3,2-b]pyridine; 5-(N-(tert-butyl)sulfonyl)amino-3-(octahydroindolizin-7-yl)pyrrolo[3,2-b]pyridine; 5-(N,N-diethylaminosulfonyl)amino-3-(octahydro-2H-quinolizin-2-yl)pyrrolo[3,2-b]pyridine; N-ethyl-N′-(3-(1,2,3,4,5,8-hexahydroindolizin-7-yl)pyrrolo[3,2-b]pyridin-5-yl)thiourea; N-isopropyl-N′-(3-(1,4,5,6,7,8,9-heptahydroquinolizin-2-yl)pyrrolo[3,2-b]pyridin-5-yl)thiourea; N-(3-methoxy)phenyl-N′-(3-(1-azabicyclo[5.4.0]undec-3-en-4-yl)pyrrolo[3,2-b]pyridin-5-yl)thiourea; N-(2-bromo-3-iodo)phenyl-N′-(3-(1-azabicyclo[5.4.0]undec-3-en-4-yl)pyrrolo[3,2-b]pyridin-5-yl)urea; N-(3,4-difluoro)phenyl-N′-(3-(1,2,3,4,5,8-hexahydroindolizin-7-yl)pyrrolo[3,2-b]pyridin-5-yl)urea; N-(3-chloro-4-bromo)phenyl-N′-(3-(1,4,5,6,7,8,9-heptahydroquinolizin-2-yl)pyrrolo[3,2-b]pyridin-5-yl)urea; N-(2-bromo-4-fluoro)phenyl-N′-(3-(1-azabicyclo[5.4.0]undec-3-en-4-yl)pyrrolo[3,2-b]pyridin-5-yl)urea; N-methyl-N-propyl-N′-(3-(1,4,5,6,7,8,9-heptahydroquinolizin-2-yl)pyrrolo[3,2-b]pyridin-5-yl)urea; N-ethyl-N-isopropyl-N′-(3-(1-azabicyclo[5.4.0]undec-3-en-4-yl)pyrrolo[3,2-b]pyridin-5-yl)urea; N,N-dipropyl-N′-(3-(1,2,3,4,5,8-hexahydroindolizin-7-yl)pyrrolo[3,2-b]pyridin-5-yl)urea; N-butyl-N-propyl-N′-(3-(1,4,5,6,7,8,9-heptahydroquinolizin-2-yl)pyrrolo[3,2-b]pyridin-5-yl)urea; N-butyl-N-isopropyl-N′-(3-(1-azabicyclo[5.4.0]undec-3-en-4-yl)pyrrolo[3,2-b]pyridin-5-yl)urea; N-hexyl-N′-(3-(octahydroindolizin-7-yl)pyrrolo[3,2-b]pyridin-5-yl)urea; N-(2-buten-4-yl)-N′-(3-(octahydro-2H-quinolizin-2-yl)pyrrolo[3,2-b]pyridin-5-yl)urea; N-cyclopentyl-N′-(3-(octahydroindolizin-7-yl)pyrrolo[3,2-b]pyridin-5-yl)urea; 5-isopropoxycarbonylamino-3-(1,2,3,4,5,8-hexahydroindolizin-7-yl)-2-ethylpyrrolo[3,2-b]pyridine; 5-(3-bromophenoxy)carbonylamino-3-(1,2,3,4,5,8-hexahydroindolizin-7-yl)pyrrolo[3,2-b]pyridine; 5-(acetyl)amino-3-(1,4,5,6,7,8,9-heptahydroquinolizin-2-yl)pyrrolo[3,2-b]pyridine; 5-(butyroyl)amino-3-(1-azabicyclo[5.4.0]undec-3-en-4-yl)pyrrolo[3,2-b]pyridine; 5-(pentanoyl)amino-3-(1,2,3,4,5,8-hexahydroindolizin-7-yl)pyrrolo[3,2-b]pyridine glycollate; 5-(cyclooctylcarbonyl)amino-3-(1,4,5,6,7,8,9-heptahydroquinolizin-2-yl)pyrrolo[3,2-b]pyridine; 5-(4-phenylbutanoyl)amino-3-(1-azabicyclo[5.4.0]undec-3-en-4-yl)pyrrolo[3,2-b]pyridine; 5-(phenoxyacetyl)amino-3-(1,2,3,4,5,8-hexahydroindolizin-7-yl)pyrrolo[3,2-b]pyridine; 5-benzoylamino-3-(1-azabicyclo[5.4.0]undec-3-en-4-yl)pyrrolo[3,2-b]pyridine; 5-(4-fluorobenzoyl)amino-3-(1,2,3,4,5,8-hexahydroindolizin-7-yl)pyrrolo[3,2-b]pyridine fumarate; 5-(4-fluorobenzoyl)amino-3-(1,4,5,6,7,8,9-heptahydroquinolizin-2-yl)pyrrolo[3,2-b]pyridine; 5-(2-thienoyl)amino-3-(1,2,3,4,5,8-hexahydroindolizin-7-yl)pyrrolo[3,2-b]pyridine; 5-(2-furoyl)amino-3-(1-azabicyclo[5.4.0]undec-3-en-4-yl)pyrrolo[3,2-b]pyridine; 5-(4-phenylbutanoyl)amino-3-(octahydro-2H-quinolizin-2-yl)pyrrolo[3,2-b]pyridine; 5-(3-furoyl)amino-3-(1-azabicyclo[5.4.0]undecan-4-yl)pyrrolo[3,2-b]pyridine; 5-(3-furoyl)amino-3-(octahydroindolizin-7-yl)pyrrolo[3,2-b]pyridine; and 5-(3-furoyl)amino-3-(octahydro-2H-quinolizin-2-yl)pyrrolo[3,2-b]pyridine.

The following HTR_{1F} agonists are disclosed in PCT publication No. WO 98/20875: N-propyl-N′-(3-(1-(2-pentyl)-1,2,3,4-tetrahydropyridin-4-yl)pyrrolo[3,2-b]pyridin-5-yl)thiourea decanoate; N-butyl-N¹-(3-(1-isopropylpiperidin-4-yl)pyrrolo[3,2-b]pyridin-5-yl)thiourea acrylate; N-(2-methoxy)phenyl-N′-(3-(1-(sec-butyl)-1,2,3,4-tetrahydropyridin-4-yl)pyrrolo[3,2-b]pyridin-5-yl)thiourea formate; N-(4-propoxy)phenyl-N′-(3-(1-ethylpiperidin-4-yl)-pyrrolo[3,2-b]pyridin-5-yl)thiourea isobutyrate; N-(2-butoxy)phenyl-N′-(3-(1-hexylpiperidin-4-yl)pyrrolo[3,2-b]pyridin-5-yl)thiourea fumarate; N-(2,3-dibromo)phenyl-N′-(3-(1-isopropylpiperidin-4-yl)pyrrolo[3,2-b]pyridin-5-yl)thiourea caproate; N-(2-bromo-3-iodo)phenyl-N′-(3-(1-(2-pentyl)piperidin-4-yl)pyrrolo[3,2-b]pyridin-5-yl)thiourea heptanoate; N-(3-phenpropyl)-N′-(3-(1-(sec-butyl)piperidin-4-yl)pyrrolo[3,2-b]pyridin-5-yl)thiourea propiolate; N-(4-trifluoromethyl)phenyl-N′-(3-(1-neopentyl-1,2,3,4-tetrahydropyridin-4-yl)pyrrolo[3,2-b]pyridin-5-yl)thiourea oxalate; N-(4-phenyl)phenyl-N′—(3-(1-pentylpiperidin-4-yl)pyrrolo[3,2-b]pyridin-5-yl)thiourea malonate; N-hexyl-N′-(3-(1-propylpiperidin-4-yl)pyrrolo[3,2-b]pyridin-5-yl)urea succinate; N-(2-buten-4-yl)-N′-(3-(1-butylpiperidin-4-yl)pyrrolo-[3,2-b]pyridin-5-yl)urea suberate; N-(3-hexen-6-yl)-N′-(3-(1-(3-pentyl)piperidin-4-yl)-pyrrolo [3,2-b]pyridin-5-yl)urea sebacate; N-cyclopropyl-N′-(3-(1-hexyl-1,2,3,4-tetrahydropyridin-4-yl)pyrrolo[3,2-b]pyridin-5-yl)urea fumarate; N-cyclopentyl-N′-(3-(1-(3-pentyl)piperidin-4-yl)-pyrrolo[3,2-b]pyridin-5-yl)urea maleate; N-cyclooctyl-N′-(3-(1-(tert-butyl)piperidin-4-yl)-pyrrolo[3,2-b]pyridin-5-yl)urea malonate; N-(2-chloro)phenyl-N′-(3-(1-butylpiperidin-4-yl)-pyrrolo[3,2-b]pyridin-5-yl)urea mandelate; N-(3-phenyl)phenyl-N′-(3-(1-propyl-1,2,3,4-tetrahydro-pyridin-4-yl)pyrrolo[3,2-b]pyridin-5-yl)urea butyne-1,4-dioate; N-(2-ethoxy)phenyl-N′-(3-(1-neopentylpiperidin-4-yl)-pyrrolo[3,2-b]pyridin-5-yl)urea hexyne-1,6-dioate; N-(4-isopropoxy)phenyl-N′-(3-(1-isobutylpiperidin-4-yl)pyrrolo[3,2-b]pyridin-5-yl)urea benzoate; N-(2-formyl)phenyl-N′-(3-(1-(3-pentyl)piperidin-4-yl)pyrrolo[3,2-b]pyridin-5-yl)urea 4-chlorobenzoate; N-(3-propanoyl)phenyl-N′-(3-(1-pentylpiperidin-4-yl)pyrrolo[3,2-b]pyridin-5-yl)urea 2-methylbenzoate; N-(3-ethylthio)phenyl-N′-(3-(1-propylpiperidin-4-yl)pyrrolo[3,2-b]pyridin-5-yl)urea 2,4-dinitrobenzoate; N-(3-isopropylthio)phenyl-N′-(3-(1-isopropylpiperidin-4-yl)pyrrolo [3,2-b]pyridin-5-yl)urea 3-hydroxybenzoate; N-(2-methyl)phenyl-N′-(3-(1-propylpiperidin-4-yl)-pyrrolo[3,2-b]pyridin-5-yl)urea 4-methoxybenzoate; N-(3-isopropyl)phenyl-N′-(3-(1-isopropylpiperidin-4-yl)pyrrolo[3,2-b]pyridin-5-yl)urea phthalate; N-(2-ethoxycarbonyl)phenyl-N′-(3-(1-(tert-butyl)-piperidin-4-yl)pyrrolo[3,2-b]pyridin-5-yl)urea sulfonate; N-(2-butoxycarbonyl)phenyl-N′-(3-(1-propylpiperidin-4-yl)pyrrolo[3,2-b]pyridin-5-yl)urea; N-(3,4-difluoro)phenyl-N′-(3-(1-butylpiperidin-4-yl)-pyrrolo[3,2-b]pyridin-5-yl)urea; N-(3-chloro-4-bromo)phenyl-N′-(3-(1-(3-pentyl)piperi-din-4-yl)pyrrolo[3,2-b]pyridin-5-yl)urea; N-(3-phenpropyl)-N′-(3-(1-propylpiperidin-4-yl)-pyrrolo[3,2-b]pyridin-5-yl)urea; N-ethyl-N-phenyl-N′-(3-(1-butylpiperidin-4-yl)pyrrolo-[3,2-b]pyridin-5-yl)urea; N-isopropyl-N-phenyl-N′-(3-(1-(sec-butyl)piperidin-4-yl)pyrrolo[3,2-b]pyridin-5-yl)urea; N-ethyl-N-methyl-N′-(3-(1-(2-pentyl)piperidin-4-yl)-pyrrolo[3,2-b]pyridin-5-yl)urea; N-methyl-N-isopropyl-N′-(3-(1-neopentylpiperidin-4-yl)-pyrrolo[3,2-b]pyridin-5-yl)urea; N,N-diisopropyl-N′-(3-(1-butylpiperidin-4-yl)pyrrolo-[3,2-b]pyridin-5-yl)urea; 5-butoxycarbonylamino-3-(1-propyl-1,2,3,4-tetrahydropyridin-4-yl)pyrrolo[3,2-b]pyridine; 5-(2-buten-4-yloxy)carbonylamino-3-(-1,2,3,4-tetrahydropyridin-4-yl)pyrrolo[3,2-b]pyridine; 5-(2-penten-5-yloxy)carbonylamino-3-(1-(sec-butyl)-piperidin-4-yl)pyrrolo[3,2-b]pyridine; 5-(3-chlorophenoxy) carbonylamino-3-(1-hexylpiperidin-4-yl)pyrrolo[3,2-b]pyridine; 5-(2-methoxyphenoxy)carbonylamino-3-(1-hexylpiperidin-4-yl)-2-pyrrolo[3,2-b]pyridine citrate; 5-(3-butoxyphenoxy)carbonylamino-3-(1-neopentylpiperi-din-4-yl)pyrrolo[3,2-b]pyridine; 5-cyclopropoxycarbonylamino-3-(1-(tert-butyl)piperidin-4-yl)pyrrolo[3,2-b]pyridine; 5-cyclohexyloxycarbonylamino-3-(1-isobutylpiperidin-4-yl)pyrrolo[3,2-b]pyridine; 5-cyclooctyloxycarbonylamino-3-(1-isopropylpiperidin-4-yl)pyrrolo[3,2-b]pyridine; 5-(propoxyethoxy)carbonylamino-3-(1-ethylpiperidin-4-yl)pyrrolo[3,2-b]pyridine; 5-(4-methoxybutoxy)carbonylamino(1-ethylpiperidin-4-yl)pyrrolo[3,2-b]pyridine; 5-(propanoyl)amino-3-(1-neopentylpiperidin-4-yl)-pyrrolo[3,2-b]pyridine mandelate; 5-(2-methylpropanoyl)amino-3-(1-(3-pentyl)piperidin-4-yl)pyrrolo[3,2-b]pyridine; 5-(2-methyl-4-butyn-1-oyl)amino-3-(1-(tert-butyl)pi-peridin-4-yl)pyrrolo[3,2-b]pyridine; 5-(2-methylbutanoyl)-N-methylamino-3-(1-ethylpiperidin-4-yl)pyrrolo[3,2-b]pyridine phenylacetate; 5-(hex-3-enoyl)amino-3-(1-propylpiperidin-4-yl)-pyrrolo[3,2-b]pyridine; 5-(cyclohexaneacetyl)amino-3-(1-isopropylpiperidin-4-yl)pyrrolo[3,2-b]pyridine; 5-(cycloheptylcarbonyl)amino-3-(1-butylpiperidin-4-yl)-pyrrolo[3,2-b]pyridine phenylpropionate; 5-(4-phenylbutanoyl)amino-3-(1-isopropyl-1,2,3,4-tetrahydropyridin-4-yl)pyrrolo[3,2-b]pyridine phenylbutyrate; 5-(5-phenoxypentanoyl)amino-3-(1-neopentyl-1,2,3,4-tetrahydropyridin-4-yl)pyrrolo[3,2-b]pyridine citrate; 5-(5-methoxypentanoyl)amino-3-(1-isopropylpiperidin-4-yl)pyrrolo[3,2-b]pyridine lactate; 5-((3-propoxycarbonyl)propanoyl)amino-3-(1-isobutyl-piperidin-4-yl)pyrrolo[3,2-b]pyridine; 5-((5-methoxycarbonyl)pentanoyl)amino-3-(1-isopropyl-piperidin-4-yl)pyrrolo[3,2-b]pyridine p-hydroxybutyrate; 5-(benzoyl-N-ethyl)amino-3-(1-ethylpiperidin-4-yl)-pyrrolo[3,2-b]pyridine glycollate; 5-benzoylamino-3-(1-propylpiperidin-4-yl)pyrrolo[3,2-b]pyridine tartrate; 5-benzoylamino-3-(1-isopropylpiperidin-4-yl)pyrrolo-[3,2-b]pyridine; 5-benzoylamino-3-(1-(tert-butyl)-1,2,3,4-tetrahydro-pyridin-4-yl)pyrrolo[3,2-b]pyridine hydrochloride; 5-(4-fluorobenzoyl)amino-3-(1-ethyl-1,2,3,4-tetrahydropyridin-4-yl)pyrrolo[3,2-b]pyridine; 5-(4-(formyl) benzoyl)amino-3-(1-(sec-butyl)piperidin-4-yl)pyrrolo[3,2-b]pyridine; 5-(3-(butanoyl)benzoyl)amino-3-(1-methylpiperidin-4-yl)pyrrolo[3,2-b]pyridine; 5-(2-(butanoyl)benzoyl)amino-3-(1-neopentylpiperidin-4-yl)pyrrolo[3,2-b]pyridine; 5-(2-(benzoyl)benzoyl)amino-3-(1-pentylpiperidin-4-yl)pyrrolo[3,2-b]pyridine; 5-(2-(methanesulfonyl)benzoyl)amino-3-(1-butylpiperi-din-4-yl)pyrrolo[3,2-b]pyridine; 5-(3-phenylbenzoyl)amino-3-(1-(tert-butyl)piperidin-4-yl)pyrrolo[3,2-b]pyridine; 5-(2,3-dibromo)benzoyl-N-isopropylamino-3-(1-isoprop-ylpiperidin-4-yl)pyrrolo[3,2-b]pyridine; 5-(2-bromo-3-iodo)benzoylamino-3-(1-(2-pentyl)piperi-din-4-yl)pyrrolo[3,2-b]pyridine; 5-(2-thiophenecarbonyl)-N-butylamino-3-(1-ethylpiperi-din-4-yl)pyrrolo[3,2-b]pyridine; 5-(2-thiophenecarbonyl)amino-3-(1-isopropylpiperidin-4-yl)pyrrolo[3,2-b]pyridine; 5-(2-thiophenecarbonyl)amino-3-(1-(tert-butyl)piperi-din-4-yl)pyrrolo[3,2-b]pyridine; 5-(2-thiophenecarbonyl)amino-3-(1-hexylpiperidin-4-yl)-pyrrolo[3,2-b]pyridine; 5-(3-thiophenecarbonyl)amino-3-(1-ethylpiperidin-4-yl)pyrrolo[3,2-b]pyridine; 5-(2-furoyl)amino-3-(1-isopropyl-1,2,3,4-tetrahydro-pyridin-4-yl)pyrrolo[3,2-b]pyridine; 5-(2-furoyl)amino-3-(1-butyl-1,2,3,4-tetrahydropyridin-4-yl)pyrrolo[3,2-b]pyridine; 5-(2-furoyl)amino-3-(1-neopentylpiperidin-4-yl)-pyrrolo[3,2-b]pyridine; 5-(3-furoyl)amino-3-(1-butylpiperidin-4-yl)pyrrolo [3,2-b]pyridine; 5-(2-pyridinecarbonyl)amino-3-(1-butylpiperidin-4-yl)-pyrrolo[3,2-b]pyridine; 5-(2-chloro-4-pyridinecarbonyl)amino-3-(1-methyl-piperidin-4-yl)pyrrolo[3,2-b]pyridine; 5-(3-pyrrolecarbonyl)amino-3-(1-methylpiperidin-4-yl)-pyrrolo[3,2-b]pyridine; 5-(2-oxazolecarbonyl) amino-3-(1-hexylpiperidin-4-yl)-pyrrolo[3,2-b]pyridine; 5-(2-methyl-4-oxazolecarbonyl)amino-3-(1-methyl-piperidin-4-yl)pyrrolo[3,2-b]pyridine; 5-(4-pyrazolecarbonyl)amino-3-(1-methylpiperidin-4-yl)-pyrrolo[3,2-b]pyridine; 5-(5-isoxazolecarbonyl)amino-3-(1-methylpiperidin-4-yl)pyrrolo[3,2-b]pyridine; 5-(3-imidazolecarbonyl)amino-3-(1-methylpiperidin-4-yl)pyrrolo[3,2-b]pyridine; 5-(2-methoxy-4-pyrimidinecarbonyl)amino-3-(1-methyl-piperidin-4-yl)pyrrolo[3,2-b]pyridine; 5-(2-quinolinecarbonyl)amino-3-(1-methylpiperidin-4-yl)pyrrolo[3,2-b]pyridine; and 5-(2-cyano-5-quinolinecarbonyl)amino-3-(1-methyl-piperidin-4-yl)pyrrolo[3,2-b]pyridine.

The following HTR_{1F} agonists are disclosed in PCT publication No. WO 99/25348: 5-fluoro-3-(1-hexylpiperidin-4-yl)pyrrolo[3,2-b]pyridine; 5-bromo-3-(1-benzylpiperidin-4-yl)pyrrolo[3,2-b]pyridine; 5-iodo-3-(1-(1-phenyleth-2-yl)piperidin-4-yl)pyrrolo-[3,2-b]pyridine; 5-ethoxy-3-(1-pentylpiperidin-4-yl)pyrrolo [3,2-b]pyridine; 5-propo-3-cy-3-(1-butylpiperidin-4-yl)pyrrolo [3,2-b]pyridine; 5-isopropoxy-3-(1-isobutylpiperidin-4-yl)pyrrolo[3,2-b]pyridine; 5-(N-[methyl]amino)-3-(1-isopropylpiperidin-4-yl)pyrrolo[3,2-b]pyridine; 5-(N-[propyl]amino)-3-(1-ethylpiperidin-4-yl)pyrrolo-[3,2-b]pyridine; 5-(N-[isopropyl]amino)-3-(1-methylpiperidin-4-yl)pyrrolo[3,2-b]pyridine; 5-(N-[butyl]amino)-3-(1-benzylpiperidin-4-yl)pyrrolo-[3,2-b]pyridine; 5-(N-[phenethyl]amino)-3-(1-(1-phenyleth-2-yl)piperidin-4-yl)pyrrolo[3,2-b]pyridine; 5-(N-[phenpropyl]amino)-3-(1-methylpiperidin-4-yl)pyrrolo[3,2-b]pyridine; 5-(N-[phenbutyl]amino)-3-(1-methylpiperidin-4-yl)pyrrolo[3,2-b]pyridine; 5-(N-[3-furylmethyl]amino)-3-(1-methylpiperidin-4-yl)pyrrolo[3,2-b]pyridine; 5-(N-[pyridin-2-ylethyl]amino)-3-(1-methylpiperidin-4-yl)pyrrolo[3,2-b]pyridine; 5-(N-[3-(pyridin-4-yl-N-oxide)prop-1-yl]amino)-3-(1-methylpiperidin-4-yl)pyrrolo[3,2-b]pyridine; 5-(N-[4-(2-pyrrolyl) but-1-yl]amino)-3-(1-methylpiperidi-4-yl)pyrrolo[3,2-b]pyridine; 5-(N-[3-furylmethyl]amino)-3-(1-methylpiperidin-4-yl) pyrrolo[3,2-b]pyridine; 5-(N-[(oxazol-2-yl)methyl]amino)-3-(1-methylpiperidin-4-yl)pyrrolo[3,2-b]pyridine; 5-(N-[(pyrimidin-4-yl)methyl]amino)-3-(1-methylpiperidin-4-yl)pyrrolo[3,2-b]pyridine; 5-(N-[(indol-4-yl)methyl]amino)-3-(1-methylpiperidin-4-yl)pyrrolo[3,2-b]pyridine; 5-(N-[(quinolin-6-yl)methyl]amino)-3-(1-methylpiperidin-4-yl)pyrrolo[3,2-b]pyridine; N-[3-furyl]-3-(1-methylpiperidin-4-yl)pyrrolo[3,2-b]pyridine-5-carboxamide; N-[pyridin-2-yl]-3-(1-methylpiperidin-4-yl)pyrrolo[3,2-b]pyridine-5-carboxamide; N-[pyridin-4-yl-N-oxide]-3-(1-methylpiperidin-4-yl)pyrrolo[3,2-b]pyridine-5-carboxamide; N-[pyrrol-2-yl]-3-(1-methylpiperidin-4-yl)pyrrolo[3,2-b]pyridine-5-carboxamide; N-[pyrazol-3-yl]-3-(1-methylpiperidin-4-yl)pyrrolo[3,2-b]pyridine-5-carboxamide; N-[oxazol-2-yl]-3-(1-methylpiperidin-4-yl)pyrrolo[3,2-b]pyridine-5-carboxamide; N-[pyrimidin-4-yl]-3-(1-methylpiperidin-4-yl)pyrrolo-[3,2-b]pyridine-5-carboxamide; N-[indol-4-yl]-3-(1-methylpiperidin-4-yl)pyrrolo[3,2-bl pyridine-5-carboxamide; N-[quinolin-6-yl]-3-(1-methylpiperidin-4-yl)pyrrolo-[3,2-b]pyridine-5-carboxamide; 5-fluoro-3-(1-hexyl-1,2,3,6-tetrahydropyridin-4-yl)pyrrolo[3,2-b]pyridine; 5-bromo-3-(1-benzyl-1,2,3,6-tetrahydropyridin-4-yl)pyrrolo[3,2-b]pyridine; 5-iodo-3-(1-(1-phenyleth-2-yl)-1,2,3,6-tetrahydro-pyridin-4-yl)pyrrolo-[3,2-b]pyridine; 5-ethoxy-3-(1-pentyl-1,2,3,6-tetrahydropyridin-4-yl)pyrrolo[3,2-b]pyridine; 5-propoxy-3-(1-butyl-1,2,3,6-tetrahydropyridin-4-yl)pyrrolo[3,2-b]pyridine; 5-isopropoxy-3-(1-isobutyl-1,2,3,6-tetrahydropyridin-4-yl)pyrrolo[3,2-b]pyridine; 5-(N-[methyl]amino)-3-(1-isopropyl-1,2,3,6-tetrahydro-pyridin-4-yl)pyrrolo[3,2-b]pyridine; 5-(N-[propyl]amino)-3-(1-ethyl-1,2,3,6-tetrahydro-pyridin-4-yl)pyrrolo-[3,2-b]pyridine; 5-(N-[isopropyl]amino)-3-(1-methyl-1,2,3,6-tetrahydro-pyridin-4-yl)pyrrolo[3,2-b]pyridine; 5-(N-[butyl]amino)-3-(1-benzyl-1,2,3,6-tetrahydro-pyridin-4-yl)pyrrolo-[3,2-b]pyridine; 5-(N-[phenethyl]amino)-3-(1-(1-phenyleth-2-yl)-1,2,3,6-tetrahydripyridin-4-yl)pyrrolo[3,2-b]pyridine; 5-(N-[phenpropyl]amino)-3-(1-methyl-1,2,3,6-tetrahydro-pyridin-4-yl)pyrrolo[3,2-b]pyridine; 5-(N-[phenbutyl]amino)-3-(1-methyl-1,2,3,6-tetrahydro-pyridin-4-yl)pyrrolo[3,2-b]pyridine; 5-(N-[3-furylmethyl]amino)-3-(1-methyl-1,2,3,6-tetrahydropyridin-4-yl)pyrrolo[3,2-b]pyridine; 5-(N-[pyridin-2-ylethyl]amino)-3-(1-methyl-1,2,3,6-tetrahydropyridin-4-yl)pyrrolo[3,2-b]pyridine; 5-(N-[3-(pyridin-4-yl-N-oxide)prop-1-yl]amino)-3-(1-methyl-1,2,3,6-tetrahydropyridin-4-yl)pyrrolo[3,2-b]pyridine; 5-(N-[4-(2-pyrrolyl)but-1-yl]amino)-3-(1-methyl-1,2,3,6-tetrahydropyridin-4-yl)pyrrolo[3,2-b]pyridine; 5-(N-[3-furylmethyl]amino)-3-(1-methyl-1,2,3,6-tetrahydropyridin-4-yl)pyrrolo[3,2-b]pyridine; 5-(N-[(oxazol-2-yl)methyl]amino)-3-(1-methyl-1,2,3,6-tetrahydropyridin-4-yl)pyrrolo[3,2-b]pyridine; 5-(N-[(pyrimidin-4-yl)methyl]amino)-3-(1-methyl-1,2,3,6-tetrahydropyridin-4-yl)pyrrolo[3,2-b]pyridine; 5-(N-[(indol-4-yl)methyl]amino)-3-(1-methyl-1,2,3,6-tetrahydropyridin-4-yl)pyrrolo[3,2-b]pyridine; 5-(N-[(quinolin-6-yl)methyl]amino)-3-(1-methyl-1,2,3,6-tetrahydropyridin-4-yl)pyrrolo[3,2-b]pyridine; N-[3-furyl]-3-(1-methyl-1,2,3,6-tetrahydropyridin-4-yl)pyrrolo[3,2-b]pyridine-5-carboxamide; N-[pyridin-2-yl]-3-(1-methyl-1,2,3,6-tetrahydropyridin-4-yl)pyrrolo[3,2-b]pyridine-5-carboxamide; N-[pyridin-4-yl-N-oxide]-3-(1-methyl-1,2,3,6-tetrahydropyridin-4-yl)pyrrolo[3,2-b]pyridine-5-carboxamide; N-[pyrrol-2-yl]-3-(1-methyl-1,2,3,6-tetrahydropyridin-4-yl)pyrrolo[3,2-b]pyridine-5-carboxamide; N-[pyrazol-3-yl]-3-(1-methyl-1,2,3,6-tetrahydropyridin-4-yl)pyrrolo[3,2-b]pyridine-5-carboxamide; N-[oxazol-2-yl]-3-(1-methyl-1,2,3,6-tetrahydropyridin-4-yl)pyrrolo[3,2-b]pyridine-5-carboxamide; N-[pyrimidin-4-yl]-3-(1-methyl-1,2,3,6-tetrahydropyridin-4-yl)pyrrolo-[3,2-b]pyridine-5-carboxamide; N-[indol-4-yl]-3-(1-methyl-1,2,3,6-tetrahydropyridin-4-yl)pyrrolo[3,2-b]pyridine-5-carboxamide; and N-[quinolin-6-yl]-3-(1-methyl-1,2,3,6-tetrahydro-pyridin-4-yl)pyrrolo-[3,2-b]pyridine-5-carboxamide;

The following HTR_{1F} agonists are disclosed in PCT publication No. WO 00/00487: 5-phenyl-3-(1-methylpiperidin-4-yl)benzofuran; 5-(4-fluorophenyl)-3-(octahydroindolizin-7-yl)-2-methylbenzothiophene; 5-(2-chlorophenyl)-2-chloroethenyl)-3-(1-ethylpiperidin-4-yl)-2-ethyl-1H-indole; 5-(3-methoxyphenyl)-3-(octahydro-2H-quinolizin-2-yl)-1H-indazole; 5-(3,5-trifluorophenyl)-3-(1-propylpiperidin-4-yl)-2-propyl-4-aza-1H-indole; 5-(thien-2-yl)-3-(1-azabicyclo[5.4.0]undecan-4-yl)-2-cyclopropylbenzofuran; 5-(thien-3-yl)-3-(1-isopropylpiperidin-4-yl)-2-n-butylbenzothiophene; 5-(benzamidazol-2-yl)-3-(octahydroindolizin-7-yl)-2-s-butyl-1H-indole; 5-(naphth-1-yl)-3-(1-n-butylpiperidin-4-yl)1H-indazole; 5-(pyrazin-2-yl)-3-(octahydro-2H-quinolizin-2-yl)-2-t-butyl-4-aza-IH-indole; 5-(oxazol-2-yl)-3-(1-s-butylpiperidin-4-yl)-2-cyclobutylbenzofuran; 5-(quinolin-4-yl)-3-(1-azabicyclo[5.4.0]undecan-4-yl)benzothiophene; 5-(isothiazol-5-yl)-3-(1-t-butylpiperidin-4-yl)1H-indole; 5-(pyrimidin-2-yl)-3-(octahydroindolizin-7-yl)1H-indazole; 5-(isoxazol-4-yl)-3-(1-cyclopropylpiperidin-4-yl)4-aza-1H-indole; 5-(benzimidazol-2-yl)-3-(octahydro-2H-quinolizin-2-yl)benzofuran; 5-(5-fluorobenzimidazol-2-yl)-3-(1-cyclobutylpiperidin-4-yl)benzothiophene; 5-(5-methoxybenzimidazol-2-yl)-3-(1-azabicyclo[5.4.0]undecan-4-yl)1H-indole; 5-(naphth-2-yl)-3-(1-methylpiperidin-4-yl)1H-indazole; 5-(5-fluoronaphth-2-yl)-3-(octahydroindolizin-7-yl)4-aza-IH-indole; 5-(7-methoxynaphth-1-yl)-3-(1-methylpiperidin-4-yl)-2-methylbenzofuran; 5-(3-chloronaphth-1-yl)-3-(1,2,3,4,5,8-hexahydroindolizin-7-yl)-2-ethylbenzothiophene; 5-(4-trifluoromethylnaphth-2-yl)-3-(1-ethylpiperidin-4-yl)-2-propyl-IH-indole; 5-(3,5-difluoro-4-methoxyphenyl)-3-(1,4,5,6,7,8,9-heptahydroquinolizin-2-yl)-1H-indazole; 5-(2-carboxamidonaphth-1-yl)-3-(1-propylpiperidin-4-yl)-2-cyclopropyl-4-aza-1H-indole; and 5-(thiazol-2-yl)-3-(1-azabicyclo[5.4.0]undec-3-en-4-yl)-2-isopropylbenzofuran.

The following HTR_{1F} agonists are disclosed in PCT publication No. WO 98/08502: N,N-dimethyl-8-hydroxy-1,2,3,4-tetrahydro-2-dibenzo-furanamine; N,N-dimethyl-8-amino-1,2,3,4-tetrahydro-2-dibenzo-furanamine hydrochloride; N,N-diethyl-8-fluoro-1,2,3,4-tetrahydro-2-dibenzo-furanamine; N-ethyl-8-chloro-1,2,3,4-tetrahydro-2-dibenzofuranamine sulfate; N-methyl-N-benzyl-8-bromo-1,2,3,4-tetrahydro-2-dibenzofuranamine; N,N-dipropyl-8-iodo-1,2,3,4-tetrahydro-2-dibenzo-furanamine hydrobromide; N—(N,N-dimethyl-1,2,3,4-tetrahydro-2-aminodibenzo-fur-8-yl)acetamide N—(N,N-dimethyl-1,2,3,4-tetrahydro-2-aminodibenzo-fur-8-yl)propanamide; (−)—N—(N,N-dimethyl-1,2,3,4-tetrahydro-2-aminodi-benzofur-8-yl)hexanamide phosphate; N—{N,N-dimethyl-1,2,3,4-tetrahydro-2-aminodibenzo-fur-8-yl)acrylamide; N—(N,N-dimethyl-1,2,3,4-tetrahydro-2-aminodibenzo-fur-8-yl)cyclobutanamide acetate; N—(N,N-dimethyl-1,2,3,4-tetrahydro-2-aminodibenzo-fur-8-yl)cyclohexanamide; N—(N,N-dimethyl-1,2,3,4-tetrahydro-2-aminodibenzo-fur-8-yl)benzamide; N—(N-methyl-N-isopropyl-1,2,3,4-tetrahydro-2-amino-dibenzofur-8-yl)naphth-1-ylamide decanoate; N—(N,N-dimethyl-1,2,3,4-tetrahydro-2-aminodibenzo-fur-8-yl)naphth-2-ylamide; N—(N,N-dimethyl-1,2,3,4-tetrahydro-2-aminodibenzo-fur-8-yl)phenylacetamide; N—(N,N-dimethyl-1,2,3,4-tetrahydro-2-aminodibenzo-fur-8-yl)-3-methoxythien-2-ylacetamide; N—(N,N-dimethyl-1,2,3,4-tetrahydro-2-aminodibenzo-fur-8-yl)pyrrole-2-carboxamide; N—(N,N-dimethyl-1,2,3,4-tetrahydro-2-aminodibenzo-fur-8-yl)-5-methyloxazole-2-carboxamide acrylate; N—(N,N-dimethyl-1,2,3,4-tetrahydro-2-aminodibenzo-fur-8-yl)oxazole-4-carboxamide; N—(N,N-dimethyl-1,2,3,4-tetrahydro-2-aminodibenzo-fur-8-yl)isoxazole-3-carboxamide; N—(N,N-dimethyl-1,2,3,4-tetrahydro-2-aminodibenzo-fur-8-yl)pyrazole-3-carboxamide formate; N-(1,2,3,4-tetrahydro-2-aminodibenzofur-8-yl)pyrazole-4-carboxamide; N—(N,N-dimethyl-1,2,3,4-tetrahydro-2-aminodibenzo-fur-8-yl)imidazole-2-carboxamide malonate; N—(N,N-dimethyl-1,2,3,4-tetrahydro-2-aminodibenzo-fur-8-yl)imidazole-4-carboxamide; N—(N,N-dimethyl-1,2,3,4-tetrahydro-2-aminodibenzo-fur-8-yl)-1,2,3-triazole-4-carboxamide fumarate; N—(N,N-dimethyl-1,2,3,4-tetrahydro-2-aminodibenzo-fur-8-yl)-5-chloropyrimidine-2-carboxamide; N—(N,N-dimethyl-1,2,3,4-tetrahydro-2-aminodibenzo-fur-8-yl)pyrimidine-4-carboxamide butyne-1,4-dioate; N—(N,N-dirnethyl-1,2,3,4-tetrahydro-2-aminodibenzo-fur-8-yl)pyrazine-2-carboxamide benzoate; N—(N-hexyl-1,2,3,4-tetrahydro-2-aminodibenzofur-8-yl)pyridazine-3-carboxamide; N—(N,N-dimethyl-1,2,3,4-tetrahydro-2-aminodibenzo-fur-8-yl)pyridazine-4-carboxamide 4-chlorobenzoate; N—(N-methyl-N-phenethyl-1,2,3,4-tetrahydro-2-amino-dibenzofur-8-yl)quinoline-2-carboxamide phthalate; N—(N-isobutyl-1,2,3,4-tetrahydro-2-aminodibenzofur-8-yl)quinoline-4-carboxamide p-toluenesulfonate; N—(N,N-dimethyl-1,2,3,4-tetrahydro-2-aminodibenzo-fur-8-yl)quinoline-5-carboxamide methanesulfonate; N—(N,N-dimethyl-1,2,3,4-tetrahydro-2-aminodibenzo-fur-8-yl)isoquinoline-1-carboxamide dichloroacetate; N—(N,N-dimethyl-1,2,3,4-tetrahydro-2-aminodibenzo-fur-8-yl)isoquinoline-3-carboxamide trifluoroacetate; N—(N,N-dimethyl-1,2,3,4-tetrahydro-2-aminodibenzo-fur-8-yl)benzo[b]furan-2-carboxamide citrate; N—(N,N-dimethyl-1,2,3,4-tetrahydro-2-aminodibenzo-fur-8-yl)benzo[b]furan-3-carboxamide tartrate; (+)—N—(N,N-dimethyl-1,2,3,4-tetrahydro-2-amino-dibenzofur-8-yl)benzo[c]furan-4-carboxamide; N—(N-methyl-N-butyl-1,2,3,4-tetrahydro-2-amino-dibenzofur-8-yl)-5-bromobenzo[b]thien-2-carboxamide; N—(N,N-dimethyl-1,2,3,4-tetrahydro-2-aminodibenzo-fur-8-yl)benzo[b]thien-3-carboxamide; (−)—N—(N,N-dimethyl-1,2,3,4-tetrahydro-2-amino-dibenzofur-8-yl)benzo[c]thien-4-carboxamide hippurate; N—(N,N-dimethyl-1,2,3,4-tetrahydro-2-aminodibenzo-fur-8-yl)benzo[b]thien-6-carboxamide naphthalene-1-sulfonate; N—(N,N-dimethyl-1,2,3,4-tetrahydro-2-aminodibenzo-fur-8-yl)-6-ethoxyindole-2-carboxamide; N—(N,N-dimethyl-1,2,3,4-tetrahydro-2-aminodibenzo-fur-8-yl)-7-fluoroindole-3-carboxamide; N—(N,N-dimethyl-1,2,3,4-tetrahydro-2-aminodibenzo-fur-8-yl)indole-5-carboxamide; N′-methyl-N,N-dimethyl-1,2,3,4-tetrahydro-2-dibenzo-furanamine-8-carboxamide; N′-isobutyl-N,N-dimethyl-1,2,3,4-tetrahydro-2-dibenzofuranamine-8-carboxamide; N′-hexyl-N,N-dimethyl-1,2,3,4-tetrahydro-2-dibenzo-furanamine-8-carboxamide; N′-propenyl-1,2,3,4-tetrahydro-2-dibenzofuranamine-8-carboxamide; N′-cyclopropyl-N-methyl-N-benzyl-1,2,3,4-tetrahydro-2-dibenzofuranamine-8-carboxamide; (+)—N′-cyclohexyl-N,N-dimethyl-1,2,3,4-tetrahydro-2-dibenzofuranamine-8-carboxamide; N′-phenyl-N,N-dimethyl-1,2,3,4-tetrahydro-2-dibenzo-furanamine-8-carboxamide; N′-(4-fluorophenyl)-N-butyl-N-ethyl-1,2,3,4-tetra-hydro-2-dibenzofuranamine-8-carboxamide; N′-(naphth-1-yl)-N,N-dimethyl-1,2,3,4-tetrahydro-2-dibenzofuranamine-8-carboxamide; N′-(fur-2-yl)-N,N-dimethyl-1,2,3,4-tetrahydro-2-dibenzofuranamine-8-carboxamide; N′-(thien-3-yl)-N,N-dimethyl-1,2,3,4-tetrahydro-2-dibenzofuranamine-8-carboxamide; N′-(pyridin-4-yl)-N,N-dimethyl-1,2,3,4-tetrahydro-2-dibenzofuranamine-8-carboxamide; N′-(6-chloropyridin-3-yl)-N,N-dimethyl-1,2,3,4-tetrahydro-2-dibenzofuranamine-8-carboxamide; N′-(pyrrol-2-yl)-N,N-dimethyl-1,2,3,4-tetrahydro-2-dibenzofuranamine-8-carboxamide; N′-(oxazol-4-yl)-N,N-dimethyl-1,2,3,4-tetrahydro-2-dibenzofuranamine-8-carboxamide; N′-(isoxazol-3-yl)-N,N-dimethyl-1,2,3,4-tetrahydro-2-dibenzofuranamine-8-carboxamide; N′-(pyrazol-3-yl)-N,N-dimethyl-1,2,3,4-tetrahydro-2-dibenzofuranamine-8-carboxamide; N′-(imidazol-2-yl)-N,N-dimethyl-1,2,3,4-tetrahydro-2-dibenzofuranamine-8-carboxamide; N′-(triazol-4-yl)-N,N-dimethyl-1,2,3,4-tetrahydro-2-dibenzofuranamine-8-carboxamide; N′-(pyrimidin-2-yl)-N,N-dimethyl-1,2,3,4-tetrahydro-2-dibenzofuranamine-8-carboxamide; N′-(pyrazin-2-yl)-N,N-dimethyl-1,2,3,4-tetrahydro-2-dibenzofuranamine-8-carboxamide; N′-(pyridazin-4-yl)-N,N-dimethyl-1,2,3,4-tetrahydro-2-dibenzofuranamine-8-carboxamide; N′-(quinolin-3-yl)-N,N-dimethyl-1,2,3,4-tetrahydro-2-dibenzofuranamine-8-carboxamide; N′-(isoquinolin-5-yl)-N,N-dimethyl-1,2,3,4-tetra-hydro-2-dibenzofuranamine-8-carboxamide; N′-(benzofur-2-yl)-N,N-dimethyl-1,2,3,4-tetrahydro-2-dibenzofuranamine-8-carboxamide; N′-(benzo[b]thien-6-yl)-N,N-dimethyl-1,2,3,4-tetrahydro-2-dibenzofuranamine-8-carboxamide; N′-(indol-2-yl)-N,N-dimethyl-1,2,3,4-tetrahydro-2-dibenzofuranamine-8-carboxamide; N—(N,N-dimethyl-1,2,3,4-tetrahydro-2-aminodibenzo-fur-8-yl)methanesulfonamide; N—(N,N-dimethyl-1,2,3,4-tetrahydro-2-aminodibenzo-fur-8-yl)ethanesulfonamide; N—(N,N-dimethyl-1,2,3,4-tetrahydro-2-aminodibenzo-fur-8-yl)benzenesulfonamide; N—(N,N-dimethyl-1,2,3,4-tetrahydro-2-aminodibenzo-fur-8-yl)-3-chlorobenzenesulfonamide; N—(N,N-dimethyl-1,2,3,4-tetrahydro-2-aminodibenzo-fur-8-yl)-4-methylbenzenesulfonamide; N,N-dimethyl-9-hydroxy-2-aminocyclohepta[b]ben-zofuran; N,N-dimethyl-9-amino-2-aminocyclohepta[b]benzofuran hydrochloride; N,N-diethyl-9-fluoro-2-aminocyclohepta[b]benzofuran; N-ethyl-9-chloro-2-aminocyclohepta[b]benzofuran sulfate; N-methyl-N-benzyl-9-bromo-2-aminocyclohepta[b]benzo-furan; N,N-dipropyl-9-iodo-2-aminocyclohepta[b]benzofuran hydrobromide; N—(N,N-dimethyl-2-aminocyclohepta[b]benzofur-9-yl)acetamide; N—(N,N-dimethyl-2-aminocyclohepta[b]benzofur-9-yl)propanamide; (−)—N—(N,N-dimethyl-2-aminocyclohepta[b]benzofur-9-yl)hexanamide phosphate; N—(N,N-dimethyl-2-aminocyclohepta[b]benzofur-9-yl)acrylamide; N—(N,N-dimethyl-2-aminocyclohepta[b]benzofur-9-yl)cyclobutanamide acetate; N—(N,N-dimethyl-2-aminocyclohepta[b]benzofur-9-yl)cyclohexanamide N—(N,N-dimethyl-2-aminocyclohepta[b]benzofur-9-yl)benzamide; N—(N-methyl-N-isopropyl-2-amino cyclohepta[b]ben-zofur-9-yl)naphth-1-ylamide decanoate; N—(N,N-dimethyl-2-aminocyclohepta [b]benzofur-9-yl)naphth-2-ylamide; N—(N,N-dimethyl-2-aminocyclohepta[b]benzofur-9-yl)phenylacetamide; N—(N,N-dimethyl-2-aminocyclohepta[b]benzofur-9-yl)thien-2-ylacetamide; N—(N,N-dimethyl-2-aminocyclohepta[b]benzofur-9-yl)pyrrole-2-carboxamide; N—(N,N-dimethyl-2-aminocyclohepta[b]benzofur-9-yl)oxazole-2-carboxamide aerylate; N—(N,N-dimethyl-2-aminocyclohepta[b]benzofur-9-yl)2-propyloxazole-4-carboxamide; N—(N,N-dimethyl-2-aminocyclohepta[b]benzofur-9-yl)isoxazole-3-carboxamide; N—(N,N-dimethyl-2-aminocyclohepta[b]benzofur-9-yl)pyrazole-3-carboxamide formate; N-(2-aminocyclohepta[b]benzofur-9-yl)pyrazole-4-carboxamide; N—(N,N-dimethyl-2-aminocyclohepta[b]benzofur-9-yl)imidazole-2-carboxamidemalonate; N—(N,N-dimethyl-2-aminocyclohepta[b]benzofur-9-yl)imidazole-4-carboxamide; N—(N,N-dimethyl-2-amin-cyclohepta[b]benzofur-9-yl)-1,2,4-triazole-3-carboxamide fumarate; N—(N,N-dimethyl-2-aminocyclohepta[b]benzofur-9-yl)pyrimidine-2-carboxamide; N—(N,N-dimethyl-2-aminocyclohepta[b]benzofur-9-yl)pyrimidine-4-carboxamidebutyne-1,4-dioate; N—(N-isopropyl-N-benzyl-2-aminocyclohepta[b]benzofur-9-yl)pyrazine-2-carboxamide benzoate; N—(N,N-dimethyl-2-aminocyclohepta[b]benzofur-9-yl)pyridazine-3-carboxamide; N-(2-aminocyclohepta[b]benzofur-9-yl)pyridazine-4-carboxamide 4-chlorobenzoate; N—(N-methyl-N-phenethyl-2-aminocyclohepta[b]benzo-fur-9-yl)quinoline-2-carboxamide phthalate; N—(N,N-dimethyl-2-aminocyclohepta[b]benzofur-9-yl)quinoline-4-carboxamide p-toluenesulfonate; N—(N,N-dibutyl-2-aminocyclohepta[b]benzofur-9-yl)2-methylquinoline-6-carboxamide methanesulfonate; N—(N,N-dimethyl-2-aminocyclohepta[b]benzofur-9-yl)isoquinoline-1-carboxamide trifluoromethanesulfonate; N—(N,N-dimethyl-2-aminocyclohepta[b]benzofur-9-yl)isoquinoline-3-carboxamide trifluoroacetate; N—(N,N-dimethyl-2-aminocyclohepta[b]benzofur-9-yl)isoquinoline-6-carboxamide citrate; N—(N,N-dimethyl-2-aminocyclohepta[b]benzofur-9-yl)benzo[b]furan-2-carboxamidemandelate; N—(N,N-dimethyl-2-aminocyclohepta[b]benzofur-9-yl)5-fluorobenzo[b]furan-3-carboxamide tartrate; (+)—N—(N,N-dibenzyl-2-aminocyclohepta[b]benzofur-9-yl)benzo[b]furan-4-carboxamide; N—(N,N-dimethyl-2-aminocyclohepta[b]benzofur-9-yl)benzo[b]thien-2-carboxamide; N—(N,N-dimethyl-2-aminocyclohepta[b]benzofur-9-yl)benzo[b]thien-3-carboxamide; (−)—N-(N,N-dimethyl-2-aminocyclohepta[b]benzofur-9-yl)benzo[b]thien-4-carboxamide hippurate; N—(N-isopropyl-2-aminocyclohepta[b]benzofur-9-yl)-4-methoxyindole-2-carboxamide; N—(N,N-dimethyl-2-aminocyclohepta[b]benzofur-9-yl)indole-3-carboxamide; N—(N,N-dimethyl-2-aminocyclohepta[b]benzofur-9-yl)indole-7-carboxamide; N′-methyl-N,N-dimethyl-2-aminocyclohepta[b]benzofur-9-ylcarboxamide; N′-ethyl-N,N-dimethyl-2-aminocyclohepta[b]benzofur-9-ylcarboxamide; N′-isopropyl-N,N-dimethyl-2-aminocyclohepta[b]benzo-fur-9-ylcarboxamide; N′-propenyl-N,N-dimethyl-2-aminocyclohepta[b]benzo-fur-9-ylcarboxamide; N′-cyclopropyl-N,N-dimethyl-2-aminocyclohepta[b]-benzofur-9-ylcarboxamide; (+)-N′-cyclohexyl-N,N-dimethyl-2-aminocyclohepta-[b]benzofur-9-ylcarboxamide; N′-phenyl-N,N-dimethyl-2-aminocyclohepta[b]benzofur-9-ylcarboxamide; N′-(4-fluorophenyl)-N,N-dimethyl-2-aminocyclohepta-[b]benzofur-9-ylcarboxamide; N′-(naphth-1-yl)-N,N-dimethyl-2-aminocyclohepta[b]-benzofur-9-ylcarboxamide; N′-(fur-2-yl)-N,N-dimethyl-2-aminocyclohepta[b]-benzofur-9-ylcarboxamide; N′-(thien-3-yl)-N,N-dimethyl-2-aminocyclohepta[b]-benzofur-9-ylcarboxamide; N′-(pyridin-4-yl)-N,N-dimethyl-2-aminocyclohepta-[b]benzofur-9-ylcarboxamide; N′-(6-chloropyridin-3-yl)-N,N-dimethyl-2-aminocyclo-hepta[b]benzofur-9-ylcarboxamide; N′-(pyrrol-2-yl)-N,N-dimethyl-2-aminocyclohepta[b]-benzofur-9-ylcarboxamide; N′-(oxazol-4-yl)-N,N-dimethyl-2-aminocyclohepta[b]-benzofur-9-ylcarboxamide; N′-(isoxazol-3-yl)-N,N-dimethyl-2-aminocyclohepta-[b]benzofur-9-ylcarboxamide; N′-(pyrazol-3-yl)-N,N-dimethyl-2-aminocyclohepta-[b]benzofur-9-ylcarboxamide; N′-(imidazol-2-yl)-N,N-dimethyl-2-aminocyclohepta-[b]benzofur-9-ylcarboxamide; N′-(triazol-4-yl)-N,N-dimethyl-2-aminocyclohepta-[b]benzofur-9-ylcarboxamide; N′-(pyrimidin-2-yl)-N,N-dimethyl-2-aminocyclohepta-[b]benzofur-9-ylcarboxamide; N′-(pyrazin-2-yl)-N,N-dimethyl-2-aminocyclohepta-[b]benzofur-9-ylcarboxamide; N′-(pyridazin-4-yl)-N,N-dimethyl-2-aminocyclohepta-[b]benzofur-9-ylcarboxamide; N′-(quinolin-3-yl)-N,N-dimethyl-2-aminocyclohepta-[b]benzofur-9-ylcarboxamide; N′-(isoquinolin-5-yl)-N,N-dimethyl-2-aminocyclohep-ta[b]benzofur-9-ylcarboxamide; N′-(benzofur-2-yl)-N,N-dimethyl-2-aminocyclohepta-[b]benzofur-9-ylcarboxamide; N′-(benzo[b]thien-6-yl)-N,N-dimethyl-2-aminocyclo-hepta[b]benzofur-9-ylcarboxamide; N′-(indol-2-yl)-N,N-dimethyl-2-aminocyclohepta[b]-benzofur-9-ylcarboxamide; N—(N,N-dimethyl-2-aminocyclohepta[b]benzofur-9-yl)methanesulfonamide; N—(N,N-dimethyl-2-aminocyclohepta[b]benzofur-9-yl)ethanesulfonamide; N—(N,N-dimethyl-2-aminocyclohepta[b]benzofur-9-yl)benzenesulfonamide; N—(N,N-dimethyl-2-aminocyclohepta[b]benzofur-9-yl)-3-chlorobenzenesulfonamide; N—(N,N-dimethyl-2-aminocyclohepta[b]benzofur-9-yl)-4-methylbenzenesulfonamide.

The following HTR_{1F} agonists are disclosed in PCT publication No. WO 98/55115, N-(4-methylthien-2-yl)-3-(propyl)amino-1,2,3,4-tetrahydro-9H-carbazole-6-carboxamide hydrochloride; (+)-N-(thien-3-yl)-3-(dimethyl)amino-1,2,3,4-tetrahydro-9H-carbazole-6-carboxamide sulfate; N-(4-chlorofur-2-yl)-3-(propyl)amino-1,2,3,4-tetrahydro-9H-carbazole-6-carboxamide; N-(fur-3-yl)-3-(diethyl)amino-1,2,3,4-tetrahydro-9H-carbazole-6-carboxamide; N-(pyridin-3-yl)-3-(diethyl) amino-1,2,3,4-tetrahydro-9H-carbazole-6-carboxamide phosphate; N-(3-chloropyridin-4-yl)-3-(diethyl) amino-1,2,3,4-tetrahydro-9H-carbazole-6-carboxamide; N-(oxazol-5-yl)-3-(propyl)amino-1,2,3,4-tetrahydro-9H-carbazole-6-carboxamide D-toluenesulfonate; (+)-N-(isoxazol-4-yl)-3-(dimethyl) amino-1,2,3,4-tetrahydro-9H-carbazole-6-carboxamide; N-(pyrazol-4-yl)-3-(propyl)amino-1,2,3,4-tetrahydro-9H-carbazole-6-carboxamide methanesulfonate; N-(cyclobutyl)-3-(diethyl)amino-1,2,3,4-tetrahydro-9H-carbazole-6-carboxamide oxalate; N-(cyclohexyl)-3-(propyl)amino-1,2,3,4-tetrahydro-9H-carbazole-6-carboxamide; (+)-N-(2-methylpyrimidin-5-yl)-3-(dimethyl)amino-1,2,3,4-tetrahydro-9H-carbazole-6-carboxamide formate; (-)-N-(thien-2-yl)-4-(methyl)amino-10H-cyclohepta[7,6-b]indole-7-carboxamide; N-(thien-3-yl)-4-(dimethyl)amino-10H-cyclohepta[7,6-b]indole-7-carboxamide butyne-1,4-dioate; N-(fur-2-yl)-4-(propyl)amino-10H-cyclohepta[7,6-b]indole-7-carboxamide; N-(fur-3-yl)-4-(diethyl)amino-10H-cyclohepta[7,6-b]-indole-7-carboxamide trifluoroacetate; N-(pyridin-3-yl)-4-(diisopropyl)amino-10H-cyclohepta-[7,6-b]indole-7-carboxamide; N-(3-chloropyridin-4-yl)-4-(dibutyl)amino-10H-cyclohepta[7,6-b]indole-7-carboxamide; (−)-N-(pyrrol-3-yl)-4-(methyl)amino-10H-cyclohepta[7,6-b]indole-7-carboxamide tartrate; N-(2-isopropyloxazol-5-yl)-4-(dimethyl)amino-10H-cyclohepta[7,6-b]indole-7-carboxamide; N-(3-bromo-4-methylisoxazol-5-yl)-4-(propyl)amino-10H-cyclohepta[7,6-b]indole-7-carboxamide cinnamate; N-(3-ethylpyrazol-4-yl)-4-(diethyl)amino-10H-cyclohepta[7,6-b]indole-7-carboxamide; N-(cyclopropyl)-4-(diisopropyl)amino-10H-cyclohepta-[7,6-b]indole-7-carboxamide; N-(cyclohexyl)-4-(dibutyl)amino-10H-cyclohepta[7,6-b]indole-7-carboxamide mandelate; (−)-N-(2-methoxypyrimidin-5-yl)-4-(methyl)amino-10H-cyclohepta [7,6-b]indole-7-carboxamide; N-(2-fluoropyrazin-4-yl)-4-(dimethyl) amino-10H-cyclohepta[7,6-b]indole-7-carboxamide; N-(cyclobutyl)-4-(propyl)amino-10H-cyclohepta[7,6-b]indole-7-carboxamide; N-(cyclopentyl)-4-(diethyl)amino-10H-cyclohepta[7,6-b]indole-7-carboxamide 4-methoxybenzoate; and N-(cyclohexyl)-4-(diisopropyl)amino-10H-cyclohepta[7,6-b]indole-7-carboxamide.

The following HTR_{1F} agonists are disclosed in PCT publication No. WO 2003/084949: 1) 4-Fluoro-N-[6(1-methyl-piperidine-4-carbonyl)-pyridin-2-yl]-benzamide; 2) 2,4-Difluoro-N-[6(I-methyl-piperidine-4-carbonyl)-pyridin-2-yl]-benzamide; 3)N-[6(1-Methyl-piperidine-4-carbonyl)-pyridin-2-yl]-benzamide; 4) 2-Chloro-4-fluoro-N-[6(1-methyl-piperidine-4-carbonyl)-pyridin-2-yl]-benzamide; 5) 2-Chloro-N-[6(1-methyl-piperidine-4-carbonyl)-pyridin-2-yl]-benzamide; 6) 2,4,6-Trifluoro-N-[6-(piperidine-4-carbonyl)-pyridin-2-yl]-benzamide; 7) 1H-5-Trifluoromethyl-indole-3-carboxylic acid [6-(I-methyl-piperidine-4-carbonyl)-pyridin-2-yl]-amide; 8)N-[6-(1-Methyl-piperidine-4-carbonyl)-pyridin-2-yl]-2-trifluoromethoxy-benzamide; 9) 3-Bromo-thiophene-2-carboxylic acid [6-(1-methyl-piperidine-4-carbonyl)-pyridin-2-yl]-amide; 10) 4-Fluoro-N-[6-(1-methyl-piperidine-4-carbonyl)-pyridin-2-yl]-2-trifluoromethyl-benzamide; 11) 2,4,6-Trifluoro-N-[6-(1-methyl-piperidine-4-carbonyl)-pyridin-2-yl]-benzamide; 12) 2-Chloro-6-fluoro-N-[6-(1-methyl-piperidine-4-carbonyl)-pyridin-2-yl]-benzamide; 13) 2,4,6-Trifluoro-N-methyl-N-[6-(1-methyl-piperidine-4-carbonyl)-pyridin-2-yl]-benzamide; 14) 2,4,6-Trifluoro-N-methyl-N-[6-(piperidine-4-carbonyl)-pyridin-2-yl]-benzamide; 15) 2,4,6-Trifluoro-N-methy]-N-[6-(1-methyl-piperidine-4-carbonyl)-pyridin-2-yl]-benzamide; 16) 2,4,6-Trifluoro-N-ethyl-N-[6-(I-methyl-piperidine-4-carbony])-pyridin-2-yl]-benzamide; 17) 2-Chloro-4-fluoro-N-[6-(piperidin-4-carbonyl)-pyridin-2-yl]-benzamide; 18) 2-Chloro-4-fluoro-N-methyl-N-[6-(1-methyl-piperidin-4-carbonyl)-pyridin-2-yl]-benzamide; 19) 1H-5-Fluoro-indole-3-carboxylic acid [6-(1-methyl-piperidine-4-carbonyl)-pyridin-2-yl]-amide; 20) Cyclopropanecarboxylic acid [6-(1-methyl-piperidine-4-carbonyl)-pyridin-2-yl]-amide; 21) 3-Methyl-N-[6-(1-methyl-piperidine-4-carbonyl)-pyridin-2-yl]-butanamide; 22) Thiophene-2-carboxylic acid [6-(1-methyl-piperidine-4-carbonyl)-pyridin-2-yl]-amide; 23) Furan-2-carboxylic acid [6-(1-methyl-piperidine-4-carbonyl)-pyridin-2-yl]-amide; 24) 2-Chloro-N-[6-(1-methyl-piperidine-4-carbonyl)-pyridin-2-yl]-benzamide; 25) Furan-3-carboxylic acid [6-(1-methyl-piperidine-4-carbonyl)-pyridin-2-yl]-amide; 26) 3,4-Difluoro-N-[6-(1-methyl-piperidine-4-carbonyl)-pyridin-2-yl]-benzamide; 27)N-[6-(1-Methyl-piperidine-4-carbonyl)-pyridin-2-yl]-isonicotinamide; 28) 2-Methyl-N-[6-(1-methyl-piperidine-4-carbonyl)-pyridin-2-yl]-benzamide; 29) 2-Bromo-N-[6-(1-methyl-piperidine-4-carbonyl)-pyridin-2-yl]-benzamide; 30) Thiophene-3-carboxylic acid [6-(1-methyl-piperidine-4-carbonyl)-pyridin-2-yl]-amide; 31) 2-Fluoro-N-[6-(1-methyl-piperidine-4-carbonyl)-pyridin-2-yl]-isonicotinamide; 32) 4-Chloro-2-methoxy-N-[6-(1-methyl-piperidine-4-carbonyl)-pyridin-2-yl]-benzamide; 33) 2-Ethoxy-N-[6-(1-methyl-piperidine-4-carbonyl)-pyridin-2-yl]-benzamide; 34)N-[6-(1-Methyl-piperidine-4-carbonyl)-pyridin-2-yl]-2-phenoxy-benzamide; 35) 5-Chloro-2-methoxy-N-[6-(1-methyl-piperidine-4-carbonyl)-pyridin-2-yl]-benzamide; 36) 2-Methoxy-N-[6-(1-methyl-piperidine-4-carbonyl)-pyridin-2-yl]-4-methylsulfanyl-benzamide; 37) 2,3-Dihydro-benzofuran-7-carboxylic acid [6-(1-methyl-piperidine-4-carbonyl)-pyridin-2-yl]-amide; 38) 2-Benzyloxy-N-[6-(1-methyl-piperidine-4-carbonyl)-pyridin-2-yl]-benzamide; 39)N-[6-(1-Methyl-piperidine-4-carbony])-pyridin-2-yl]-2-propoxy-benzamide; 40) 2,2-Difluoro-benzo[1,3]dioxole-4-carboxylic acid [6-(1-methyl-piperidine-4-carbonyl)-pyridin-2-yl]-amide; 41) 4-Methoxy-2-(2-methoxy-ethoxy)-N-[6-(1-methyl-piperidine-4-carbonyl)-pyridin-2-yl]-benzamide; 42) 5-Bromo-2-methoxy-N-[6-(1-methyl-piperidine-4-carbonyl)-pyridin-2-yl]-benzamide; 43) 2-(4,6-Dimethoxy-pyrimidin-2-yloxy)-N-[6-(1-methyl-piperidine-4-carbonyl)-pyridin-2-yl]-benzamide; 44)N-[6-(1-Methyl-piperidine-4-carbonyl)-pyridin-2-yl]-butanamide; 45) Cyclohexanecarboxylic acid [6-(1-methyl-piperidine-4-carbonyl)-pyridin-2-yl]-amide; 46)N-[6-(1-Methyl-piperidine-4-carbonyl)-pyridin-2-yl]-3-phenyl-propionamide; 47) 2,6-Difluoro-N-[6-(1-methyl-piperidine-4-carbony])-pyridin-2-yl]-benzamide; 48) 2-Ethoxy-N-[6-(1-methyl-piperidine-4-carbonyl)-pyridin-2-yl]-nicotinamide; 49)N-[6-(1-Methyl-piperidine-4-carbonyl)-pyridin-2-yl]-2-phenoxy-nicotinamide; 50) 3-Acetyl-thiazolidine-4-carboxylic acid [6-(1-methyl-piperidine-4-carbonyl)-pyridin-2-yl]-amide; 51)N-[6-(1-Methyl-piperidine-4-carbonyl)-pyridin-2-yl]-2-phenylsulfanyl-nicotinamide; 52) 5-Methoxy-N-[6-(1-methyl-piperidine-4-carbonyl)-pyridin-2-yl]-2-(2,2,2-trifluoro-ethoxy)-benzamide; 53) 2-Methoxy-6-methyl-N-[6-(1-methyl-piperidine-4-carbonyl)-pyridin-2-yl]-benzamide; 54)N-[6-(1-Methyl-piperidine-4-carbonyl)-pyridin-2-yl]-terephthalamic acid methyl ester; 55) Cyclobutanecarboxylic acid [6-(1-methyl-piperidine-4-carbonyl)-pyridin-2-yl]-amide; 56) 2-(2-Chloro-1,2-trifluoro-ethoxy)-N-[6-(1-methyl-piperidine-4-carbonyl)-pyridin-2-yl]-benzamide 57) 2-Chloro-N-[6-(1-methyl-piperidine-4-carbonyl)-pyridin-2-yl]-benzamide; 58) 2,5-Difluoro-N-[6-(1-methyl-piperidine-4-carbonyl)-pyridin-2-yl]-benzamide; 59) 3,4-Difluoro-N-[6-(1-methyl-piperidine-4-carbonyl)-pyridin-2-yl]-benzamide; 60) 4-Fluoro-N-[6-(1-methyl-piperidine-4-carbonyl)-pyridin-2-yl]-2-trifluoromethyl-benzamide; 61) 2-Fluoro-N-[6-(1-methyl-piperidine-4-carbonyl)-pyridin-2-yl]-6-trifluoromethyl-benzamide; 62) 2,3,4-Trifluoro-N-[6-(1-methyl-piperidine-4-carbonyl)-pyridin-2-yl]-benzamide; 63) 2,4,5-Trifluoro-N-[6-(1-methyl-piperidine-4-carbonyl)-pyridin-2-yl]-benzamide; 64) 3-Chloro-thiophene-2-carboxylic acid [6-(1-methyl-piperidine-4-carbonyl)-pyridin-2-yl]-amide; 65) 2,6-Dichloro-N-[6-(1-methyl-piperidine-4-carbonyl)-pyridin-2-yl]-benzamide; 66) 2-Fluoro-N-[6-(1-methyl-piperidine-4-carbonyl)-pyridin-2-yl]-4-trifluoromethyl-benzamide; 67) Cyclopentanecarboxylic acid [6-(1-methyl-piperidine-4-carbonyl)-pyridin-2-yl]-amide; 69)N-[6-(1-Methyl-piperidine-4-carbonyl)-pyridin-2-yl]-nicotinamide.

Additional HTR_{1F} agonists are disclosed in the following PCT publications: WO 98/15545, WO 00/00490, WO 00/47559, WO 00/50426, and WO 00/34266. The HTR_{1F} agonists disclosed in these documents are incorporated herein by reference.

The term “THRB agonist” as used herein refers to an agent that has the ability to bind to and activate THRB, e.g., that has the ability to activate or increase signaling that occurs downstream of THRB. The THRB agonist may be a small molecule, a peptide, a polypeptide or an antibody or antigen-binding fragment thereof. Examples of THRB agonist include the thyroid hormones triiodothyronine (T3) and tetraiodothyronine (T4) and metabolites thereof such as 3,5-didiodothyronine (T2); thyronamines (particularly 3-iodothyronamine (T1AM) and non-iodinated thyronamines (TOAM)) and thyroacetic acids (particularly 3,5,3′,5′-thyroacetic acid (TA4), 3,5,3′-thyroacetic acid (TA3) and 3-thyroacetic acid (TA1)), Liothyronine sodium, Resmetirom (MGL-3196), Eprotirome (KB-2115), 2-[3,5-dichloro-4-(4-hydroxy-3-propan-2-ylphenoxy)phenyl]acetic acid (KB-141), Sobetirome (GC1), Sob-AM2, VK2809 (MB-08711), MB-07344, MGL-3745, IS25 and TG68 (see, e.g., Saponaro et al., Front Med (Lausanne). 2020; 7: 331).

PCT publication No. WO 2017/184811 discloses a THRB agonist of the following structure:

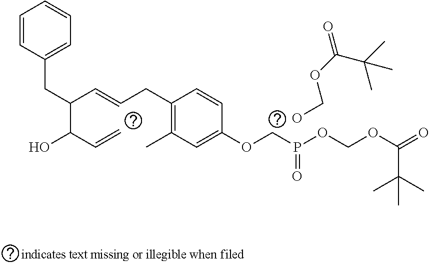

WO 2021/050945 discloses THRB agonists of the following structures:

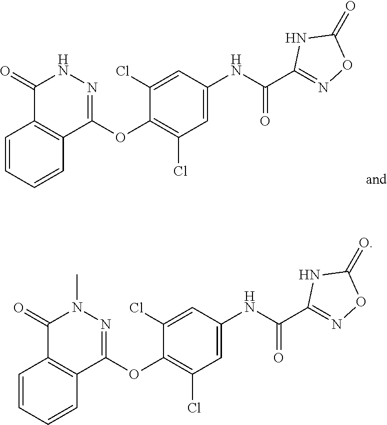 [SMILES: O=C(Nc1cc(Cl)c(Oc2n[nH]c(=O)c3ccccc23)c(Cl)c1)c1noc(=O)[nH]1 ; Cn1nc(Oc2c(Cl)cc(NC(=O)c3noc(=O)[nH]3)cc2Cl)c2ccccc2c1=O]

WO 2021/041237 discloses THRB agonists of the following structures:

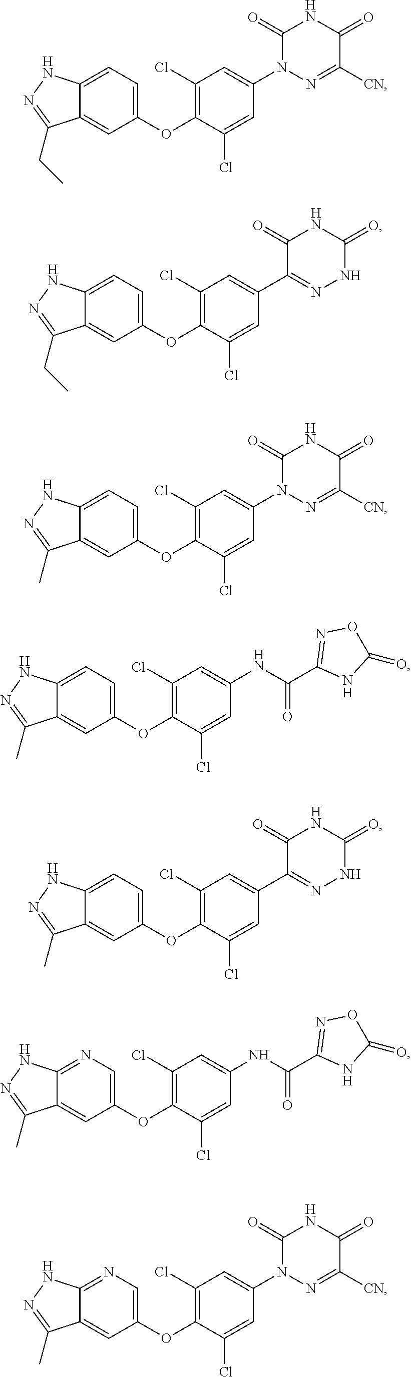

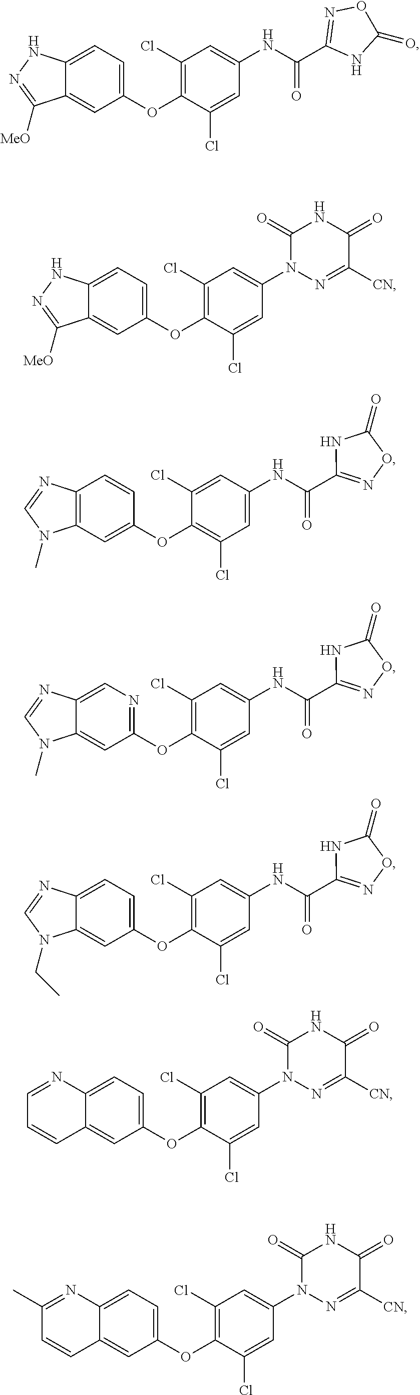

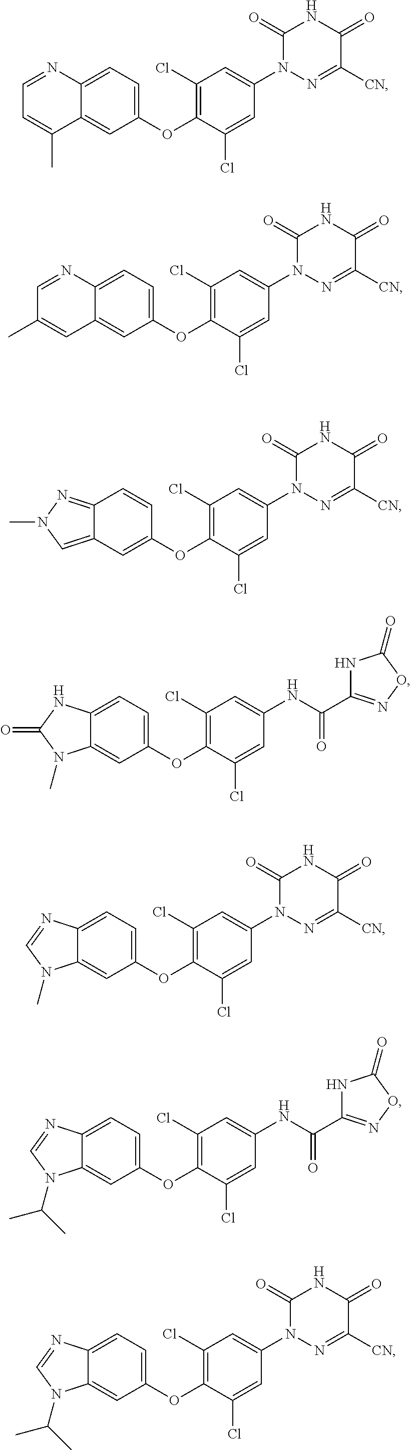

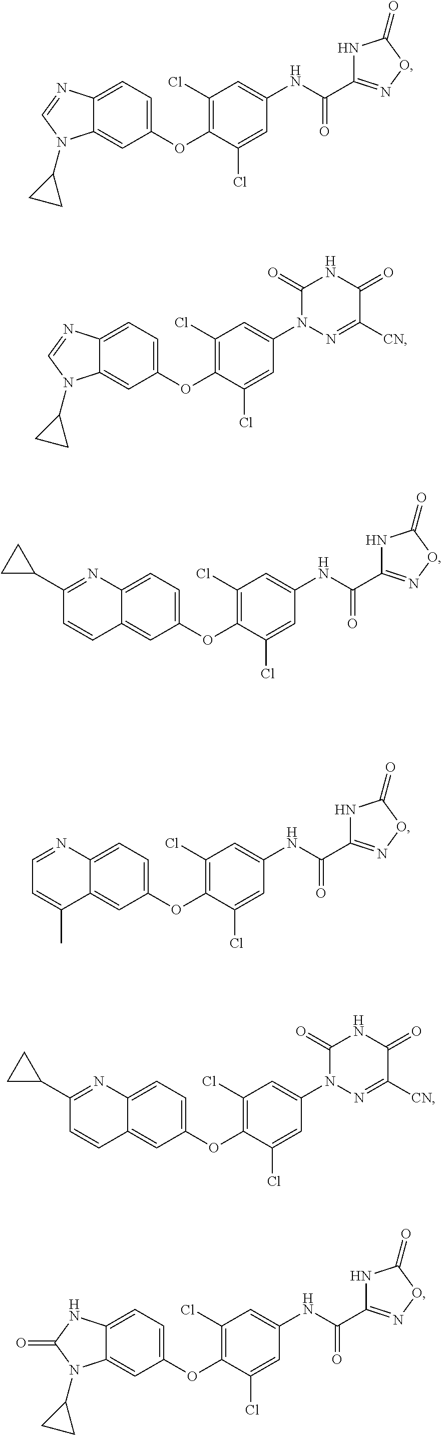 [SMILES: O=C(Nc1cc(Cl)c(Oc2ccc3ncn(C4CC4)c3c2)c(Cl)c1)c1noc(=O)[nH]1 ; N#Cc1nn(-c2cc(Cl)c(Oc3ccc4ncn(C5CC5)c4c3)c(Cl)c2)c(=O)[nH]c1=O ; O=C(Nc1cc(Cl)c(Oc2ccc3nc(C4CC4)ccc3c2)c(Cl)c1)c1noc(=O)[nH]1 ; Cc1ccnc2ccc(Oc3c(Cl)cc(NC(=O)c4noc(=O)[nH]4)cc3Cl)cc12 ; N#Cc1nn(-c2cc(Cl)c(Oc3ccc4nc(C5CC5)ccc4c3)c(Cl)c2)c(=O)[nH]c1=O ; O=C(Nc1cc(Cl)c(Oc2ccc3[nH]c(=O)n(C4CC4)c3c2)c(Cl)c1)c1noc(=O)[nH]1]

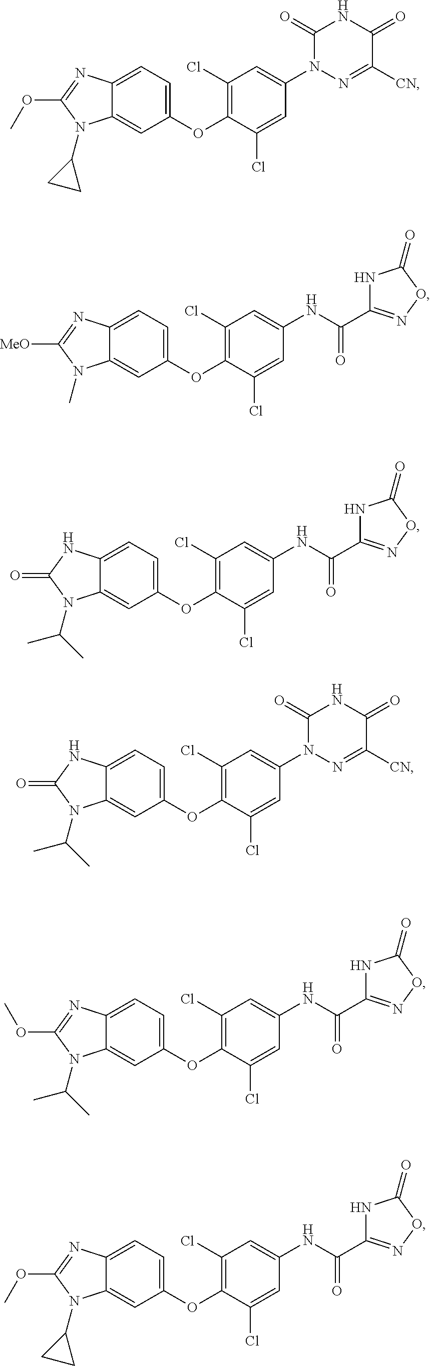 [SMILES: COc1nc2ccc(Oc3c(Cl)cc(-n4nc(C#N)c(=O)[nH]c4=O)cc3Cl)cc2n1C1CC1 ; COc1nc2ccc(Oc3c(Cl)cc(NC(=O)c4noc(=O)[nH]4)cc3Cl)cc2n1C ; CC(C)n1c(=O)[nH]c2ccc(Oc3c(Cl)cc(NC(=O)c4noc(=O)[nH]4)cc3Cl)cc21 ; CC(C)n1c(=O)[nH]c2ccc(Oc3c(Cl)cc(-n4nc(C#N)c(=O)[nH]c4=O)cc3Cl)cc21 ; COc1nc2ccc(Oc3c(Cl)cc(NC(=O)c4noc(=O)[nH]4)cc3Cl)cc2n1C(C)C ; COc1nc2ccc(Oc3c(Cl)cc(NC(=O)c4noc(=O)[nH]4)cc3Cl)cc2n1C1CC1]

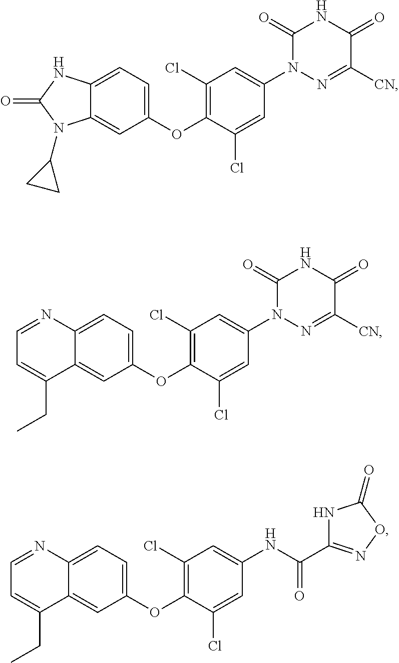 [SMILES: N#Cc1nn(-c2cc(Cl)c(Oc3ccc4[nH]c(=O)n(C5CC5)c4c3)c(Cl)c2)c(=O)[nH]c1=O ; CCc1ccnc2ccc(Oc3c(Cl)cc(-n4nc(C#N)c(=O)[nH]c4=O)cc3Cl)cc12 ; CCc1ccnc2ccc(Oc3c(Cl)cc(NC(=O)c4noc(=O)[nH]4)cc3Cl)cc12]

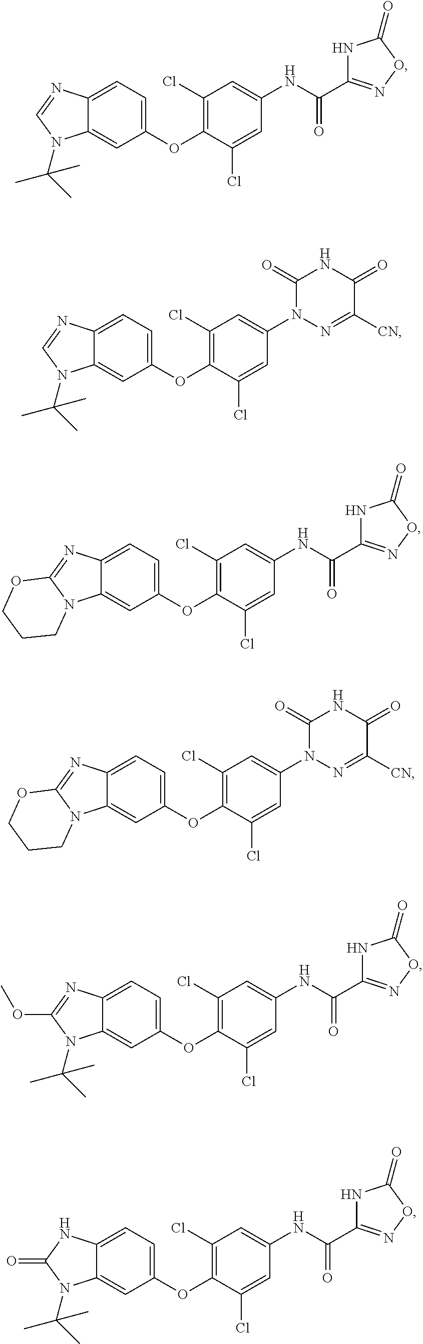 [SMILES: CC(C)(C)n1cnc2ccc(Oc3c(Cl)cc(NC(=O)c4noc(=O)[nH]4)cc3Cl)cc21 ; CC(C)(C)n1cnc2ccc(Oc3c(Cl)cc(-n4nc(C#N)c(=O)[nH]c4=O)cc3Cl)cc21 ; O=C(Nc1cc(Cl)c(Oc2ccc3nc4n(c3c2)CCCO4)c(Cl)c1)c1noc(=O)[nH]1 ; N#Cc1nn(-c2cc(Cl)c(Oc3ccc4nc5n(c4c3)CCCO5)c(Cl)c2)c(=O)[nH]c1=O ; COc1nc2ccc(Oc3c(Cl)cc(NC(=O)c4noc(=O)[nH]4)cc3Cl)cc2n1C(C)(C)C ; CC(C)(C)n1c(=O)[nH]c2ccc(Oc3c(Cl)cc(NC(=O)c4noc(=O)[nH]4)cc3Cl)cc21]

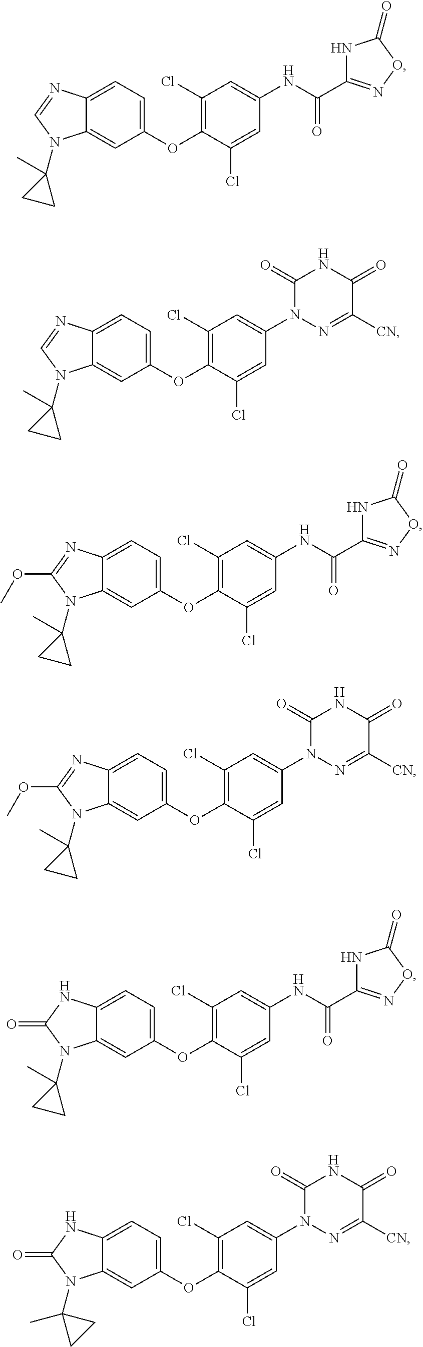 [SMILES: CC1(n2cnc3ccc(Oc4c(Cl)cc(NC(=O)c5noc(=O)[nH]5)cc4Cl)cc32)CC1 ; CC1(n2cnc3ccc(Oc4c(Cl)cc(-n5nc(C#N)c(=O)[nH]c5=O)cc4Cl)cc32)CC1 ; COc1nc2ccc(Oc3c(Cl)cc(NC(=O)c4noc(=O)[nH]4)cc3Cl)cc2n1C1(C)CC1 ; COc1nc2ccc(Oc3c(Cl)cc(-n4nc(C#N)c(=O)[nH]c4=O)cc3Cl)cc2n1C1(C)CC1 ; CC1(n2c(=O)[nH]c3ccc(Oc4c(Cl)cc(NC(=O)c5noc(=O)[nH]5)cc4Cl)cc32)CC1 ; CC1(n2c(=O)[nH]c3ccc(Oc4c(Cl)cc(-n5nc(C#N)c(=O)[nH]c5=O)cc4Cl)cc32)CC1]

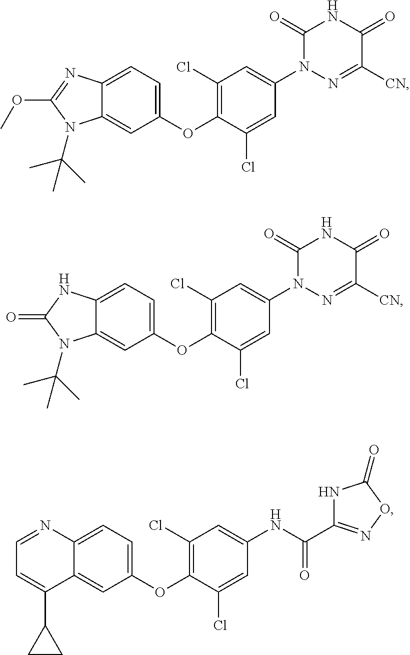 [SMILES: COc1nc2ccc(Oc3c(Cl)cc(-n4nc(C#N)c(=O)[nH]c4=O)cc3Cl)cc2n1C(C)(C)C ; CC(C)(C)n1c(=O)[nH]c2ccc(Oc3c(Cl)cc(-n4nc(C#N)c(=O)[nH]c4=O)cc3Cl)cc21 ; O=C(Nc1cc(Cl)c(Oc2ccc3nccc(C4CC4)c3c2)c(Cl)c1)c1noc(=O)[nH]1]

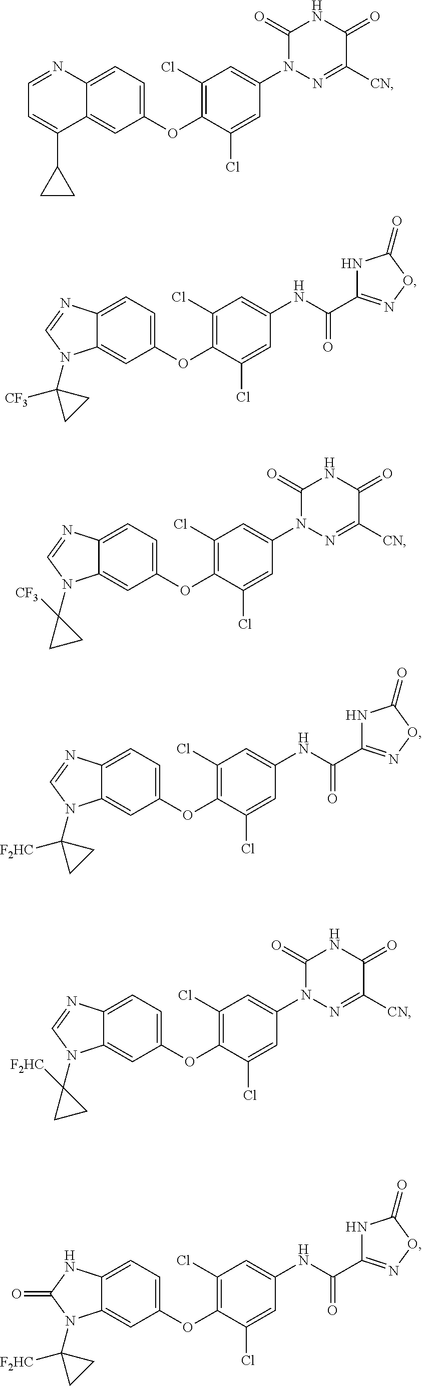 [SMILES: N#Cc1nn(-c2cc(Cl)c(Oc3ccc4nccc(C5CC5)c4c3)c(Cl)c2)c(=O)[nH]c1=O ; O=C(Nc1cc(Cl)c(Oc2ccc3ncn(C4(C(F)(F)F)CC4)c3c2)c(Cl)c1)c1noc(=O)[nH]1 ; N#Cc1nn(-c2cc(Cl)c(Oc3ccc4ncn(C5(C(F)(F)F)CC5)c4c3)c(Cl)c2)c(=O)[nH]c1=O ; O=C(Nc1cc(Cl)c(Oc2ccc3ncn(C4(C(F)F)CC4)c3c2)c(Cl)c1)c1noc(=O)[nH]1 ; N#Cc1nn(-c2cc(Cl)c(Oc3ccc4ncn(C5(C(F)F)CC5)c4c3)c(Cl)c2)c(=O)[nH]c1=O ; O=C(Nc1cc(Cl)c(Oc2ccc3[nH]c(=O)n(C4(C(F)F)CC4)c3c2)c(Cl)c1)c1noc(=O)[nH]1]

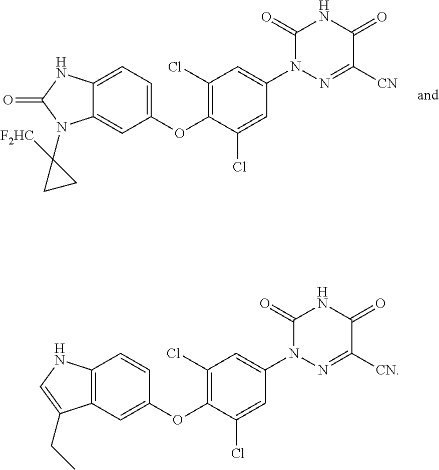 [SMILES: N#Cc1nn(-c2cc(Cl)c(Oc3ccc4[nH]c(=O)n(C5(C(F)F)CC5)c4c3)c(Cl)c2)c(=O)[nH]c1=O ; CCc1c[nH]c2ccc(Oc3c(Cl)cc(-n4nc(C#N)c(=O)[nH]c4=O)cc3Cl)cc12]

WO 2020/041741 discloses THRB agonists of the following structures:

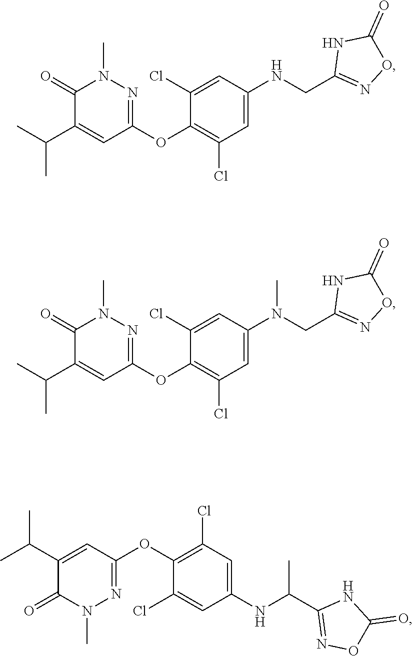 [SMILES: CC(C)c1cc(Oc2c(Cl)cc(NCc3noc(=O)[nH]3)cc2Cl)nn(C)c1=O ; CC(C)c1cc(Oc2c(Cl)cc(N(C)Cc3noc(=O)[nH]3)cc2Cl)nn(C)c1=O ; CC(C)c1cc(Oc2c(Cl)cc(NC(C)c3noc(=O)[nH]3)cc2Cl)nn(C)c1=O]

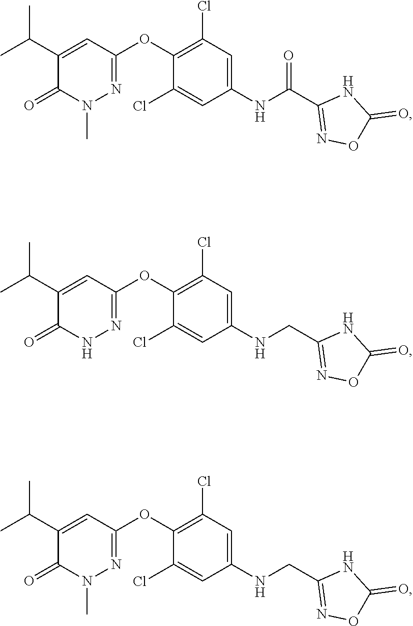 [SMILES: CC(C)c1cc(Oc2c(Cl)cc(NC(=O)c3noc(=O)[nH]3)cc2Cl)nn(C)c1=O ; CC(C)c1cc(Oc2c(Cl)cc(NCc3noc(=O)[nH]3)cc2Cl)n[nH]c1=O ; CC(C)c1cc(Oc2c(Cl)cc(NCc3noc(=O)[nH]3)cc2Cl)nn(C)c1=O]

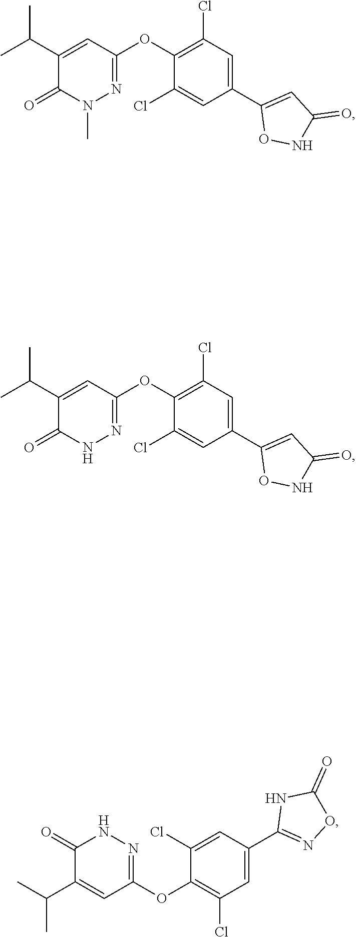 [SMILES: CC(C)c1cc(Oc2c(Cl)cc(-c3cc(=O)[nH]o3)cc2Cl)nn(C)c1=O ; CC(C)c1cc(Oc2c(Cl)cc(-c3cc(=O)[nH]o3)cc2Cl)n[nH]c1=O ; CC(C)c1cc(Oc2c(Cl)cc(-c3noc(=O)[nH]3)cc2Cl)n[nH]c1=O]

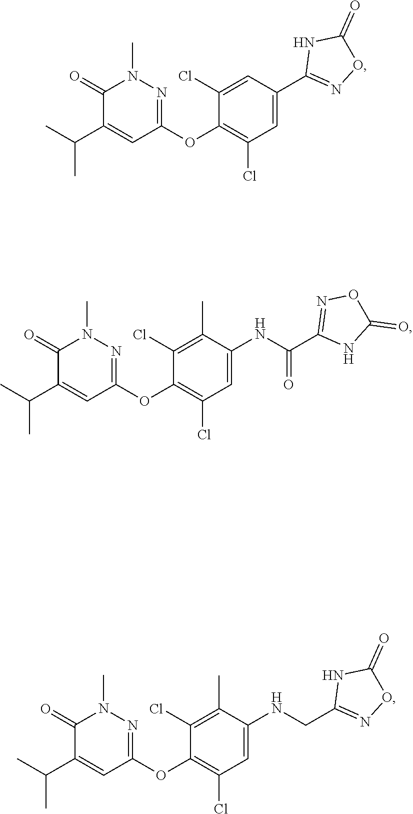 [SMILES: CC(C)c1cc(Oc2c(Cl)cc(-c3noc(=O)[nH]3)cc2Cl)nn(C)c1=O ; Cc1c(NC(=O)c2noc(=O)[nH]2)cc(Cl)c(Oc2cc(C(C)C)c(=O)n(C)n2)c1Cl ; Cc1c(NCc2noc(=O)[nH]2)cc(Cl)c(Oc2cc(C(C)C)c(=O)n(C)n2)c1Cl]

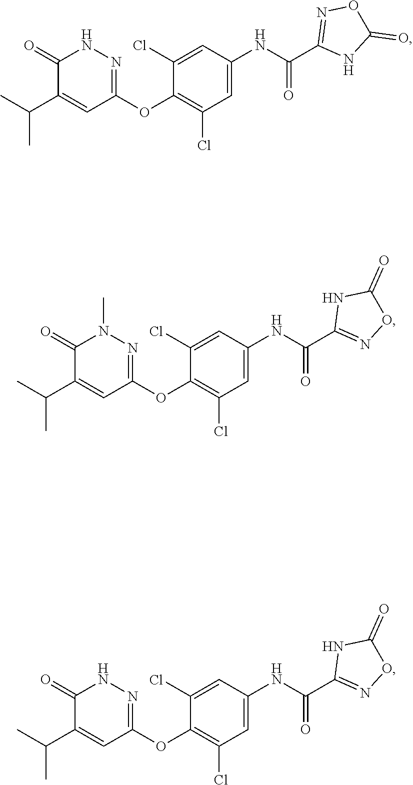 [SMILES: CC(C)c1cc(Oc2c(Cl)cc(NC(=O)c3noc(=O)[nH]3)cc2Cl)n[nH]c1=O ; CC(C)c1cc(Oc2c(Cl)cc(NC(=O)c3noc(=O)[nH]3)cc2Cl)nn(C)c1=O ; CC(C)c1cc(Oc2c(Cl)cc(NC(=O)c3noc(=O)[nH]3)cc2Cl)n[nH]c1=O]

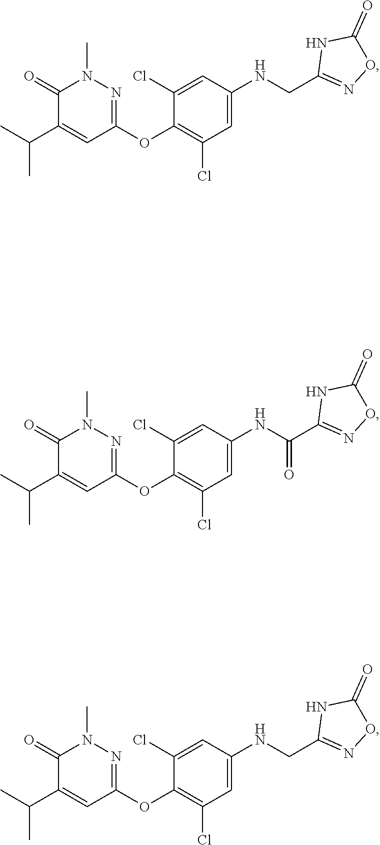 [SMILES: CC(C)c1cc(Oc2c(Cl)cc(NCc3noc(=O)[nH]3)cc2Cl)nn(C)c1=O ; CC(C)c1cc(Oc2c(Cl)cc(NC(=O)c3noc(=O)[nH]3)cc2Cl)nn(C)c1=O ; CC(C)c1cc(Oc2c(Cl)cc(NCc3noc(=O)[nH]3)cc2Cl)nn(C)c1=O]

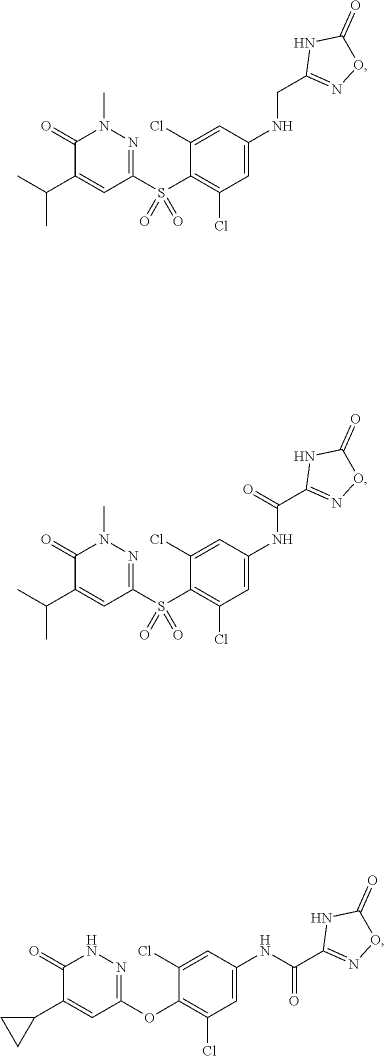 [SMILES: CC(C)c1cc(S(=O)(=O)c2c(Cl)cc(NCc3noc(=O)[nH]3)cc2Cl)nn(C)c1=O ; CC(C)c1cc(S(=O)(=O)c2c(Cl)cc(NC(=O)c3noc(=O)[nH]3)cc2Cl)nn(C)c1=O ; O=C(Nc1cc(Cl)c(Oc2cc(C3CC3)c(=O)[nH]n2)c(Cl)c1)c1noc(=O)[nH]1]

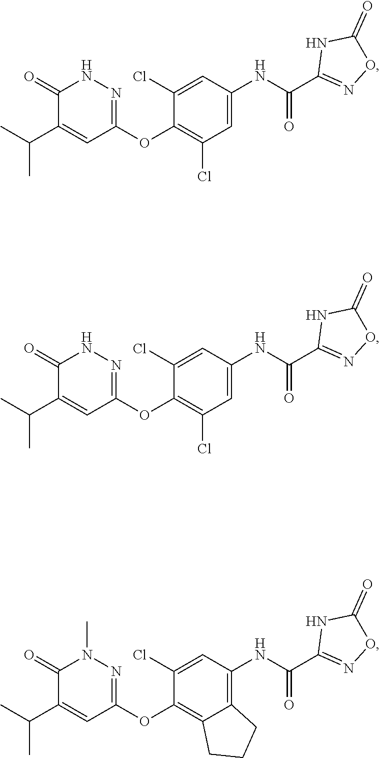 [SMILES: CC(C)c1cc(Oc2c(Cl)cc(NC(=O)c3noc(=O)[nH]3)cc2Cl)n[nH]c1=O ; CC(C)c1cc(Oc2c(Cl)cc(NC(=O)c3noc(=O)[nH]3)cc2Cl)n[nH]c1=O ; CC(C)c1cc(Oc2c(Cl)cc(NC(=O)c3noc(=O)[nH]3)c3c2CCC3)nn(C)c1=O]

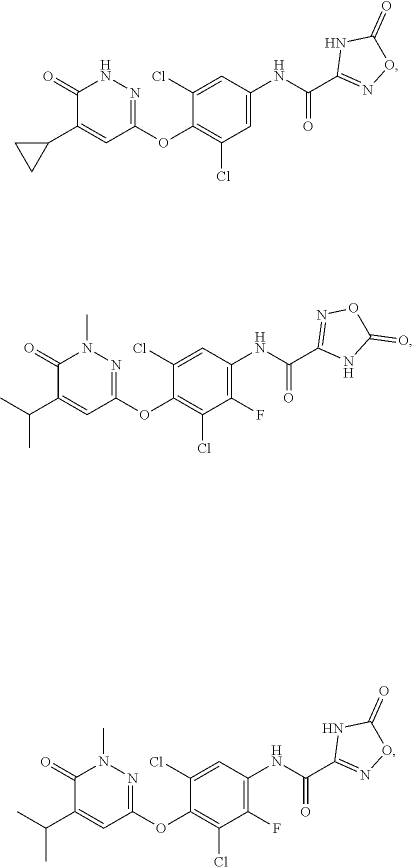 [SMILES: O=C(Nc1cc(Cl)c(Oc2cc(C3CC3)c(=O)[nH]n2)c(Cl)c1)c1noc(=O)[nH]1 ; CC(C)c1cc(Oc2c(Cl)cc(NC(=O)c3noc(=O)[nH]3)c(F)c2Cl)nn(C)c1=O ; CC(C)c1cc(Oc2c(Cl)cc(NC(=O)c3noc(=O)[nH]3)c(F)c2Cl)nn(C)c1=O]

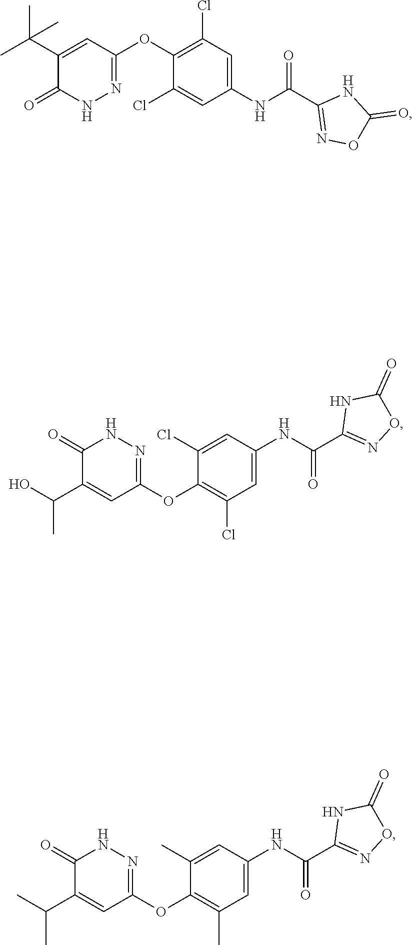 [SMILES: CC(C)(C)c1cc(Oc2c(Cl)cc(NC(=O)c3noc(=O)[nH]3)cc2Cl)n[nH]c1=O ; CC(O)c1cc(Oc2c(Cl)cc(NC(=O)c3noc(=O)[nH]3)cc2Cl)n[nH]c1=O ; Cc1cc(NC(=O)c2noc(=O)[nH]2)cc(C)c1Oc1cc(C(C)C)c(=O)[nH]n1]

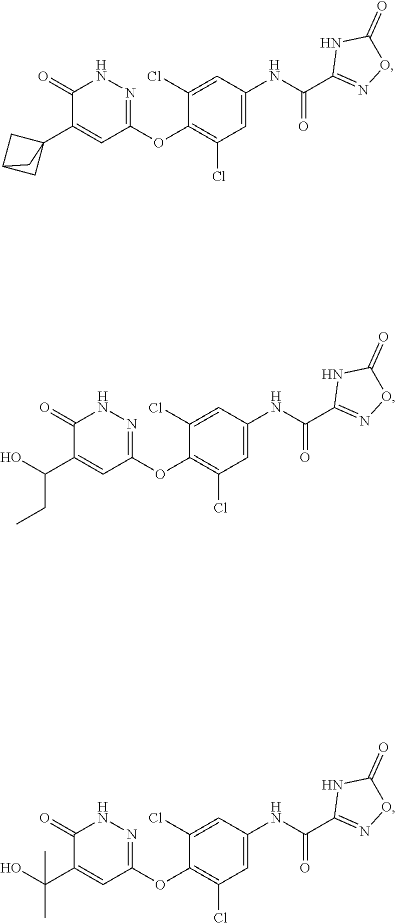 [SMILES: O=C(Nc1cc(Cl)c(Oc2cc(C34CC(C3)C4)c(=O)[nH]n2)c(Cl)c1)c1noc(=O)[nH]1 ; CCC(O)c1cc(Oc2c(Cl)cc(NC(=O)c3noc(=O)[nH]3)cc2Cl)n[nH]c1=O ; CC(C)(O)c1cc(Oc2c(Cl)cc(NC(=O)c3noc(=O)[nH]3)cc2Cl)n[nH]c1=O]

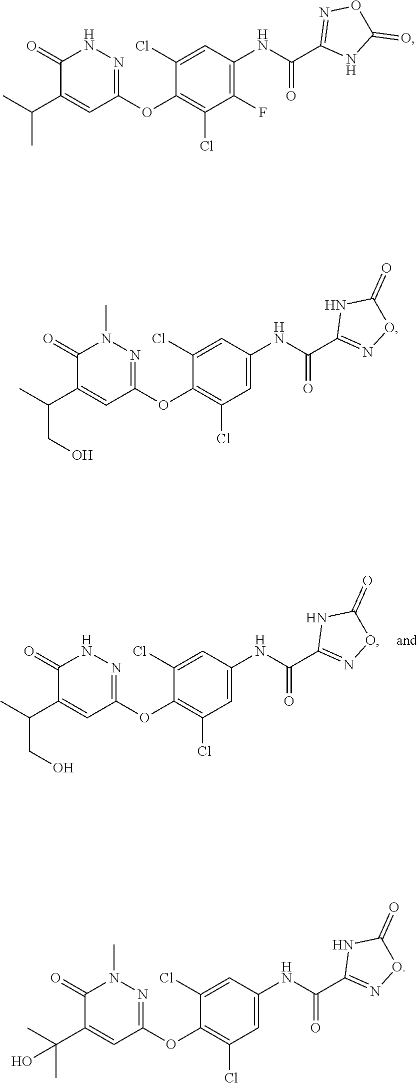 [SMILES: CC(C)c1cc(Oc2c(Cl)cc(NC(=O)c3noc(=O)[nH]3)c(F)c2Cl)n[nH]c1=O ; CC(CO)c1cc(Oc2c(Cl)cc(NC(=O)c3noc(=O)[nH]3)cc2Cl)nn(C)c1=O ; CC(CO)c1cc(Oc2c(Cl)cc(NC(=O)c3noc(=O)[nH]3)cc2Cl)n[nH]c1=O ; Cn1nc(Oc2c(Cl)cc(NC(=O)c3noc(=O)[nH]3)cc2Cl)cc(C(C)(C)O)c1=O]

WO 2020/077123 discloses THRB agonists of the following structures:

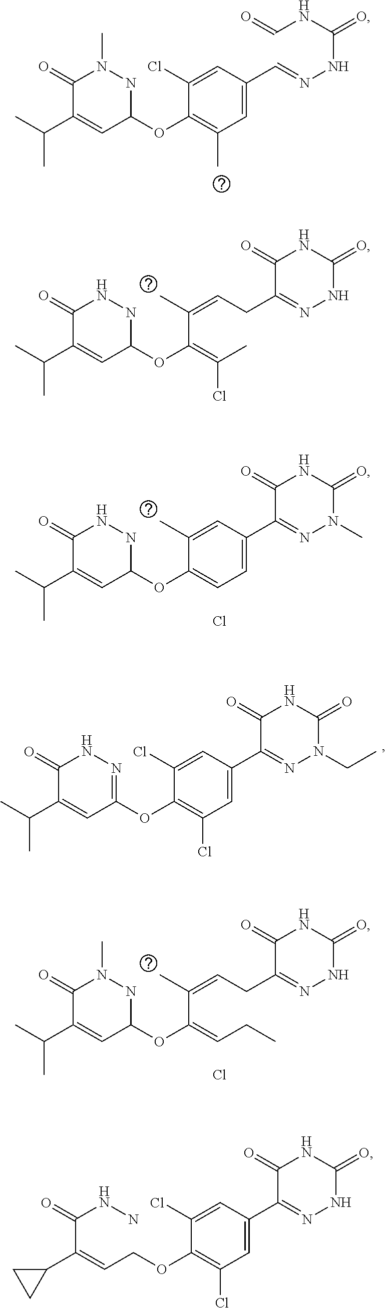

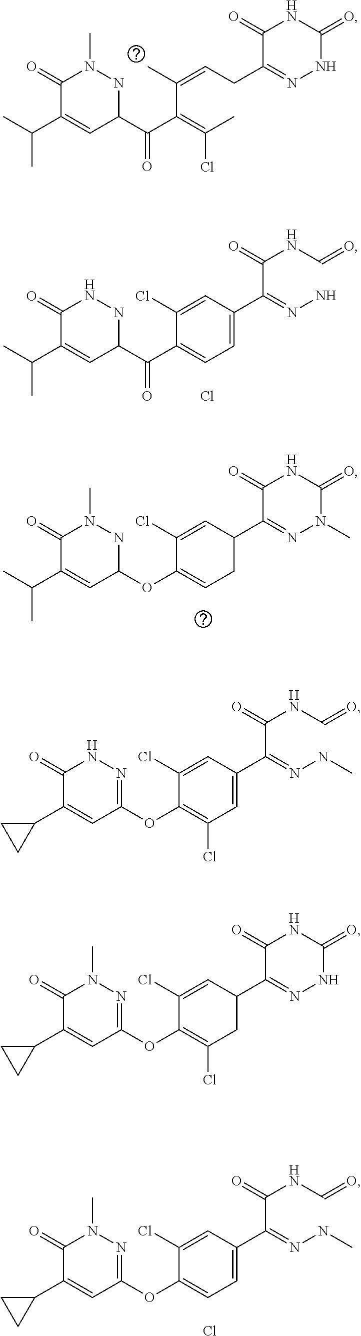

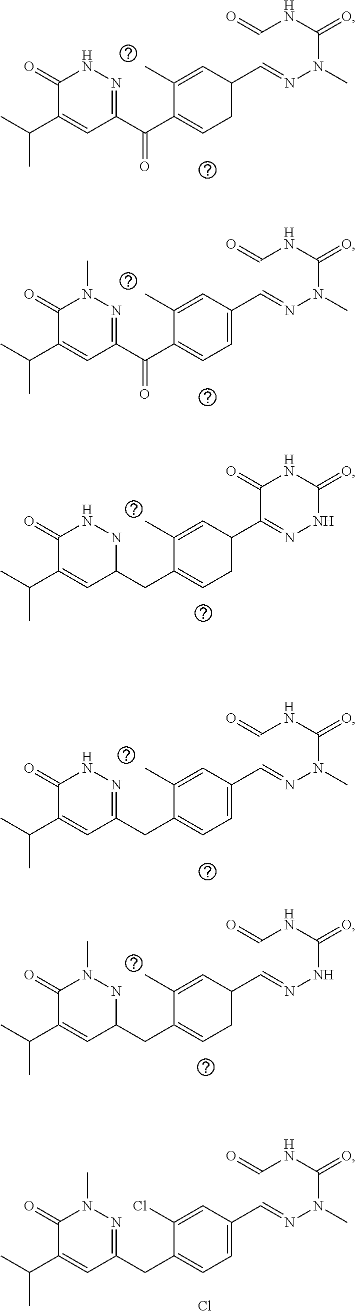

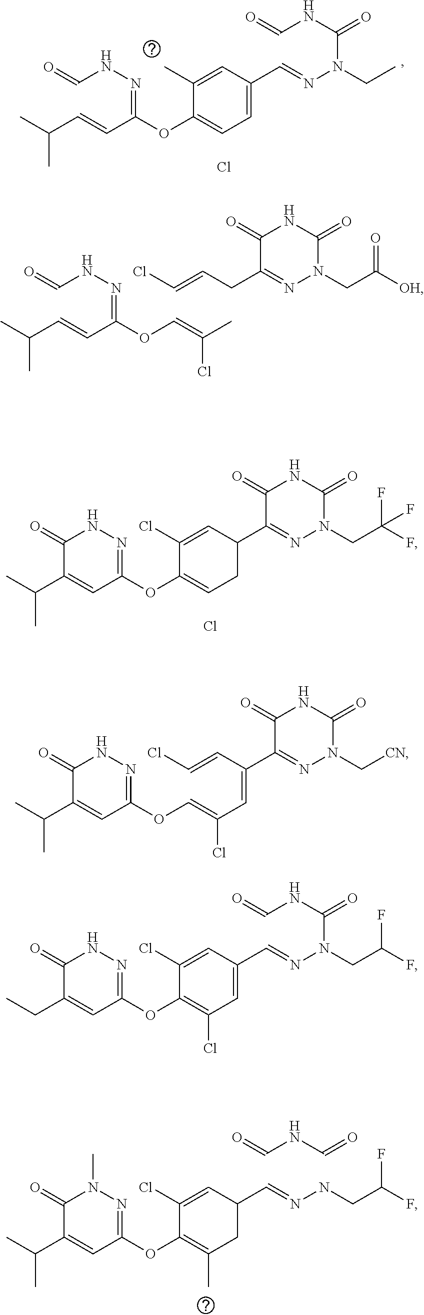

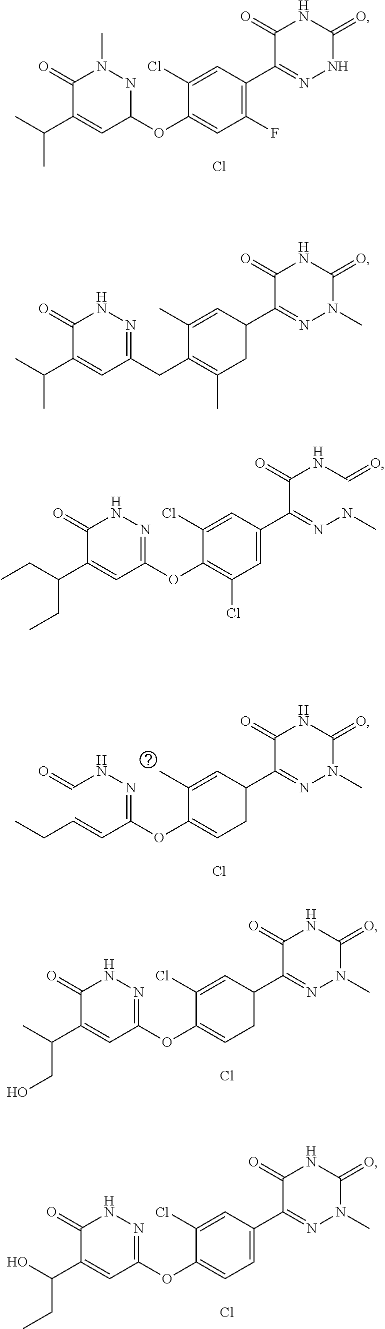

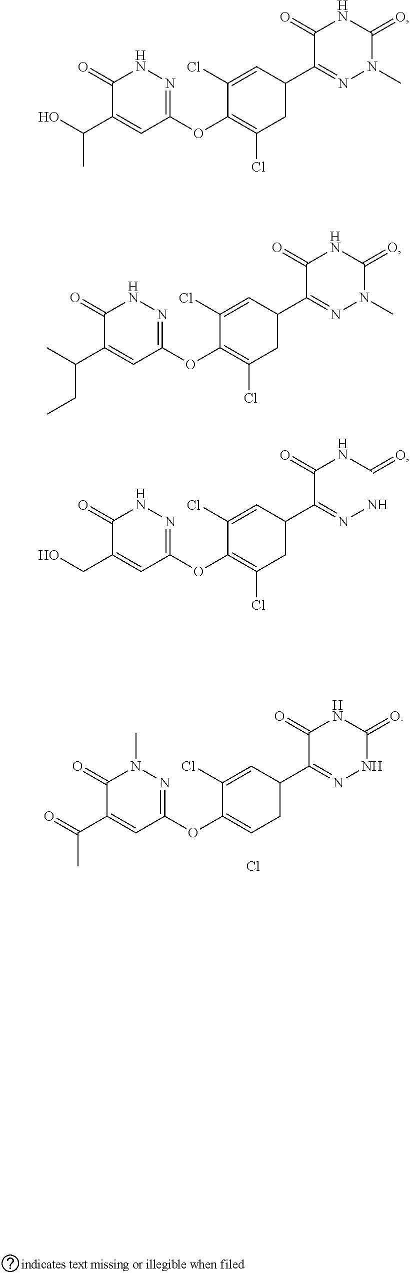

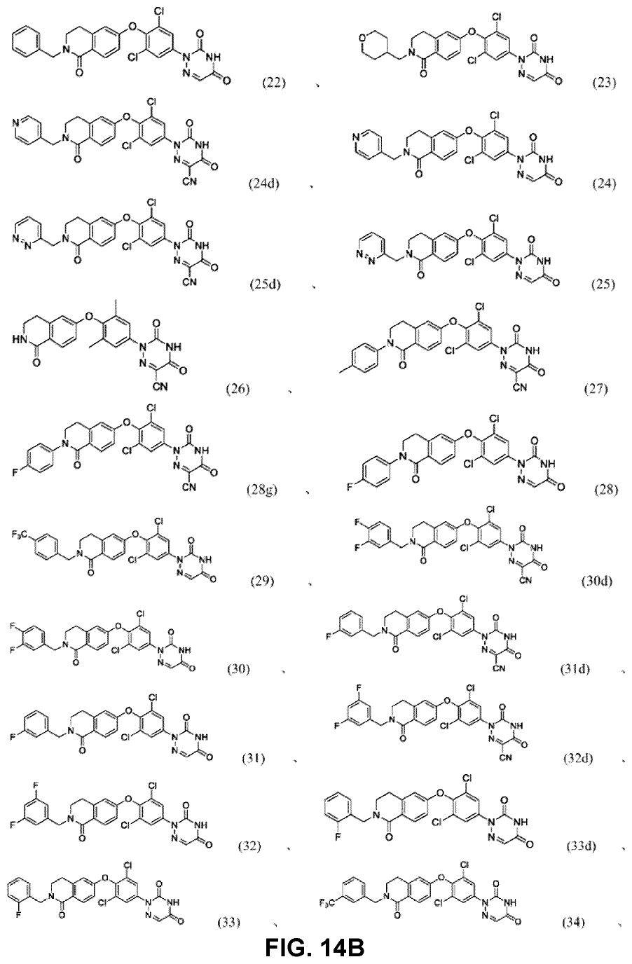

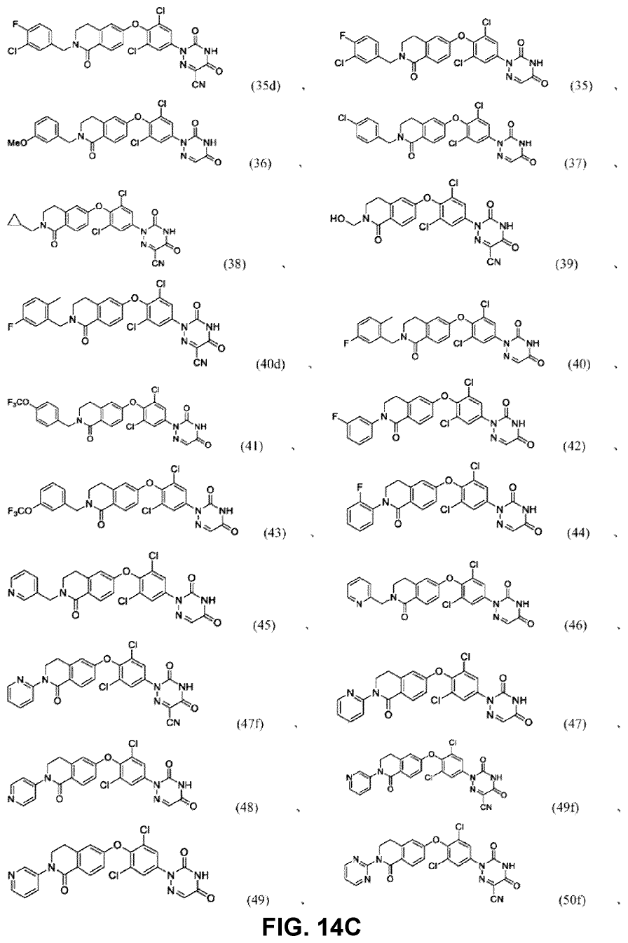

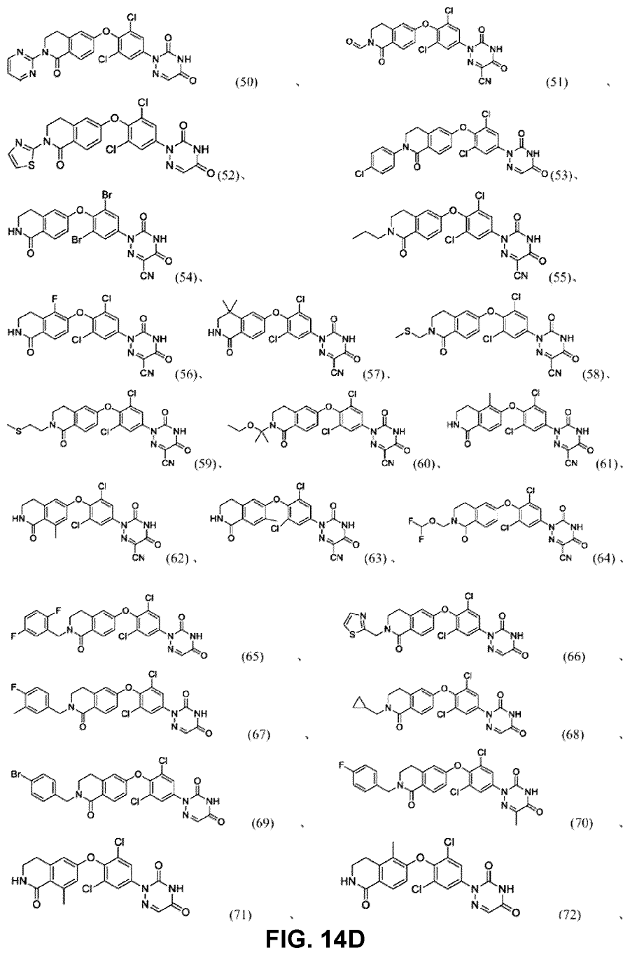

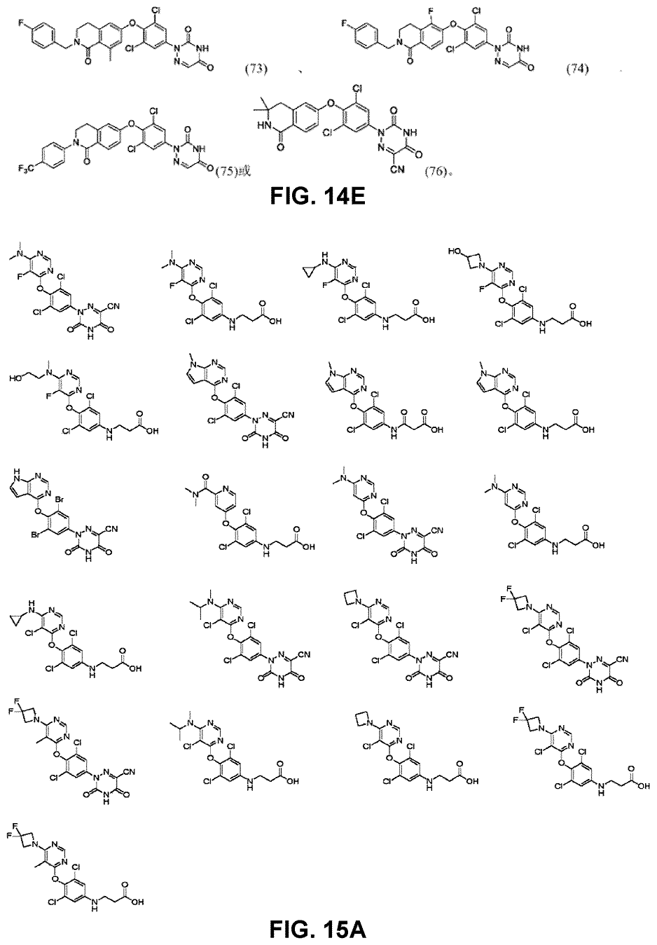

WO 2022/057845 discloses THRB agonists having the structures disclosed in FIGS. 14A-14E.

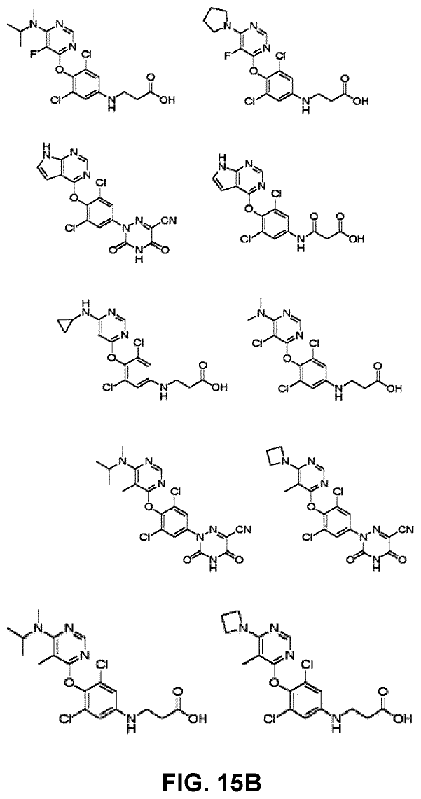

WO 2021/018226 discloses THRB agonists having the structures disclosed in FIGS. 15A-15B.

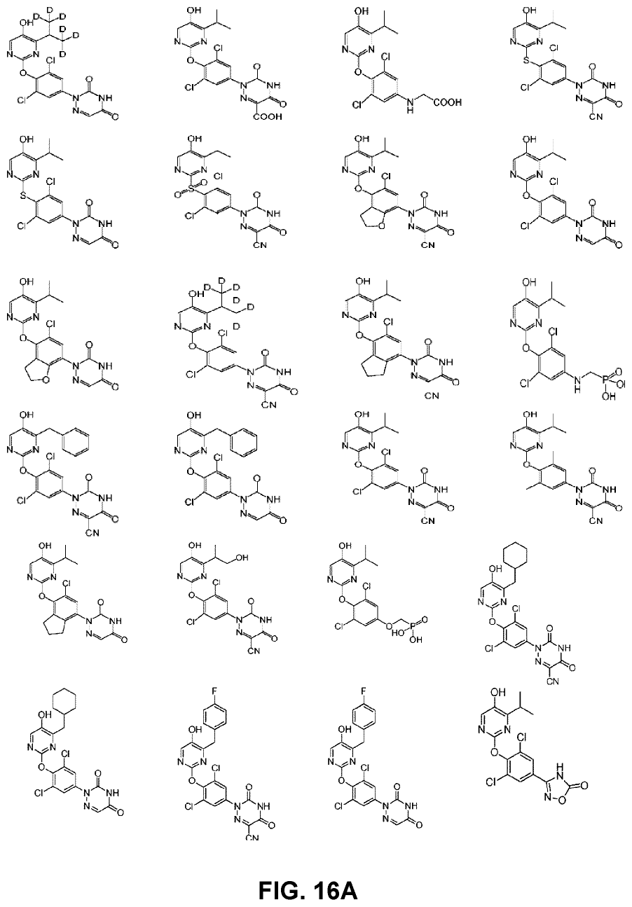

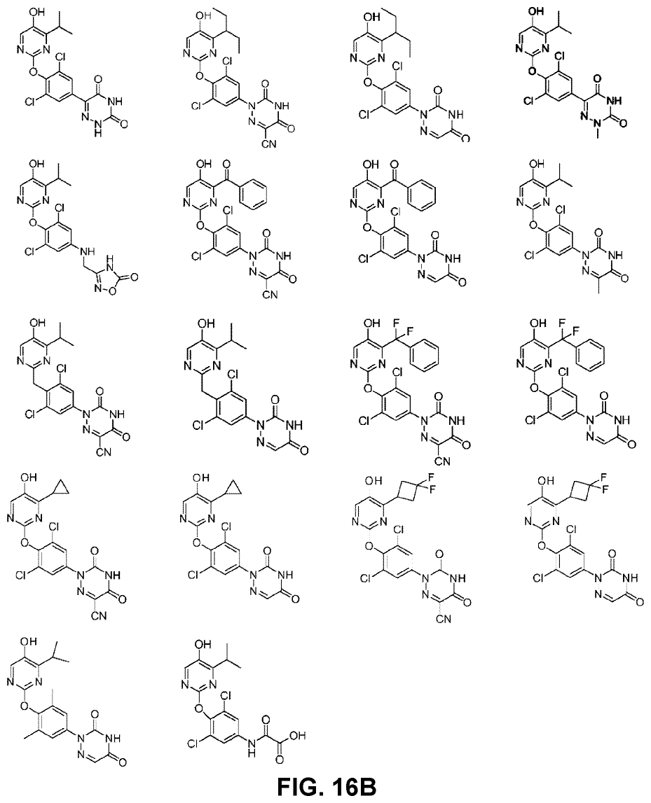

WO 2021/244582 discloses THRB agonists having the structures disclosed in FIGS. 16A-16B.

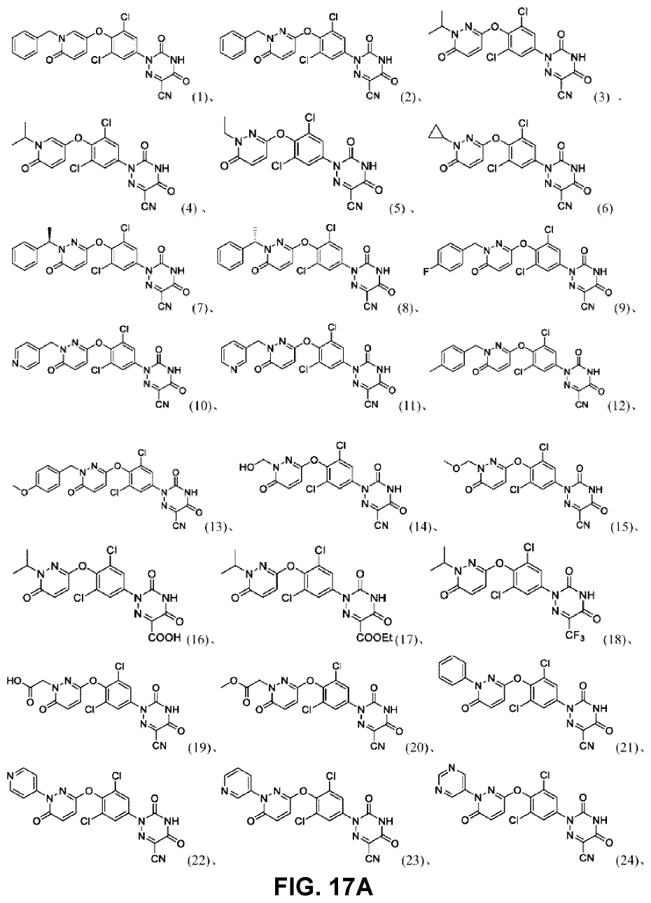

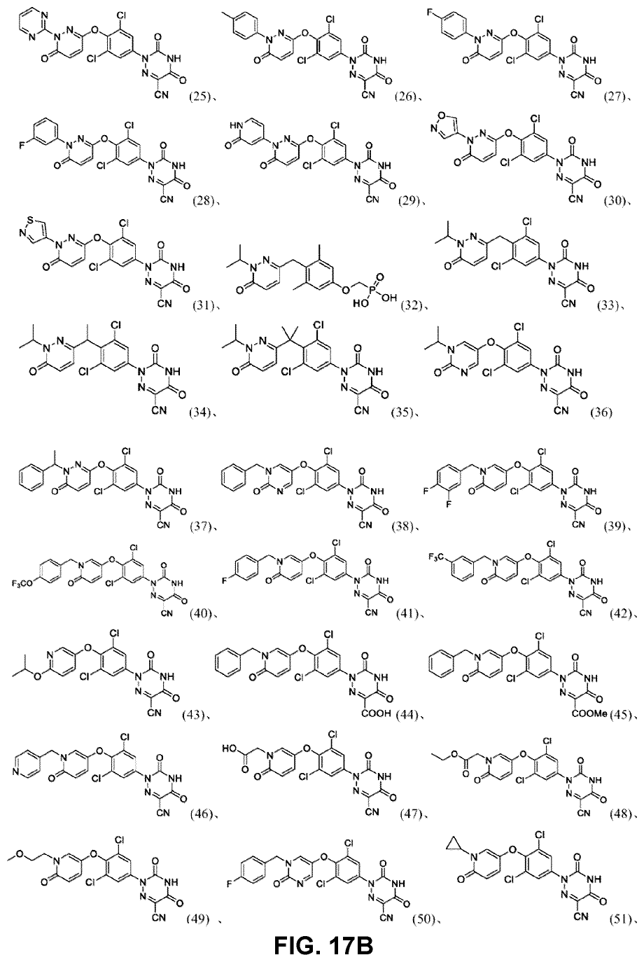

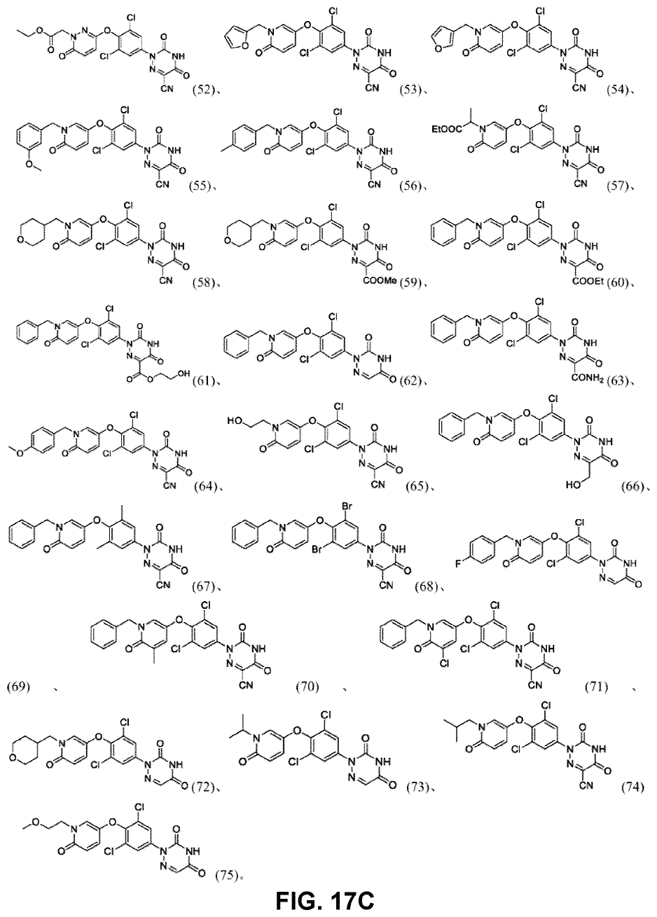

WO 2021/057791 discloses THRB agonists having the structures disclosed in FIGS. 17A-17C.

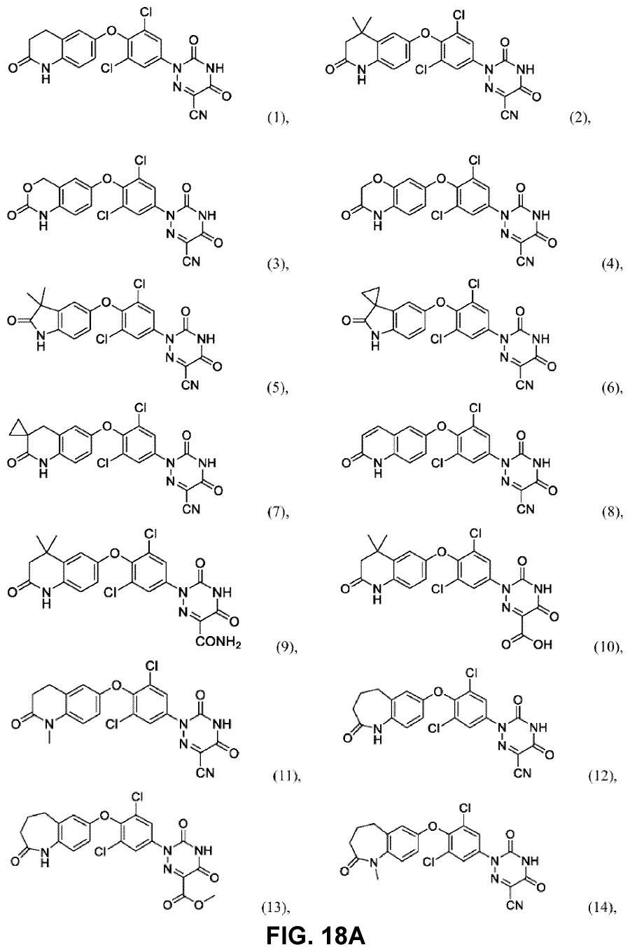

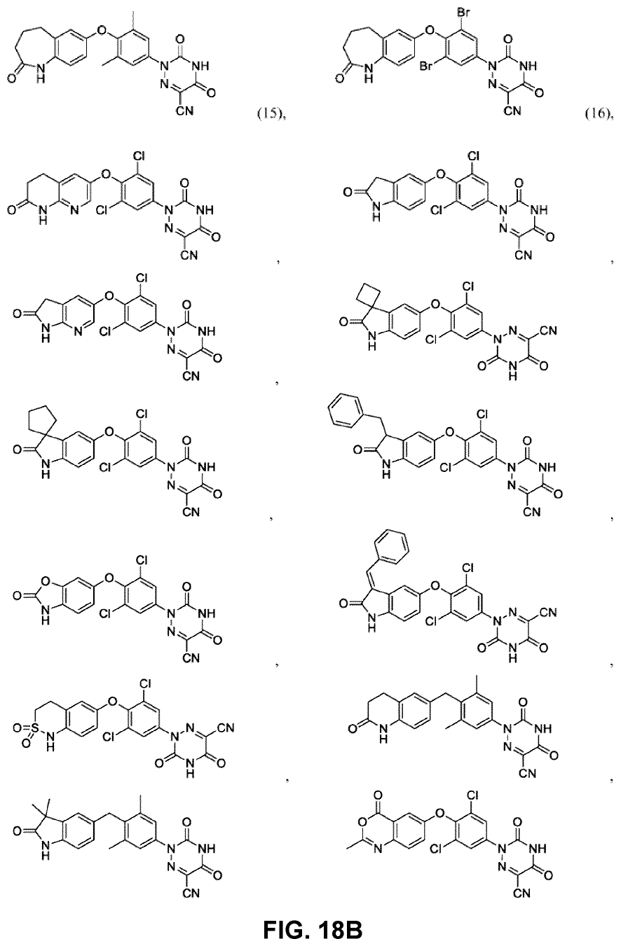

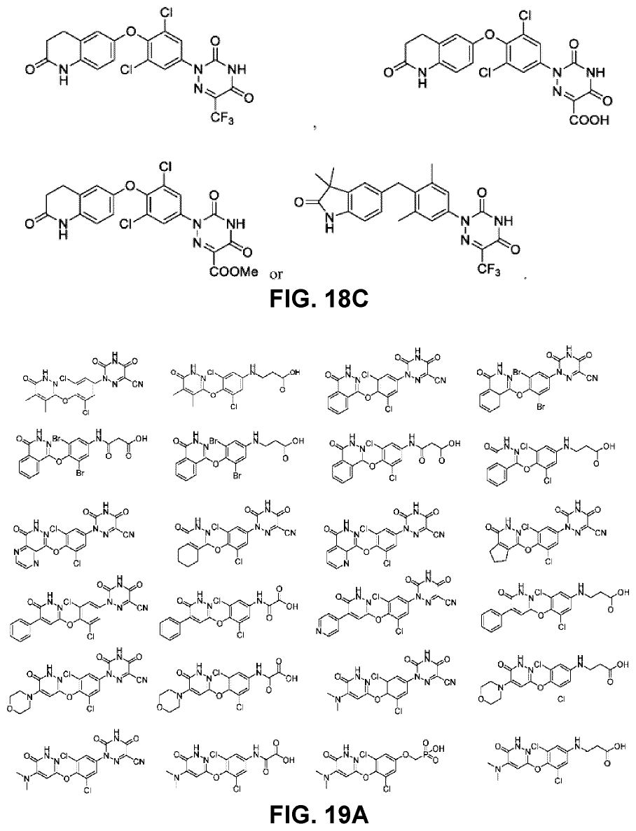

WO 2021/043185 discloses THRB agonists having the structures disclosed in FIGS. 18A-18C.

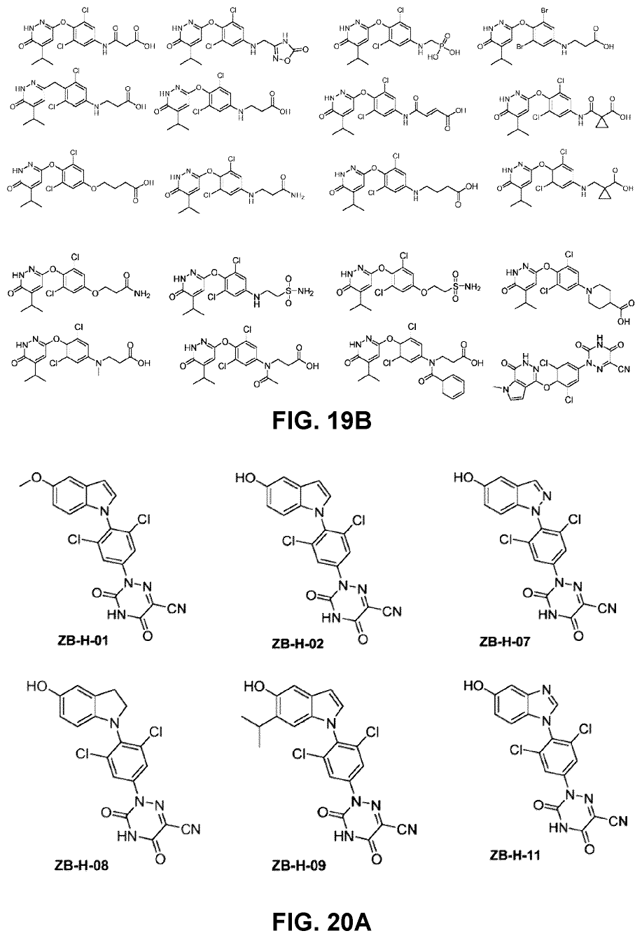

WO 2020/228577 discloses THRB agonists having the structures disclosed in FIGS. 19A-19B.

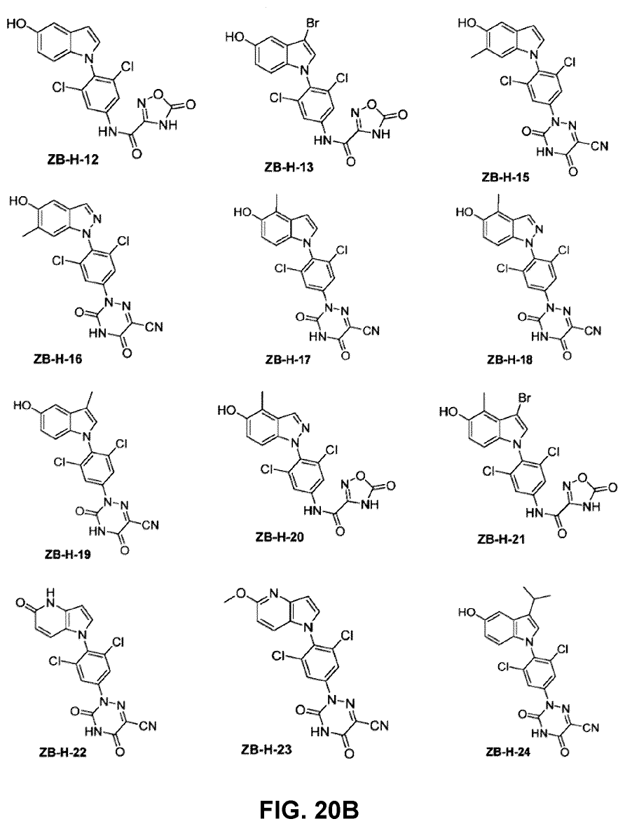

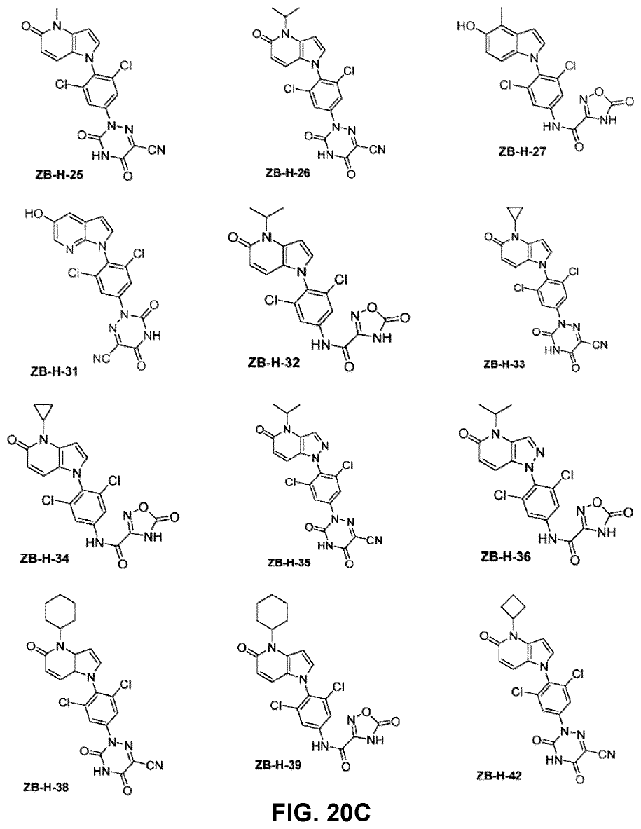

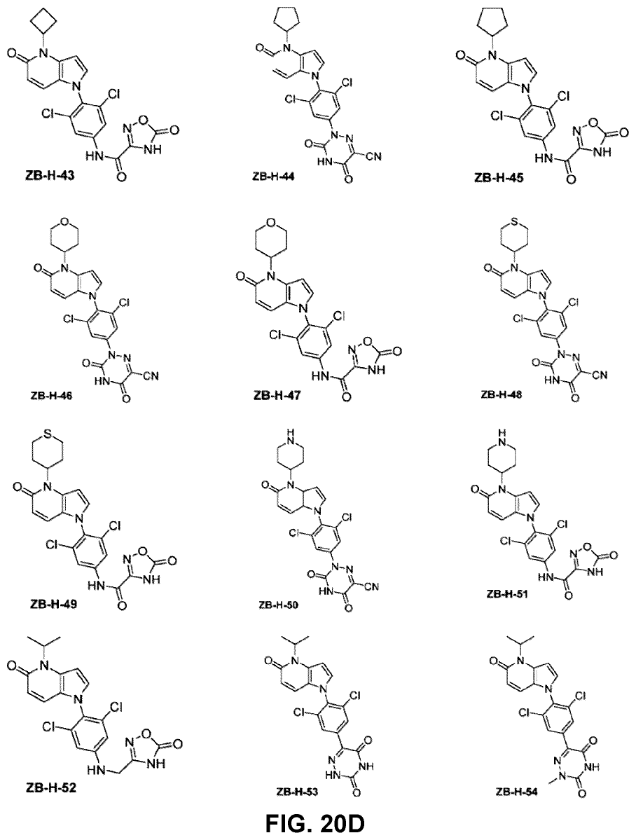

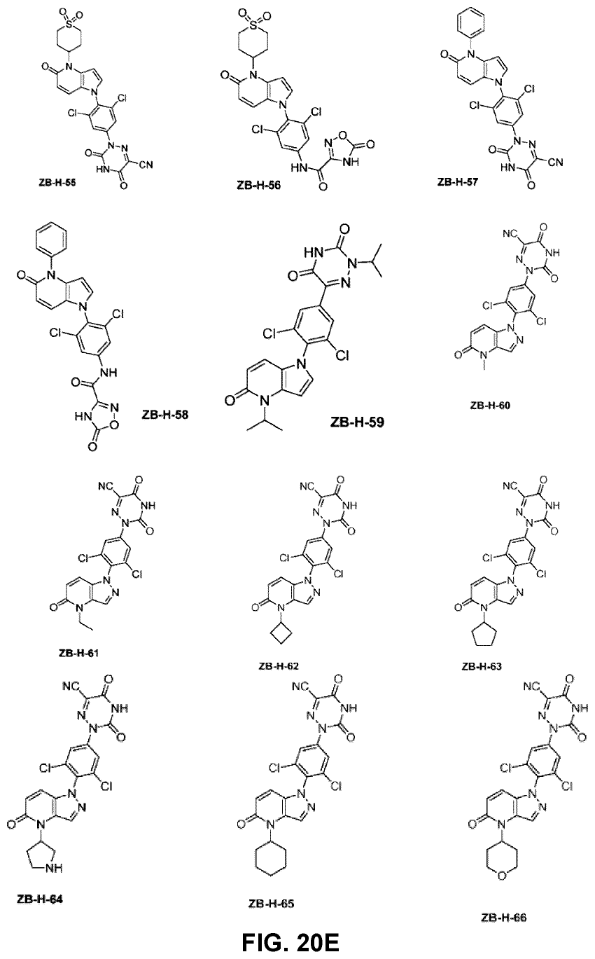

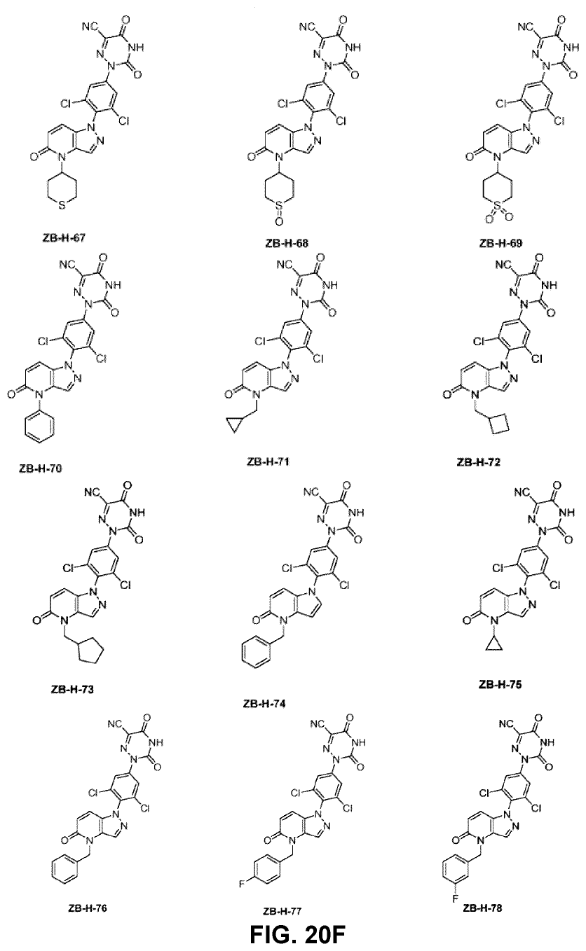

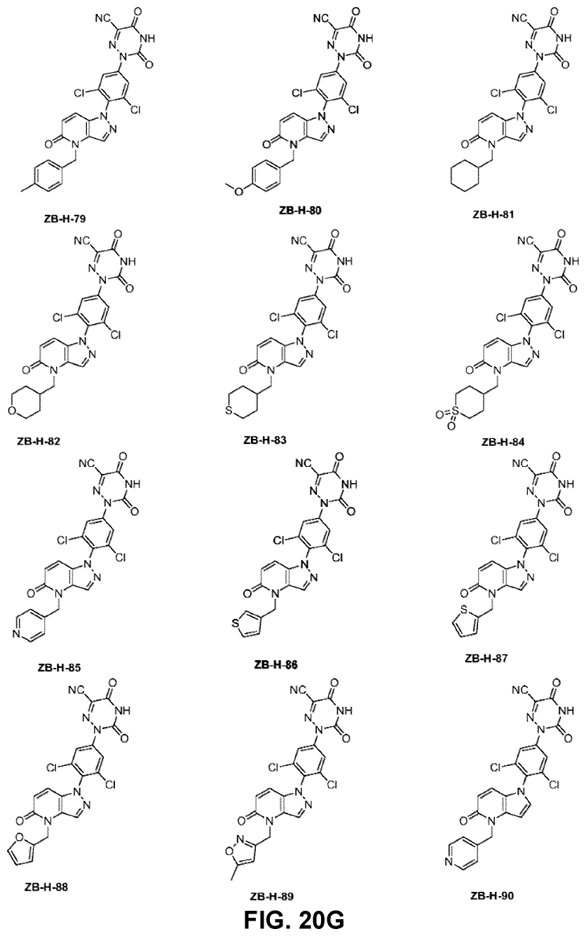

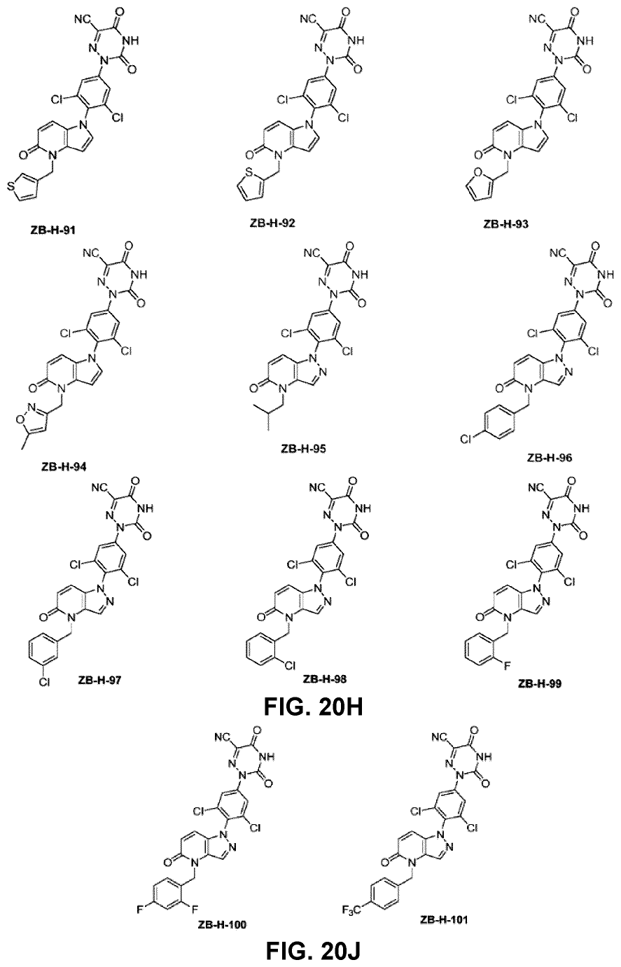

WO 2022/127699 discloses THRB agonists having the structures disclosed in FIGS. 20A-20J.

As used herein, the term “hematopoietic stem cells” or “HSCs” is intended to mean cells having both pluripotency which allows them to differentiate into functional mature cells such as granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages), and the ability to regenerate while maintaining their pluripotency (self-renewal). The term “hematopoietic progenitor cells” or “HPCs” refers to cells that originate from HSCs and further differentiate to create the specialized blood cell types described above. HSCs and HPCs are often collectively referred to as Hematopoietic stem and progenitor cells, HSPCs.

Hematopoietic stem cells and hematopoietic progenitor cells can be obtained from any donor. The hematopoietic stem cells and hematopoietic progenitor cells can be healthy, wild type cells or they can have mutations that can adversely affect their function. Any such cells can be cultured or expanded according to the methods described herein. The methods described herein can be used to detect such mutations, and in some cases repair the mutations so that genetically repaired cells can then be expanded. In some cases, the hematopoietic stem cells and hematopoietic progenitor cells are autologous to a person or patient who may later receive an expanded population of the hematopoietic stem cells and/or hematopoietic progenitor cells.

In an embodiment, the above-mentioned method comprises (a) providing a cell population comprising HSCs and/or HPCs and (b) culturing said cell population ex vivo under suitable conditions for expanding HSCs and/or HPCs. The cell population (e.g., umbilical cord blood sample, bone marrow sample, peripheral blood sample, spleen sample) may first be subjected to enrichment or purification steps, including negative and/or positive selection of cells based on specific cellular markers (e.g., CD34+, CD38−, CD123, TIM3, CD96, etc.) in order to provide a starting cell population. Methods for isolating said starting cell population based on specific cellular markers may use fluorescent activated cell sorting (FACS) technology or solid or insoluble substrate to which is bound antibodies or ligands that interact with specific cell surface markers (e.g., anti-CD34 antibodies). For example, cells may be contacted with a solid substrate (e.g., column of beads, flasks, magnetic particles) containing the antibodies and any unbound cells are removed. When a solid substrate comprising magnetic or paramagnetic beads is used, cells bound to the beads can be readily isolated by a magnetic separator. In an embodiment, the starting cell population comprising HSC and/or HPC is enriched in CD34⁺ cells. Methods for enriching samples such as blood cell population in CD34⁺ cells include kits commercialized by Miltenyi Biotec™ (CD34⁺ direct isolation kit, Miltenyi Biotec™, Bergisch, Gladbach, Germany) or by Baxter (Isolex™ 3000). In an embodiment, the starting cell population comprising HSC and/or HPC is derived from neonatal umbilical cord blood cells which have been enriched in CD34⁺ cells. In a related embodiment, the starting cell population is derived from one or two umbilical cord blood units. In another embodiment, the starting cell population comprising HSCs and/or HPCs is derived from human mobilized peripheral blood cells which have been enriched in CD34⁺ cells. In a related embodiment, the starting cell population comprising HSCs and/or HPCs is derived from human mobilized peripheral blood cells isolated from only one patient.

Hematopoietic stem and progenitor cells can be mobilized and obtained from the peripheral blood. Mobilization is a process whereby the cells are stimulated out of the bone marrow space (e.g., from the hip bones and the chest bone) into the bloodstream so they are available for collection. Mozobil® (plerixafor) injection can be used for such mobilization. In some cases, Mozobil (plerixafor) can be used in combination with granulocyte-colony stimulating factor (G-CSF) to mobilize hematopoietic stem and progenitor cells to the peripheral blood. After collection the cells can be expanded immediately, or frozen and stored until expansion is desired.

The hematopoietic stem cells and/or hematopoietic progenitor cells can be collected from donors suffering from a disease or condition, and then modified to alleviate the symptoms or progression of the disease or condition. For example, when a genetic defect is correlated with a disease and condition, hematopoietic stem cell and/or hematopoietic progenitor cell can be collected from a subject (donor) with the disease or condition, the genetic defect can be repaired, and the genetically-repaired hematopoietic stem cells and/or hematopoietic progenitor cells can be expanded using the method described herein. A population of the expanded, repaired cells can then be administered to the subject (donor). Cells can be genetically modified by any convenient method. Nonlimiting examples of methods of introducing a modification into the genome of a cell can include use of microinjection, viral delivery, recombinase technologies (e.g., Cre-lox system), homologous recombination, TALENS, CRISPR, and/or ZFN, see, e.g., Clark and Whitelaw, Nature Reviews Genetics 4:825-833 (2003). For example, nucleases such as Cas nucleases, zinc finger nucleases (ZFNs), transcription activator-like effector nucleases (TALENs), and/or meganucleases can be employed with a guide nucleic acid that allows the nuclease to target the genomic locus associated with a genetic disease or a condition. Thus, in another aspect, the present disclosure provides a method of treating a subject suffering from a disease comprising a genetic defect affecting hematopoietic cells comprising harvesting HSCs and/or HPCs from the subject, culturing the HSCs and/or HPCs according to the methods of this disclosure; genetically manipulating the expanded HSCs and/or HPCs to correct the genetic defect; and administering the genetically-manipulated HSCs and/or HPCs to the subject. Examples of diseases caused by a genetic defect that affects hematopoietic cells, for example, Fanconi Anemia (FA), sickle cell anemia, severe combined immunodeficiency, β-thalassemia, Wiskott-Aldrich syndrome, adenosine deaminase SCID (ADA SCID), HIV, Diamond-Blackfan anemia, Schwachman-Diamond syndrome, metachromatic leukodystrophy, and leukodystrophy.

A targeting vector can be used to repair a genetic defect. A “targeting vector” is a vector generally has a 5′ flanking region and a 3′ flanking region homologous to segments of the gene of interest. The 5′ flanking region and a 3′ flanking region can surround a DNA sequence comprising a modification and/or a repair DNA sequence to be inserted/substituted within a selected genetic defect. In some cases, the targeting vector does not comprise a selectable marker. However, such a selectable marker can facilitate identification and selection of cells with desirable genetic modifications. Examples of suitable selectable markers include antibiotics resistance genes such as chloramphenicol resistance, gentamycin resistance, kanamycin resistance, spectinomycin resistance (SpecR), neomycin resistance gene (NEO), and/or the hygromycin β-phosphotransferase genes. The 5′ flanking region and the 3′ flanking region can be homologous to regions within the gene, or to regions flanking the gene to be deleted, modified, or replaced with the unrelated DNA sequence.

The targeting vector is contacted with the native gene of interest within the cell under conditions that favor homologous recombination. For example, the cell can be contacted with the targeting vector under conditions that result in transformation of the cell(s) with the targeting vector.

A typical targeting vector contains nucleic acid fragments of not less than about 0.1 kb nor more than about 10.0 kb from both the 5′ and the 3′ ends of the genomic locus which encodes the gene or locus to be modified. These two fragments are separated by an intervening fragment of nucleic acid which encodes the modification to be introduced. When the resulting construct recombines homologously with the chromosome at this locus, it results in the introduction of the modification.

The cell culture for expanding HSCs and/or HPCs may be carried out in natural medium, a semi-synthetic medium or a synthetic medium in terms of composition, and may be a solid medium, a semisolid medium or a liquid medium in terms of shape, and any nutrient medium used for cell culture, which may be supplemented with a mixture of cell expanding factors. Such medium typically comprises sodium, potassium, calcium, magnesium, phosphorus, chlorine, amino acids, vitamins, cytokines, hormones, antibiotics, serum, fatty acids, saccharides or the like. In the culture, other chemical components or biological components may be incorporated singly or in combination, as the case requires. Such components to be incorporated in the medium may be fetal calf serum, human serum, horse serum, insulin, transferrin, lactoferrin, cholesterol, ethanolamine, sodium selenite, monothioglycerol, 2-mercaptoethanol, bovine serum albumin (or albumin replacement agents such as PVA), sodium pyruvate, polyethylene glycol, various vitamins, various amino acids, agar, agarose, collagen, methylcellulose, various cytokines, various growth factors or the like. For example, the medium may be supplemented with a combination of bovine serum albumin, insulin, transferrin (BIT). Examples of such basal medium appropriate for a method of culturing cells without limitation, Dulbecco's Modified Eagles's Medium (DMEM), Ham's Nutrient Mixture H12 Mixture F12, McCoy's 5A medium, Eagles's Minimum Essential Medium (EMEM), aMEM medium (alpha Modified Eagles's Minimum Essential Medium), RPMI1640® medium, Isocove's Modified Dulbecco's Medium (IMDM), StemPro34 (Invitrogen®), X-VIVO 10 (Cambrex), X-VIVO 15 (Cambrex®) and Stemline® II (Sigma-Aldrich), StemSpan® Serum-Free Expansion Medium (SFEM) (STEMCELL Technologies®, Vancouver, Canada), StemSpan® H3000-Defined Medium (STEMCELL Technologies®, Vancouver, Canada), CellGro®, SCGM (CellGenix®, Freiburg Germany), and StemPro®-34 SFM (Invitrogen®). The medium may also include various growth factors and cytokines typically used in stem cell cultures such as SCF, TPO, IL-6 and/or FLT3L. The medium may also be free of cytokines, as described in Sakurai et al., Nature volume 615, pages 1 27-133 (2023)

In another aspect, the present disclosure provides a hematopoietic stem cell (HSC) and/or hematopoietic progenitor cell (HPC) culture medium comprising a selective 5-Hydroxytryptamine Receptor 1F (HTR_{1F}) agonist and/or of a Thyroid Hormone Receptor Beta (THRB) agonist. The culture medium may further comprise one or more components, such as the components listed above.

In an embodiment, the culture is for a period of at least 5 days. In an embodiment, the culture is for a period of 7 to 14 days. In an embodiment, the culture is for a period of 8, 9, 10, 11 or 12 days. In a further embodiment, the culture is for a period of 10 days.

In an embodiment, the method described herein expands the hematopoietic stem cells and/or hematopoietic progenitor cells by at least 3-, 4- or 5-fold. In an embodiment, the method described herein expands the hematopoietic stem cells and/or hematopoietic progenitor cells by at least 10-fold. In an embodiment, the method described herein expands the hematopoietic stem cells and/or hematopoietic progenitor cells by at least 15-fold. In an embodiment, the method described herein expands the hematopoietic stem cells and/or hematopoietic progenitor cells by at least 20-fold. In an embodiment, the method described herein expands the hematopoietic stem cells and/or hematopoietic progenitor cells by at least 30-, 40- or 50-fold.

Populations of cells containing HSCs and/or HPCs provided herein confer the same or similar advantages of stem cells found in cord blood. A person of skill in the art would readily recognize the characteristics of stem cells from cord blood and the advantageous properties therein. In some embodiments, at least 10%, 20%, 30%, 40%, 50%, 60%, 70%, 80%, 90%, or 95% of the populations of cells containing HSCs and/or HPCs provided herein are expanded HSCs and/or HPCs. In some embodiments, the expanded HSCs and/or HPCs in the populations of cells have retained their stem cell phenotype for an extended period of time. For example, in some embodiments, populations of cells containing HSCs and/or HPCs include expanded HSCs and/or HPCs with cell surface phenotypes that include CD34+, CD45+, CD133+, CD90+, CD45RA−, and/or CD38 low/−.

Administration/Use of Expanded Hematopoietic Stem and/or Progenitor Cells

Expanded HSCs and/or HPCs generated as described herein can be employed for regeneration and engraftment in a human patient or other subjects in need of such treatment.

Thus, in another aspect, the present disclosure provides a method for treating a patient in need of stem-cell transplantation (SCT), the method comprising administering to the subject an effective amount of HSCs and/or HPCs obtained by the method for expanding HSCs and/or HPCs described herein. In another aspect, the present disclosure provides the use of HSCs and/or HPCs obtained by the method for expanding HSCs and/or HPCs described herein for the treatment of a patient in need of stem-cell transplantation (SCT). In another aspect, the present disclosure provides the use of HSCs and/or HPCs obtained by the method for expanding HSCs and/or HPCs described herein for the manufacture of a medicament for the treatment of a patient in need of stem-cell transplantation (SCT). In another aspect, the present disclosure provides HSCs and/or HPCs obtained by the method for expanding HSCs and/or HPCs described herein for use in the treatment of a patient in need of stem-cell transplantation (SCT).

The present disclosure also provides a method for increasing the number of immune cells or for reconstituting the immune system (e.g., for treating an immunodeficiency or immunosuppression) in a subject in need thereof comprising administering to the subject an effective amount of HSCs and/or HPCs obtained by the method described herein. The present disclosure also provides the use of HSCs and/or HPCs obtained by the method for expanding HSCs and/or HPCs described herein for increasing the number of immune cells or for reconstituting the immune system (e.g., for treating an immunodeficiency or immunosuppression) in a subject. The present disclosure also provides the use of HSCs and/or HPCs obtained by the method for expanding HSCs and/or HPCs described herein for the manufacture of a medicament for increasing the number of immune cells or for reconstituting the immune system (e.g., for treating an immunodeficiency or immunosuppression) in a subject. In another aspect, the present disclosure provides HSCs and/or HPCs obtained by the method for expanding HSCs and/or HPCs described herein for use in increasing the number of immune cells or for reconstituting the immune system (e.g., for treating an immunodeficiency or immunosuppression) in a subject.

The cells are administered in a manner that permits them to graft and reconstitute or regenerate a functional immune system within a subject or recipient. Devices are available that can be adapted for administering cells, for example, intravascularly. Expanded HSCs and/or HPCs can be administered to by systemic injection, catheter, implantable device, or the like. In some embodiments, the cells can be administered parenterally by injection into a blood vessel or into a convenient cavity. A population of expanded cells can be administered in any physiologically acceptable excipient or carrier that does not adversely affect the cells.

A population of expanded HSCs and/or HPCs can be supplied in the form of a pharmaceutical composition. Such a composition can include an isotonic excipient prepared under sufficiently sterile conditions for human administration (see, e.g., Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000). The choice of the cellular excipient and any accompanying constituents of the composition that includes a population of expanded cells can be adapted to optimize administration by the route and/or device employed. A composition that includes a population of expanded hematopoietic stem and progenitor cells can also include or be accompanied by one or more other ingredients that facilitate engraftment or functional mobilization of the expanded cells.

In certain embodiments, the expanded HSCs and/or HPCs, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments. In some embodiments, for example, where the subject is a human, the dose of HSCs and/or HPCs is at least 1×10², 1×10³, 1×10⁴ or 1×10⁵ cells, for example in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the expanded HSCs and/or HPCs are administered as part of a combination treatment, such as simultaneously with or sequentially with, in any order, another therapeutic intervention, such as an antibody or engineered cell or receptor or agent, such as a cytotoxic or therapeutic agent. The expanded HSCs and/or HPCs in some embodiments are co-administered with one or more additional therapeutic agents or in connection with another therapeutic intervention, either simultaneously or sequentially in any order. In some contexts, the expanded HSCs and/or HPCs are co-administered with another therapy sufficiently close in time such that the cell populations enhance the effect of one or more additional therapeutic agents, or vice versa. In some embodiments, the expanded HSCs and/or HPCs are administered prior to the one or more additional therapeutic agents. In some embodiments, the expanded HSCs and/or HPCs are administered after the one or more additional therapeutic agents. In some embodiments, the one or more additional agents include a cytokine, such as IL-2, for example, to enhance persistence. In some embodiments, the methods comprise administration of a chemotherapeutic agent.

The expanded HSCs and/or HPCs may be used in combination with other therapy such as other chemotherapy, immunotherapy, radiotherapy, or surgery, according to the disease to be treated.

Administration/Use of HTR_{1F} and/or THRB Agonists for Promoting or Stimulating Expansion of HSCs and/or HPCs In Vivo

In another aspect, the present disclosure provides a method for promoting or stimulating the expansion of HSCs and/or HPCs in a subject in need thereof, the method comprising administering to the subject an effective amount of an HTR_{1F} agonist, such as a selective HTR_{1F} agonist, to the subject. The present disclosure also provides the use of an HTR_{1F}, such as a selective HTR_{1F}agonist, and/or a THRB agonist, for promoting or stimulating the expansion of HSCs and/or HPCs in a subject in need thereof. The present disclosure also provides the use of an HTR_{1F} agonist, such as a selective HTR_{1F} agonist, and/or a THRB agonist, for the manufacture of a medicament for promoting or stimulating the expansion of HSCs and/or HPCs in a subject in need thereof. The present disclosure also provides an HTR_{1F} agonist, such as a selective HTR_{1F} agonist, and/or a THRB agonist, for use in promoting or stimulating the expansion of HSCs and/or HPCs in a subject in need thereof.

In another aspect, the present disclosure provides a method for increasing the number of immune cells or for reconstituting the immune system (e.g., for treating an immunodeficiency or immunosuppression) in a subject in need thereof, the method comprising administering to the subject an effective amount of an HTR_{1F} agonist, such as a selective HTR_{1F} agonist, to the subject. The present disclosure also provides the use of an HTR_{1F}, such as a selective HTR_{1F} agonist, and/or a THRB agonist, for increasing the number of immune cells or for reconstituting the immune system (e.g., for treating an immunodeficiency or immunosuppression) in a subject in need thereof. The present disclosure also provides the use of an HTR_{1F} agonist, such as a selective HTR_{1F} agonist, and/or a THRB agonist, for the manufacture of a medicament for increasing the number of immune cells or for reconstituting the immune system (e.g., for treating an immunodeficiency or immunosuppression) in a subject in need thereof. The present disclosure also provides an HTR_{1F}agonist, such as a selective HTR_{1F} agonist, and/or a THRB agonist, for use in increasing the number of immune cells or for reconstituting the immune system (e.g., for treating an immunodeficiency or immunosuppression) in a subject in need thereof.

In an embodiment, the HTR_{1F} and/or THRB agonist is/are formulated into a pharmaceutical composition. Such compositions may be prepared in a manner well known in the pharmaceutical art. Supplementary active compounds can also be incorporated into the compositions. As used herein “pharmaceutically acceptable carrier” or “excipient” or “diluent” includes any and all solvents, buffers, dispersion media, coatings, antibacterial and antifungal agents, isotonic and absorption delaying agents, and the like that are physiologically compatible. The carrier can be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration (see Remington: The Science and Practice of Pharmacy by Alfonso R. Gennaro, 2003, 21^{th} edition, Mack Publishing Company).

Formulations suitable for oral administration can consist of (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds/compositions of the invention include ethylenevinyl acetate copolymer particles, osmotic pumps, implantable infusion systems, and liposomes. Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

For preparing pharmaceutical compositions comprising the HTR_{1F} and/or THRB agonist, pharmaceutically acceptable carriers are either solid or liquid. Solid form preparations include powders, tablets, pills, capsules, cachets, suppositories, and dispersible granules. A solid carrier can be one or more substance, which may also act as diluents, flavoring agents, binders, preservatives, tablet disintegrating agents, or an encapsulating material.

In powders, the carrier is a finely divided solid, which is in a mixture with the finely divided active component. In tablets, the active component (HTR_{1F} and/or THRB agonist) is mixed with the carrier having the necessary binding properties in suitable proportions and compacted in the shape and size desired. The powders and tablets may typically contain from 5% or 10% to 70% of the active compound/composition. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The present disclosure also relates to the use of an HTR_{1F} and/or THRB agonist and compositions as described herein and as defined herein in the preparation of a medicament for the treatment of a subject (or patient) suffering from the following non-limiting list of disorders: autologous or allogeneic transplantation or treatment of a subject (or patient) suffering from the above-mentioned disorders or from auto-immune disorders. Examples of hematological malignancies/disorders and congenital diseases may include, without limitation, acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin's lymphoma, Hodgkin's disease, aplastic anemia, pure red cell aplasia, hemoglobinuria, Fanconi anemia, thalassemia, sickle cell anemia, Blackfan-Diamond syndrome, globoid cell leukodystrophy, Wiskott-Aldrich syndrome, Hunter's syndrome, Hurler's syndrome, Lesch Nyhan syndrome, inborn errors of metabolism (such as Gaucher disease among others). Examples of immunological disorders that may benefit from transplantation are numerous and include multiple sclerosis, lupus, certain forms or arthritis, severe combined immunodeficiencies, and the like. HTR_{1F} and/or THRB agonist and compositions as described herein may be used for the treatment suffering from immunosuppression or immunodeficiency, e.g., primary or secondary (acquired) immunosuppression or immunodeficiency. Immunosuppression and immunodeficiencies may be caused by various diseases and conditions including congenital deficiencies (e.g., common variable immunodeficiency (CVID), severe combined immunodeficiency (SCID), chronic granulomatous disease (CGD), severe burns, chemotherapy, immunosuppressive drugs (e.g., corticosteroids, calcineurin inhibitors, antiproliferative agents, mTOR inhibitors), radiation, diabetes mellitus, malnutrition infectious diseases (e.g., AIDS, viral hepatitis), and cancers (e.g., hematological cancers such as leukemia, lymphoma, myeloma).

The HTR_{1F} and/or THRB agonist and compositions as described herein can be used in the following non-limiting settings: autologous or allogeneic transplantation or treatment of a subject (or patient) suffering from the above-mentioned disorders or from auto-immune disorders. Examples of hematological malignancies/disorders and congenital diseases may include, without limitation, acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin's lymphoma, Hodgkin's disease, aplastic anemia, pure red cell aplasia, hemoglobinuria, Fanconi anemia, thalassemia, sickle cell anemia, Wiskott-Aldrich syndrome, metachromatic leukodystrophy (MLD), Leukocyte adherence deficiency, X-linked CGD, inborn errors of metabolism (such as Gaucher disease among others), adrenoleukodystrophy, Mucopolysaccharidosis IIIA, as well as immunodeficiencies such as severe combined immunodeficiency (SCID) and adenosine deaminase (ADA) deficiency. Examples of immunological disorders that may benefit from transplantation are numerous and include multiple sclerosis, lupus, certain forms or arthritis, severe combined immunodeficiencies, and the like.

Thus, the present disclosure encompasses administration, to a patient suffering from any one of the above-mentioned disorders/malignancies, HSCs and/or HPCs that are expanded using the method of expansion described herein.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, the terms “subject” or “patient” refers to any animal, such as a domesticated animal, a zoo animal, or a human. The “subject” or “patient” can be a mammal like a dog, cat, bird, livestock, or a human. Specific examples of “subjects” and “patients” include, but are not limited to, individuals with a hematological or immunological disease, condition, or disorder, and individuals with hematological disorder-related or immunological disorder-related characteristics or symptoms.

In an embodiment, the subject is an elderly subject.

### Screening Assays

The present inventors have shown that HTR_{1F} and THRB constitute pharmacological targets for promoting or stimulating the expansion of HSCs and/or HPCs ex vivo and in vivo, and for increasing the number of immune cells or for reconstituting the immune system in a subject.

In another aspect, the present disclosure provides a method for identifying an agent useful for expanding hematopoietic stem cells (HSCs) and/or hematopoietic progenitor cells (HPCs) ex vivo or in vivo, and/or for increasing the number of immune cells or for reconstituting the immune system in a subject, the method comprising determining whether the agent is a selective 5-Hydroxytryptamine Receptor 1F (HTR_{1F}) agonist and/or of a Thyroid Hormone Receptor Beta (THRB) agonist. If the agent is a HTR_{1F} and/or THRB agonist, it is indicative that it may be useful for promoting or stimulating the expansion of HSCs and/or HPCs, and/or for increasing the number of immune cells or for reconstituting the immune system in a subject.

In embodiments, the method comprises measuring HTR_{1F} and/or THRB activity in the presence and absence of the test agent, wherein a higher activity in the presence of the test agent relative to the absence thereof is indicative that the test compound may be useful for promoting or stimulating the expansion of HSCs and/or HPCs, and/or for increasing the number of immune cells or for reconstituting the immune system in a subject. Such method may involve contacting a cell expressing HTR_{1F} and/or THRB with the test agent, and measuring a signal or marker associated with HTR_{1F} and/or THRB activation. In an embodiment, the signal is the activation of a signaling pathway associated with HTR_{1F} and/or THRB.

In an embodiment, the method is an in vitro method.

The assay systems used to perform the screening method may comprise a variety of means to enable and optimize useful assay conditions. Such means may include but are not limited to: suitable buffer solutions, for example, for the control of pH and ionic strength and to provide any necessary components for optimal HTR_{1F} and/or THRB activity and stability (e.g., protease inhibitors), temperature control means for optimal HTR_{1F} and/or THRB activity and or stability, and detection means to enable the detection of the HTR_{1F} and/or THRB activity. A variety of such detection means may be used, including but not limited to one or a combination of the following: radiolabelling (e.g., ³²P, ¹⁴C, ³H), antibody-based detection, fluorescence, chemiluminescence, spectroscopic methods (e.g., generation of a product with altered spectroscopic properties), various reporter enzymes or proteins (e.g., horseradish peroxidase, green fluorescent protein), specific binding reagents (e.g., biotin/streptavidin), detecting of phosphorylated proteins, and others.

The above-noted assays may be applied to a single test agent or to a plurality or “library” of such agents (e.g., a combinatorial library). Any such compound may be utilized as lead compound and further modified to improve its therapeutic, prophylactic and/or pharmacological properties for promoting or stimulating the expansion of HSCs and/or HPCs, and/or for increasing the number of immune cells or for reconstituting the immune system in a subject.

### EXAMPLES

The present disclosure is illustrated in further details by the following non-limiting examples.

### Example 1: Air-Liquid-Interface Culture Method as a Means of Amplification and Conservation of Engraftment Capacities

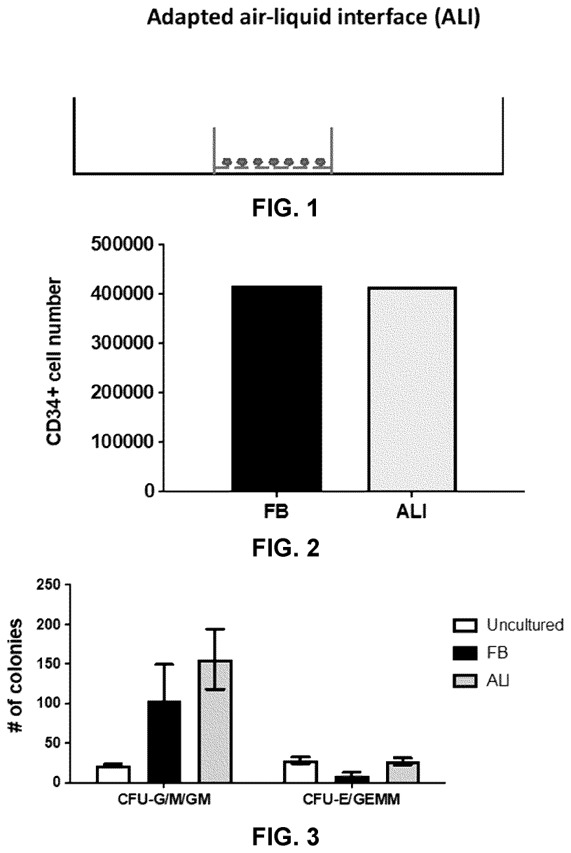

An experimental setup was designed where oxygen exchange in the media would be maximized, while adopting the principles of fed-batch cultures (FB) in which medium is renewed and in sufficient quantity to provide nutrients while diluting cell waste. As illustrated in FIG. 1, HSC isolated from cord blood were cultured using a modified air-liquid interface (ALI) method (37° C. humidified incubator, 5% CO₂ and ambient air). In doing so, the maximum of cells was placed directly at the air-liquid interface so that gas exchanges are more efficient. Briefly, HSC were enriched based on CD34 expression marker, from cord-blood units using the Miltenyi CD34 MicroBead Kit UltraPure according to manufacturer's instruction. Purity was determined by flow cytometry using monoclonal CD34-PE antibodies (Clone 581, BD Pharmingen, 555822). Between 5,000 and 50,000 CD34⁺ were plated in a 3.14 cm² polycarbonate cell culture insert (pore size 0.4 μM; Nunc #137060/Fisher #140640). The volume of cell suspension was adjusted to be just sufficient to cover the surface of the round insert. This insert was then placed on top of serum-free culture media supplemented with factors necessary to maintain HSC (see schematic representation in FIG. 1). The volume of culture media was adjusted to be large enough so the media spread in large surface in order to allow more air-liquid gas exchanges. Typically, 10 mL of media was added inside a 10-cm petri dish for the first 5 days of culture, on top of which an insert was placed to float or to be in direct contact with the underlying media. If cultures longer than 5 days were performed, an additional 5 mL of freshly-prepared media was added to the lower chamber (10-cm petri dish). In principle, any nutrient can freely cross the pores of the insert by diffusion to reach the cells and any by-product produced by the cells can diffuse to the lower media compartment and get diluted in the large media volume.

Basic expansion media consisted in StemSpan SFEM I (STEMCELL Technologies)+5 mg/mL LDL or StemSpan SFEM II, supplemented with 200 mM GlutaMAX I, penicillin/streptomycin, 100 ng/mL SCF, 50 ng/mL TPO and 100 ng/mL FLT3L (all from Peprotech). Where indicated expansion media also contained 40 nM UM171 or 500 nM UM729.

With this design, any local accumulation of secreted factors is avoided while nutrient availability does not become limiting due to the relatively large volume of underlying media. In addition, the demanding routine of frequent media changes and time-consuming steps of monitoring cell density associated with manual fed-batch cultures was avoided. Taking into account the proliferation and behavior of HSC from different sources, it was determined that up to 3 inserts, each containing a maximum of 15,000 CD34⁺ cells can be placed in a dish containing 15 mL of expansion media during 10 days.

Using this culture method, the expansion of the stem cell population (defined as CD34⁺ cells) in the culture wells after 10 days was first assessed. Flow cytometry characterization and enumeration showed that the expansion of CD34⁺ was comparable between the ALI culture method together with UM171 and UM171 in classical culture conditions (FB culture) (FIG. 2). Indeed, with both methods, an approximatively 55-fold expansion in CD34⁺ population was achieved.

As cell expansion is often linked to loss of pluripotency¹³, it was then assessed whether the CD34⁺ cells obtained after this culture have kept their differentiation capacity. To do so, the effect of culture methods on the number of colonies formed on methylcellulose media was evaluated using MethoCult media according to manufacturer's instructions (Stem Cell Technologies) (FIG. 3). Results show that CD34⁺ cells obtained from both methods of culture display a similar potential of differentiation. Thus, the ALI method does not impair the differentiation capacity of cultured HSCs.

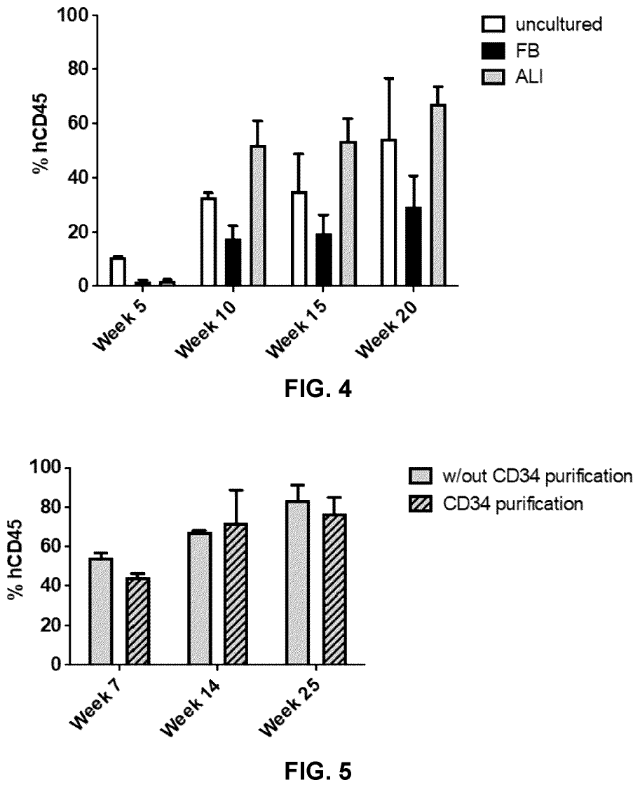

Since the ultimate aim is to improve the expansion of engraftable human HSC, the validation methods largely rely on the measurement of immune reconstitution, both short-term and long-term, after transplantation in a humanized mice model. Briefly, NSG mice (Jackson Laboratories, strain 005557) were sublethally irradiated at 2Gy 4 to 24 hours prior to CD34⁺ cells intravenous (IV) injection. All harvested cells from 1 culture well were injected into one mouse. Wells from all conditions started with equal numbers of CD34⁺ cells from the same source/donor. Therefore, all the progeny of a culture setting was injected into mice. Peripheral blood of humanized mice was harvested every 5 weeks, and human engraftment and immune reconstitution were analyzed by flow cytometry. The earliest point where human engraftment in mice was measured was after 5 weeks and the latest after 35 weeks. The impact of the ALI culture system on HSC engraftment capacity was assessed using base media purchased from STEMCELL Technologies (StemSpan SFEM). A dramatic effect of the ALI methodology on the engraftment of cord-blood HSC was observed relative to the published manual fed-batch culture methodology⁴, which includes SCF, TPO, FLT3L and UM171 (FIG. 4).

It was then explored which cells obtained after the 10-day culture were responsible for the engraftment in mice. Although in this first step the entire progeny of the culture, which contains CD34-positive (CD34⁺) and CD34-negative cells, was injected, it was assessed if the CD34⁺ subpopulation, or other cells present in the culture, were responsible for the observed immune reconstitution. The immune reconstitution of irradiated adult NSG mice (2 mice) following the injection of a fraction of the HSC culture (ALI method) magnetically enriched for CD34⁺ cells was compared to the injection of the entire progeny (without purification of CD34⁺ cells). Immune reconstitution data showed no difference between purified or bulk culture injection (FIG. 5), suggesting that CD34⁺ cells were the main cells responsible for immune cell reconstitution after ALI ex vivo culture.

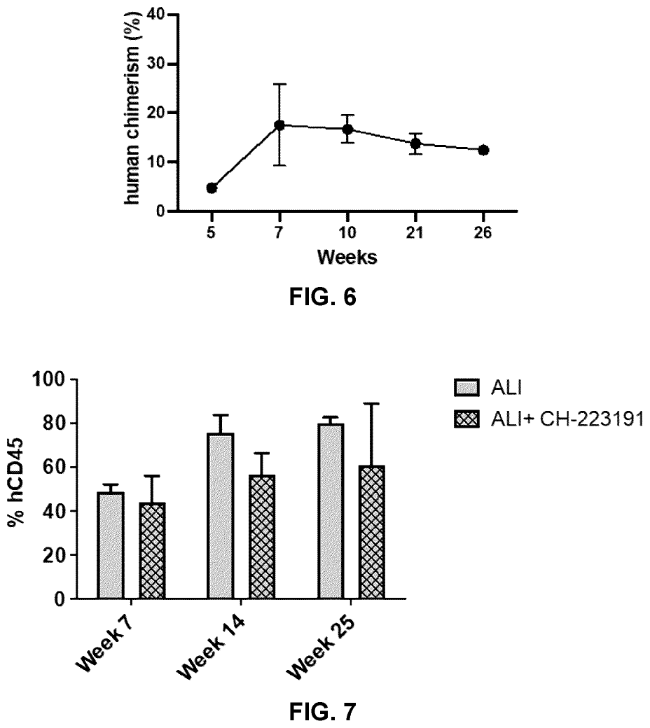

To assess if the ALI culture method was able to maintain long-term reconstitution capacity of cultured HSCs, a secondary transplantation was performed. Briefly, bone marrow cells isolated from a mice humanized with ALI-cultured CD34⁺ cells, 31 weeks post-humanization, were injected to 2 new irradiated NSG mice. Mice received approximately 3.6 million hCD45⁺ each, of which 3.8% were CD34⁺CD45RA⁻ and 1.8% CD34⁺CD45RA⁻. Immune chimerism measured in the peripheral blood at 5, 7, 10, 21, and 26 weeks post-transplantation confirmed that long-term progenitor cells are conserved in the ALI culture method (FIG. 6).

As aryl hydrocarbon receptor antagonists (AhR), such as CH-223191, have been described to promote HSC expansion while keeping their engraftment properties, its impact on engraftment when used in the media of the ALI culture (including UM729) was explored.² The addition of CH-223191 did not further increase the engraftment capacity of cultured HSC using the ALI method (FIG. 7).

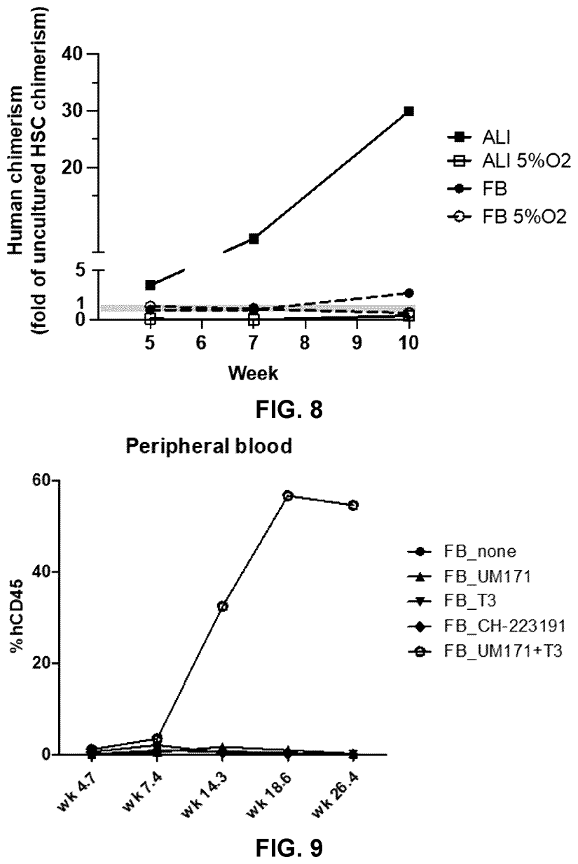

To understand if the impact of the ALI culture method on CD34 engraftment capacity was related to the relative amount of oxygen available to cells, the cells were cultured in ambient O₂ concentration (approximatively 20.9% v/v) or under hypoxic conditions (5% O₂), either with the fed-batch culture method or ALI. Cultured cells were then injected into irradiated NSG as described above. Blood draws revealed that oxygen concentration is important for achieving the effect of ALI culture on the preservation (and increase) of engraftment characteristics since ALI culture under hypoxic conditions (5% O₂) loses its potency (FIG. 8). Overall, these studies show that HSCs cultured with the ALI method increased CD34⁺ cells engraftment in a mouse model, likely through an increase of oxygen exchange.

### Example 2: THRB-Specific Agonist Triiodothyronine (T3) as a Promoter of HSC Engraftment and Post-Graft Immune Reconstitution

The effect of an agonist of Thyroid Hormone Receptor Beta (THRB), triiodothyronine (T3), on HSC engraftment when added to ex vivo HSC culture. Addition of T3 (45 ng/mL) in cytokine-supplemented (SCF, TPO, FLT3L) StemSpan SFEM media containing UM171 increased the engraftment of HSC in humanized mice relative to other conditions, including UM171 alone or in combination with the AhR receptor antagonist CH-223191 (FIG. 9).

### Example 3: Ex Vivo HTR_{1F} Agonists Increase HSCs Engraftment and Immune Reconstitution

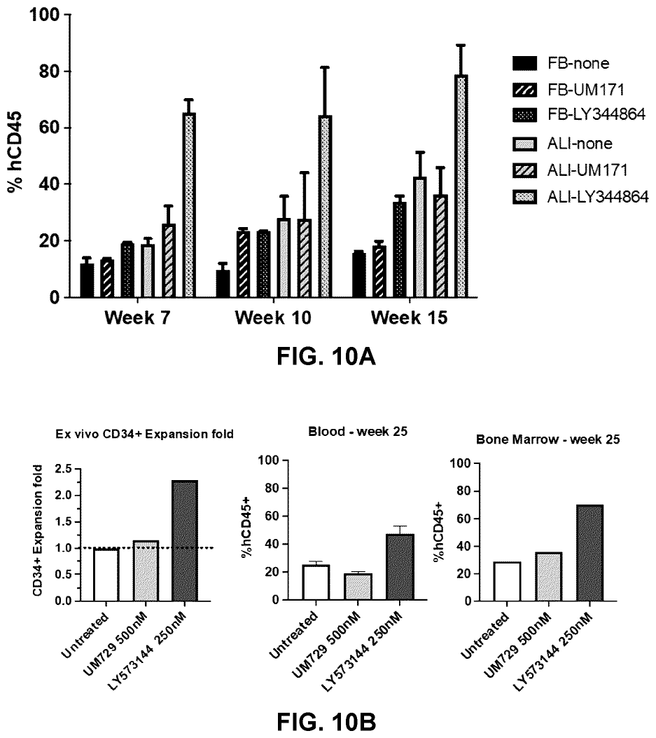

The effect of an agonist of HTR_{1F}, LY344864, on HSC engraftment when added to ex vivo HSC culture. When used during HSC expansion culture ex vivo, the addition of LY344864 at a concentration of 500 nM, in serum-free Stemspan SFEM II-based culture media (supplemented in SCF, TPO, FLT-3L as described above), greatly improved the peripheral blood engraftment compared to media alone, specifically in the context of ALI culture method (FIG. 10A).

A second highly specific HTR_{1F} agonist, LY573144 (Lasmiditan, commercialized under the name Reyvow by Eli Lilly in the United States) was used during ex vivo expansion of HSCs. The addition of HTR_{1F} agonist LY573144 to the culture increased the expansion of the CD34⁺ population by two-fold after 6 days of culture (FIG. 10B). When the progenies of the different cultures were injected in irradiated NSG mice, higher percentages of hCD45⁺ cell in the blood and in the bone marrow of the mice was found in the LY573144 condition 25 weeks after injection (FIG. 10B)

### Example 4: In Vivo HTR_{1F} Agonists Increase HSCs Engraftment and Immune Reconstitution

Building on these results, and the availability of a highly specific HTR_{1F} agonist that is currently approved in the United States for the treatment of migraine, LY573144 (Lasmiditan, commercialized under the name Reyvow by Eli Lilly) was tested as an in vivo administered drug to promote HSC engraftment and immune reconstitution after transplantation.

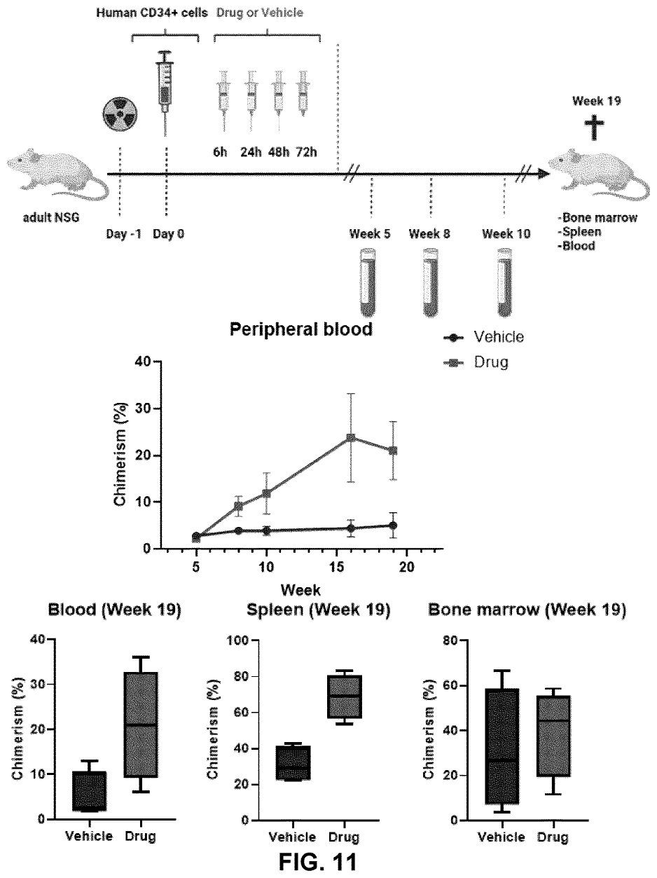

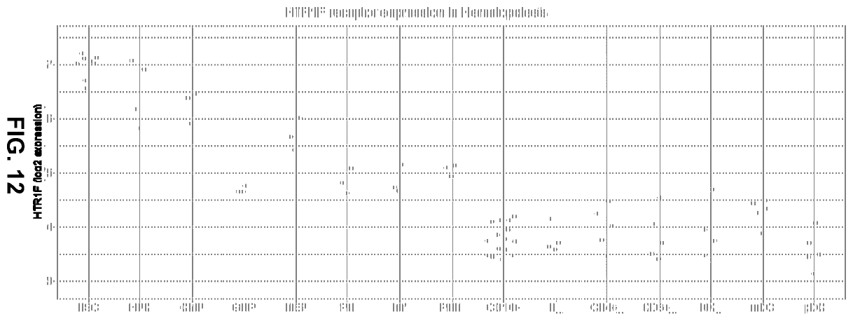

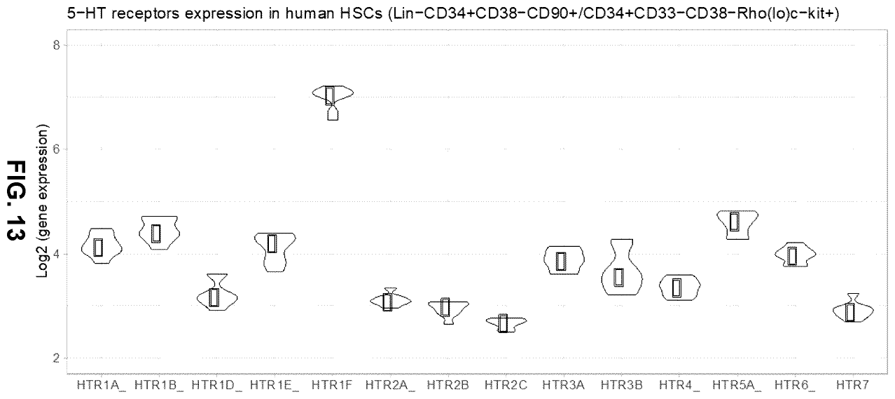

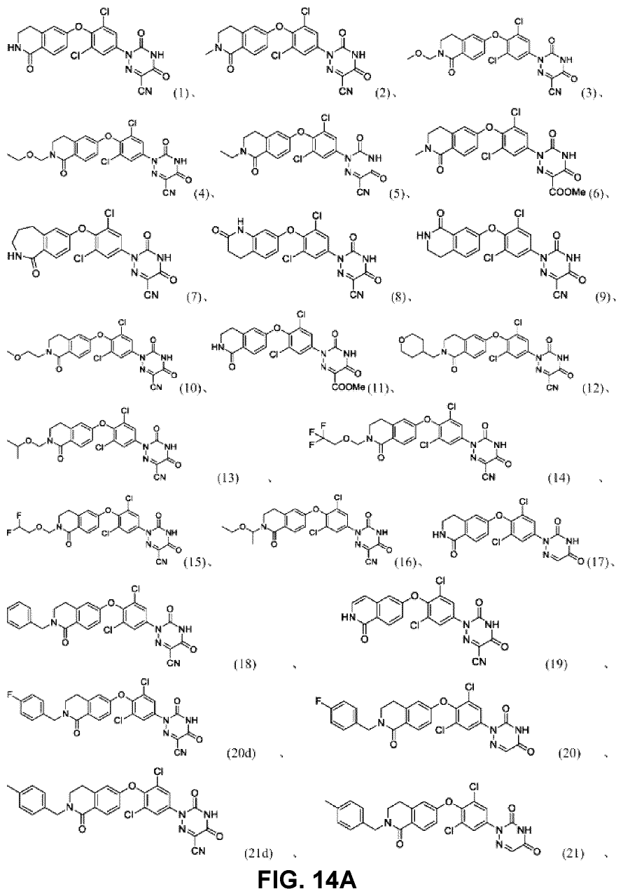

Lasmiditan, purchased from AdooQ Bioscience (product number A14198), was dissolved in DMSO and ethanol solution. Mice (all males) were humanized each with 38,000 uncultured CD34⁺ cells isolated from cord blood (female cells) using the Miltenyi CD34 purification kit as described above. Purity and viability were assessed by flow cytometry using the following staining: CD34-APC (Biolegend 343510), CD19-PE (Biolegend, 302208), CD3-PE-Cy7 (Biolegend, 344816), CD45-APC-Cy7 (Biolegend, 368516), 7-AAD (BD Biosciences, 559925), 1 μL AnnV-FITC (Biolegend, 640906). Treated mice were given a dose of 0.3 mg (dose of 10 mg/kg, in 300 μL, 0.5% DMSO) of Lasmiditan intraperitoneally at 6 h, 24 h, 48 h and 72 h post-cell injection as depicted in FIG. 11. The dose was determined based on previous work with LY344864 (used ex vivo above, FIG. 10) in kidney injury recovery studies¹⁶ and is consistent with the dosage of Reyvow® (Lasmiditan) for the treatment of migraine. Control mice received 4 injections of vehicle. Human chimerism was assessed on peripheral blood (using mCD45-FITC from BD Pharmingen-553080, hCD45-PE from BD Pharmingen-555483, hCD14-APC-Cy7 from Biolegend-325620, CD19-APC from Biolegend-302212, CD3-PE-Cy7 from Biolegend-344816 and 7-AAD from BD Biosciences-559925), until 19 weeks post-transplantation as described above, after which time mice were sacrificed, and spleen and bone marrow were analyzed in the same manner. FIG. 10 shows that mice treated with the specific agonist of HTR1F, Lasmiditan, achieved a more robust reconstitution of the immune compartment, in a context of really low doses of CD34⁺ cells (38,000 cells). Human immune cells (hCD45⁺) were found in blood, spleen and bone marrow of the treated animals.

### Example 5: Expression Pattern of 5-HT Receptors in Hematopoietic and Immune Cells

Lastly, publicly-available databases were interrogated for differential gene expression of 5-HT receptors in hematopoietic and immune cells (https://www.ncbi.nlm.nih.gov/geo/). Consistent with the data presented herein, it was found that HTR_{1F}, which is bound by LY344864 and LY573144, is expressed at higher levels in hematopoietic stem cells (HSC) and hematopoietic progenitor cells (HPC) as well as in common myeloid progenitors (CMP) and Megakaryocyte-erythroid progenitor (MEP) (FIG. 12). Interestingly, the expression of the different 5-HT receptors was also compared in HSC and it was found that HTR_{1F} was the most expressed by HSCs (FIG. 13). These relative expression data are coherent with the effect of LY344864 and LY573144 on HSCs ex vivo and in vivo.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Zhang Y, Shen B, Guan X, et al. Safety and efficacy of ex vivo expanded CD34(+) stem cells in murine and primate models. Stem Cell Res Ther 2019; 10:173.
2. Boitano A E, Wang J, Romeo R, et al. Aryl hydrocarbon receptor antagonists promote the expansion of human hematopoietic stem cells. Science 2010; 329:1345-8.
3. Delaney C, Heimfeld S, Brashem-Stein C, et al. Notch-mediated expansion of human cord blood progenitor cells capable of rapid myeloid reconstitution. Nat Med 2010; 16:232-6.
4. Fares I, Chagraoui J, Gareau Y, et al. Cord blood expansion. Pyrimidoindole derivatives are agonists of human hematopoietic stem cell self-renewal. Science 2014; 345:1509-12.
5. Miller P H, Knapp D J, Beer P A, et al. Early production of human neutrophils and platelets posttransplant is severely compromised by growth factor exposure. Exp Hematol 2016; 44:635-40.
6. Porada C D, Atala A J, Almeida-Porada G. The hematopoietic system in the context of regenerative medicine. Methods 2016; 99:44-61.
7. Guenechea G, Segovia J C, Albella B, et al. Delayed engraftment of nonobese diabetic/severe combined immunodeficient mice transplanted with ex vivo-expanded human CD34(+) cord blood cells. Blood 1999; 93:1097-105.
8. Mobest D, Goan S R, Junghahn I, et al. Differential kinetics of primitive hematopoietic cells assayed in vitro and in vivo during serum-free suspension culture of CD34⁺ blood progenitor cells. Stem Cells 1999; 17:152-61.
9. Tisdale J F, Hanazono Y, Sellers S E, et al. Ex vivo expansion of genetically marked rhesus peripheral blood progenitor cells results in diminished long-term repopulating ability. Blood 1998; 92:1131-41.
10. Ahmed F, Ings S J, Pizzey A R, et al. Impaired bone marrow homing of cytokine-activated CD34+ cells in the NOD/SCID model. Blood 2004; 103:2079-87.
11. Csaszar E, Kirouac D C, Yu M, et al. Rapid expansion of human hematopoietic stem cells by automated control of inhibitory feedback signaling. Cell Stem Cell 2012; 10:218-29.
12. Magnusson M, Sierra M I, Sasidharan R, et al. Expansion on stromal cells preserves the undifferentiated state of human hematopoietic stem cells despite compromised reconstitution ability. PLoS One 2013; 8:e53912.
13. Suda T, Takubo K, Semenza G L. Metabolic regulation of hematopoietic stem cells in the hypoxic niche. Cell Stem Cell 2011; 9:298-310.
14. Fulcher M L, Randell S H. Human nasal and tracheo-bronchial respiratory epithelial cell culture. Methods Mol Biol 2013; 945:109-21.
15. Gerber J M, Gucwa J L, Esopi D, et al. Genome-wide comparison of the transcriptomes of highly enriched normal and chronic myeloid leukemia stem and progenitor cell populations. Oncotarget 2013; 4:715-28.
16. Garrett S M, Whitaker R M, Beeson C C, et al. Agonism of the 5-hydroxytryptamine 1F receptor promotes mitochondrial biogenesis and recovery from acute kidney injury. J Pharmacol Exp Ther 2014; 350:257-64.
17. Yang M, Li K, Ng P C, et al. Promoting effects of serotonin on hematopoiesis: ex vivo expansion of cord blood CD34⁺ stem/progenitor cells, proliferation of bone marrow stromal cells, and antiapoptosis. Stem Cells 2007; 25:1800-6.
18. Sibon D, Coman T, Rossignol J, et al. Enhanced Renewal of Erythroid Progenitors in Myelodysplastic Anemia by Peripheral Serotonin. Cell Rep 2019; 26:3246-3256 e4.
19. Villanueva I, Alva-Sanchez C, Pacheco-Rosado J. The role of thyroid hormones as inductors of oxidative stress and neurodegeneration. Oxid Med Cell Longev 2013; 2013:218145.
20. Lv J, Wang L, Gao Y, et al. 5-hydroxytryptamine synthesized in the aorta-gonad-mesonephros regulates hematopoietic stem and progenitor cell survival. J Exp Med 2017; 214:529-545.
21. Morgan R A, Gray D, Lomova A, et al. Hematopoietic Stem Cell Gene Therapy: Progress and Lessons Learned. Cell Stem Cell 2017; 21:574-590.

1. A method for expanding hematopoietic stem cells (HSCs) and/or hematopoietic progenitor cells (HPCs) ex vivo comprising culturing said hematopoietic stem cells and/or hematopoietic progenitor cells under conditions suitable for HSC and/or HPC expansion (a) in the presence of a selective 5-Hydroxytryptamine Receptor 1F (HTR1F) agonist and/or of a Thyroid Hormone Receptor Beta (THRB) agonist; and/or (b) in an air-liquid interface (ALI) culture system.

2. The method of claim 1, wherein the HSCs and/or HPCs are obtained from donor bone marrow, umbilical donor cord blood, or donor peripheral blood.

3. The method of claim 1, wherein the HSCs and/or HPCs are from a subject suffering from a disease or condition.

4. The method of claim 3, wherein the disease is cancer, an immunodeficiency, myelodysplastic syndrome (MDS) or anemia.

5. The method of claim 4, wherein the cancer is a hematological cancer.

6. The method of claim 1, further comprising genetically modifying the HSCs and/or HPCs to generate genetically-modified HSCs and/or HPCs.

7. The method of claim 1, wherein said method comprises culturing the HSCs and/or HPCs in an ALI culture system.

8. The method of claim 1, wherein said conditions suitable for HSC and/or HPC expansion comprises culturing the HSCs and/or HPCs in the presence of cytokines and/or growth factors.

9. The method of claim 8, wherein the cytokines and/or growth factors comprises SCF, TPO, FLT3L and/or IL-6.

10. (canceled)

11. The method of claim 1, wherein the selective HTR1F agonist is N-[(3R)-3-(Dimethylamino)-2,3,4,9-tetrahydro-1H-carbazol-6-yl]-4-fluorobenzamide hydrochloride (LY 344864), 4-fluoro-N-(3-(1-methyl-4-piperidinyl)-1H-indol-5-yl)benzamide (LY 334370), 2,4,6-Trifluoro-N-[6-[(1-methyl-4-piperidinyl)carbonyl]-2-pyridinyl]benzamide (LY 573144, Lasmiditan), or 5-N-butyryloxy-N,N-dimethyltryptamine.

12. The method of claim 1, wherein the THRB agonist is triiodothyronine (T3).

13-20. (canceled)

21. A method for (i)expanding hematopoietic stem cells (HSCs) and/or hematopoietic progenitor cells (HPCs) and/or (ii) increasing the number of immune cells or for reconstituting the immune system in a subject in need thereof comprising administering to the subject an effective amount of a selective 5-Hydroxytryptamine Receptor 1F (HTR1F) agonist and/or of a Thyroid Hormone Receptor Beta (THRB) agonist.

22. The method of claim 21, wherein the selective HTR1F agonist is N-[(3R)-3-(Dimethylamino)-2,3,4,9-tetrahydro-1H-carbazol-6-yl]-4-fluorobenzamide hydrochloride (LY 344864), 4-fluoro-N-(3-(1-methyl-4-piperidinyl)-1H-indol-5-yl)benzamide (LY 334370), 2,4,6-Trifluoro-N-[6-[(1-methyl-4-piperidinyl)carbonyl]-2-pyridinyl]benzamide (LY 573144, Lasmiditan), or 5-N-butyryloxy-N,N-dimethyltryptamine.

23. The method of claim 21, wherein the THRB agonist is triiodothyronine (T3).

24. The method of claim 21, wherein the subject is a stem-cell transplant recipient.

25. The method of claim 21, wherein the subject suffers from cancer, an immunodeficiency, myelodysplastic syndrome (MDS) or anemia.

26. The method of claim 25, wherein the cancer is a hematological cancer.

27. The method of claim 24, wherein the stem-cell transplant comprises hematopoietic stem cells (HSCs) and/or hematopoietic progenitor cells (HPCs) obtained by an ex vivo comprising culturing said HSCs and/or HPCs under conditions suitable for HSC and/or HPC expansion (a) in the presence of a selective 5-Hydroxytryptamine Receptor 1F (HTR1F) agonist and/or of a Thyroid Hormone Receptor Beta (THRB) agonist; and/or (b) in an air-liquid interface (ALI) culture system.

28-61. (canceled)

62. A hematopoietic stem cell (HSC) and/or hematopoietic progenitor cell (HPC) culture medium comprising a selective 5-Hydroxytryptamine Receptor 1F (HTR1F) agonist and/or of a Thyroid Hormone Receptor Beta (THRB) agonist.

63. The culture medium of claim 62, wherein the selective HTR1F agonist is N-[(3R)-3-(Dimethylamino)-2,3,4,9-tetrahydro-1H-carbazol-6-yl]-4-fluorobenzamide hydrochloride (LY 344864), 4-fluoro-N-(3-(1-methyl-4-piperidinyl)-1H-indol-5-yl)benzamide (LY 334370), 2,4,6-Trifluoro-N-[6-[(1-methyl-4-piperidinyl)carbonyl]-2-pyridinyl]benzamide (LY 573144, Lasmiditan), or 5-N-butyryloxy-N,N-dimethyltryptamine, and wherein the THRB agonist is triiodothyronine (T3).
